(19) United States
(12) Patent Application Publication
(10) Pub. No.: US 2025/0032700 A1
(43) Pub. Date: Jan. 30, 2025

(54) SYSTEM FOR REMOTE MEDICAL PROCEDURE

(71) Applicant: Imperative Care, Inc., Campbell, CA (US)

(72) Inventors: Steve Burion, San Francisco, CA (US); Lilip Lau, Los Altos, CA (US); Ling Xu, Belmont, CA (US); Theodore Jourdan, Mt. Pleasant, SC (US)

(21) Appl. No.: 18/784,630

(22) Filed: Jul. 25, 2024

Related U.S. Application Data
(60) Provisional application No. 63/529,039, filed on Jul. 26, 2023.

(51) Int. Cl. A61M 5/00; A61M 1/00; A61M 5/168

(52) U.S. Cl. CPC A61M 5/007; A61M 1/74; A61M 5/16827

(57) ABSTRACT

A robotic medical system for performing a vascular procedure includes a remote system and a local system. The local system includes one or more computing devices, a robotic drive system in communication with the one or more computing devices, the remote system located remotely from the robotic drive system, a fluidics system in communication with the one or more computing devices, and a plurality of interventional devices at least partially arranged in a concentric nested configuration, each of the plurality of interventional devices being independently moveable along a common axial direction by the robotic drive system, wherein one or more of the plurality of interventional devices are in fluid communication with the fluidics system. The local system is configured to receive first information from the remote system, determine movement control signals for moving the plurality of interventional devices based on the first information, and provide the movement control signals to the robotic drive system for independently moving one or more of the plurality of interventional devices. The local system is also configured to receive second information from the remote system, determine fluidics control signals from the second information, and provide the fluidics control signals to the fluidics system for independently providing one or more fluids and/or vacuum to one or more of the plurality of interventional devices.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application claims priority to U.S. Application No. 63/529,039, filed Jul. 26, 2023, titled CONTROL SYSTEM FOR TELEOPERATION FOR REMOTE MEDICAL PROCEDURE, the entire content of which is incorporated by reference herein for all purposes and forms a part of this specification.

### BACKGROUND

### Field

This disclosure relates to neurovascular procedures, and more particularly, to catheter assemblies and robotic control systems for neurovascular site access.

### Description of the Related Art

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neuro interventions. Neuro interventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovasculature is challenging to achieve, especially Type III arches. Once supra-aortic access is achieved, adapting the system for neurovascular treatments is time consuming and requires guidewire and access catheter removal and addition of a procedure catheter (and possibly one or more additional catheters) to the stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

Described herein are embodiments related to a surgical system and components thereof. The surgical system can include a “local” (or “bedside”) robotic surgical system and a “remote” control console that resides in a location which is typically at a distance from the robotic surgical system. The control console is configured such that a physician can operate the robotic surgical system from a remote location (i.e., teleoperate the robotic surgical system) to perform a procedure on a patient who is in the same location as the robotic surgical system. The surgical system facilitates safe and effective surgical procedures (e.g., removing a clot in vasculature of the patient) to be performed by a remotely located physician using the control console to operate the robotic surgical system, instead of being performed by a locally present physician. Such a remotely located control console may, at any given time, be configured to communicate with one of a plurality of robotic surgical systems which may each be at a different location, thus allowing a physician using the control console to perform surgical procedures at any one of a number of remote locations. Advantageously, this allows for such robotic surgical systems to be deployed in numerous locations without the need for a locally positioned physician to perform the surgical procedure, thus leveraging a skilled physician to treat people in many distant locations. In addition, when such robotic surgical systems are deployed in many areas where a properly skilled physician is not located, many patients can be provided quicker access to lifesaving surgical procedures without having to travel vast distances to places where such skilled physicians are located.

For purposes of this disclosure, “positioned remotely,” “remotely,” or “remote” can mean separated from a patient or one or more bedside devices including a local system (for example, separated from a robotic drive system and/or a fluidics system). In some embodiments, “positioned remotely,” “remotely,” or “remote” can mean located on another side of a fluoro barrier, in another room in the same building, in another building, in another part of a local geographical area (for example, another part of a town or city), or in another part of a global geographical area (for example, another state or country).

There is provided in accordance with one aspect of the present disclosure a supra-aortic access robotic control system. The system includes a guidewire hub configured to adjust each of an axial position and a rotational position of a guidewire; a guide catheter hub configured to adjust a guide catheter in an axial direction; and an access catheter hub configured to adjust each of an axial position and a rotational position of an access catheter. The access catheter hub may also laterally deflect a distal deflection zone of the access catheter. The guidewire hub may additionally be configured to laterally deflect a distal portion of the guidewire.

There may also be provided a procedure catheter hub configured to manipulate a procedure catheter. Following robotic placement of the guidewire, access catheter and guide catheter such that the guide catheter achieves supra aortic access, the guidewire and access catheter may be proximally withdrawn and the procedure catheter advanced through and beyond the guide catheter, with or without guidewire support (said guidewire may be smaller in diameter and/or more flexible than the guidewire used to gain supra aortic access), to reach a more distal neurovascular treatment site. The procedure catheter may be an aspiration catheter; an embolic deployment catheter; a stent deployment catheter; a flow diverter deployment catheter, an access catheter; a diagnostic angiographic catheter; a guiding catheter, an imaging catheter, a physiological sensing/measuring catheter, an infusion or injection catheter, an ablation catheter, an RF ablation catheter or guidewire, a balloon catheter, or a microcatheter used to deliver a stent retriever, a balloon catheter or a stent retriever, a catheter for the delivery of a valve, a temporary or permanently implanted sensing and stimulation electrode, or a distal anchoring device used to distally move the catheter.

The control system may further include a driven magnet on each of a guidewire hub, an access catheter hub and a guide catheter hub, configured to cooperate with corresponding drive magnets such that the driven magnet moves in response to movement of the corresponding drive magnet. The drive magnets may each be independently axially movably carried by a support table. The drive magnets may be located outside of the sterile field, separated from the driven magnets by a barrier, and the driven magnets may within the sterile field. The barrier may include a tray made from a thin polymer membrane, or any membrane of non-ferromagnetic material.

The control system may further include a control console which may be connected to the support table or may be located remotely from the support table. The position of each driven magnet and corresponding hub is movable in response to manual manipulation of a guidewire drive control, access catheter drive control, or procedure catheter drive control on the console or on a particular control device not associated with the console.

The control system may further include a processor for controlling the position of the drive magnets. The processor may be in wired communication with the control console, or in wireless communication with the control console. The driven magnets may be configured to remain engaged with the corresponding drive magnets until application of an axial disruption force of at least about 300 grams.

There is also provided a robotically driven interventional device. The device includes an elongate, flexible body, having a proximal end and a distal end. A hub is provided on the proximal end. At least one rotatable roller is provided on a first surface of the hub; and at least one magnet is provided on the first surface of the hub. The roller may extend further away from the first surface than the magnet. The hub may be further provided with at least a second roller.

Any of the guidewire hub, access catheter hub and procedure catheter hub may be further provided with a rotational drive, for rotating the corresponding interventional device with respect to the hub. The hub may be further provided with an axial drive mechanism to distally advance or proximally retract a control element extending axially through the interventional device, to adjust a characteristic such as shape or flexibility of the interventional device. In some embodiments, at least one control element may be an axially movable tubular body or fiber, ribbon, or wire such as a pull wire extending through the interventional device to, for example, a distal deflection zone. In some embodiments, any number of control elements may be advanced, retracted, or otherwise moved in a similar manner. In some embodiments, the diameter of an interventional device may be adjusted. In some embodiments, the guidewire hub, access catheter hub, and procedure catheter hub may provide temperature control for controlling an interventional device having a temperature controlled shape memory.

There is also provided a control system for controlling movement of interventional devices. In one configuration, the control system includes a guidewire control, configured to control axial travel and rotation of a guidewire; an access catheter control, configured to control axial and rotational movement of an access catheter; and a guide catheter control, configured to control axial movement and/or rotation of a guide catheter.

The control system may further include a deflection control, configured to control deflection of the access catheter or procedure catheter, and may be configured for wired or wireless communication with a robotic catheter drive system.

The control system may be configured to independently control the three or more hubs in a variety of modes. For example, two or more hubs may be selectively controlled together (i.e., as a set of hubs) so that a single control input from a control device drives the set of hubs, and correspondingly drives the respective interventional devices that are coupled to the hubs, simultaneously and with the same motion (e.g., same axial and/or rotational motion). Alternatively, the control system may be configured to drive respective devices simultaneously but with different motions.

The control system may further include a physician interface for operating the control system. The physician interface may be carried by a support table having a robotic interventional device drive system. Alternatively, the physician interface for operating the control system may be carried on a portable, handheld device or desktop computer, and may be located in the same room as the patient, the same facility as the patient, or in a remote facility.

The control system may further include a graphical user interface with at least one display for indicating the status of at least one device parameter, and/or indicating the status of at least one patient parameter.

There is also provided a sterile packaging assembly for transporting interventional devices to a robotic surgery site. The packaging assembly may include a base and a sterile barrier configured to enclose a sterile volume. At least one interventional device may be provided within the sterile volume, the device including a hub and an elongate flexible body. The hub may include at least one magnet and at least one roller configured to roll on the base.

In one implementation, the sterile barrier is removably attached to the base to define the enclosed volume between the sterile barrier and the base. In another implementation, the sterile barrier is in the form of a tubular enclosure for enclosing the sterile volume. The tubular enclosure may surround the base and the at least one interventional device, which are within the sterile volume.

The hub may be oriented within the packaging such that the roller and the magnet face the base. Alternatively, the base may be in the form of a tray having an elongate central axis. An upper, sterile field side of the tray may have an elongate support surface for supporting and permitting sliding movement of one or more hubs. At least one and optionally two elongate trays may be provided, extending parallel to the central axis. At least one hub and interventional device may be provided in the tray, and the sterile tray with sterile hub and interventional device may be positioned in a sterile volume defined by a sterile barrier.

The base may be configured to reside on a support table adjacent a patient, with an upper surface of the base within a sterile field and a lower surface of the base outside of the sterile field.

Any of the hubs disclosed herein may further include a fluid injection port and/or a wireless RF transceiver for communications and/or power transfer. The hub may include a visual indicator, for indicating the presence of a clot. In some embodiments, the hub may include a clot camera or be arranged so that the presence of a clot can be viewed by a clot camera. In some embodiments, the hub may also include wired electrical communications and power port. The visual indicator may include a clot chamber having a transparent window. A filter may be provided in the clot chamber.

Any of the hubs disclosed herein may further include a sensor for detecting a parameter of interest, for example, the presence of a clot. The sensor, in some instances, may be positioned on a flexible body. The sensor may include a pressure sensor or an optical sensor. In some embodiments, the sensor may include one or more of a force sensor, a positioning sensor, a temperature sensor, and/or an oxygen sensor. In some embodiments, the sensor may include a Fiber Bragg grating sensor. For example, a Fiber Bragg grating sensor (e.g., an optical fiber) may detect strain locally that can facilitate the detection and/or determination of force being applied. The device may further include a plurality of sensors. The plurality of sensors may each include one or more of any type of sensor disclosed herein. In some embodiments, a plurality (e.g., 3 or more) of sensors (e.g., Fiber Bragg grating sensors) may be distributed around a perimeter to facilitate the detection and/or determination of shape. The position of the device, in some instance, may be determined through the use of one or more sensors to detect and/or determine the position. For example, one or more optical encoders may be located in or proximate to one or more the motors that drive linear motion such that the optical encoders may determine a position.

There is also provided a method of performing a neurovascular procedure, in which a first phase includes robotically achieving supra-aortic access, and a second phase includes manually or robotically performing a neurovascular procedure via the supra-aortic access. The method includes the steps of providing an access catheter having an access catheter hub; coupling the access catheter hub to a hub adapter movably carried by a support table; driving the access catheter in response to movement of the hub adapter along the table until the access catheter is positioned to achieve supra-aortic access. The access catheter and access catheter hub may then be decoupled from the hub adapter; and a procedure catheter hub having a procedure catheter may then be coupled to the hub adapter.

The method may additionally include advancing the procedure catheter hub to position a distal end of the procedure catheter at a neurovascular treatment site. The driving the access catheter step may include driving the access catheter distally through a guide catheter. The driving the access catheter step may include the step of laterally deflecting a distal region of the access catheter to achieve supra-aortic access. In some embodiments, the driving the access catheter step may also include rotating the access catheter.

There is also provided a method of performing a neurovascular procedure, comprising the steps of providing an access assembly comprising a guidewire, access catheter and guide catheter. The access assembly may be releasably coupled to a robotic drive system. The access assembly may be driven by the robotic drive system to achieve access to a desired point, such as to achieve supra-aortic access. The guidewire and the access catheter may then be decoupled from the access assembly, leaving the guide catheter in place. A procedure assembly may be provided, comprising at least a guidewire and a first procedure catheter. The procedure assembly may be releasably coupled to the robotic drive system; and a neurovascular procedure may be accomplished using the procedure assembly. A second procedure catheter may also be provided, for extending through the first procedure catheter to a treatment site.

The coupling the access assembly step may include magnetically coupling a hub on each of the guidewire, access catheter and guide catheter, to separate corresponding couplers carrying corresponding drive magnets independently movably carried by the drive table. The procedure assembly may include a guidewire, a first catheter and a second catheter. The guidewire and first catheter may be positioned concentrically within the second catheter. The procedure assembly may be advanced as a unit through at least a portion of the length of the guide catheter, and the procedure may include a neurovascular thrombectomy.

There is also provided a method of performing a neurovascular procedure. The method includes the steps of providing a multi-catheter assembly including an access catheter, a guide catheter, and a procedure catheter, coupling the assembly to a robotic drive system, driving the assembly to achieve supra-aortic access, driving a subset of the assembly to a neurovascular site, wherein the subset includes the guide catheter and the procedure catheter, proximally removing the access catheter, and performing a neurovascular procedure using the procedure catheter.

The neurovascular procedure can include a neurovascular thrombectomy. The assembly may further include a guidewire, wherein each of the guidewire, the access catheter, the guide catheter, and the procedure catheter are configured to be adjusted by a respective hub. Coupling the assembly to the robotic drive system can include magnetically coupling a first hub of the guidewire to a first drive magnet, magnetically coupling a second hub of the access catheter to a second drive magnet, magnetically coupling a third hub of the guide catheter to a third drive magnet, and magnetically coupling a fourth hub of the procedure catheter to a fourth drive magnet. The first drive magnet, the second drive magnet, the third drive magnet, and the fourth drive magnet can each be independently movably carried by a drive table. The procedure catheter can be an aspiration catheter. The procedure catheter can be an embolic deployment catheter. The procedure catheter can be a stent deployment catheter. The procedure catheter can be a flow diverter deployment catheter. The procedure catheter can be a diagnostic angiographic catheter. The procedure catheter can be a stent retriever catheter. The procedure catheter can be a clot retriever. The procedure catheter can be a balloon catheter. The procedure catheter can be a catheter to facilitate percutaneous valve repair or replacement. The procedure catheter can be an ablation catheter.

There is also provided a method of performing a neurovascular procedure. The method includes the steps of providing an assembly including a guidewire, an access catheter, a guide catheter, and a procedure catheter coaxially moveably assembled into a single multi-catheter assembly, coupling the assembly to a drive system, driving the assembly to achieve supra-aortic access, driving a subset of the assembly to an intracranial site, wherein the subset includes the guidewire, the guide catheter, and the procedure catheter, and performing a neurovascular procedure using the subset of the assembly.

Each of the guidewire, the access catheter, the guide catheter, and the procedure catheter can be configured to be adjusted by a respective hub. Coupling the assembly to the drive system can include magnetically coupling a first hub of the guidewire to a first drive magnet, magnetically coupling a second hub of the access catheter to a second drive magnet, magnetically coupling a third hub of the guide catheter to a third drive magnet, and magnetically coupling a fourth hub of the procedure catheter to a fourth drive magnet. The drive system can be a robotic drive system, and the first drive magnet, the second drive magnet, the third drive magnet, and the fourth drive magnet can each be independently movably carried by a drive table associated with the robotic drive system. The first drive magnet, the second drive magnet, the third drive magnet, and the fourth drive magnet can each be independently movably carried by a drive table.

There is also provided a method of performing a neurovascular procedure. The method includes providing an assembly including a guidewire having a guidewire hub, an access catheter having an access catheter hub, and a guide catheter having a guide catheter hub. The method also includes coupling the guidewire hub to a first hub adapter, the access catheter hub to a second hub adapter, and the guide catheter hub to a third hub adapter, wherein each of the first hub adapter, the second hub adapter and the third hub adapter is movably carried by a support table. The method also includes driving the assembly in response to movement of each of the first hub adapter, the second hub adapter and the third hub adapter along the support table until the assembly is positioned to achieve supra-aortic vessel access.

The method can include the step of driving a subset of the assembly along the support table until the subset of the assembly is positioned to perform a neurovascular procedure at a neurovascular treatment site, wherein the subset of the assembly includes the guidewire, the guide catheter, and a procedure catheter. The neurovascular procedure can include a thrombectomy. Coupling the guidewire hub to the first hub adapter can include magnetically coupling the guidewire hub to a first drive magnet. Coupling the access catheter hub to the second hub adapter can include magnetically coupling the access catheter hub to a second drive magnet. Coupling the guide catheter hub to the third hub adapter can include magnetically coupling the guide catheter hub to a third drive magnet. The first drive magnet, the second drive magnet and the third drive magnets can be independently movably carried by the support table. The first drive magnet can be coupled to a first driven magnet across a sterile field barrier. The second drive magnet can be coupled to a second driven magnet across the sterile field barrier. The third drive magnet can be coupled to a third driven magnet across the sterile field barrier. Coupling the guidewire hub to the first hub adapter can include mechanically coupling the guidewire hub to a first drive. Coupling the access catheter hub to the second hub adapter can include mechanically coupling the access catheter hub to a second drive. Coupling the guide catheter hub to the third hub adapter can include mechanically coupling the guide catheter hub to a third drive. The guidewire and the guide catheter can be advanced as a unit along at least a portion of a length of the access catheter after supra-aortic access is achieved. The guidewire hub can be configured to adjust an axial position and a rotational position of the guidewire. The assembly can further include a procedure catheter having a procedure catheter hub. The procedure catheter hub can be configured to adjust an axial position and a rotational position of the procedure catheter. The procedure catheter hub can be further configured to laterally deflect a distal deflection zone of the procedure catheter. The guidewire hub can be configured to adjust an axial position and a rotational position of the guidewire. The procedure catheter hub can be configured to adjust an axial position and a rotational position of the procedure catheter. The guide catheter hub can be configured to adjust an axial position of the guide catheter. The access catheter hub can be configured to adjust an axial position and a rotational position of the access catheter. The procedure catheter hub can be further configured to laterally deflect a distal deflection zone of the procedure catheter. The access catheter hub can be further configured to laterally deflect a distal deflection zone of the access catheter. The guide catheter hub can be configured to adjust an axial position of the guide catheter. The access catheter hub can be configured to adjust an axial position and a rotational position of the access catheter. The access catheter hub can be further configured to laterally deflect a distal deflection zone of the access catheter.

There is also provided a drive system for achieving supra-aortic access and neurovascular treatment site access. The system includes a guidewire hub configured to adjust an axial position and a rotational position of a guidewire, a procedure catheter hub configured to adjust an axial position and a rotational position of a procedure catheter, a guide catheter hub configured to adjust an axial position of a guide catheter, and an access catheter hub configured to adjust an axial position and a rotational position of an access catheter, the access catheter further configured to laterally deflect a distal deflection zone of the access catheter.

The procedure catheter hub can be further configured to laterally deflect a distal deflection zone of the procedure catheter. The guidewire hub can be configured to couple to a guidewire hub adapter by magnetically coupling the guidewire hub to a first drive magnet. The access catheter hub can be configured to couple to an access catheter hub adapter by magnetically coupling the access catheter hub to a second drive magnet. The guide catheter hub can be configured to couple to a guide catheter hub adapter by magnetically coupling the guide catheter hub to a third drive magnet. The procedure catheter hub can be configured to couple to a procedure catheter hub adapter by magnetically coupling the procedure catheter hub to a fourth drive magnet. The first drive magnet, the second drive magnet, the third drive magnet, and the fourth drive magnet can be independently movably carried by a drive table. The system can include first driven magnet on the guidewire hub configured to cooperate with the first drive magnet such that the first driven magnet moves in response to movement of the first drive magnet. The first drive magnet can be configured to move outside of a sterile field while separated from the first driven magnet by a sterile field barrier while the first driven magnet is within the sterile field. A position of the first drive magnet can be movable in response to manipulation of a procedure drive control on a control console in electrical communication with the drive table. The system can include a second driven magnet on the access catheter hub configured to cooperate with the second drive magnet such that the second driven magnet is configured to move in response to movement of the second drive magnet, wherein the second drive magnet is configured to move outside of the sterile field while separated from the second driven magnet by the barrier while the second driven magnet is within the sterile field. The system can include a third driven magnet on the guide catheter hub configured to cooperate with the third drive magnet such that the third driven magnet is configured to move in response to movement of the third drive magnet, wherein the third drive magnet is configured to move outside of the sterile field while separated from the third driven magnet by the barrier while the third driven magnet is within the sterile field. The system can include a fourth driven magnet on the procedure catheter hub configured to cooperate with the fourth drive magnet such that the fourth driven magnet is configured to move in response to movement of the fourth drive magnet, wherein the fourth drive magnet is configured to move outside of the sterile field while separated from the fourth driven magnet by the barrier while the fourth driven magnet is within the sterile field. The procedure catheter can be an aspiration catheter. The procedure catheter can be an embolic deployment catheter. The procedure catheter can be a stent deployment catheter. The procedure catheter can be a flow diverter deployment catheter. The procedure catheter can be a diagnostic angiographic catheter. The procedure catheter can be a stent retriever catheter. The procedure catheter can be a balloon catheter. The procedure catheter can be a catheter to facilitate percutaneous valve repair or replacement. The procedure catheter can be an ablation catheter.

There is also provided method of achieving supra-aortic access and neurovascular treatment site access. The method includes the steps of providing a drive system including a guidewire hub configured to adjust an axial position and a rotational position of a guidewire, a procedure catheter hub configured to adjust an axial position and a rotational position of a procedure catheter; a guide catheter hub configured to adjust an axial position of a guide catheter, and an access catheter hub configured to adjust an axial position and a rotational position of an access catheter, the access catheter further configured to laterally deflect a distal deflection zone of the access catheter, and moving at least one of the guidewire hub, the procedure catheter hub, the guide catheter hub, and the access catheter hub to drive movement of at least one of the guidewire, the procedure catheter, the guide catheter, and the access catheter. The method can further include controlling the procedure catheter hub to laterally deflect a distal deflection zone of the procedure catheter.

There is also provided a method of achieving supra aortic access. The method includes the steps of providing an assembly including a guidewire, an access catheter and a guide catheter, coaxially moveably assembled into a single multi-catheter assembly, coupling the assembly to a drive system, driving the assembly to an aortic arch, and advancing the access catheter to achieve supra-aortic access to a branch vessel off of the aortic arch.

The method can further include driving a subset of the assembly to an intracranial site, and performing a neurovascular procedure using the subset of the assembly. The subset can include the guidewire, the guide catheter, and a procedure catheter. The procedure catheter can be an aspiration catheter. The procedure catheter can be an embolic deployment catheter.

The procedure catheter can be a stent deployment catheter. The procedure catheter can be a flow diverter deployment catheter. The procedure catheter can be a diagnostic angiographic catheter. The procedure catheter can be a stent retriever catheter. The procedure catheter can be a clot retriever. The procedure catheter can be a balloon catheter. The procedure catheter can be a catheter to facilitate percutaneous valve repair or replacement. The procedure catheter can be an ablation catheter. The intracranial procedure can include an intracranial thrombectomy. The neurovascular procedure can include a neurovascular thrombectomy. At least one of the guidewire, the access catheter, and the guide catheter can include a hub configured to couple to a robotic drive system. Coupling the assembly to the drive system can include magnetically coupling a guide catheter hub to the drive system. Coupling the assembly to the drive system can include mechanically coupling a guide catheter hub to the drive system. The drive system can be a robotic drive system, and at least a first drive magnet, a second drive magnet, and a third drive magnet are each independently movably carried by a drive table associated with the robotic drive system.

There is also provided a method of priming an interventional device assembly. The method includes providing the interventional device assembly, the interventional device assembly including a first interventional device coupled to a first hub and a second interventional device coupled to a second hub arranged in a concentric stack, the second interventional device being positioned within a lumen of the first interventional device. The method includes coupling the interventional device assembly to a drive system while arranged in the concentric stack, axially advancing the first interventional device and the first hub relative to the second hub to decrease a depth of insertion of the second interventional device within the lumen of the first interventional device while maintaining a distal end of the second interventional device within the lumen of the first interventional device, and flushing the first interventional device with fluid after decreasing the depth of insertion of the second interventional device within the lumen of the first interventional device. The method may lower the fluid resistance through the annular space of the first interventional device.

The drive system can be a robotic drive system. Axially advancing the first interventional device and the first hub relative to the second hub can include axially moving a first robotic drive coupled to the first hub relative to a second robotic drive coupled to the second hub. Axially advancing the first interventional device and the first hub relative to the second hub can include axially advancing the first interventional device and the first hub relative to the second hub in response to a control signal. The first interventional device can be a first catheter and the second interventional device can be a second catheter. The first catheter can be a guide catheter, the first hub can be a guide catheter hub, the second catheter can be a procedure catheter, and the second hub can be a procedure catheter hub. The interventional device assembly can include an access catheter coupled to an access catheter hub arranged in the concentric stack, the access catheter being positioned within a lumen of the procedure catheter. The method can include returning the guide catheter to an initial position relative to the procedure catheter after flushing the guide catheter with fluid, axially advancing the guide catheter, the guide catheter hub, the procedure catheter, and the procedure catheter hub relative to the access catheter hub to decrease a depth of insertion of the access catheter within the lumen of the procedure catheter while maintaining a distal end of the access catheter within the lumen of the procedure catheter and substantially maintaining a relative position between the guide catheter and the procedure catheter, and flushing the procedure catheter with fluid after decreasing the depth of insertion of the access catheter within the lumen of the procedure catheter. The interventional device assembly can include a guidewire coupled to a guidewire hub arranged in the concentric catheter stack, the guidewire being positioned within a lumen of the access catheter. The method can include returning the guide catheter and the procedure catheter to an initial position relative to the access catheter after flushing the procedure catheter with fluid, axially advancing the guide catheter, the guide catheter hub, the procedure catheter, the procedure catheter hub, the access catheter, and the access catheter hub relative to the guidewire hub to decrease a depth of insertion of the guidewire within the lumen of the access catheter while maintaining a distal end of the guidewire within the lumen of the access catheter and substantially maintaining relative positions between the guide catheter, the procedure catheter, and the access catheter, and flushing the access catheter with fluid after decreasing the depth of insertion of the guidewire within the lumen of the access catheter. The method can include flushing the second catheter with fluid, wherein the steps of flushing the first catheter and flushing the second catheter are performed simultaneously. The fluid can be saline, contrast media, or a combination of saline and contrast media. The first interventional device can be a catheter and the second interventional device can be a guidewire. The method can include reciprocally moving at least one of the first interventional device and the second interventional device relative to the other of the first interventional device and the second interventional device while flushing the first interventional device with fluid after decreasing the depth of insertion of the second interventional device within the lumen of the first interventional device. The method may advantageously increase fluid shear in the annular space between shafts for dislodging air bubbles.

There is also provided a method of priming a multi-catheter assembly. The method includes providing the multi-catheter assembly, the multi-catheter assembly including a guidewire, an access catheter, a procedure catheter, and a guide catheter in a concentric stacked configuration, coupling the multi-catheter assembly to a drive system, translating the guide catheter distally relative to the guidewire, the access catheter, and the procedure catheter, flushing the guide catheter with fluid, and translating the guide catheter proximally towards the guidewire, the access catheter, and the procedure catheter.

The drive system can be a robotic drive system. The method can include translating the procedure catheter and the guide catheter distally relative to the guidewire and the access catheter, flushing the procedure catheter with fluid, and translating the procedure catheter and the guide catheter proximally towards the guidewire and the access catheter. The method can include translating the access catheter, the procedure catheter, and the guide catheter distally relative to the guidewire, flushing the access catheter with fluid, and translating the access catheter, the procedure catheter, and the guide catheter proximally towards the guidewire. The fluid can be saline, contrast media, or a combination of saline and contrast media. The guidewire can coupled to a guidewire hub. The access catheter can be coupled to an access catheter hub. The procedure catheter can be coupled to a procedure catheter hub. The guide catheter can be coupled to a guide catheter hub. In the concentric stacked configuration, the procedure catheter is positioned within a lumen of the guide catheter, the access catheter is positioned within a lumen of the procedure catheter, and the guidewire is positioned within a lumen of the access catheter. The method can include reciprocally moving at least one of the guide catheter and the procedure catheter relative to the other of the guide catheter and the procedure catheter while flushing the guide catheter with fluid.

There is also provided a method of priming an interventional device assembly. The method includes providing the interventional device assembly, the interventional device assembly comprising a first interventional device and a second interventional device, the second interventional device being positioned within the first interventional device, and reciprocally moving at least one of the first interventional device and the second interventional device relative to the other of the first interventional device and the second interventional device while flushing a lumen between the first interventional device and the second interventional device with fluid to remove microbubbles from the lumen.

Reciprocally moving at least one of the first interventional device and the second interventional device relative to the other of the first interventional device and the second interventional device can include reciprocally moving at least one of the first interventional device and the second interventional device relative to the other of the first interventional device and the second interventional device in response to a control signal. Reciprocally moving at least one of the first interventional device and the second interventional device relative to the other of the first interventional device and the second interventional device can include reciprocally moving at least one of a first robotic drive coupled to the first interventional device and a second robotic drive coupled to the second interventional device relative to the other of the first robotic drive and the second robotic drive. Reciprocally moving at least one of the first interventional device and the second interventional device relative to the other of the first interventional device and the second interventional device can include axially reciprocally moving at least one of the first interventional device and the second interventional device relative to the other of the first interventional device and the second interventional device. Reciprocally moving at least one of the first interventional device and the second interventional device relative to the other of the first interventional device and the second interventional device can further include rotationally reciprocally moving at least one of the first interventional device and the second interventional device relative to the other of the first interventional device and the second interventional device. Axially reciprocally moving at least one of the first interventional device and the second interventional device relative to the other of the first interventional device and the second interventional device can include axially reciprocally moving at least one of the first interventional device and the second interventional device relative to the other of the first interventional device and the second interventional device over a stroke length between about 10 mm and about 250 mm. Axially reciprocally moving at least one of the first interventional device and the second interventional device relative to the other of the first interventional device and the second interventional device can include axially reciprocally moving at least one of the first interventional device and the second interventional device relative to the other of the first interventional device and the second interventional device over a stroke length between about 25 mm and about 125 mm. Axially reciprocally moving at least one of the first interventional device and the second interventional device relative to the other of the first interventional device and the second interventional device can include axially reciprocally moving at least one of the first interventional device and the second interventional device relative to the other of the first interventional device and the second interventional device over a stroke length greater than 20 mm. Axially reciprocally moving at least one of the first interventional device and the second interventional device relative to the other of the first interventional device and the second interventional device can include axially reciprocally moving at least one of the first interventional device and the second interventional device relative to the other of the first interventional device and the second interventional device at a reciprocation frequency of no more than about 5 Hz. The reciprocation frequency can be no more than about 1 Hz. Reciprocally moving at least one of the first interventional device and the second interventional device relative to the other of the first interventional device and the second interventional device can include rotationally reciprocally moving at least one of the first interventional device and the second interventional device relative to the other of the first interventional device and the second interventional device. Reciprocally moving at least one of the first interventional device and the second interventional device relative to the other of the first interventional device and the second interventional device can include reciprocally moving both the first interventional device and the second interventional device relative to one another. Reciprocally moving at least one of the first interventional device and the second interventional device relative to the other of the first interventional device and the second interventional device can be performed by a robotic drive table. The first interventional device can be a first catheter and the second interventional device can be a second catheter. The first interventional device can be a catheter and the second interventional device can be a guidewire.

There is also provided a method of priming a multi-catheter assembly. The method includes providing the multi-catheter assembly, the multi-catheter assembly including a guidewire, an access catheter, a procedure catheter, and a guide catheter arranged in a concentric catheter stack, wherein the guidewire is positioned within a lumen of the access catheter, the access catheter is positioned within a lumen of the procedure catheter, and the procedure catheter is positioned within a lumen of the guide catheter, and flushing the guide catheter with saline while reciprocally moving at least one of the guide catheter and the procedure catheter relative to the other of the guide catheter and the procedure catheter.

Flushing the guide catheter with saline while reciprocally moving at least one of the guide catheter and the procedure catheter relative to the other of the guide catheter and the procedure catheter can include reciprocally moving at least one of the guide catheter and the procedure catheter relative to the other of the guide catheter and the procedure catheter in response to a control signal. Flushing the guide catheter with saline while reciprocally moving at least one of the guide catheter and the procedure catheter relative to the other of the guide catheter and the procedure catheter can include reciprocally moving at least one of a first robotic drive coupled to the guide catheter and a second robotic drive coupled to the procedure catheter relative to the other of the first robotic drive and the second robotic drive. Flushing the guide catheter with saline while reciprocally moving at least one of the guide catheter and the procedure catheter relative to the other of the guide catheter and the procedure catheter can include axially reciprocally moving, rotationally reciprocally moving, or both axially and rotationally reciprocally moving at least one of the guide catheter and the procedure catheter relative to the other of the guide catheter and the procedure catheter. The method can include flushing the procedure catheter with saline while reciprocally moving at least one of the procedure catheter and the access catheter relative to the other of the procedure catheter and the access catheter. Flushing the procedure catheter with saline while reciprocally moving at least one of the procedure catheter and the access catheter relative to the other of the procedure catheter and the access catheter can include axially reciprocally moving, rotationally reciprocally moving, or both axially and rotationally reciprocally moving at least one of the procedure catheter and the access catheter relative to the other of the procedure catheter and the access catheter. The method can include flushing the access catheter with saline while reciprocally moving at least one of the access catheter and the guidewire relative to the other of the access catheter and the guidewire. Flushing the access catheter with saline while reciprocally moving at least one of the access catheter and the guidewire can include axially reciprocally moving, rotationally reciprocally moving, or both axially and rotationally reciprocally moving at least one of the access catheter and the guidewire relative to the other of the access catheter and the guidewire. The steps of flushing the guide catheter with saline while reciprocally moving at least one of the guide catheter and the procedure catheter relative to the other of the guide catheter and the procedure catheter, flushing the procedure catheter with saline while reciprocally moving at least one of the procedure catheter and the access catheter relative to the other of the procedure catheter and the access catheter, and flushing the access catheter with saline while reciprocally moving at least one of the access catheter and the guidewire relative to the other of the access catheter and the guidewire can be performed simultaneously.

There is also provided a control system. The control system includes a remote system located remotely from an operating room, and a local system in communication with the remote system and located within the operating room, the local system further including: one or more computing devices; a robotic drive system in communication with the one or more computing devices; a fluidics system in communication with the one or more computing devices, the fluidics system having a saline subsystem, a contrast subsystem, and an aspiration subsystem; and a plurality of interventional devices configured to be independently moved in an axial direction by the robotic drive system and in fluid communication with the fluidics system, the plurality of interventional devices configured to be at least partially positioned in a concentric nested configuration and moved independently in the axial direction when concentrically nested; wherein the local system receives first information from the remote system, processes the first information to determine movement control signals, and provides the movement control signals to the robotic drive system for moving the plurality of interventional devices; and wherein the local system receives second information from the remote system, processes the second information to determine fluidics control signals, and provides the fluidics control signals to the fluidics system for independently providing saline, contrast, and/or vacuum to the plurality of interventional devices. The remote system further includes a control console having at least one monitor and control devices for moving each of the plurality of interventional devices. The local system further includes a user interface wherein the plurality of interventional devices are axially and rotationally movable based on inputs from the user interface. The plurality of interventional devices include three catheters and a guidewire, wherein each catheter is in fluid communication with a distinct subsystem of the fluidics system. The first information is generated by the remote system, represents a movement of the control devices, and is indicative of a desired movement for one or more of the plurality of interventional devices. The second information is generated by the remote system and is indicative of a desired fluidics action to occur at fluidics system and one or more of the plurality of interventional devices. The remote system receives third information from the local system, the third information comprising fluoroscopic images of a working area. The third information includes media streams of the operating room to be displayed on the at least one monitor and/or instructions to manipulate the one or more control devices. The control system further includes one or more hardware processors configured to execute instructions to detect and manipulate latency in the control system.

In some aspects, the control system includes a remote system located remotely from an operating room. The remote system further includes a control console. The control system includes a local system in communication with the control console and located within the operating room, the local system further includes one or more computing devices; a robotic drive system in communication with the one or more computing devices; a fluidics system in communication with the one or more computing devices, the fluidics system having a saline subsystem, a contrast subsystem, and an aspiration subsystem; and a plurality of interventional devices configured to be independently moved in an axial direction by the robotic drive system and in fluid communication with the fluidics system. The plurality of interventional devices further include a distinct catheter in fluid communication with each subsystem of the fluidics system and a guidewire. The local system can provide a graphical user interface for displaying setup guidance information and perform patient setup. The remote system can receive the information from one or more of the plurality of interventional devices. The system local system may have an additional camera aimed at the clot chamber described above. This camera view can be transmitted to the remote system. The same view can be displayed on the local system. The third information can include media streams of the clot chamber. In some embodiments, the third information can include media streams of a drip chamber. The plurality of interventional devices are configured to be at least partially positioned in a concentric nested configuration. The remote system is configured to communicate first information to the local system. The local system is configured to communicate second information to the remote system. A streaming system can be provided in communication with the imaging (fluoroscopic) system and the control system. One or more hardware processors are configured to execute instructions to detect and manipulate latency in the control system. The one or more hardware processors inject a time delay into the control system when the latency is less than a predetermined threshold. The first information and the second information include electronic signals and/or data selected from the group consisting of: control signals, fluoroscopic images, or media streams. The remote and local systems communicate information based on an associated priority of the information when the latency is greater than a predetermined threshold. Control signals are prioritized over fluoroscopic images and media stream, and fluoroscopic images are prioritized over media streams. The one or more hardware processors reduce the resolution of communicated information when the latency is greater than a predetermined threshold.

In one aspect, the control system for remotely operating on a patient includes a remote system located remotely from an operating room. The remote system further includes a control console. The control console further includes a control device; a first set of one or more monitors; and a first set of one or more communication devices. The control system further includes a local system in communication with the remote system and located within the operating room. The local system includes one or more computing devices; a robotic drive system in communication with the one or more computing devices; a fluidics system in communication with the one or more computing devices, the fluidics system having a saline subsystem, a contrast subsystem, and an aspiration subsystem; a plurality of interventional devices configured to be independently moved in an axial direction by the robotic drive system and in fluid communication with the fluidics system; a second set of one or more monitors; and a second set of one or more communication devices. The remote system transmits first information representing a movement of the control device to the local system for causing a corresponding movement of one or more of the plurality of interventional devices. The remote system transmits second information representing media streams captured by the first set of one or more communication devices to the local system for displaying the second information on the second set of one or more monitors. The local system transmits third information representing media streams captured by the second set of one or more communication devices to the remote system for displaying the third electronic signal and/or data on the first set of one or more monitors. For example, the local system can transmit fluoroscopy images to the remote system. The one or more communication devices are imaging devices and microphones. The one or more communication devices include a plurality of imaging devices uniquely oriented and each having a distinct view.

There is also provided a robotic medical system for performing a vascular procedure. The robotic medical system includes a remote system and a local system. The local system includes one or more computing devices, a robotic drive system in communication with the one or more computing devices, a fluidics system in communication with the one or more computing devices; and a plurality of interventional devices. The plurality of interventional devices are at least partially arranged in a concentric nested configuration. Each of the plurality of interventional devices are independently movable along a common axial direction by the robotic drive system, wherein one or more of the plurality of interventional devices are in fluid communication with the fluidics system. The remote system is located remotely from the robotic drive system. The local system is configured to receive first information from the remote system, determine movement control signals for moving the plurality of interventional devices based on the first information, and provide the movement control signals to the robotic drive system for independently moving one or more of the plurality of interventional devices. The local system is further configured to receive second information from the remote system, determine fluidics control signals from the second information, and provide the fluidics control signals to the fluidics system for independently providing one or more fluids and/or vacuum to one or more of the plurality of interventional devices.

In some aspects, the fluidics system can include a saline subsystem, a contrast subsystem, and an aspiration subsystem, wherein the plurality of interventional devices include three catheters and a guidewire, and wherein the fluidics system is configured to independently provide saline, contrast, and vacuum to the three catheters based on the fluidics control signals. The local system can further include three mounts, wherein each mount is coupled to one of the catheters, wherein each mount is movable in the axial direction by the robotic drive system, and wherein each mount includes saline, contrast, and vacuum channels for providing saline, contrast, and vacuum to the catheter coupled thereto. The plurality of interventional devices can include two or more catheters, and wherein the second information can indicate one or more of the two or more catheters to provide fluids and/or vacuum to. The remote system can include a control console having at least one monitor and one or more control devices in communication with the control console, wherein the remote system is configured to generate control signals for moving one or more of the plurality of interventional devices in response to actuation of one or more controls of the one or more control devices. The local system can further include a user interface, and wherein each of the plurality of interventional devices are axially and/or rotationally movable based on inputs from the user interface. The plurality of interventional devices can include at least two catheters, and the fluidics control signals configure the fluidics system to provide contrast or vacuum to one of the at least two catheters. The plurality of interventional devices can include at least two catheters, and the fluidics control signals configure the fluidics system to provide contrast or vacuum to both of the at least two catheters. The plurality of interventional devices can include at least three catheters, and the fluidics control signals configure the fluidics system to provide contrast or vacuum to one of the at least three catheters. The plurality of interventional devices can include three catheters and a guidewire, and wherein each catheter is in fluid communication with the fluidics system to independently receive saline, contrast, and vacuum from a saline subsystem, a contrast subsystem, and aspiration subsystem, respectively, based on the fluidics control signals. The remote system can receive third information from the local system, the third information including one or more fluoroscopic images of a working area. The first information can be generated by the remote system, represents a movement of a control of control device, and can be indicative of a desired movement for one or more of the plurality of interventional devices. The second information can be generated by the remote system and can be indicative of a desired fluidics action to occur at the fluidics system and one or more of the plurality of interventional devices. The remote system can receive third information from the local system wherein the third information includes media streams of an operating room to be displayed on at least one monitor. The remote system can receive third information from the local system, wherein the third information includes instructions to manipulate one or more control devices. The robotic medical system can further includes one or more hardware processors configured to execute instructions to detect and manipulate latency in robotic medical system. The remote system can be separated from the robotic drive system by a fluoroscopic barrier. The remote system and the robotic drive system can be located in separate rooms. The remote system and the robotic drive system can be located in separate geographic areas. The local system can include a streaming system in communication with the remote system.

There is also provided a robotic medical system for performing a vascular procedure. The robotic medical system includes a remote system and a local system. The remote system includes a control console and the local system in is communication with the control console. The local system includes one or more computing devices, a robotic drive system in communication with the one or more computing devices, a fluidics system in communication with the one or more computing devices, and a plurality of interventional devices at least partially arranged in a concentric nested configuration. Each of the plurality of interventional devices is independently movable along a common axial direction by the robotic drive system. The plurality of interventional devices further includes one or more catheters in fluid communication with the fluidics system and a guidewire. The remote system is configured to communicate first information to the local system. The local system is configured to communicate second information to the remote system. The remote system is located remotely from the robotic drive system

In some aspects, the robotic medical system can further include one or more hardware processors configured to execute instructions to detect and manipulate latency in the robotic medical system. The one or more hardware processors can inject a time delay into the robotic medical system when the latency is less than a predetermined threshold. The first information and the second information can include electronic signals and/or data selected from a group consisting of: control signals, fluoroscopic images, or media streams. The remote and local systems can communicate information based on an associated priority of the information when the latency is greater than a predetermined threshold. Control signals can be prioritized over fluoroscopic images and media stream, and fluoroscopic images can be prioritized over media streams. The one or more hardware processors can reduce a resolution of communicated information when the latency is greater than a predetermined threshold. The fluidics system can include a saline subsystem, a contrast subsystem, and an aspiration subsystem.

There is also provided a robotic medical system for performing a vascular procedure. The robotic medical system includes a remote system and a local system in communication with the remote system. The remote system includes a control device, a first set of one or more monitors, and a first set of one or more communication devices. The local system includes one or more computing devices, a robotic drive system in communication with the one or more computing devices, a fluidics system in communication with the one or more computing devices, a plurality of interventional devices at least partially arranged in a concentric nested configuration, a second set of one or more monitors, and a second set of one or more communication devices. Each of the plurality of interventional devices being independently moveable along a common axial direction by the robotic drive system. One or more of the plurality of interventional devices are in fluid communication with the fluidics system. The remote system is located remotely from the robotic drive system. The remote system transmits first information representing a movement of a control of the control device to the local system for causing a corresponding movement of one or more of the plurality of interventional devices. The remote system transmits second information representing media streams captured by the first set of one or more communication devices to the local system for displaying the second information on the second set of one or more monitors. The local system transmits third information representing media streams captured by the second set of one or more communication devices to the remote system for displaying the third information on the first set of one or more monitors.

In some aspects, the second set of one or more communication devices can be imaging devices and microphones. The second set of one or more communication devices can include a plurality of imaging devices uniquely oriented and each having a distinct view. The fluidics system includes a saline subsystem, a contrast subsystem, and an aspiration subsystem.

### BRIEF DESCRIPTION OF THE DRAWINGS

FIG. 1 is a schematic perspective view of an interventional setup having an imaging system, a patient support table, and a robotic drive system in accordance with the present disclosure.

FIG. 2 is a longitudinal cross section showing the concentric relationship between a guidewire having two degrees of freedom, an access catheter having 3 degrees of freedom and a guide catheter having one degree of freedom.

FIG. 3A is an exploded schematic view of interventional device hubs separated from a support table by a sterile barrier.

FIGS. 3B-3F show an alternate sterile barrier in the form of a shipping tray having one or more storage channels for carrying interventional devices.

FIGS. 3G-3K show embodiments of an alternate sterile barrier having a convex drive surface.

FIGS. 3L and 3M depict an example of a hub that may be used with the sterile barriers of FIGS. 3G-3K.

FIG. 4 is a schematic elevational cross section through a hub adapter having a drive magnet separated from an interventional device hub and driven magnet by a sterile barrier.

FIGS. 5A and 5B schematically illustrate a three interventional device and

a four interventional device assembly.

FIG. 6 is a perspective view of a support table.

FIG. 7 is a close-up view of the motor drive end of a support table.

FIG. 8 is an elevational cross section through a motor and belt drive assembly.

FIG. 9 is a close-up view of a pulley end of the support table.

FIG. 10 is an elevational cross section through a belt pulley.

FIG. 11 is a side elevational cross-section through a distal portion of a catheter such as any of those shown in FIGS. 5A and 5B.

FIGS. 12A and 12B schematically illustrate a force sensor integrated into the sidewall of the catheter.

FIGS. 13A and 13B schematically illustrate a sensor for measuring elastic forces at the magnetic coupling between the hub and corresponding carriage.

FIG. 14 schematically illustrates a dual encoder torque sensor for use with a catheter of the present disclosure.

FIG. 15 illustrates a clot capture and visualization device that can be integrated into a hub and/or connected to an aspiration line.

FIGS. 16A-16C illustrate an example control mechanism for manipulating interventional devices driven by respective hubs.

FIG. 17 illustrates a side elevational schematic view of an interventional device assembly for supra-aortic access and neuro-interventional procedures.

FIGS. 18A-18E depict an example sequence of steps of introducing a catheter assembly configured to achieve supra-aortic access and neurovascular site access.

FIG. 19 schematically illustrates an embodiment of a mechanical coupling between a drive mechanism and a driven mechanism.

FIGS. 20A-20C depict an example sequence of steps of priming a catheter assembly in a stacked configuration.

FIGS. 21A-21B depict an example sequence of steps of priming a catheter assembly in a stacked configuration.

FIG. 22 depicts an example test system for the priming process depicted in FIGS. 21A-21B.

FIG. 23A shows an example of a catheter assembly.

FIG. 23B shows an example of a catheter assembly after a priming procedure.

FIG. 23C shows an example of a catheter assembly after a priming procedure including relative movement between adjacent catheters.

FIGS. 23D-23F illustrate the example catheter assembly of FIGS. 23A-23C.

FIG. 24 depicts a schematic of a control system.

FIG. 25A depicts a schematic of a control system.

FIG. 25B depicts a system diagram of a control system.

FIG. 26A depicts a first part of a system diagram of a control system.

FIG. 26B depicts a second part of a system diagram of the control system of FIG. 26A.

FIG. 27 depicts a system diagram of a tele-operated control system.

FIG. 28 depicts a schematic of a handshake workflow between a remote control system and a local control system.

FIG. 29 depicts a schematic of a network connection between a remote control system and a local control system, and a data center.

FIG. 30 depicts a schematic of a remote communication service that can be configured to transmit the one or more commands between a remote control system and a local control system.

FIG. 31 depicts a schematic implementing an adaptive streaming algorithm.

FIG. 32A depicts a perspective view of a drive table.

FIG. 32B depicts a perspective view of the drive table of FIG. 32A with a drive assembly.

FIG. 32C depicts a perspective view of the drive table and drive assembly of FIG. 32B proximal to an access point.

FIG. 32D depicts a perspective view of the drive table and drive assembly of FIG. 32C.

FIG. 33A depicts a perspective view of a plurality of hub assemblies being mounted to a drive table.

FIG. 33B depicts a perspective view of a plurality of hub assemblies being mounted to a drive table.

FIG. 34A depicts a top view of a plurality of hub adapters aligning with a corresponding hub assembly.

FIG. 34B depicts a top view of a plurality of hub adapters aligning with a corresponding hub assembly.

FIG. 35A depicts a side view of a plurality of hub adapters aligning with a corresponding hub assembly.

FIG. 35B depicts a side view of a plurality of hub adapters aligning with a corresponding hub assembly.

FIG. 35C depicts a side view of a plurality of hub adapters aligning with a corresponding hub assembly.

FIG. 36 depicts a perspective view of a hub assembly.

FIG. 37A depicts a front view of a control mechanism for manipulating interventional devices driven by respective hub assemblies.

FIG. 37B depicts a top view of the control mechanism of FIG. 33A.

FIG. 37C depicts an example of a user interface for controlling interventional devices.

FIG. 38A is a schematic illustrating an example of a fluidics assembly (e.g., a disposable or at least partially recyclable fluidics assembly) that includes a cassette that couples to a pump station and further couples to a saline source, a contrast source, and a vacuum source, and also includes a plurality of fluid communication channels to provide saline, contrast, and vacuum to mounts, and to catheters that are coupled to the mounts.

FIG. 38B is a schematic illustrating an example of a fluidics assembly that includes a cassette, a splitter, and one or more mounts, a first tubing set coupled to the cassette and splitter, and a second tubing set coupled between the splitter and the one or more mounts, the first tubing set having channels for providing saline, contrast and vacuum to the splitter, and the second tubing set having groups of channels (or subchannels) providing saline, contrast, and vacuum to each of the one or more mounts, the first and second tubing set and the splitter also providing electrical connections between the cassette and one or more connected mounts.

FIG. 38C is a schematic illustrating an example of a cassette of the fluidics assembly, the cassette including a housing that can support, all or part of a saline subsystem, a contrast subsystem, and a vacuum subsystem.

FIG. 38D is a schematic illustrating an example of a fluidic system components in one or more mounts, which can be coupled to the second tubing set, this particular example including three mounts each having a fluidic connection for saline, contrast, and vacuum, and an electrical connection that includes one or more electrical leads, the electrical connection coupled to one or more sensors in the mounts to communicate signals from the one or more sensors to the cassette, and ultimately a station (e.g., a pump station) when the cassette is coupled to the station.

### DETAILED DESCRIPTION

In certain embodiments, a system is provided for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations, the system may additionally be configured to robotically gain intra-cranial vascular access and to perform an aspiration thrombectomy or other neuro vascular procedure.

A drive table can be positioned over or alongside the patient, and configured to axially advance, retract, and in some cases rotate and/or laterally deflect two or three or more different (e.g., concentrically or side by side oriented) intravascular devices. The hub is moveable along a path along the surface of the drive table to advance or retract the interventional device as desired. Each hub may also contain mechanisms to rotate or deflect the device as desired, and is connected to fluid delivery tubes (not shown) of the type conventionally attached to a catheter hub. Each hub can be in electrical communication with an electronic control system, either via hard wired connection, RF wireless connection or a combination of both.

Each hub is independently movable across the surface of a sterile field barrier membrane carried by the drive table. Each hub is releasably magnetically coupled to a unique drive carriage on the table side of the sterile field barrier. The drive system independently moves each hub in a proximal or distal direction across the surface of the barrier, to move the corresponding interventional device proximally or distally within the patient's vasculature.

The carriages on the drive table, which magnetically couple with the hubs to provide linear motion actuation, are universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

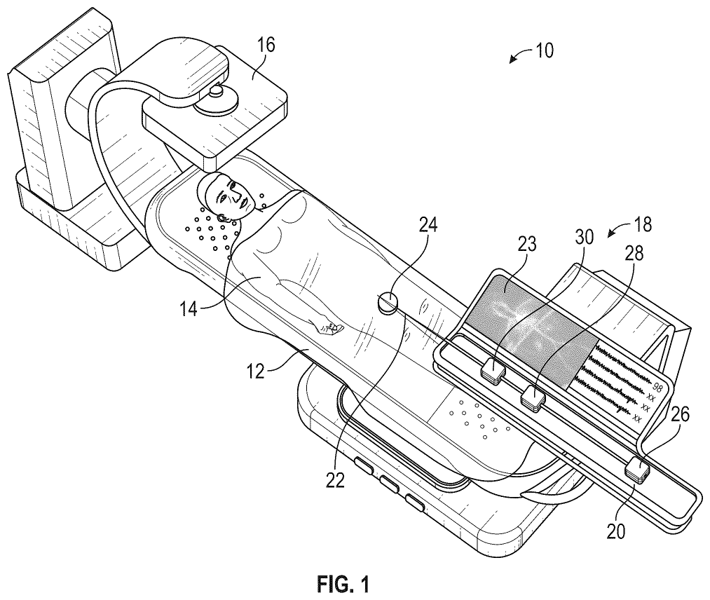

FIG. 1 is a schematic perspective view of an interventional setup 10 having a patient support table 12 for supporting a patient 14. An imaging system 16 may be provided, along with a robotic interventional device drive system 18 in accordance with the present disclosure.

The drive system 18 may include a support table 20 for supporting, for example, a guidewire hub 26, an access catheter hub 28 and a guide catheter hub 30. In the present context, the term ‘access’ catheter can be any catheter having a lumen with at least one distally facing or laterally facing distal opening, that may be utilized to aspirate thrombus, provide access for an additional device to be advanced therethrough or therealong, or to inject saline or contrast media or therapeutic agents.

More or fewer interventional device hubs may be provided depending upon the desired clinical procedure. For example, in certain embodiments, a diagnostic angiogram procedure may be performed using only a guidewire hub 26 and an access catheter hub 28 for driving a guidewire and an access catheter (in the form of a diagnostic angiographic catheter), respectively. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures).

A display 23 such as for viewing fluoroscopic images, catheter data (e.g., fiber Bragg grating fiber optics sensor data or other force or shape sensing data) or other patient data may be carried by the support table 20 and or patient support 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An access catheter hub 28 is also carried by the support table 20 and is movable along the table to advance the access catheter into and out of the patient 14. The access catheter hub may also be configured to rotate the access catheter in response to manipulation of a rotation control, and may also be configured to laterally deflect a deflectable portion of the access catheter, in response to manipulation of a deflection control.

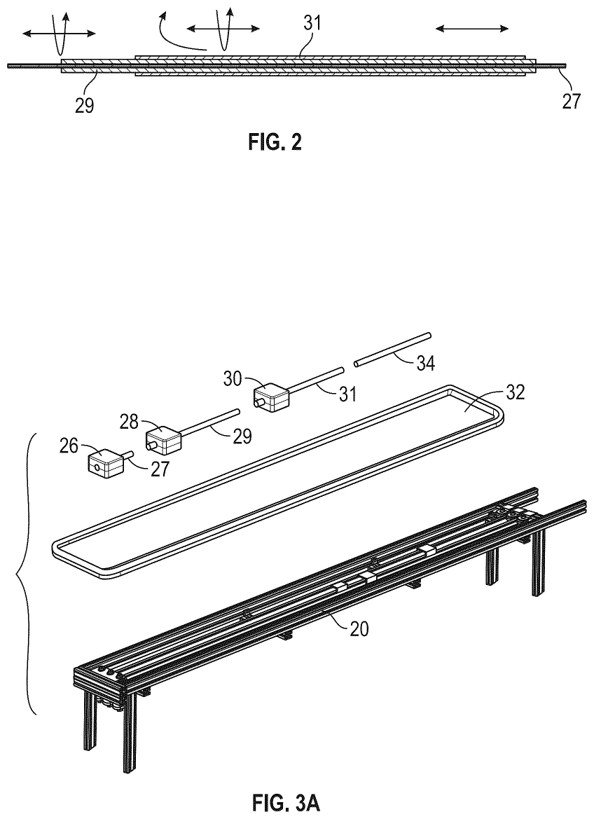

FIG. 2 is a longitudinal cross section schematically showing the motion relationship between a guidewire 27 having two degrees of freedom (axial and rotation), an access catheter 29 having three degrees of freedom (axial, rotational and lateral deflection) and a guide catheter 31, having one degree of freedom (axial).

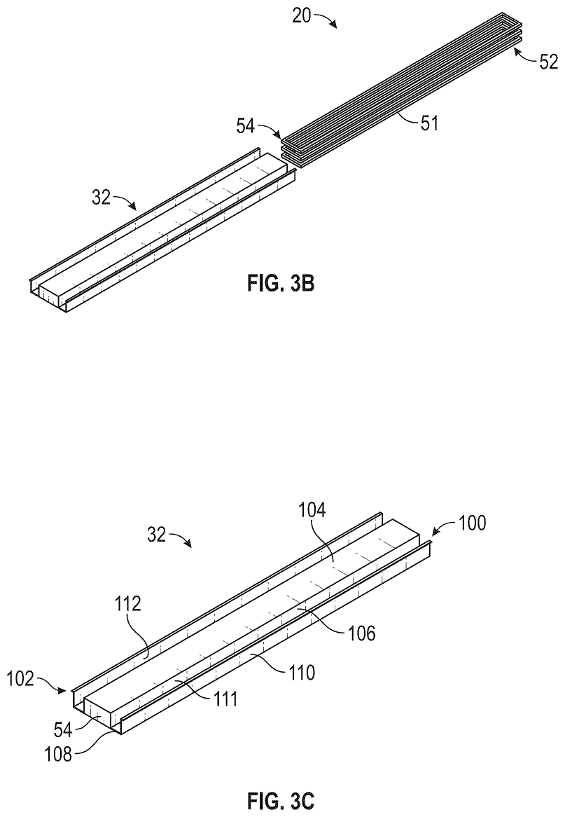

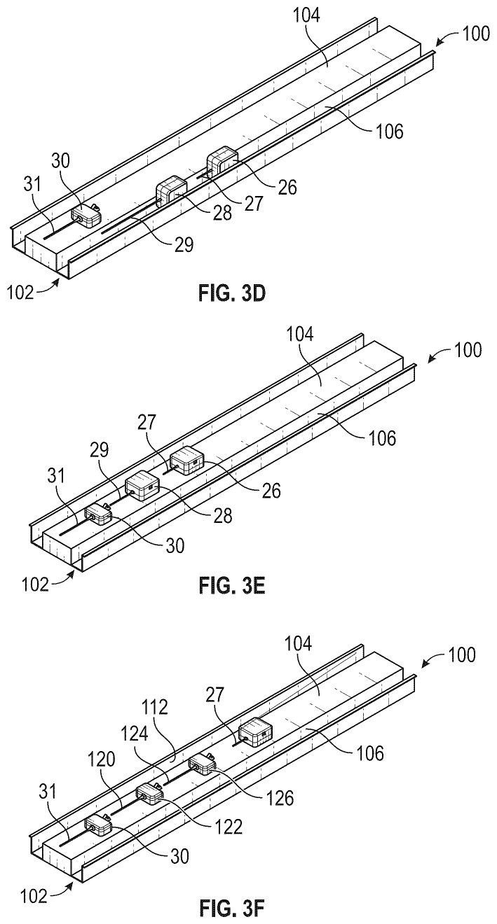

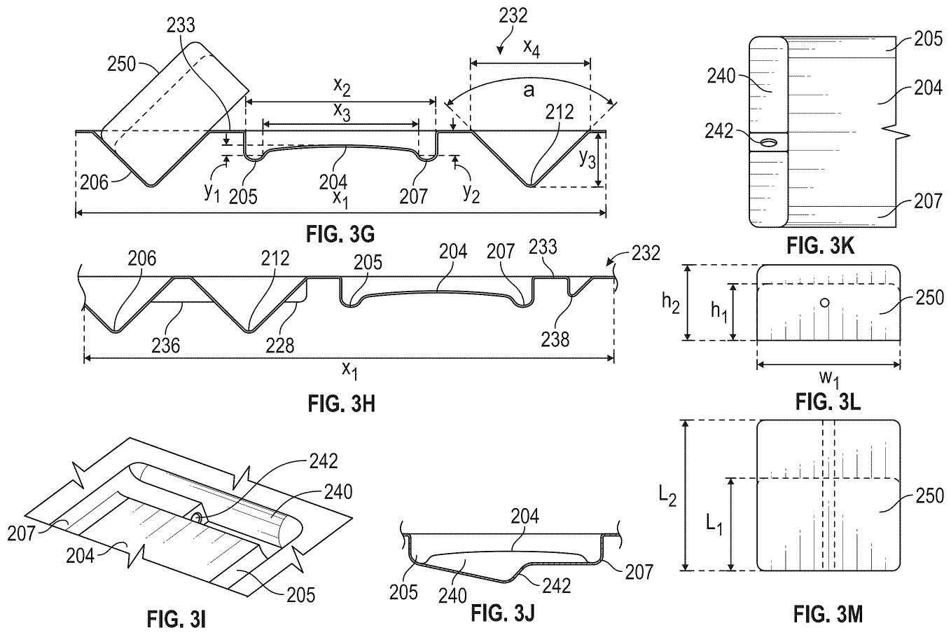

Referring to FIG. 3A, the support table 20 includes a drive mechanism described in greater detail below, to independently drive the guidewire hub 26, access catheter hub 28, and guide catheter hub 30. An anti-buckling feature 34 may be provided in a proximal anti-buckling zone for resisting buckling of the portion of the interventional devices spanning the distance between the support table 20 and the femoral artery access point 24. The anti-buckling feature 34 may include a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 centimeters or 10 centimeters but typically no more than about 120 centimeters or 100 centimeters to support the device between the hub and the access point 24 on the patient. Reinforcement may be accomplished by using metal or polymer tubing or embedding at least one or two or more axially extending elements into the wall of the device shafts, such as elongate wires or ribbons. In some implementations, the extending element may be hollow and protect from abrasion, buckling, or damage at the inputs and outputs of the hubs. In some embodiments, the hollow extending element may be a hollow and flexible coating attached to a hub. The hollow, extending element (e.g., a hollow and flexible coating) may cover a portion of the device shaft when threaded through the hubs. In some embodiments in which the hollow extending element is a coating, the coating may be attached to a portion of a hub such that threading the catheter device through the hub 26, 28, or 30 threads the catheter device through the coating as well. In some implementations, an anti-buckling device may be installed on or about or surrounding a device shaft to avoid misalignment or insertion angle errors between hubs or between a hub and an insertion point. The anti-buckling device may be a laser cut hypotube, a spring, telescoping tubes, tensioned split tubing, or the like.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts.

Alternatively, thin tubular stiffening structures can be embedded within or carried over the outside of the device wall, such as a tubular polymeric extrusion or length of hypo-tube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, a proximal segment of one or more of the device shafts may be constructed as a tubular hypo tube, which may be machined (e.g., with a laser) so that its mechanical properties vary along its length. This proximal segment may be formed of stainless steel, nitinol, and/or cobalt chrome alloys, optionally in combination with polymer components which may provide for lubricity and hydraulic sealing. In some embodiments, this proximal segment may be formed of a polymer, such as polyether ether ketone (PEEK). Alternatively, the wall thickness or diameter of the interventional device can be increased in the anti-buckling zone.

In certain embodiments, a device shaft having advanced stiffness (e.g., axially and torsionally) may provide improved transmission of motion from the proximal end of the device shaft to the distal end of the device shaft. For example, the device shafts may be more responsive to motion applied at the proximal end. Such embodiments may be advantageous for robotic driving in the absence of haptic feedback to a user.

In some embodiments, a flexible coating can be applied to a device shaft and/or hub to reduce frictional forces between the device shaft and/or hub and a second device shaft when the second device shaft passes therethrough.

The interventional device hubs may be separated from the support table 20 by sterile barrier 32. Sterile barrier 32 may include a thin plastic membrane such as polyethylene terephthalate (PET), polyethylene terephthalate glycol (PETG), polyethylene terephthalate (PETE), high-density polyethylene (HDPE), polyvinyl chloride (PVC), low-density polyethylene (LDPE), polypropylene (PP), polystyrene (PS), styrene, fluoropolymers such as polytetrafluoroethylene (PTFE), expanded PTFE such as GORE-TEX®, fluorinated ethylene propylene (FEP), synthetic flashspun high-density polyethylene fibers such as Tyvek®, or composites such as fiberglass and/or carbon fiber reinforced epoxy. This allows the support table 20 and associated drive system to reside on a non-sterile (lower) side of sterile barrier 32. The guidewire hub 26, access catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices. The sterile barrier may also act as a sterile work surface for preparation of catheters or other devices during a procedure.

Referring to FIGS. 3B and 3C, there is illustrated a sterile barrier 32 in the form of a pre-shaped tray, for fitting over an elongate support table 20. In use, the elongate support table 20 would be positioned below the sterile barrier 32. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration.

The length of support surface 104 will typically be at least about 100 centimeters and within the range of from about 100 centimeters to about 2.7 meters. Shorter lengths may be utilized in a system configured to advance the drive couplers along an arcuate path. In some embodiments, two or more support surfaces may be used instead of a single support surface 104. The two or more support surfaces may have a combined length between 100 centimeters to about 2.7 meters. The width of the linear drive table is preferably no more than about 30 to about 80 centimeters.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs (for example, by providing lateral support to prevent dislodgment of the hubs when forces are applied to the hubs). First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure as well as to collect fluids and function as wash basins for catheters and related devices.

Referring to FIG. 3D, the guide catheter hub 30 is shown positioned on the upper support surface 104, and magnetically coupled to the corresponding coupler holding the drive magnets, positioned beneath the sterile barrier 32. The access catheter hub 28 and access catheter 29, and guidewire hub 26 and guidewire 27 are illustrated residing within the first channel 106 such as before introduction through the guide catheter 31 or following removal from the guide catheter 31.

The interventional devices may be positioned within the channel 106 and enclosed in a sterile barrier for shipping. At the clinical site, an upper panel of the sterile barrier may be removed, or a tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E, the interventional devices configured in an access assembly for aortic access, following coupling of the access assembly to the corresponding carriages beneath the sterile barrier. The access assembly may be preassembled with the guidewire fully advanced through the access catheter which is in turn fully advanced through the guide catheter. In embodiments in which the access catheter or other catheters are pre-shaped (i.e., pre-curved or not straight), the guidewire and/or outer catheters may be positioned so that relatively stiff sections are not superimposed with curved stiffer sections of the pre-shaped catheter, for example, to avoid creep or straightening of the pre-shaped catheter and/or introduction of a curve into an otherwise straight catheter. This access assembly may be lifted out of the channel 106 and positioned on the support surface 104 for coupling to the respective drive magnets and introduction into the patient. The guide catheter hub 30 is the distal most hub. Access catheter hub 28 is positioned proximally of the guide catheter hub, so that the access catheter 29 can extend distally through the guide catheter. The guidewire hub 26 is positioned most proximally, in order to allow the guidewire 27 to advance through the access catheter 29 and guide catheter 31.

A procedure assembly is illustrated in FIG. 3F following introduction of the procedure assembly through the guide catheter 31 that was used to achieve supra-aortic access. In this implementation, guide catheter 31 remains the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guidewire 27 extends through at least a portion of the second procedure catheter 124 in a rapid exchange version of second procedure catheter 124, or the entire length of second procedure catheter 124 in an over the wire implementation.

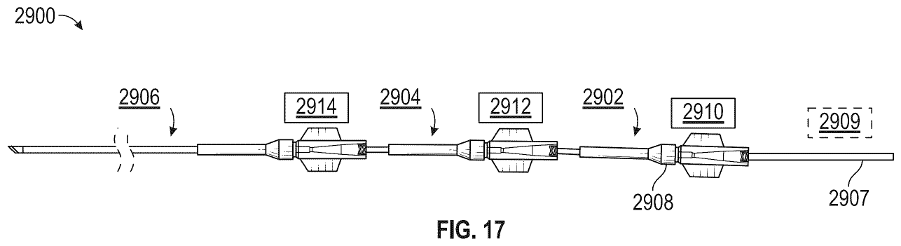

As is discussed in greater detail in connection with FIG. 17, the multi-catheter stack may be utilized to achieve both access and the intravascular procedure without the need for catheter exchange. this may be accomplished in either a manual or a robotically driven procedure. In one example, the guide catheter 31 may include a catheter having an inner diameter of at least about 0.08 inches and in one implementation about 0.088 inches. The first procedure catheter 120 may include a catheter having an inner diameter within the range of from about 0.065 inches to about 0.075 inches and in one implementation catheter 120 has an inner diameter of about 0.071 inches. The second procedure catheter 124 may be an access catheter having an OD sized to permit advance through the first procedure catheter 120. The second procedure catheter maybe steerable, having a deflection control 2908 configured to laterally deflect a distal end of the catheter. The second procedure (access) catheter may also have an inner lumen sized to allow an appropriately sized guidewire to remain inside the second procedure catheter while performing contrast injections through the second procedure catheter.

In certain embodiments, the catheter 31 may be a ‘large bore’ access catheter or guide catheter having a diameter of at least about 0.075 or at least about 0.080 inches in diameter. The catheter 120 may be an aspiration catheter having a diameter within the range of from about 0.060 to about 0.075 inches. The catheter 124 may be a steerable catheter with a deflectable distal tip, having a diameter within the range of from about 0.025 to about 0.050 inches. The guidewire 27 may have a diameter within the range of from about 0.014 to about 0.020 inches. In one example, the catheter 31 may have a diameter of about 0.088 inches, the catheter 120 about 0.071 inches, the catheter 124 about 0.035 inches, and the guidewire 27 may have a diameter of about 0.018 inches.

In one commercial execution, a preassembled access assembly (guide catheter, access catheter and guidewire) may be carried within a first channel on the sterile barrier tray and a preassembled procedure assembly (one or two procedure catheters and a guidewire) may be carried within the same or a different, second channel on the sterile barrier tray. One or two or more additional catheters or interventional tools may also be provided, depending upon potential needs during the interventional procedure.

FIGS. 3G-3K illustrate embodiments of an alternate sterile barrier having a convex drive surface (e.g., a convex, crowned road like drive surface). FIG. 3G is a cross-sectional view of a sterile barrier 232. The sterile barrier 232 includes a convex upper support surface 204. Fluid channels 205 and 207 are positioned laterally of and below the support surface 204 for self-clearing or draining of fluids from the support surface 204 (for example, during an interventional procedure). The fluid channels 205 and 207 may extend axially at least a portion of the length of the sterile barrier.

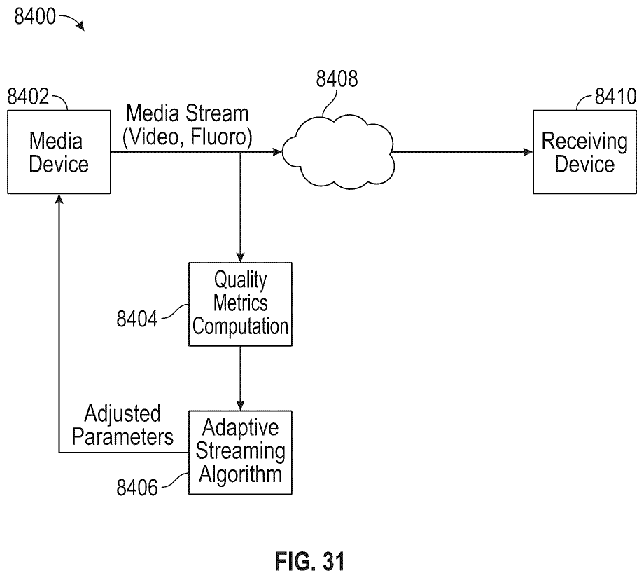

FIGS. 31, 3J, and 3K illustrate a sectional perspective view, a cross-sectional view, and a top sectional view, respectively, of a proximal end of the sterile barrier 232. As shown, in FIGS. 31-3K, the sterile barrier 232 can include a trough 240 in communication with the fluid channels 205 and 207. The trough 240 can receive fluids from the channels 205 and 207 (for example, during an interventional procedure). The trough 240 may be positioned at least partially below the fluid channels 205 and 207 so that fluid within the channels 205 and 207 flows into the trough 240. In certain embodiments, the fluid channels 205 and 207 may be angled relative to a horizontal plane (for example, may decline from an end of the channel furthest from the trough 240 to the trough 240) so that fluid within the channels 205 and 207 is directed to the trough 240. For example, the channels 205 and 207 may increase in depth from an end of the channels furthest from the trough 240 to the trough 240. Alternatively, the sterile barrier 232 and/or support table may be positioned at an angle relative to a horizontal plane, during part of or an entirety of an interventional procedure, such that the end of the channels 205 and 207 furthest from the trough 240 is positioned higher than the trough 240. For example, the sterile barrier 232 and/or support table may be constructed or arranged in an angled arrangement so that an end of the sterile barrier 232 and/or support table opposite the trough 240 is positioned higher than the trough 240. Alternatively or additionally, a drive mechanism may temporarily tilt the sterile barrier 232 and/or support table so that an end of the sterile barrier 232 and/or support table opposite the trough 240 is positioned higher than the trough 240 (for example, by lifting an end of the sterile barrier and/or support table opposite the trough 240 or lowering an end of the sterile barrier 232 and/or support table at which the trough 240 is positioned) so that fluids within the channels 205 and 207 flow into the trough 240.

The trough 240 can include a drain hole 242. The trough 240 can be shaped, dimensioned, and/or otherwise configured so that fluid within the trough 240 empties to the drain hole 242. The drain hole 242 can include tubing, a barb fitting, and/or an on-off valve for removal of fluids from the trough 240. As shown in FIGS. 31-3K, the trough 240 can be positioned at the proximal end of the sterile barrier 232. In alternate embodiments, the trough 240 may be positioned at a distal end of the sterile barrier 232. In some embodiments, the sterile barrier 232 can include a first trough 240 at the proximal end and a second trough 240 at the distal end. In some embodiments, the trough 240 can also be used as a wash basin.

A first channel 206 may extend axially at least a portion of the length of the sterile barrier 232. The channel 206 can have a sufficient length to hold the interventional devices, and sufficient width and depth to hold the corresponding hubs (for example, by providing support to prevent dislodgement of the hubs when forces are applied to the hubs). Optionally, a second channel 212 may be provided. The second channel 212 may be located on the same side or the opposite side of the upper support surface 204 from the first channel 206. FIG. 3G illustrates the channel 212 located on the opposite side of the support surface 204 from the channel 206. FIG. 3H is a cross-sectional view illustrating an alternate embodiment of the sterile barrier 232 in which the channel 212 is on the same side of the support surface 204 as the channel 206.

As shown in FIGS. 3G and 3H, the channels 206 and 212 can have generally triangular, wedge-shaped, or otherwise angled cross-sections, so as to hold the hubs at an angle relative to a horizontal plane. Holding the hubs at an angle relative to the horizontal plane can allow for smaller width of the sterile barrier 232.

Two or three or more additional recesses such as additional channels or wells may be provided, to hold additional medical devices or supplies that may be useful during the interventional procedure as well as to collect fluids and function as wash basins for catheters and related devices.

In some embodiments, the sterile barrier 232 can include one or more structural ribs 236. The sterile barrier 232 can further include one or more frame support bosses 228 and 238.

In the embodiment of the sterile barrier 232 shown in FIG. 3G, a width x₁ can be 14 in, about 14 in, between 12 in and 16 in, between 10 in and 18 in, or any other suitable width. In the embodiment of the sterile barrier 232 shown in FIG. 3H, the width x₁ can be 15 in, about 15 in, between 13 in and 17 in, between 11 in and 19 in, or any other suitable width. A height y₁ of the support surface 204 can be 0.125 in, about 0.125 in, between 0.1 and 0.15 in, or any other suitable height. In some embodiments, the support surface 204 can be recessed from a top surface 233 of the sterile barrier 232. A height y₂ between a bottom of the support surface 204 and the top surface 233 can be 0.5 in, about 0.5 in, between 0.25 in and 0.75 in, or any other suitable height. A width x₂ from a lateral edge of the channel 205 to a lateral edge of the channel 207 can be 5 in, about 5 in, between 4 in and 6 in, or any other suitable width. A width x₃ of the support surface 204 can be 4 in, about 4 in, between 3 in and 5 in, or any other suitable width. A height y₃ of the channel 206 and/or channel 212 can be 1.5 in, about 1.5 in, between 1 in and 2 in, or any other suitable height. A width x₄ of the channel 206 and/or channel 212 can be 3 in, about 3 in, between 2 in and 4 in, or any other suitable width. The channel 206 and/or channel 212 can be defined by an arc angle α of 90°, about 90°, between 80° and 100°, or any other suitable angle, and a radius of curvature of 0.125 in, about 0.125 in, between 0.1 and 0.15 in, or any other suitable radius of curvature. In certain embodiments, an arc angle α of 90° or about 90° may be used to hold a hub having a rectangular or generally rectangular cross-section. The support surface 204 can be defined by a radius of curvature of 13 in, about 13 in, between 11 in and 15 in, or any other suitable radius of curvature. The channel 205 and/or channel 207 can be defined by a radius of curvature of 0.25 in, about 0.25 in, between 0.15 in and 0.35 in, or any other suitable radius of curvature.

FIGS. 3L and 3M depict example dimensions of a hub 250 that may be used with the sterile barrier 232 as shown in FIGS. 3G-3K. The hub 250 may be any of the hubs described herein. In certain embodiments, the hub 250 can have a width w₁ of 3.75 in, about 3.75 in, between 3.25 in and 4.25 in, or any other suitable width. The hub 250 can have a height h1 of 1.5 in, about 1.5 in, between 1.25 in and 1.75 in, or any other suitable height. Alternatively, the hub 250 can have a height h₂ of 2 in, about 2 in, between 1.75 in and 2.25 in, or any other suitable height. In some embodiments, the hub 250 can have a length L₁ of 2.5 in, about 2.5 in, between 2 in and 3 in or any other suitable length. Alternatively, the hub 250 can have a length L₂ of 4 in, about 4 in, between 3.25 in and 4.75 in, or any other suitable length.

In some embodiments, a top surface of the support table can include surface features that generally correspond to those of the sterile barrier 232. For example, the support table can include a convex surface configured to correspond to the shape, size, and location of the support surface 204 and/or one or more recesses configured to correspond to the shape, size, and location of the channels 205 and 207.

In alternate embodiments, a planar support surface (for example, support surface 104 of sterile barrier 32) can be positioned at an angle to a horizontal plane to facilitate the draining of fluids. In some embodiments, the sterile barrier and/or support table may be positioned, during part of or the entirety of an interventional procedure, at an angle to a horizontal plane to facilitate the draining of fluids. For example, the sterile barrier and/or support table may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the sterile barrier and/or support table (for example, so that one lateral side of the planar support surface is positioned higher than the other lateral side of the planar support surface, the proximal end is higher than the distal end, or the distal end is higher than the proximal end) to facilitate the drainage of fluids. For example, the drive mechanism may raise or lower one lateral side of the sterile barrier and/or support table, the proximal end of the sterile barrier and/or support table, and/or the distal end of the sterile barrier and/or support table.

In certain embodiments, a support surface (for example, support surface 104 of sterile barrier 32) can be positioned in a vertical configuration instead in the horizontal configuration shown, for example, in FIGS. 3A-3F. For example, the support surface 104 can be positioned at about 90 degrees (or any other suitable angle) from a horizontal plane (e.g., rotated 90 degrees about a long axis of the support surface 104 relative to the embodiment shown in of FIGS. 3A-3F). A vertical configuration may provide for easier interaction with the drive system 18 by a physician, A vertical configuration may also provide for a lower axis of catheter travel closer to a patient without adding standoff height to the drive system 18.

In some embodiments, the drive system 18 may be positioned, during part of or the entirety of an interventional procedure, at an angle to a horizontal plane to facilitate the draining of fluids. For example, the drive system 18 may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the drive system 18 (for example, so that one lateral side of the drive system 18 is positioned higher than the other lateral side of the drive system 18, the proximal end is higher than the distal end, or the distal end is higher than the proximal end) to facilitate the drainage of fluids. For example, the drive mechanism may raise or lower one lateral side of the system 18, the proximal end of the drive system 18, and/or the distal end of the drive system 18. In some embodiments, the drive system 18 may be angled so that it extends at an angle away from axis point 24 (for example, so that the proximal end is higher than the distal end), for example, to allow for clearance of a patient's feet.

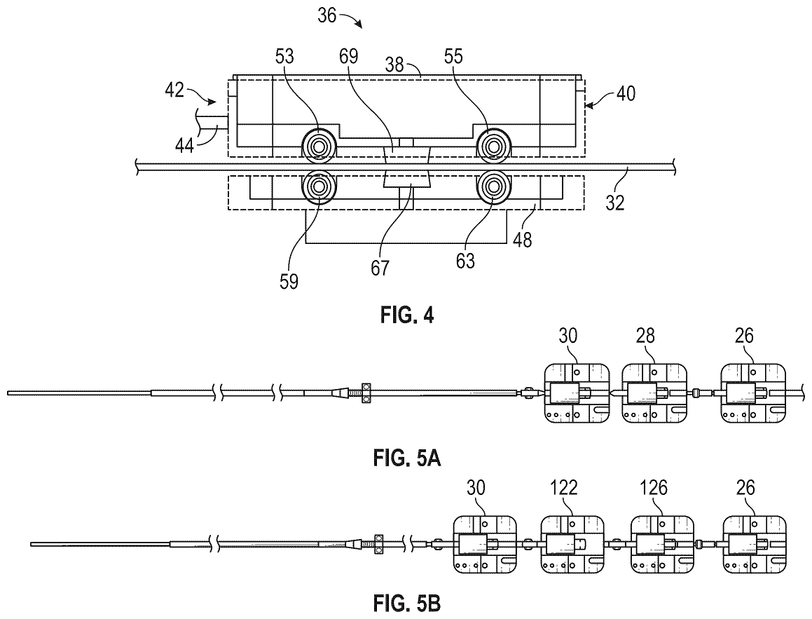

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 or carriage acts as a shuttle by advancing proximally or distally along a track in response to operator instructions or control device manipulations. The hub adapter 48 includes at least one drive magnet 67 configured to couple with a driven magnet 69 carried by the hub 36. This provides a magnetic coupling between the drive magnet 67 and driven magnet 69 through the sterile barrier such that the hub 36 is moved across the top of the sterile barrier 32 in response to movement of the hub adapter 48 outside of the sterile field. Movement of the hub adapter is driven by a drive system carried by the support table and described in additional detail below. The hub adapter may act as a robotic drive for an interventional device coupled thereto.

To reduce friction in the system, the hub 36 may be provided with at least a first roller 53 and a second roller 55 which may be in the form of wheels or rotatable balls or drums. The rollers space the sterile barrier apart from the surface of the driven magnet 69 by at least about 0.02 centimeters (about 0.008 inches) and generally no more than about 0.08 centimeters (about 0.03 inches). In some implementations, the space is within the range of from about 0.03 centimeters (about 0.010 inches) and about 0.041 centimeters (about 0.016 inches). The space between the drive magnet 67 and driven magnet 69 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 48 may similarly be provided with at least a first hub adapter roller 59 and the second hub adapter roller 63, which may be positioned opposite the respective first roller 53 and second roller 55 as illustrated in FIG. 4.

Referring to FIG. 6, there is schematically illustrated one example of a low-profile linear drive support table 20. Support table 20 includes an elongated frame 51 extending between a proximal end 52 and a distal end 54. At least one support table support 56 is provided to stabilize the support table 20 with respect to the patient (not illustrated). Support 56 may include one or more legs or preferably an articulating arm configured to allow movement and positioning of the frame 51 over or adjacent to the patient.

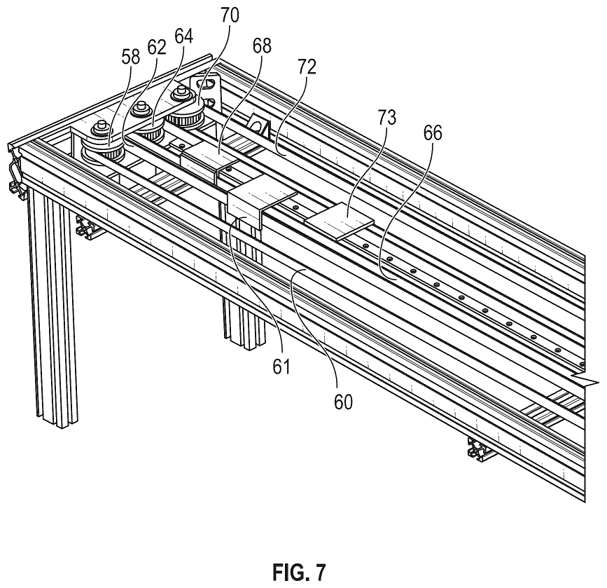

One example of a linear drive table 20 illustrated in FIG. 7 includes three distinct drives. However, two drives or four or more drives (e.g., up to eight drives) may be included depending upon the desired clinical performance. A first drive pulley 58 engages a first drive belt 60. A first carriage bracket 61 is secured to the first drive belt 60 such that rotation of the first drive pulley 58 causes rotation of the first drive belt 60 through an elongate closed loop path. The first carriage bracket 61 may be advanced in a proximal or distal direction along the longitudinal axis of the support table 20 depending upon the direction of rotation of the drive pulley 58. In the illustrated implementation, the drive pulley 58 is provided with surface structures such as a plurality of drive pulley teeth 62 for engaging complementary teeth on the first drive belt 60.

A second drive pulley 64 may engage a second drive belt 66 configured to axially move a second carriage bracket 68 along an axial path on the support table 20. A third drive pulley 70 may be configured to drive a third drive belt 72, to advance a third carriage bracket 73 axially along the support table 20. Each of the carriage brackets may be provided with a drive magnet assembly discussed previously but not illustrated in FIG. 7, to form couplers for magnetically coupling to a corresponding driven magnet within the hub of an interventional device as has been discussed.

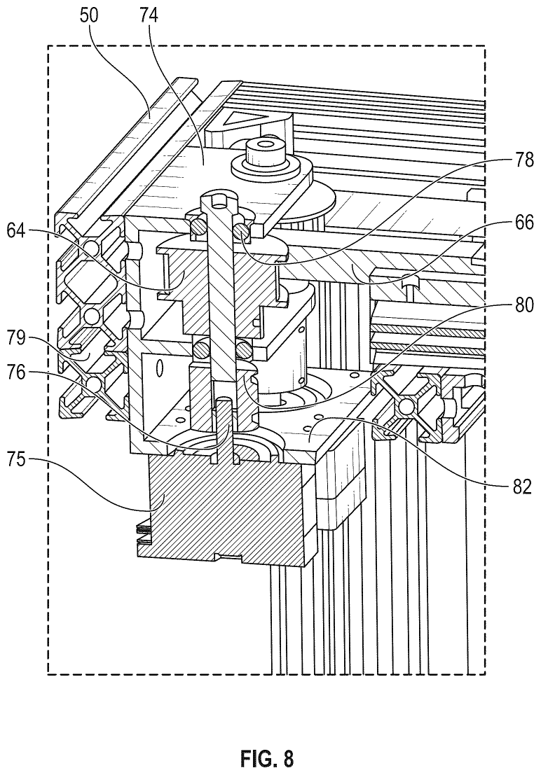

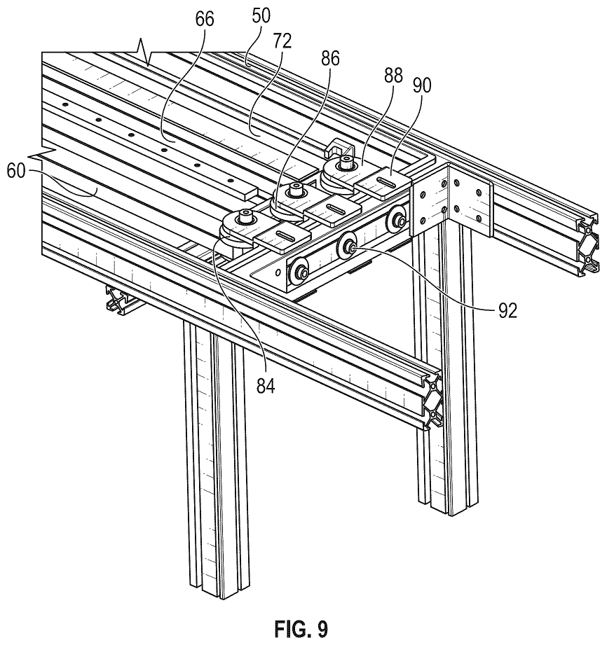

A detailed view of a drive system is shown schematically in FIG. 8. A drive support 74 may be carried by the frame 51 for supporting the drive assembly. The second drive pulley 64 is shown in elevational cross section as rotationally driven by a motor 75 via a rotatable shaft 76. The rotatable shaft 76 may be rotatably carried by the support 74 via a first bearing 78, a shaft coupling 80 and second bearing 79. Motor 75 may be stabilized by a motor bracket 82 connected to the drive support 74 and or the frame 51. The belt drive assemblies for the first drive belt 60 and third drive belt 72 maybe similarly constructed and are not further detailed herein. In some embodiments, the drive systems described herein may be a rack and pinion drive table system that is foldable. In such embodiments, motors 75 may be attached to and move with the carriages.

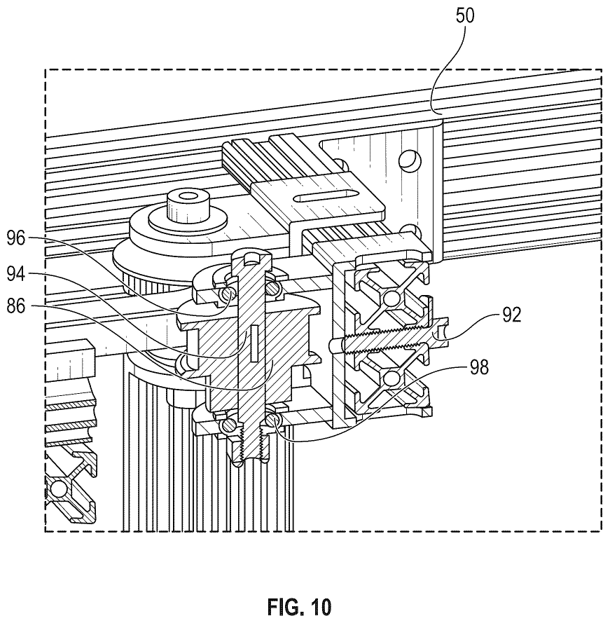

Referring to FIGS. 9 and 10, each of the first second and third drive belts extends around a corresponding first idler pulley 84 second idler pulley 86 and third idler pulley 88. Each idler pulley may be provided with a corresponding tensioning bracket 90, configured to adjust the idler pulleys in a proximal or distal direction in order to adjust the tension of the respective belt. Each tensioning bracket 90 is therefore provided with a tensioning adjustment 92 such as a rotatable screw.

As seen in FIG. 10, the second idler pulley 86, for example, may be carried by a rotatable shaft 94, rotatably secured with respect to the mounting bracket by a first bearing 96 and second bearing 98.

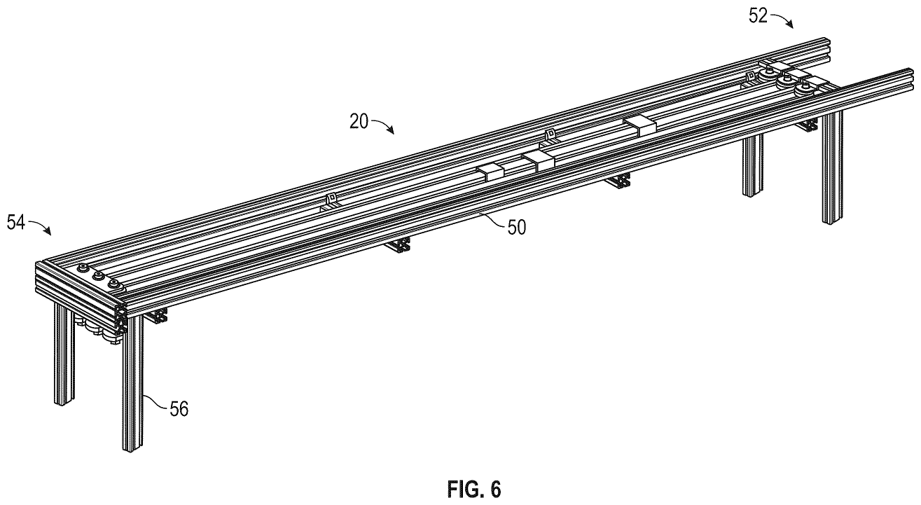

Any of the catheters illustrated, for example, in FIG. 5A, 5B or 11 generally include an elongate tubular body extending between a proximal end and a distal functional end. The length and diameter of the tubular body depends upon the desired application. For example, lengths in the area of from about 90 centimeters to about 195 centimeters or more are typical for use in femoral access percutaneous transluminal coronary applications. Intracranial or other applications may call for a different catheter shaft length depending upon the vascular access site.

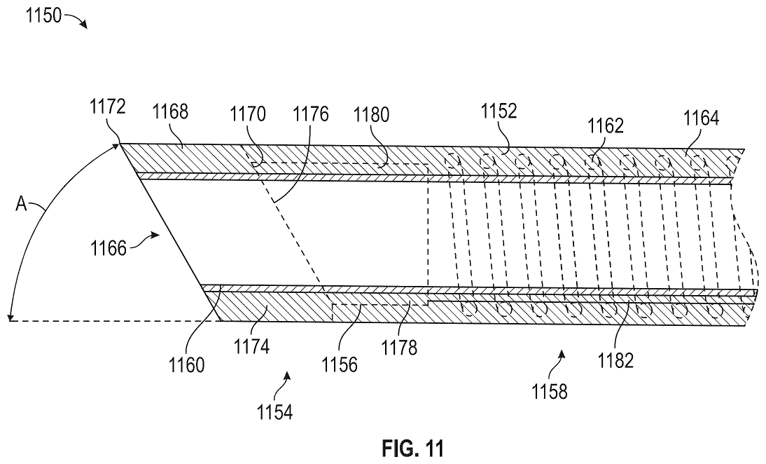

Any of the catheters disclosed herein may be provided with an inclined distal tip. Referring to FIG. 11, distal catheter tip 1150 includes a tubular body 1152 which includes an advance segment 1154, a marker band 1156 and a proximal segment 1158. An inner tubular liner 1160 may extend throughout the length of the distal catheter tip 1150, and may include dip coated or extruded PTFE or other lubricious material.

A reinforcing element 1162 such as a braid and/or spring coil is embedded in an outer tubular jacket 1164 which may extend the entire length of the catheter.

The advance segment 1154 terminates distally in an angled face 1166, to provide a leading side wall portion 1168 having a length measured between the distal end 130 of the marker band 1156 and a distal tip 1172. In some embodiments, the entire distal tip may be shaped to avoid snagging the tip in areas of arterial bifurcation. A trailing side wall portion 1174 of the advance segment 1154, has an axial length in the illustrated embodiment of approximately equal to the axial length of the leading side wall portion 1168 as measured at approximately 180 degrees around the catheter from the leading side wall portion 1168. The leading side wall portion 1168 may have an axial length within the range of from about 0.1 millimeters to about 5 millimeters and generally within the range of from about 1 to 3 millimeters. The trailing side wall portion 1174 may be equal to or at least about 0.1 or 0.5 or 1 millimeter or 2 millimeters or more shorter than the axial length of the leading side wall portion 1168, depending upon the desired performance.

The angled face 1166 inclines at an angle A within the range of from about 45 degrees to about 80 degrees from the longitudinal axis of the catheter. For certain implementations, the angle is within the range of from about 55 degrees to about 65 degrees from the longitudinal axis of the catheter. In one implementation, the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105 percent, and no more than about 130 percent, in some implementations within the range of from about 110 percent and about 125 percent, and in one example is about 115 percent of the area of the corresponding circular port (angle A is 90 degrees).

In the illustrated embodiment, the axial length of the advance segment is substantially constant around the circumference of the catheter, so that the angled face 1166 is approximately parallel to the distal surface 1176 of the marker band 1156. The marker band 1156 has a proximal surface approximately transverse to the longitudinal axis of the catheter, producing a marker band 1156 having a right trapezoid configuration inside elevational view. A short sidewall 1178 is rotationally aligned with the trailing side wall portion 1174, and has an axial length within the range of from about 0.2 millimeters to about 4 millimeters, and typically from about 0.5 millimeters to about 2 millimeters. An opposing long sidewall 1180 is rotationally aligned with the leading side wall portion 1168. Long sidewall 1180 of the marker band 1156 is generally at least about 10 percent or 20 percent longer than short sidewall 1178 and may be at least about 50 percent or 70 percent or 90 percent or more longer than short sidewall 1178, depending upon desired performance. Generally, the long sidewall 1180 will have a length of at least about 0.5 millimeters or 1 millimeter and less than about 5 millimeters or 4 millimeters.

The marker band may be a continuous annular structure, or may have at least one and optionally two or three or more axially extending slits throughout its length. The slit may be located on the short sidewall 1178 or the long sidewall 1180 or in between, depending upon desired bending characteristics. The marker band may include any of a variety of radiopaque materials, such as a platinum/iridium alloy, with a wall thickness preferably no more than about 0.003 inches and in one implementation is about 0.001 inches.

The fluoroscopic appearance of the marker bands may be unique or distinct for each catheter size or type when a plurality of catheters is utilized so that the marker bands can be distinguishable from one another by a software algorithm. Distinguishing the marker bands of a plurality of catheters may be advantageous when the multiple catheters are used together, for example, in a multi-catheter assembly or stack as described herein. In some embodiments, the marker band of a catheter may be configured so that a software algorithm can detect motion of the catheter tip.

The marker band zone of the assembled catheter may have a relatively high bending stiffness and high crush strength, such as at least about 50 percent or at least about 100 percent less than proximal segment 18 but generally no more than about 200 percent less than proximal segment 1158. The high crush strength may provide radial support to the adjacent advance segment 1154 and particularly to the leading side wall portion 1168, to facilitate the functioning of distal tip 1172 as an atraumatic bumper during transluminal advance and to resist collapse under vacuum. The proximal segment 1158 preferably has a lower bending stiffness than the marker band zone, and the advance segment 1154 preferably has even a lower bending stiffness and crush strength than the proximal segment 1158.

The advance segment 1154 may include a distal extension of the outer tubular jacket 1164 and optionally the inner tubular liner 1160, without other internal supporting structures distally of the marker band 1156. Outer tubular jacket 1164 may include extruded polyurethane, such as Tecothane®. The advance segment 1154 may have a bending stiffness and radial crush stiffness that is no more than about 50 percent, and in some implementations no more than about 25 percent or 15 percent or 5 percent or less than the corresponding value for the proximal segment 1158.

The catheter may further include an axial tension element or support such as a ribbon or one or more filaments or fibers for increasing the tension resistance and/or influencing the bending characteristics in the distal zone. The tension support may include one or more axially extending mono strand or multi strand filaments. The one or more tension element 1182 may be axially placed inside the catheter wall near the distal end of the catheter. The one or more tension element 1182 may serve as a tension support and resist tip detachment or elongation of the catheter wall under tension (e.g., when the catheter is being proximally retracted through a kinked outer catheter or tortuous or narrowed vasculature).

At least one of the one or more tension element 1182 may proximally extend along the length of the catheter wall from within about 1.0 centimeters from the distal end of the catheter to less than about 10 centimeters from the distal end of the catheter, less than about 20 centimeters from the distal end of the catheter, less than about 30 centimeters from the distal end of the catheter, less than about 40 centimeters from the distal end of the catheter, or less than about 50 centimeters from the distal end of the catheter.

The one or more tension element 1182 may have a length greater than or equal to about 40 centimeters, greater than or equal to about 30 centimeters, greater than or equal to about 20 centimeters, greater than or equal to about 10 centimeters, or greater than or equal to about 5 centimeters.

At least one of the one or more tension element 1182 may extend at least about the most distal 50 centimeters of the length of the catheter, at least about the most distal 40 centimeters of the length of the catheter, at least about the most distal 30 centimeters or 20 centimeters or 10 centimeters of the length of the catheter.

In some implementations, the tension element extends proximally from the distal end of the catheter along the length of the coil 24 and ends proximally within about 5 centimeters or 2 centimeters or less either side of a transition between a distal coil and a proximal braid. The tension element may end at the transition without overlapping with the braid.

The one or more tension element 1182 may be placed near or radially outside the inner tubular liner 1160. The one or more tension element 1182 may be placed near or radially inside the braid and/or the coil. The one or more tension element 1182 may be carried between the inner tubular liner 1160 and the helical coil, and may be secured to the inner liner or other underlying surface by an adhesive prior to addition of the next outer adjacent layer such as the coil. Preferably, the tension element 1182 is secured to the marker band 1156 such as by adhesives or by mechanical interference. In one implementation, the tension element 1182 extends distally beyond the marker band on a first (e.g., inside) surface of the marker band, then wraps around the distal end of the marker band and extends along a second (e.g., outside) surface in either or both a proximal inclined or circumferential direction to wrap completely around the marker band.

When more than one tension element 1182 or filament bundles are spaced circumferentially apart in the catheter wall, the tension elements 1182 may be placed in a radially symmetrical manner. For example, the angle between two tension elements 1182 with respect to the radial center of the catheter may be about 180 degrees. Alternatively, depending on desired clinical performances (e.g., flexibility, trackability), the tension elements 1182 may be placed in a radially asymmetrical manner. The angle between any two tension elements 1182 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 1182 may include materials such as Vectran®, Kevlar®, Polyester®, Spectra®, Dyneema®, Meta-Para-Aramide®, or any combinations thereof. At least one of the one or more tension element 1182 may include a single fiber or a multi-fiber bundle, and the fiber or bundle may have a round or rectangular (e.g., ribbon) cross section. The terms fiber or filament do not convey composition, and they may include any of a variety of high tensile strength polymers, metals or alloys depending upon design considerations such as the desired tensile failure limit and wall thickness. The cross-sectional dimension of the one or more tension element 1182, as measured in the radial direction, may be no more than about 2 percent, 5 percent, 8 percent, 15 percent, or 20 percent of that of the catheter 10.

The cross-sectional dimension of the one or more tension element 1182, as measured in the radial direction, may be no more than about 0.03 millimeters (about 0.001 inches), no more than about 0.0508 millimeters (about 0.002 inches), no more than about 0.1 millimeters (about 0.004 inches), no more than about 0.15 millimeters (about 0.006 inches), no more than about 0.2 millimeters (about 0.008 inches), or about 0.38 millimeters (about 0.015 inches).

The one or more tension element 1182 may increase the tensile strength of the distal zone of the catheter before failure under tension (e.g., marker band detachment) to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

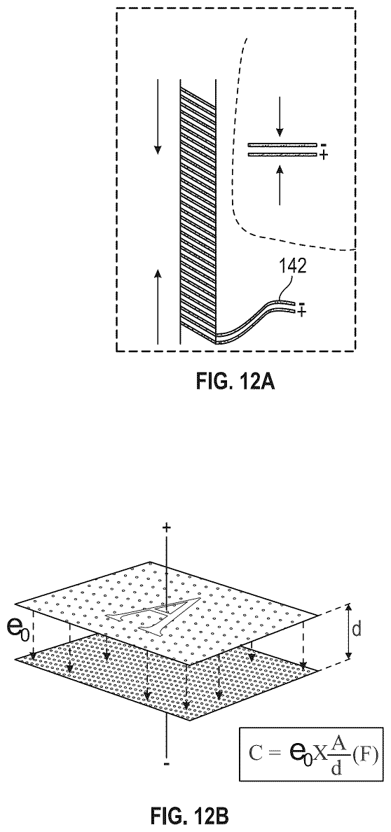

Any of a variety of sensors may be provided on any of the catheters, hubs, carriages, or table, depending upon the desired data. For example, in some implementations, it may be desirable to measure axial tension or compression force applied to the catheter such as along a force sensing zone. The distal end of the catheter would be built with a similar construction as illustrated in FIG. 11, with a helical coil distal section. But instead of using a single helical coil of nitinol wire, a first conductor 140 and second conductor 142 are wrapped into intertwined helical coils and electrically isolated from each other such as by the plastic/resin of the tubular body. See FIG. 12A. Each coil is in electrical communication with the proximal hub by a unique electrical conductor such as a conductive trace or proximal extension of the wire.

This construction of double, electrically isolated helical coils creates a capacitor. This is roughly equivalent to two plates of nitinol with a plastic layer between them, illustrated in FIG. 12B. The capacitance is inversely proportional to the distance between wires. The only variable that would be changing would be d, the distance between the plates. If an axial compressive force is applied to the catheter, the wires (e.g., the first conductor 140 and the second conductor 142) will move closer together, thus increasing the capacitance. If an axial tensile force is applied, the wires will get further apart, decreasing the capacitance. This capacitance can be measured at the proximal end of the catheter, giving a measurement of the force at the helical capacitor. Although referred to as a capacitor, this sensor is measuring the electrical interaction between the two coils of wire. There may be a measurable change in inductance or other resulting change due to applied axial forces.

At least a first helical capacitor may have at least one or five or ten or more complete revolutions of each wire. A capacitor may be located within the distal most 5 or 10 or 20 centimeters of the catheter body to sense forces experienced at the distal end. At least a second capacitor may be provided within the proximal most 5 or 10 or 20 centimeters of the catheter body, to sense forces experienced at the proximal end of the catheter.

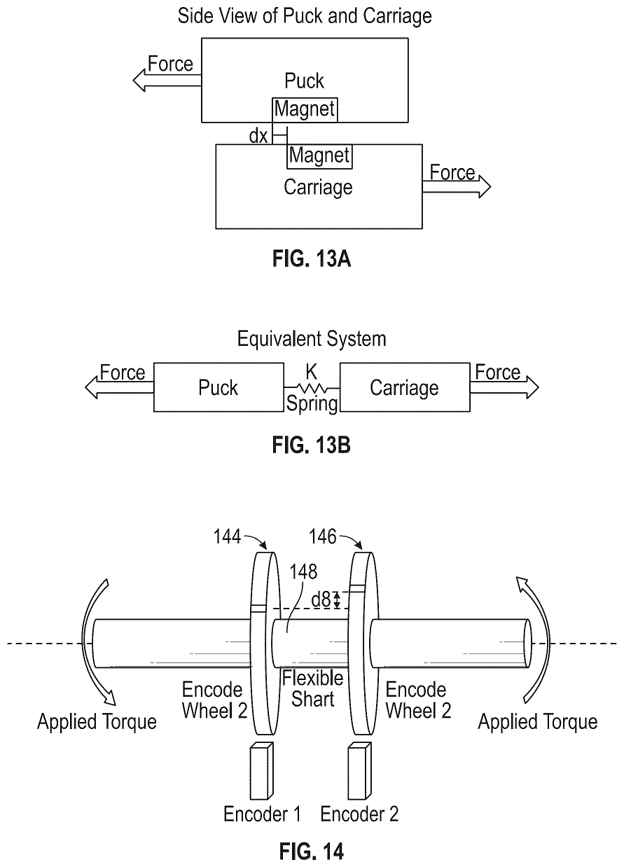

It may also be desirable to measure elastic forces across the magnetic coupling between the hub and corresponding carriage, using the natural springiness (compliance) of the magnetic coupling to measure the force applied to the hub. The magnetic coupling between the hubs and carriages creates a spring. When a force is applied to the hub, the hub will move a small amount relative to the carriage. See FIG. 13A. In robotics, this is called a series clastic actuator. This property can be used to measure the force applied from the carriage to the hub. To measure the force, the relative distance between the hub and the carriage (dx shown in FIG. 13A) is determined and characterize some effective spring constant k between the two components. See FIG. 13B.

The relative distance could be measured in multiple different ways. One method for measuring the relative distance between the hub and carriage is a magnetic sensor (e.g., a Hall effect Sensor between hub and carriage). A magnet is mounted to either the hub or carriage, and a corresponding magnetic sensor is mounted on the other device (carriage or hub). The magnetic sensor might be a hall effect sensor, a magneto-resistive sensor, or another type of magnetic field sensor. Generally, multiple sensors may be used to increase the reliability of the measurement. This reduces noise and reduces interference from external magnetic fields.

Other non-contact distance sensors can also be used. These include optical sensors, inductance sensors, and capacitance sensors. Optical sensors would preferably be configured in a manner that avoids accumulation of blood or other fluid in the interface between the hubs carriages. In some implementations, wireless (i.e., inductive) power may be used to translate movement and/or transfer information across the sterile barrier between a drive carriage and a hub, for example.

The magnetic coupling between the hub and the carriage has a shear or axial break away threshold which may be about 300 grams or 1000 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three-dimensional configuration of the catheter or guidewire during and/or following transvascular placement. Shape sensing fiber optics such as an array of FBG fibers to sense the shape of catheters and guidewires. By using multiple force sensing fibers that are a known distance from each other, the shape along the length of the catheter/guidewire can be determined.

A resistive strain gauge may be integrated into the body of the catheter or guidewire to measure force or torque. Such as at the distal tip and/or proximal end of the device.

Measurements of force and/or torque applied to the catheter or guidewire shafts can be used to determine applied force and/or torque above a safety threshold. When an applied force and/or torque exceeds a safety threshold, a warning may be provided to a user. Applied force and/or torque measurements may also be used to provide feedback related to better catheter manipulation and control. Applied force and/or torque measurements may also be used with processed fluoroscopic imaging information to determine or characterize distal tip motion.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table would have a linear array of corresponding magnetic sensors going the entire length of the table. A processor can be configured to determine the location of the magnet along the length of the linear sensor array, and display axial position information to the physician.

The foregoing may alternatively be accomplished using a non-contact inductive sensor to directly measure the position of the hubs through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

The location of the catheters and guidewires within the anatomy may also be determined by processing the fluoroscopic image with machine vision, such as to determine the distal tip position, distal tip orientation, and/or guidewire shape. Comparing distal tip position or movement or lack thereof to commanded or actual proximal catheter or guidewire movement at the hub, may be used to detect a loss of relative motion, which may be indicative of a device shaft buckling, prolapse, kinking, or a similar outcome (for example, along the device shaft length inside the body (e.g., in the aorta) or outside the body between hubs. The processing may be done in real time to provide position/orientation data at up to 30 Hertz, although this technique would only provide data while the fluoroscopic imaging is turned on. In some embodiments, machine vision algorithms can be used to generate and suggest optimal catheter manipulations to access or reach anatomical landmarks, similar to driver assist. The machine vision algorithms may utilize data to automatically drive the catheters depending on the anatomy presented by fluoroscopy.

Proximal torque applied to the catheter or guidewire shaft may be determined using a dual encoder torque sensor. Referring to FIG. 14, a first encoder 144 and a second encoder 146 may be spaced axially apart along the shaft 148, for measuring the difference in angle over a length of flexible catheter/tube. The difference in angle is interpolated as a torque, since the catheter/tube has a known torsional stiffness. As torque is applied to the shaft, the slightly flexible portion of the shaft will twist. The difference between the angles measured by the encoders (dθ) tells us the torque. T=k*dθ, where k is the torsional stiffness.

Confirming the absence of bubbles in fluid lines may also be accomplished using bubble sensors, particularly where the physician is remote from the patient. This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

Preferably, a bubble removal system is automatically activated upon detection of in line bubbles. A processor may be configured to activate a valve positioned in the flow path downstream of the bubble detector, upon the detection of bubbles. The valve diverts a column of fluid out of the flow path to the patient and into a reservoir. Once bubbles are no longer detected in the flow path and after the volume of fluid in the flow path between the detector and the valve has passed through the valve, the valve may be activated to reconnect the source of fluid with the patient through the flow path. In other embodiments, the bubble removal system can include a pump and control system upstream of the bubble detector for removal of in line bubbles. A processor may be configured to activate the pump upon detection of bubbles to reverse the fluid flow and clear the bubbles into a waste reservoir before reestablishing bubble free forward flow. The bubble removal system (e.g., bubble filtration) may be located downstream in the fluid path. In some embodiments, the bubble removal system may be located downstream in the fluid path of both saline and contrast media. In some embodiments, the bubble removal system is positioned in a hub. In some embodiments, the bubble removal system is located between a hemostasis valve in a hub and wye-connector (coupled to a saline line and a contrast line) in the hub. Positioning the bubble removal system downstream in the fluid path may advantageously minimize the risk of air embolism.

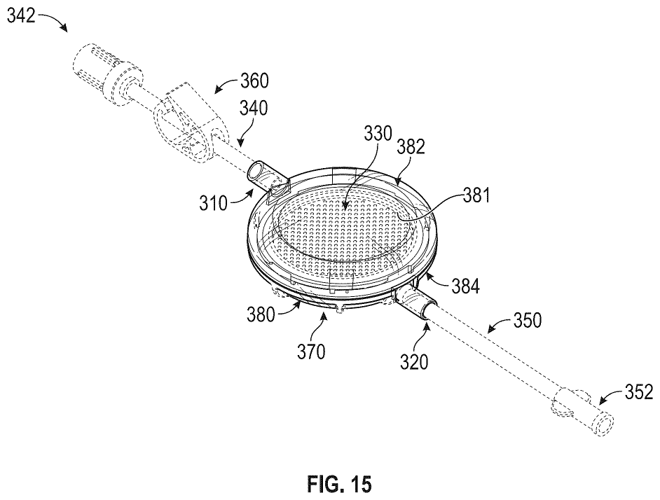

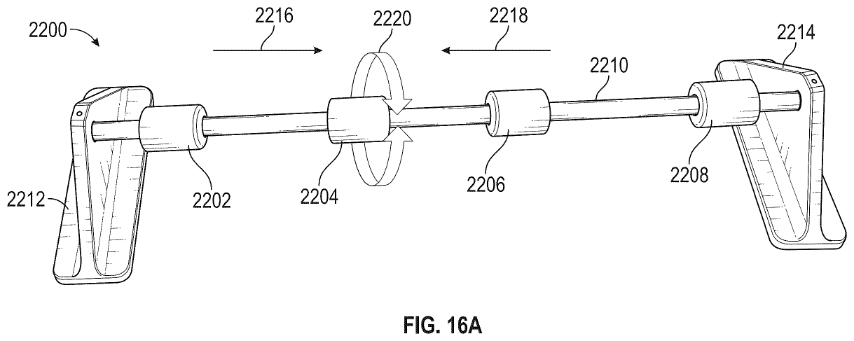

It may additionally be desirable for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump. Referring to FIG. 15, one example of a clot retrieval device 370 can include a body 380 enclosing a chamber 381 which communicates with a first port 310 and a second port 320.

In some embodiments, the body 380 includes a housing having a top portion 382 and a bottom portion 384. The body 380 may include a filter 330 positioned in the chamber 381 between the top portion 382, and the bottom portion 384. In some examples, the first port 310 is configured to connect to a first end of a first tube 340 that is fluidly connected to a proximal end of an aspiration catheter.

In an embodiment that is configured to be connected downstream from the hub, the first tube 340 includes a connector 342 positioned at a second end of the first tube 340 that is configured to engage or mate with a corresponding connector on or in communication with the hub. The first port 310 directly communicates with the chamber on the upstream (e.g., top side) of the filter, and the second port 320 directly communicates with the chamber on the downstream (e.g., bottom side) of the filter to facilitate direct visualization of material caught on the upstream side of the filter.

In an implementation configured for remote operation, any of a variety of sensors may be provided to detect clot passing through the aspiration line and/or trapped in the filter, such as an optical sensor, pressure sensor, flow rate sensor, ultrasound sensor, a clot camera or others known in the art. A clot camera can be an optical camera as described in greater detail below.

In some embodiments, the second port 320 is configured to connect to a first end of a second tube 350 that is fluidly connected to an aspiration source (e.g., a pump). In some embodiments, the second tube 350 includes a connector 352 positioned at a second end of the second tube 350 that is configured to engage or mate with a corresponding connector on the pump.

In some examples, the system 300 can include an on-off valve 360 such as a clamp. The valve 360 can be positioned in between the filter 330 and the patient, such as over the first tube 340 to allow the user to engage the clamp and provide flow control by isolating the patient from the clot retrieval device 370. Closing the on-off valve 360 and operating the remote vacuum pump (not illustrated) causes the canister associated with the vacuum pump and the chamber 381 to reach the same low pressure. Due to the short distance and small line volume of the lumen between the chamber 381 end the distal end of the catheter, a sharp negative pressure spike is experienced at the distal end of the catheter rapidly following opening of the on-off valve 360. Additional details are disclosed in U.S. Pat. No. 11,259,821 issued Mar. 1, 2022 to Buck et al., entitled Aspiration System with Accelerated Response, the entirety of which is hereby expressly incorporated by reference herein. In some embodiments, a vacuum may be cycled against a clot to retrieve the clot. The vacuum may be automatically and robotically controlled to remove the clot.

The body 380 can have a top surface spaced apart from a bottom surface by a tubular side wall. In the illustrated implementation, the top and bottom surfaces are substantially circular, and spaced apart by a cylindrical side wall. The top surface may have a diameter that is at least about three times, or five times or more than the axial length (transverse to the top and bottom surfaces) of the side wall, to produce a generally disc shaped housing. Preferably at least a portion of the top wall is optically transparent to improve clot visualization once it is trapped in the clot retrieval device 370. Additional details may be found in U.S. Provisional Application No. 63/256,743, the entirety of which is hereby incorporated by reference herein.

In some examples, the body 380 can include a flush port (not illustrated) that is configured to allow the injection of an optically transparent media such as air, saline or other fluid into the chamber 381 to clear an optical path between the window and the filter to improve clot visualization once it is trapped in the filter 330.

The foregoing represents certain specific implementations of a drive table and associated components and catheters. A wide variety of different drive table constructions can be made, for supporting and axially advancing and retracting two or three or four or more drive magnet assemblies to robotically drive interventional devices, fluid elements, and electrical umbilical elements for communicating electrical signals and fluids to the catheter hubs, as will be appreciated by those of skill in the art in view of the disclosure herein. Additional details may be found in U.S. patent application Ser. No. 17/527,393, filed Nov. 16, 2021, titled CATHETER DRIVE SYSTEM FOR SUPRA-AORTIC ACCESS, U.S. application Ser. No. 17/960,014, filed on Oct. 4, 2022, titled METHOD OF PERFORMING A MULTI CATHETER ROBOTIC NEUROVASCULAR PROCEDURE, U.S. application Ser. No. 17/816,669, filed on Aug. 1, 2022, titled METHOD OF SUPRA-AORTIC ACCESS FOR A NEUROVASCULAR PROCEDURE, and U.S. application Ser. No. 18/060,935, filed Dec. 1, 2022, titled METHOD OF PRIMING AN INTERVENTIONAL DEVICE ASSEMBLY, each of which is hereby expressly incorporated by reference in its entirety herein.

While the foregoing describes robotically driven interventional devices and manually driven interventional devices, the devices may be manually driven, robotically driven, or a combination of both manually and robotically driven interventional devices, as will be appreciated by those of skill in the art in view of the disclosure herein.

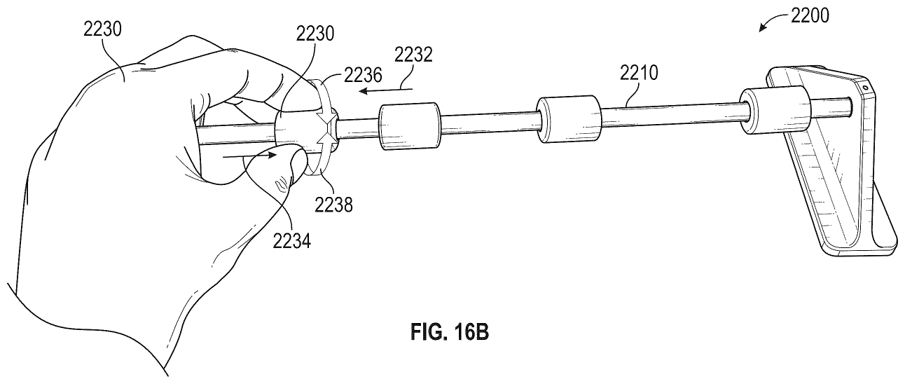

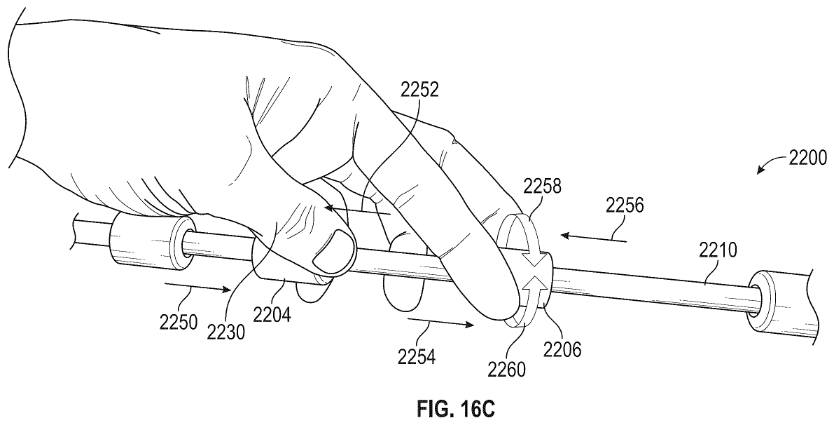

FIGS. 16A-16C illustrate an example control mechanism 2200 for manipulating interventional devices driven by (or otherwise associated with) respective hubs. For example, each hub may be manipulated and/or otherwise moved using at least one control installed in control mechanism 2200. Each control may be adapted to move a unique hub and associated interventional device during an interventional procedure.

As shown in FIG. 16A, the control mechanism 2200 include a first control 2202, a second control 2204, a third control 2206, and a fourth control 2208. More or fewer controls may be provided, depending upon the intended interventional devices configuration. Each control 2202-2208 is movably carried on a shaft 2210 that is coupled to a distal bracket 2212 and to a proximal bracket 2214. The controls 2202-2208 may advance distally or retract proximally on the shaft 2210, as indicated by arrow 2218 and arrow 2216. In addition, each control 2202-2208 may also be rotated about the shaft 2210, as indicated by arrow 2220. Each control movement may trigger a responsive movement in a corresponding carriage on the support table, which may in turn drive movement of a corresponding hub as has been discussed.

The control mechanism 2200 may be positioned on or near to a patient support table having a set of hubs and catheters/interventional devices. In some implementations, the control mechanism 2200 may be positioned remote from the support table such as behind a radiation shield or in a different room or different geographical location in a telemedicine implementation.

Each control 2202-2208 may correspond to and drive movement of a hub and/or a hub and interventional device combination. For example, the control 2202 may be configured to drive hub 30 (FIG. 3F) to move an interventional device such as an 0.088 inch guide catheter corresponding to the hub 30. Similarly, the control 2204 may be configured to drive hub 28 (122) to move an interventional device such as an 0.071 inch procedure catheter. The control 2206 may be configured to drive hub 126 to move an interventional device such as a steerable access catheter. The control 2208 may be configured to drive hub 26 to axially and rotationally move an interventional device such as a guidewire.

FIG. 16B illustrates an example of manually manipulating the control 2202 on control mechanism 2200. In operation, if the user 2230 moves the control 2202 axially along shaft 2210 and distally, as shown by arrow 2232, a corresponding coupled hub and/or interventional device may move responsively in the same direction by a same or scaled amount. If the user 2230 rotates the control 2202 about the shaft 2210 and advances the control proximally, as shown by arrow 2234, a corresponding coupled interventional device will responsively move rotationally and proximally by a same or scaled amount. If the user 2230 moves the control 2202 rotationally about the shaft 2210, as shown by arrow 2236 or arrow 2238, a corresponding coupled hub will drive the corresponding interventional device rotationally in the same direction and/or by a same or scaled amount.

Other axes and degrees of freedom may be defined to enable control 2202 to perform movements that may be translated to movement of hubs and/or interventional devices. For example, the control mechanism may be provided with one or more deflection controls configured to initiate a lateral deflection in a deflection zone on the corresponding interventional device.

Axial movement of a control may be configured to move the coupled hub on a 1:1 basis, or on a non 1:1 scaled basis. For example, if the user 2230 advances the control 2022 about 5 millimeters distally along the shaft 2210, then the corresponding hub may responsively move 5 millimeters in the distal direction.

If the user 2230 rotates the control 2022 about its rotational axis by 5 degrees, the coupled hub will cause the corresponding interventional device to rotate on a 1:1 basis or on a non 1:1 scaled basis. The scaled amount may be selected to reduce or increase the amount of distance and rotation that a hub and/or interventional device moves in accordance with the control movement.

In some implementations, the scaled amount described herein may be determined using a scale factor. The scale factor may apply to one or both translational and rotational movement. In some implementations, a first scale factor is selected for translational movement and a second scale factor, different than the first scale factor, is selected for rotational movement. The axial scaling factor may drive proximal catheter movement at a faster speed than distal catheter movement for a given proximal or distal manipulation of the control.

The rotational scale factor may be 1:1 while the axial scale factor may move the hub by a greater distance than movement of the control such that hub travel to control travel is at least about 2:1 or 5:1 or 10:1 or more depending upon the desired axial length of the control assembly.

The control mechanism 2200 may be configured to enable the clinician to adjust the scale factor for different parts of the procedure. For example, distal advance of the procedure catheter and access catheter through the guide catheter and up to the selected ostium may desirably be accomplished in a ‘fast’ mode. But more distal travel into the neuro vasculature may desirably be accomplished in a relatively slow mode by actuation of a speed control.

In another implementation, one or more controls may be configured to progressively drive advance or retraction speeds of the corresponding hub and associated catheter. For example, distal control 2202 may drive the guide catheter. A slight distal movement of the control 2202 may advance the guide catheter distally at a slow speed, while advancing the control 2202 by a greater distance distally increases the rate of distal travel of the guide catheter.

Controlling the speed of the corresponding hubs either axially or both axially and rotationally may enhance the overall speed of the procedure. For example, advance of the various devices from the femoral access point up to the aortic arch may desirably be accomplished at a faster rate than more distal navigation closer to the treatment site. Also proximal retraction of the various devices, particularly the guidewire, access catheter and procedure catheter may be desirably accomplished at a relatively higher speeds than distal advance.

FIG. 16C illustrates another example of manually manipulating a control on the control mechanism 2200 to move hubs and/or other interventional devices. In some implementations, two or more controls 2202-2208 may be moved in combination to trigger movement of one or more hubs and/or related interventional devices. In the depicted example, the user 2230 moves control 2204 and control 2206 in combination (e.g., sequentially, simultaneously) such as to simultaneously move the 0.088 guide catheter and the 0.071 aspiration catheter as a unit. Example movement of control 2204 may include axial proximal movement in the directions shown by arrows 2250. Sequentially or simultaneously, the user 2230 may move control 2206 axially in either of the directions shown by arrows 2254 and 2256 while also moving control 2206 rotationally in either of the directions shown by arrows 2258 and 2260.

In some implementations, each control mechanism and/or additional controls (not shown) may be color coded, shaped coded, tactile coded, or other coding to indicate to the user 2230 which color is configured to move which hub or interventional device. In some implementations, the control color coding may also be applied to the hubs and/or interventional devices such that a user may visually match a particular hub/device with a particular control.

In some implementations, other control operations beyond translational movement and rotational movement may be carried out using controls 2202-2208. For example, controls 2202-2208 may be configured to drive a shape change and/or stiffness change of a corresponding interventional device. Controls 2202-2208 may be toggled between different operating modes. For example, controls 2202-2208 may be toggled between movement driven by acceleration and velocity to movement that reflects actual linear displacement or rotation.

In some implementations, the control mechanism 2200 may be provided with a visual display or other indicator of the relative positions of the controls which may correspond the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

In some implementations, the control mechanism 2200 may include haptic components to provide haptic feedback to a user operating the controls. For example, if the control 2202 is triggering movement of a catheter and the catheter detects a large force at the tip, the control 2202 may generate haptic feedback to indicate to the user to stop or reverse a performed movement. In some implementations, haptic feedback may be generated at the control to indicate to the user to slow or speed a movement using the control. In some implementations, haptics may provide feedback on a large torsional strain buildup that might precede an abrupt rotation, or a large axial force buildup that may be a prelude to buckling of the catheter.

The systems described herein may compare an actual fluoroscopic image position to an input displacement from the control device. A static fluoroscopic image of the patient may be captured in which the patient's vasculature is indexed relative to bony landmarks or one or more implanted soft tissue fiducial markers. Then a real time fluoroscopic image may be displayed as an overlay, aligned with the static image by registration of the fiducial markers. Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

FIG. 17 illustrates a side elevational schematic view of a catheter assembly 2900 for combined supra-aortic access and/or neurovascular site access and procedure (e.g., aspiration), as described herein. The catheter assembly 2900 may be configured for either a manual or a robotic procedure.

The catheter assembly 2900 includes an insert or access catheter 2902, a procedure catheter 2904, and a guide catheter 2906. Other components are possible including, but not limited to, one or more guidewires (e.g., optional guidewire 2907), one or more guide catheters, an access sheath and/or one or more other procedure catheters and/or associated catheter (control) hubs. In some embodiments, the catheter assembly 2900 may also be configured with an optional deflection control 2908 for controlling deflection of one or more catheters of catheter assembly 2900.

In operation, the catheter assembly 2900 may be used without having to exchange hub components. For example, in the two stage procedure disclosed previously, a first stage for achieving supra-aortic access includes mounting an access catheter, guide catheter and guidewire to the support table. Upon gaining supra aortic access, the access catheter and guidewire were typically removed from the guide catheter. Then, a second catheter assembly is introduced through the guide catheter after attaching a new guidewire hub and a procedure catheter hub to the corresponding drive carriage on the support table.

The single catheter assembly 2900 of FIG. 17 is configured to be operated without having to remove hubs and catheters and without the addition of additional assemblies and/or hubs. Thus, the multicomponent access and procedure configuration of catheter assembly 2900 may utilize a guidewire 2907 manufactured to function as an access guidewire and a navigation guidewire to allow for sufficient access and support, and navigation to the particular distal treatment site. In a non-limiting example configured for robotic implementation, a catheter assembly may include a guidewire hub (e.g., guidewire hub 2909 or guidewire hub 26 positioned on a drive table and to the right of catheter 2902), an insert or access catheter hub 2910, a procedure catheter hub 2912, a guide catheter hub 2914 and corresponding catheters. In certain embodiments, one or more of the hubs may include or be coupled to a hemostasis valve (e.g., a rotating hemostasis valve) to accommodate introduction of interventional devices therethrough. Additional details regarding hemostasis valves are included in U.S. patent application Ser. No. 17/879,614, entitled Multi Catheter System With Integrated Fluidics Management, filed Aug. 2, 2022, which is hereby expressly incorporated by reference in its entirety herein.

Once access above the aortic arch has been achieved, the insert or access catheter 2902 (associated with insert catheter hub 2910) may be parked in the vicinity of a carotid artery ostia and the remainder or a subset of the catheter assembly may be guided more distally toward a particular site (e.g., a clot site, a surgical site, a procedure site, etc.).

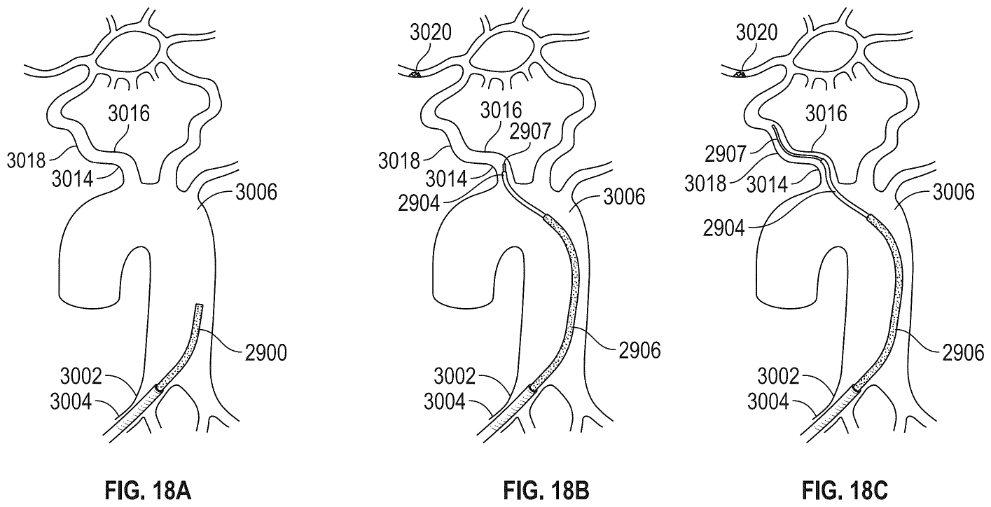

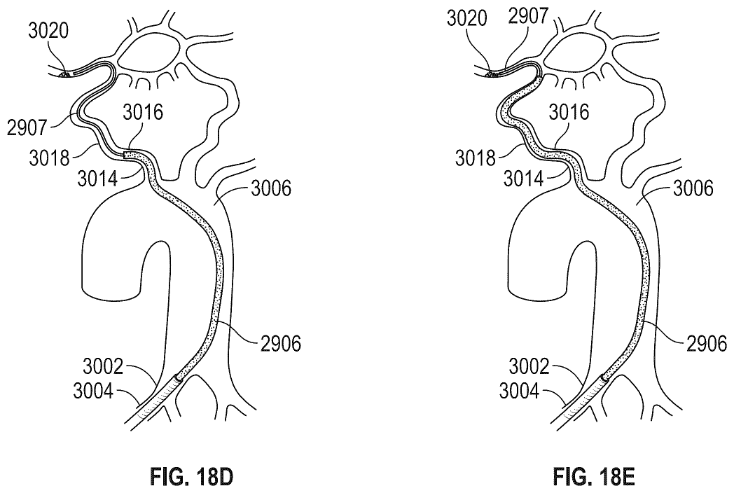

In some embodiments, other smaller procedure catheters may also be added and used at the site. As used herein for catheter assembly 2900, in a robotic configuration of catheter assembly 2900, the catheter 2906 may function as a guide catheter. The catheter 2904 may function as a procedure (e.g., aspiration) catheter. In some embodiments, the catheter 2906 may function to perform aspiration in addition to functioning as a guide catheter, either instead of or in addition to the catheter 2904. The access catheter 2902 may have a distal deflection zone and can function to access a desired ostium. One of skill in the art will appreciate from FIGS. 18A-18E that either manual manipulation or robotic manipulation of the multi-catheter stack are contemplated herein.

In some embodiments, the catheter assembly 2900 (or other combined catheter assemblies described herein) may be driven as a unit to a location. However, each catheter (or guidewire) component may instead be operated and driven independent of one another to the same or different locations.

In a non-limiting example, the catheter assembly 2900 may be used for a diagnostic angiogram procedure. In some embodiments, the catheter assembly 2900 may include only the guidewire 2907 and access catheter 2902 (in the form of a diagnostic angiographic catheter) for performing the diagnostic angiogram procedure or only the guidewire 2907 and the access catheter 2902 may be utilized during the procedure. Alternatively, the guide catheter 2906 and procedure catheter 2904 may be retracted proximally to expose the distal end of the access catheter 2902 (e.g., a few centimeters of the distal end of the access catheter) to perform the diagnostic angiography.

As shown in FIG. 17, the guide catheter 2906, procedure catheter 2904, access catheter 2902, and guidewire 2907 can be arranged concentrically. In certain embodiments, the guide catheter 2906 may be a ‘large bore’ guide catheter or access catheter having a diameter of at least about 0.075 or at least about 0.080 inches in diameter. The procedure catheter 2904 may be an aspiration catheter having a diameter within the range of from about 0.060 to about 0.075 inches. The access catheter 2902 may be a steerable catheter with a deflectable distal tip, having a diameter within the range of from about 0.025 to about 0.050 inches. The guidewire 2907 may have a diameter within the range of from about 0.014 to about 0.020 inches. In one example, the guide catheter 2906 may have a diameter of about 0.088 inches, the procedure catheter 2904 about 0.071 inches, the access catheter 2902 about 0.035 inches, and the guidewire 2907 may have a diameter of about 0.018 inches.

FIGS. 18A-18E depict an example sequence of steps of introducing a multi-catheter assembly configured to achieve access all the way to the clot, either manually or robotically. FIGS. 18A-18E may be described using the interventional device assembly of FIG. 17. Other combinations of catheters may be substituted for the interventional device assembly, as will be appreciated by those of skill in the art in view of the disclosure herein.

Referring to FIG. 18A, the catheter assembly 2900 is shown having three catheters driven through an introducer sheath 3002, up through the iliac artery 3004 and into the descending aorta. Next, the access catheter 2902, the procedure catheter 2904 (e.g., 0.071 inch) and the guide catheter 2906 (e.g., 0.088 inch) are tracked up to the aortic arch 3006, as shown in FIG. 18B. Here, the distal end of the guide catheter 2906 may be parked below the aortic arch 3006 and the procedure catheter 2904, access catheter 2902 (positioned within the procedure catheter 2904 and not visible in FIG. 18B), and a guidewire 2907 can be driven into the ostium (e.g., simultaneously or separately). In some embodiments, the access catheter 2902 is advanced out of the procedure catheter 2904 and the guide catheter 2906 to engage the ostium first. After the distal end of the access catheter 2902 is positioned within the desired ostium, the guidewire 2907 can be advanced distally into the ostium to secure access. After the access catheter 2902 and guidewire 2907 are positioned within the desired ostium, the procedure catheter 2904 and/or guide catheter 2906 can be advanced into the ostium (and, in some embodiments, beyond), while using the support of the access catheter 2902 and/or guidewire 2907 to maneuver through the aorta and into the ostium. In the embodiment shown in FIG. 18B, the procedure catheter 2904 has been advanced into the ostium while the guide catheter 2906 has remained parked below the aortic arch 3006.

Referring to FIG. 18C, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 18D, the guide catheter 2906 and the procedure catheter 2904 (positioned within the guide catheter 2906 and not visible in FIG. 18D) are both advanced (e.g., simultaneously or sequentially) over the guidewire 2907 and over the insert or access catheter 2902 (positioned within the procedure catheter 2904 and not visible in FIG. 18D) while the access catheter 2902 remains at the ostium for support. The guidewire 2907 may be further advanced past the petrous segment 3018 to the site of the clot 3020, such as the Ml segment.

Referring to FIG. 18E, the guide catheter 2906 and the procedure catheter 2904 (positioned within the guide catheter 2906 and not visible in FIG. 18E) are advanced (e.g., simultaneously or sequentially) to position the distal tip of the procedure catheter 2904 at the procedure site, for example on the face of the clot 3020. The guidewire 2907 and access catheter 2902 (positioned within the procedure catheter 2904 and not visible in FIG. 18E) are removed, and aspiration of the clot 3020 commences through the procedure catheter 2904. That is, the guidewire 2907 and the access catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procure catheter 2904 may be removed before removing the guide catheter 2906.

The catheter assembly 2900 may be used to perform a neurovascular procedure, as described in FIGS. 18A-18E. For example, the neurovascular procedure may be a neurovascular thrombectomy. The steps of the procedure may include providing an assembly that includes at least a guidewire, an access catheter, a guide catheter, and a procedure catheter. For example, the catheter assembly 2900 includes a guidewire 2907, an access (e.g., insert) catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter, a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system, and driving the assembly to achieve supra-aortic access. The steps may further include driving a subset of the assembly to a neurovascular site, and performing the neurovascular procedure using a subset of the assembly. The subset of the assembly may include the guidewire, the guide catheter, and the procedure catheter.

Each of the guidewire 2907, the access catheter 2902, the guide catheter 2906, and the procedure catheter 2904 is configured to be adjusted by a respective hub. For example, the guidewire 2907 may include (or be coupled to) a hub installed on one of the tray assemblies described herein. Similarly, the access catheter 2902 may be coupled to catheter hub 2910. The guide catheter 2906 may be coupled to the guide catheter hub 2914. The procedure catheter 2904 may be coupled to the procedure catheter hub 2912.

In general coupling of the assembly may include magnetically coupling a first hub (i.e., a guidewire hub 2909) on the guidewire 2907 to a first drive magnet, magnetically coupling a second hub (i.e., an access catheter hub 2910) on the access catheter 2902 to a second drive magnet, magnetically coupling a third hub (i.e., a procedure catheter hub 2912) on the procedure catheter 2904 to a third drive magnet, and magnetically coupling a fourth hub (i.e., a guide catheter hub 2914) on the guide catheter 2906 to a fourth drive magnet. In general, the first drive magnet, the second drive magnet, the third drive magnet, and the fourth drive magnet are each independently movably carried by a drive table, as described with respect to tray assemblies and controls described herein. In some embodiments, the first drive magnet, the second drive magnet, the third drive magnet, and the fourth drive magnet are coupled (e.g., to their respective catheter hubs) through a sterile barrier (e.g., a sterile and fluid barrier) and independently movably carried by a drive table having a plurality of driven magnets. In some embodiments, two or more drive magnets can be tethered or otherwise coupled together to move as a unit in response to commands from a single control device tethered or otherwise coupled to one of the drive magnets.

In some implementations, the steps of performing the neurovascular procedure may include driving the assembly in response to movement of each of the hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a driven magnet carried by the respective hub. This provides a magnetic coupling between the drive magnet and driven magnet through the sterile barrier such that the respective hub is moved across the top of the sterile barrier in response to movement of the hub adapter outside of the sterile field (as described in detail in FIG. 4). Movement of the hub adapter is driven by a drive system carried by the support table in which the guidewire hub 2909, the guide catheter hub 2914, the procedure catheter hub 2912, and the access catheter hub 2910 are installed upon.

The steps may further include driving a subset of the assembly in response to movement of each of the hub adapters along the support table until the subset of the assembly is positioned to perform a neurovascular procedure at a neurovascular treatment site. The subset of the assembly may include the guidewire 2907, the guide catheter 2906, and the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the catheter assembly 2900 may be part of a robotic control system for achieving supra-aortic access and neurovascular treatment site access, as described in FIGS. 18A-18E. In some embodiments, the catheter assembly 2900 may be part of a manual control system for achieving supra-aortic access and neurovascular treatment site access. In some embodiments, the catheter assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, access catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an access catheter hub 2910 configured to adjust axial and rotational movement of an access catheter 2902. The robotic control system may also include a guide catheter hub 2914 configured to control axial catheter hub 2912 configured to adjust an axial position and a rotational position of a procedure catheter 2904.

In some embodiments, the procedure catheter hub 2912 is further configured to laterally deflect a distal deflection zone of the procedure catheter 2904.

In some embodiments, the guidewire hub 2909 is configured to couple to a guidewire hub adapter by magnetically coupling the guidewire hub to a first drive magnet. The access catheter hub 2910 is configured to couple to an access catheter hub adapter by magnetically coupling the access catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes
a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail with respect to FIG. 4 above.

In some embodiments, the robotic control system includes a second driven magnet on the access catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

In some embodiments, devices (e.g., hubs, hub adapters, interventional devices, and/or trays) described herein may be used during a robotically driven procedure. For example, in a robotically driven procedure, one or more of the interventional devices may be driven through vasculature and to a procedure site. Robotically driving such devices may include engaging electromechanical components that are controlled by user input. In some implementations, users may provide the input at a control system that interfaces with one or more hubs and hub adapters.

In some embodiments, the hubs, hub adapters, interventional devices, and trays described herein may be used during a non-robotic (e.g., manually driven) procedure. Manually driving such devices may include engaging manually with the hubs to affect movement of the interventional devices.

In some embodiments, the devices described herein may be used to carry out a method of performing an intracranial procedure at an intracranial site. The method of performing the intracranial procedure may include any of the same steps as described herein for performing a neurovascular procedure. The procedure may be robotically performed, manually performed, or a hybridized combination of both.

While the foregoing describes magnetic coupling of hubs to drive magnets, in other embodiments, any of the interventional devices and/or hubs may be mechanically coupled to a drive system. Any of the methods described herein may include steps of mechanically coupling one or more interventional devices (e.g., the guidewire 2907, the access catheter 2902, the procedure catheter 2904, and/or the guide catheter 2906) and/or one or more hubs (e.g., the guidewire hub 2909, the access catheter hub 2910, the procedure catheter hub 2912, and/or the guide catheter hub 2914) with one or more drive mechanisms.

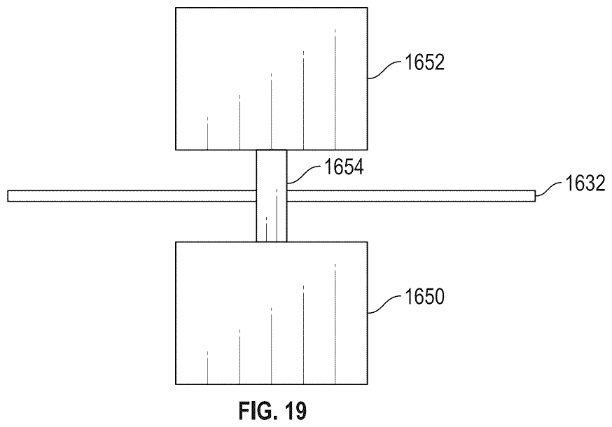

FIG. 19 illustrates a mechanical coupling mechanism 1654 between a drive mechanism 1650 and a driven mechanism 1652. Drive mechanism 1650 and driven mechanism 1652 may have any of the same or similar features or functions as the drive magnet 67 and driven magnet 69, respectively, except as otherwise described herein. The drive mechanism 1650 may be part of or coupled to a hub adapter (e.g., the hub adapter 48). The driven mechanism 1652 may be part of or coupled to a hub (e.g., the hub 36, the guidewire hub 2909, the access catheter hub 2910, the procedure catheter hub 2912, or the guide catheter hub 2914). In some instances, the mechanical coupling mechanism 1654 may include a structural support (e.g., a support rod or support strut) extending transversely through a seal in a sterile barrier 1632. The seal may permit the structural support to be advanced along a length of the sterile barrier 1632, while still maintaining a seal with the structural support to maintain the sterile field, as the drive mechanism 1650 and driven mechanism 1652 are advanced and/or retracted as described herein. For example, the seal may include a tongue and groove closure mechanism along the sterile barrier 1632 that is configured to close on either side of the structural support while permitting passage of the structural support through the sterile barrier 1632 and maintaining a seal against the structural support as the structural support is advanced along the length of the sterile barrier 1632.

In some embodiments, the structural support can extend through an elongate self closing seal between two adjacent coaptive edges of flexible material (e.g., similar in shape to a duckbill valve) that extends along an axis. As the structural support advances along the axis between the coaptive edges, the coaptive edges may permit the structural support to advance, and then may be biased back into a sealing engagement with each other as the structural support passes any given point along the axis.

In some embodiments, the drive mechanism may be a splined drive shaft (e.g., a non-sterile splined drive shaft). The mechanical coupling mechanism 1654 can include a pulley within a plate that serves as the sterile barrier 1632 and a sterile splined shaft configured to couple to the driven mechanism 1652. The driven mechanism 1652 can be a sterile pulley that receives the sterile splined shaft from the sterile barrier. In some embodiments, one or more splined drive shafts can engage and turn corresponding pulleys in the plate that serves as the sterile barrier. Each hub can have a sterile pulley that is configured to receive a sterile splined shaft from the sterile barrier plate. Rotation of the splined drive shaft can turn the pulley in the sterile barrier plate, which can, in turn, turn the sterile pulley in the hub via the sterile splined shaft.

It will be understood by one having skill in the art that any embodiment as described herein may be modified to incorporate a mechanical coupling mechanism, for example, as shown in FIG. 19.

The interventional devices described herein may be provided individually or at least some of the interventional devices can be provided in a preassembled (e.g., nested or stacked) configuration. For example, the interventional devices may be provided in the form of an interventional device assembly, such as catheter assembly 2900, in a concentric nested or stacked configuration. If provided individually, each catheter (and in some embodiments, each corresponding catheter hub) can be unpackaged and primed to remove air from its inner lumen, for example, by flushing the catheter (and in some embodiments, the corresponding catheter hub) to remove air by displacing it with a fluid, such as saline, contrast media, or a mixture of saline and contrast media. After priming, the interventional devices can be manually assembled into a stacked configuration so that they are ready for introduction into the body for a surgical procedure, for example, via an introducer sheath.

Assembling the devices into a stacked configuration can include individually inserting interventional devices into one another by order of size. For example, an interventional device having a second largest diameter can be inserted into the lumen of an interventional device having a largest diameter. An interventional device having a third largest diameter can then be inserted into the interventional device having the second largest diameter and so on.

For example, with respect to FIG. 17, assembly can be performed by first inserting a distal end of the catheter 2904 through the guide catheter hub 2914 and into the catheter 2906. The catheter 2904 can be advanced through the catheter 2906 until the distal tip of the catheter 2904 is flush with or extends beyond the distal tip of the catheter 2906, and/or until the catheter 2904 cannot be inserted any further. Then, the distal end of the catheter 2902 can be inserted through the procedure catheter hub 2912 and into the catheter 2904. The catheter 2902 can be advanced through the catheter 2904 until the distal tip of the catheter 2902 is flush with or extends beyond the distal tip of the catheter 2904, and/or until the catheter 2902 cannot be inserted any further. Then, the distal end of the guidewire 2907 can be inserted through the access catheter hub 2910 and into the catheter 2902. The guidewire 2907 can be advanced through the catheter 2902 until the distal tip of the guidewire 2907 is flush with or extends beyond the distal tip of the catheter 2902, and/or until the guidewire 2907 cannot be inserted any further.

Embodiments in which two or more of the interventional devices are packaged together as a single unit in an assembled (e.g., nested or stacked) configuration may provide efficient unpackaging and preparation prior to use and efficient assembly within a robotic control system. The interventional devices may be pre-mounted to their respective hubs prior to packaging. In certain embodiments, two or three or more interventional devices may be packaged in a fully nested (i.e., fully axially inserted) configuration or nearly fully nested configuration. In a fully nested configuration, each interventional device is inserted as far as possible into an adjacent distal hub and interventional device. Such a fully nested configuration may minimize a total length of the interventional device assembly and minimize the size of the packaging required to house the interventional device assembly.

In some embodiments, the interventional devices may also be sterilized prior to packaging while in the assembled configuration, for example, using ethylene oxide gas. In some embodiments, the interventional devices may be packaged while in the assembled configuration before sterilization with ethylene oxide gas. For interventional devices in a nested or stacked configuration, ethylene oxide gas can be provided in a space between adjacent interventional devices (for example, an annular lumen between an outer diameter of a first interventional device nested within a second interventional device and the inner diameter of the second interventional device) for sterilization. In some embodiments, the interventional device assembly can be packaged in a thermoformed tray and sealed with an HDPE (e.g., Tyvek®) lid. The interventional device assembly can be unpackaged by removal (e.g., opening or peeling off) of the lid by a user in a non-sterile field. A user in the sterile field can then remove the interventional device assembly and place it on the sterile work surface, for example, of a robotic drive table, as described herein.

Packaging the interventional devices in an assembled configuration and sterilized state can reduce the time associated with unpackaging and assembly of individual interventional devices and facilitate efficient connection to a robotic drive system. Each interventional device and hub combination may further be packaged with a fluidics connection for coupling to a fluid source and/or a vacuum source. In some embodiments, each hub or a hemostasis valve coupled to the hub may include the fluidics connection.

After the interventional device assembly is unpackaged (e.g., after the interventional device assembly is positioned on the robotic drive table), priming can be performed while the devices are concentrically nested or stacked. This is preferably accomplished in each fluid lumen, such as, for example, the annular lumen between the catheter 2906 and the catheter 2904 and in between each of the additional concentric interventional devices in the concentric stack. In certain embodiments, the fluid lumen can include a lumen between a distal hub and a proximal interventional device, such as, for example, the lumen between the guide catheter hub 2914 and the catheter 2904. In certain embodiments, priming can be performed while the devices are still in the sterile packaging.

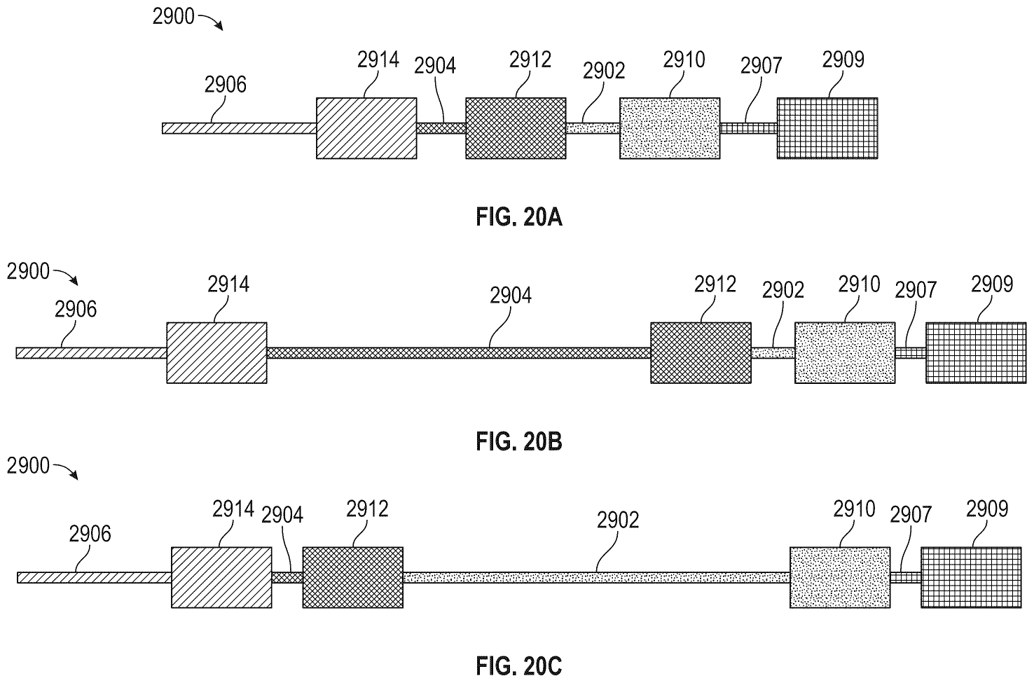

The fluidics connections can be connected to a fluidics system for delivering saline and contrast media to the catheters and providing aspiration. In some embodiments, the fluidics connections may be passed outside the sterile field for connection to the fluidics system. Once connected, the fluidics system can perform a priming sequence to flush each catheter of the interventional device assembly with fluid (e.g., saline, contrast media, or a mixture of saline and contrast media). The priming sequence may also include flushing each corresponding catheter hub with fluid. The fluid may be de-aired or de-gassed by the fluidics system prior to priming. In some embodiments, a vacuum source of the fluidics system can also be used to evacuate air from each catheter while flushing with fluid. In certain embodiments, a tip of the catheter can be placed into a container of fluid, such as saline, contrast media, or a mixture of saline and contrast media, during priming so that the fluid in the container, and not air, is aspirated through the tip of the catheter when the vacuum source is applied. In other embodiments, the tip of the catheter may be blocked (for example, using a plug) so that air is not aspirated from the tip of the catheter when the vacuum source is applied. In certain embodiments, the priming process may be automated such that a user can provide a single command and each catheter (and in some embodiments, each corresponding catheter hub) can be primed, sequentially (for example, as described with respect to FIGS. 20A-20C) or simultaneously.

Additional details regarding fluidics systems are disclosed in U.S. patent application Ser. No. 17/879,614, entitled Multi Catheter System With Integrated Fluidics Management, filed Aug. 2, 2022, which is hereby expressly incorporated by reference in its entirety herein.

Fluid resistance within a lumen may be greater when there is a reduction in cross sectional luminal area for flow, for example, when a second interventional device (e.g., a catheter or guidewire) extends within the lumen of a first interventional device. The amount of fluid resistance can be affected by the length of the cross sectional narrowing, for example, due to a depth of axial insertion of the second interventional device within the first interventional device. A second interventional device extending partially through the lumen of a first interventional device will provide a smaller length of cross-sectional narrowing, and accordingly may result in a lower fluid resistance within the lumen of the first catheter, than if the second interventional device were to extend entirely through the lumen of the first interventional device. Thus, fluid resistance can be lowered by at least partially decreasing a depth of axial insertion (i.e., axial overlap) of a second interventional device into the lumen through which fluid is to be injected (e.g., a length of the second interventional device into its concentrically adjacent lumen).

In some embodiments, over certain depths of insertion of a second interventional device within a first interventional device (for example, when the second interventional device is at or near a maximum insertion depth within the first interventional device), the size of the fluid channel between the devices (e.g., the annular lumen between the first interventional device and the second interventional device) can lead to higher than desirable amounts of fluid resistance during a priming procedure. In some embodiments, the depth of insertion of the second interventional device within the first interventional device can be decreased to reduce the pressure needed to prime the catheter and reduce internal interference.

In some embodiments, a catheter in the interventional device assembly can be separated from the other interventional devices for priming to reduce the pressure needed to prime the catheter and reduce internal interference. The catheter being primed may be separated from the interventional devices within the lumen of the catheter by proximally retracting the interventional devices within the lumen of the catheter. For example, the interventional devices within the lumen of the catheter being primed can be proximally retracted from the catheter being primed as far as possible while still maintaining a nested or stacked relationship (e.g., at least about 2 cm or 5 cm or more axial overlap) in order to minimize the pressure needed to prime the catheter and minimize internal interference. In other words, a catheter can be separated from more proximal interventional devices for priming while a distal tip of an adjacent proximal interventional device is still positioned within the lumen of the catheter. Maintaining at least some of the distal tip of an adjacent proximal interventional device within the lumen of the catheter may allow for easier reinsertion and advancement of the proximal interventional device after priming.

In some embodiments, the axial overlap may be between about 2 cm and about 20 cm, between about 2 cm and 10 cm, between about 2 cm and 5 cm, between about 5 cm and 20 cm, between about 5 cm and 10 cm, or any other suitable range. In some embodiments, the axial overlap may be at least about 2 cm, at least about 5 cm, at least about 10 cm, at least about 20 cm, no more than 2 cm, no more than 5 cm, no more than 10 cm, no more than 20 cm, about 2 cm, about 5 cm, about 10 cm, about 20 cm, or any other suitable amount.

In some embodiments, the robotic drive table can be programed to proximally retract the inner interventional device(s) from the catheter being primed as much as possible while still maintaining a nested or stacked relationship. In other embodiments, the robotic drive table can be programmed to separate inner devices from the catheter being primed to a distance sufficient to optimize the length of the unobstructed lumen and result in an amount of fluid resistance lower than a threshold value. After the catheter being primed is separated from the other interventional devices, the catheter can be primed by flushing the catheter with fluid, such as saline, contrast media, or a mixture of saline and contrast media.

After the catheter is primed, it may be returned to an initial position and a next catheter of the interventional device assembly can be separated from the other interventional devices within its lumen for priming. This sequence can be repeated for each catheter of the interventional device assembly. In other embodiments, after a catheter is primed, it may be advanced to a ready or drive position to begin insertion into the patient. While the foregoing describes separating catheters to be primed by retraction of inner interventional devices, an outer catheter may also be separated from inner interventional devices by distally axially advancing the outer catheter relative to the inner interventional devices. An example of a priming process is described with respect to FIGS. 20A-20C.

FIG. 20A depicts the catheter assembly 2900 assembled in a concentric stack and axially compressed configuration. As shown in FIG. 20A, the interventional devices can be fully nested within each other. This may be the configuration following unpackaging of the catheter assembly 2900 and placement onto the robotic drive table. A priming sequence may begin by distally axially advancing the catheter 2906 and guide catheter hub 2914 relative to the catheter 2904, procedure catheter hub 2912, catheter 2902, access catheter hub 2910, guidewire 2907, and guidewire hub 2909, for example, as far as possible while maintaining a distal tip of the catheter 2904 within the lumen of the catheter 2906, as shown in FIG. 20B, or to a distance that will result in a desirable amount of fluid resistance for priming. In some embodiments, the catheter 2906 is advanced in response to a control signal from a control system. The catheter 2906 can then be primed by introducing priming fluid using the fluidics system. In some embodiments, priming fluid is introduced in response to a control signal from a control system. Priming the catheter 2906 can include priming the guide catheter hub 2914. For example, in certain embodiments, the guide catheter hub 2914 or a hemostasis valve coupled thereto can include fluidics connections to receive priming fluid from the fluidics system. After priming, the catheter 2906 can be returned to its initial position (e.g., the fully axially compressed configuration) as shown in FIG. 20A. In some embodiments, the catheter 2906 is returned to its initial position in response to a control signal from a control system.

After the catheter 2906 is primed and returned to its initial position, the catheter 2904 and procedure catheter hub 2912 can be distally axially advanced relative to the catheter 2902, access catheter hub 2910, guidewire 2907 and guidewire hub 2909 (also distally axially advancing the catheter 2906 and guide catheter hub 2914 without changing or minimally changing their relative position with respect to catheter 2904), for example, as far as possible while maintaining a distal tip of the catheter 2902 within the lumen of the catheter 2904, as shown in FIG. 20C, or to a distance that will result in a desirable amount of fluid resistance for priming. In some embodiments, the catheter 2904 and the catheter 2906 are advanced in response to a control signal from a control system. The catheter 2904 can then be primed by introducing priming fluid using the fluidics system. In some embodiments, priming fluid is introduced in response to a control signal from a control system. Priming the catheter 2904 can include priming the procedure catheter hub 2912. For example, in certain embodiments, the procedure catheter hub 2912 or a hemostasis valve coupled thereto can include fluidics connections to receive priming fluid from the fluidics system. After priming, the catheter 2904 and catheter 2906 can be returned to their initial positions (e.g., the fully axially compressed configuration) as shown in FIG. 20A. In some embodiments, the catheter 2904 and the catheter 2906 are returned to their initial position in response to a control signal from a control system.

After the catheter 2904 is primed and returned to its initial position, the catheter 2902 and access catheter hub 2910 can be distally axially advanced relative to the guidewire 2907 and guidewire hub 2909 (also distally axially advancing the catheter 2906, guide catheter hub 2914, catheter 2904, and procedure catheter hub 2912 without changing or minimally changing their relative positions with respect to the catheter 2902), for example, as far as possible while maintaining a distal tip of the guidewire 2907 within the lumen of the catheter 2902, or to a distance that will result in a desirable amount of fluid resistance for priming. In some embodiments, the catheter 2902, the catheter 2904, and the catheter 2906 are advanced in response to a control signal from a control system. The catheter 2902 can then be primed by introducing priming fluid using the fluidics system. In some embodiments, priming fluid is introduced in response to a control signal from a control system. Priming the catheter 2902 can include priming the access catheter hub 2910. For example, in certain embodiments, the access catheter hub 2910 or a hemostasis valve coupled thereto can include fluidics connections to receive priming fluid from the fluidics system. After priming, the catheter 2902 and catheters 2904 and 2906 can be returned to their initial positions (e.g., the fully axially compressed configuration) shown in FIG. 20A. In some embodiments, the catheter 2902, the catheter 2904, and the catheter 2906 are returned to their initial position in response to a control signal from a control system.

In some embodiments, the priming procedure described with respect to FIGS. 20A-20C may be performed in response to a single control signal from a control system. In other embodiments, various steps of the priming procedure may be performed in response to unique control signals. In some embodiments, priming of each unique interventional device can be performed in response to a unique control signal.

In alternative embodiments, each of the catheters can be distally separated from one another simultaneously for priming. For example, the catheter 2902 can be distally separated from the guidewire 2907 while maintaining the distal tip of the guidewire 2907 in the lumen of the catheter 2902, the catheter 2904 can be distally separated from the catheter 2902 while maintaining the distal tip of the catheter 2902 in the lumen of the catheter 2904, and the catheter 2906 can be distally separated from the catheter 2904 while maintaining the distal tip of the catheter 2904 in the lumen of the catheter 2906 simultaneously. However, an embodiment in which only one set of adjacent hubs is separated at a time, as described with respect to FIGS. 20A-20C, can provide a smaller overall length of the assembly at any particular time, which can allow for use with a smaller robotic drive system. While separation of outer catheters from their inner interventional devices is described as distally axially advancing the catheters relative to their inner interventional devices, separation can include proximally retracting the inner interventional devices from the outer catheters.

In alternative embodiments, one or more of the catheter 2902, the catheter 2904, and the catheter 2906 can be advanced to a ready or drive position to begin insertion into the patient after priming (e.g., prior to priming a subsequent catheter). In such embodiments, the catheters may advance to the ready or drive position without returning to their initial position after priming.

As described above, in some embodiments, the catheters 2902, 2904, and 2906 may be assembled into the concentric stack orientation illustrated in FIG. 17 prior to flushing the catheters to remove air by displacing it with a fluid such as saline contrast media, or a mixture of saline and contrast media. This is preferably accomplished in each fluid lumen, such as, for example, the annular lumen between the catheter 2906 and the catheter 2904 and in between each of the additional concentric interventional devices in the concentric stack. Infusing fluid (e.g., saline, contrast media, or a mixture of saline and contrast media) under pressure may displace substantially all of the air but some small bubbles may remain, adhering to the inside wall of an outer catheter (e.g., the guide catheter 2906), the outside wall of an inner catheter (e.g., the procedure catheter 2904), or both.

While fluid is being introduced under pressure into the proximal end of the annular lumen (e.g., into a hub of the outer catheter or a hemostasis valve coupled thereto), the inner catheter may be moved with respect to the outer catheter, to disrupt the holding forces between the microbubbles and adjacent wall and allow the bubbles to be carried downstream and out through the distal opening of the lumen or removed via aspiration. The catheters may be moved axially, rotationally or both with respect to each other. In certain embodiments, the catheters may be reciprocated axially, rotationally, or both with respect to each other. In some embodiments, the catheters may be moved intermittently axially, rotationally, or both. In other embodiments, the catheters may be rotated continuously or in a constant direction.

In some implementations, a first catheter is moved reciprocally with respect to an adjacent catheter or guidewire such as axially over a stroke length in a range of from about 1 mm to about 250 mm, from about 10 mm to about 250 mm, from about 5 mm to about 125 mm, from about 25 mm to about 125 mm, from about 10 mm to about 50 mm, from about 15 mm to about 30 mm, from about 5 mm to about 30 mm, from about 15 mm to about 25 mm, from about 20 mm to about 40 mm, or any other suitable range. In some implementations, a first catheter is moved reciprocally with respect to an adjacent catheter or guidewire such as axially over a stroke length of at least 5 mm, at least 10 mm, at least 15 mm, at least 20 mm, at least 25 mm, at least 30 mm, at least 50 mm, no more than 10 mm, no more than 20 mm, no more than 25 mm, no more than 30 mm, no more than 50 mm, no more than 125 mm, no more than 150 mm, about 5 mm, about 10 mm, about 15 mm, about 20 mm, about 25 mm, about 30 mm, about 50 mm, or any other suitable stroke length.

In some implementations, a first catheter is moved reciprocally with respect to an adjacent catheter or guidewire such as axially at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as axially at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In one implementation, a first catheter is moved reciprocally with respect to the adjacent catheter or guidewire such as axially over a stroke length in a range of from about 0.5 inches to about 10 inches, or from about one inch to about 5 inches at a reciprocation frequency of no more than about 5 cycles per second or two cycles per second or less.

In some implementations, a first catheter is moved reciprocally with respect to an adjacent catheter or guidewire such as rotationally over an angle of rotation per stroke in a range of from about 5 degrees to about 180 degrees, from about 5 degrees to about 360 degrees, from about 15 degrees to about 180 degrees, from about 15 degrees to about 150 degrees, from about 15 degrees to about 120 degrees, from about 15 degrees to about 90 degrees, form about 15 degrees to about 60 degrees, from about 15 degrees to about 30 degrees, from about 30 degrees to about 180 degrees, from about 30 degrees to about 150 degrees, from about 30 degrees to about 120 degrees, from about 30 degrees to about 90 degrees, form about 30 degrees to about 60 degrees, from about 60 degrees to about 180 degrees, from about 60 degrees to about 150 degrees, from about 60 degrees to about 120 degrees, from about 60 degrees to about 90 degrees, from about 90 degrees to about 180 degrees, from about 90 degrees to about 150 degrees, from about 90 degrees to about 120 degrees, from about 120 degrees to about 180 degrees, from about 120 degrees to about 150 degrees, from about 150 degrees to about 180 degrees or any other suitable range. In some implementations, a first catheter is moved reciprocally with respect to an adjacent catheter or guidewire such as rotationally over an angle of rotation per stroke of at least 5 degrees, at least 15 degrees, at least 30 degrees, at least 60 degrees, at least 90 degrees, at least 120 degrees, at least 150 degrees, at least 180 degrees, at least 360 degrees, no more than 5 degrees, no more than 15 degrees, no more than 30 degrees, no more than 60 degrees, no more than 90 degrees, no more than 120 degrees, no more than 150 degrees, no more than 180 degrees, no more than 360 degrees, about 5 degrees, about 15 degrees, about 30 degrees, about 60 degrees, about 90 degrees, about 120 degrees, about 150 degrees, about 180 degrees, about 360 degrees, or any other suitable angle.

In some implementations, a first catheter is moved reciprocally with respect to an adjacent catheter or guidewire such as rotationally at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as rotationally at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In some implementations, a first catheter is moved reciprocally with respect to an adjacent catheter or guidewire for a number of reciprocations between 1 and 200, between 1 and 100, between 1 and 50, between 1 and 25, between 1 and 15, between 1 and 10, between 1 and 5, between 5 and 25, between 5 and 15, between 5 and 10, or any other suitable range. In some implementations, a first catheter is moved reciprocally with respect to an adjacent catheter or guidewire for at least 1 reciprocation, at least 2 reciprocations, at least 5 reciprocations, at least 10 reciprocations, at least 15 reciprocations, at least 25 reciprocations, at least 50 reciprocations, no more than 5 reciprocations, no more than 10 reciprocations, no more than 15 reciprocations, no more than 25 reciprocations, no more 50 than reciprocations, no more than 100 reciprocations, no more than 200 reciprocations, about 1 reciprocation, about 2 reciprocations, about 5 reciprocations, about 10 reciprocations, about 25 reciprocations, about 50 reciprocations, about 100 reciprocations, about 200 reciprocations, or any other suitable number. One reciprocation can include a movement (axially or rotationally) from a first position to a second position followed by a return from the second position to the first position.

In some implementations, a first catheter is moved reciprocally with respect to an adjacent catheter or guidewire over a length of time in a range of from 1 about second to about 60 seconds, from about 1 second to about 45 seconds, from about 1 second to about 30 seconds, from about 1 second to about 20 seconds, from about 1 second to about 15 seconds, from about 1 second to about 10 seconds, from about 5 seconds to about 45 seconds, from about 5 seconds to about 30 seconds, from about 5 seconds to about 20 seconds, from about 5 seconds to about 15 seconds, from about 5 seconds to about 10 seconds, from about 10 seconds to about 30 seconds, form about 10 seconds to about 20 seconds, or any other suitable range. In some implementations, a first catheter is moved reciprocally with respect to an adjacent catheter or guidewire over a length of time of at least 1 second, at least 5 seconds, at least 10 seconds, at least 15 seconds, at least 20 seconds, at least 30 seconds, at least 45 seconds, at least 60 seconds, no more than 5 seconds, no more than 10 seconds, no more than 15 seconds, no more than 20 seconds, no more than 30 seconds, no more than 45 seconds, no more than 60 seconds, about 5 seconds, about 10 seconds, about 15 seconds, about 20 seconds, about 30 seconds, about 45 seconds, about 60 seconds, or any other suitable length of time.

Reciprocation of adjacent catheters to disrupt microbubbles may be accomplished manually by grasping the corresponding catheter hubs and manually moving the catheters axially or rotationally with respect to each other while delivering pressurized fluid (e.g., saline, contrast media, or a mixture of saline and contrast media). Alternatively, such as in a robotically driven system, a processor may be configured to robotically drive at least one of two adjacent catheter hubs (for example, at least one of guide catheter hub 2914 and procedure catheter hub 2912) to achieve relative movement between the adjacent catheters thereby disrupting and expelling microbubbles, such as in response to user activation of a flush control. For example, in certain embodiments, two adjacent interventional devices may be moved relative to one another in response to a control signal from a control system. In certain embodiments, delivery of pressurized fluid may be performed in response to a control signal from a control system.

The reciprocation of adjacent catheters may generate shear forces that dislodge the air bubbles. For example, relative movement of the inner and outer surfaces of adjacent catheters may increase the fluid shear rate between the adjacent catheters during priming in comparison to static surfaces. In some embodiments, the shear force can be increased by increasing the flow rate of the solution (e.g., saline, contrast media, or a mixture of saline and contrast media) being provided by the fluidics system. In certain embodiments, both flow rate and relative movement between adjacent catheters are controlled to dislodge air bubbles.

In some embodiments, after each catheter is primed by the fluidics system, an ultrasound bubble detector may be used to confirm that the catheters are substantially free of air bubbles. For example, an ultrasound chip (such as mounted within a hub adjacent a catheter receiving lumen) may be run along the length of the catheters to confirm that no air bubbles remain in the system.

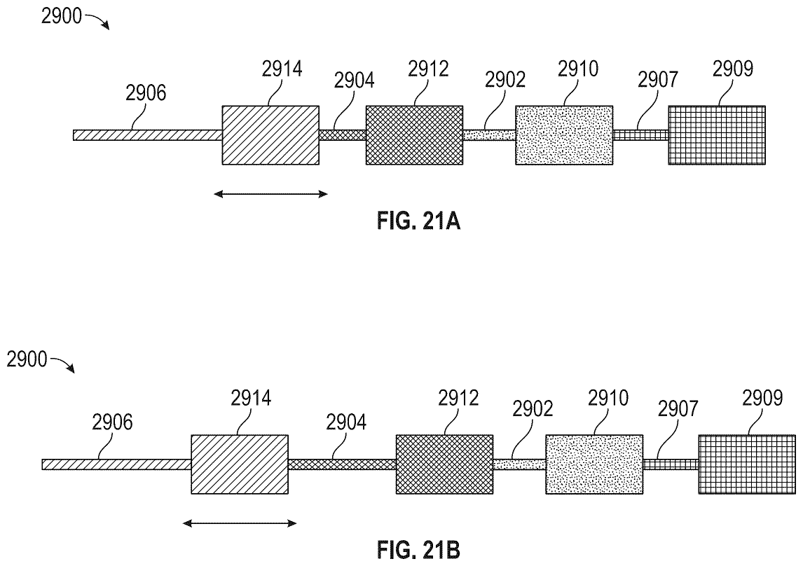

An example of a priming process including reciprocal movement of adjacent catheters is described with respect to FIGS. 21A-21B.

FIG. 21A depicts the catheter assembly 2900 assembled in a concentric stack configuration. As shown in FIG. 21A, the interventional devices can be fully nested within each other. This may be the configuration following unpackaging of the catheter assembly 2900 and placement onto the robotic drive table. Alternatively, individual interventional devices of the catheter assembly 2900 can be assembled into the catheter assembly 2900 on the drive table.

A priming sequence may begin by priming the catheter 2906. In some embodiments, the catheter 2906 can be primed by introducing fluid (e.g., saline, contrast media, or a mixture of saline and contrast media) under pressure into the lumen of the catheter 2906 while generating reciprocal movement of catheter 2906 and/or guide catheter hub 2914, axially, rotationally or both, relative to the catheter 2904. Priming the catheter 2906 can include priming the guide catheter hub 2914. For example, in certain embodiments, the guide catheter hub 2914 or a hemostasis valve coupled thereto can include fluidics connections to receive priming fluid from the fluidics system. In certain embodiments, the catheter 2906 and/or guide catheter hub 2914 can be axially agitated back and forth along a longitudinal axis of the catheter 2906 (e.g., between the position of FIG. 21A and the position of FIG. 21B). Axial and/or rotational reciprocal motion of the catheter 2906 and/or guide catheter hub 2914 can be performed manually or by a robotic drive table. Reciprocal movement may be generated in response to a control signal from a control system. Introducing fluid under pressure may be performed in response to a control signal from a control system.

In some embodiments, priming of the catheter 2906 may be performed by introducing fluid (e.g., saline, contrast media, or a mixture of saline and contrast media) under pressure into the lumen of the catheter 2906 while generating reciprocal movement of the catheter 2904 and/or procedure catheter hub 2912, axially, rotationally or both, relative to the catheter 2906. Axial and/or rotational reciprocal motion of the catheter 2904 and/or procedure catheter hub 2912 can be performed manually or by a robotic drive table. Reciprocal movement may be generated in response to a control signal from a control system. Introducing fluid under pressure may be performed in response to a control signal from a control system.

In some embodiments, priming of the catheter 2906 may be performed by introducing fluid (e.g., saline, contrast media, or a mixture of saline and contrast media) under pressure into the lumen of the catheter 2906 while generating reciprocal movement of both the catheter 2906 (and/or guide catheter hub 2914) and the catheter 2904 (and/or procedure catheter hub 2912), axially, rotationally or both, relative to one another. Reciprocal movement may be generated in response to a control signal from a control system. Introducing fluid under pressure may be performed in response to a control signal from a control system.

In some embodiments, after priming the catheter 2906, the catheter 2906 can be returned to an initial position as shown in FIG. 21A. In other embodiments, after priming the catheter 2906, the catheter 2906 can be advanced to a ready or drive position to begin insertion into the patient.

In some embodiments, after the catheter 2906 is primed, the catheter 2904 can be primed. Priming the catheter 2904 can include priming the procedure catheter hub 2912. For example, in certain embodiments, the procedure catheter hub 2912 or a hemostasis valve coupled thereto can include fluidics connections to receive priming fluid from the fluidics system. In some embodiments, the catheter 2904 can be primed by introducing fluid (e.g., saline, contrast media, or a mixture of saline and contrast media) under pressure into the lumen of the catheter 2904 while generating reciprocal movement of the catheter 2904 and/or procedure catheter hub 2912, axially, rotationally or both, relative to the catheter 2902. Reciprocal movement may be generated in response to a control signal from a control system. Introducing fluid under pressure may be performed in response to a control signal from a control system.

In some embodiments, priming of the catheter 2904 may be performed by introducing fluid (e.g., saline, contrast media, or a mixture of saline and contrast media) under pressure into the lumen of the catheter 2904 while generating reciprocal movement of the catheter 2902 and/or access catheter hub 2910, axially, rotationally or both, relative to the catheter 2904. Axial and/or rotational reciprocal motion of the catheter 2902 and/or access catheter hub 2910 can be performed manually or by a robotic drive table. Reciprocal movement may be generated in response to a control signal from a control system. Introducing fluid under pressure may be performed in response to a control signal from a control system.

In some embodiments, priming of the catheter 2904 may be performed by introducing fluid (e.g., saline, contrast media, or a mixture of saline and contrast media) under pressure into the lumen of the catheter 2904 while generating reciprocal movement of both the catheter 2904 (and/or procedure catheter hub 2912) and the catheter 2902 (and/or access catheter hub 2910), axially, rotationally or both, relative to one another. Reciprocal movement may be generated in response to a control signal from a control system. Introducing fluid under pressure may be performed in response to a control signal from a control system.

In some embodiments, after priming the catheter 2904, the catheter 2904 can be returned to an initial position as shown in FIG. 21A. In some embodiments, after priming the catheter 2904, the catheter 2904 can be advanced to a ready or drive position to begin insertion into the patient.

In some embodiments, after the catheter 2904 is primed, the catheter 2902 can be primed. Priming the catheter 2902 can include priming the access catheter hub 2910. For example, in certain embodiments, the access catheter hub 2910 or a hemostasis valve coupled thereto can include fluidics connections to receive priming fluid from the fluidics system. In some embodiments, the catheter 2902 can be primed by introducing fluid (e.g., saline, contrast media, or a mixture of saline and contrast media) under pressure into the lumen of the catheter 2902 while generating reciprocal movement of the catheter 2902 and/or access catheter hub 2910, axially, rotationally or both, relative to the guidewire 2907. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of the guidewire 2907 and/or guidewire hub 2909, axially, rotationally or both, relative to the catheter 2902. Axial and/or rotational reciprocal motion of the guidewire 2907 and/or guidewire hub 2909 can be performed manually or by a robotic drive table. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of both the catheter 2902 (and/or access catheter hub 2910) and the guidewire 2907 (and/or guidewire hub 2909), axially, rotationally or both, relative to one another. Reciprocal movement may be generated in response to a control signal from a control system. Introducing fluid under pressure may be performed in response to a control signal from a control system.

In some embodiments, after priming the catheter 2902, the catheter 2902 can be returned to an initial position as shown in FIG. 21A. In other embodiments, after priming the catheter 2902, the catheter 2902 can be advanced to a ready or drive position to begin insertion into the patient.

In some embodiments, the priming procedure described with respect to FIGS. 21A and 21B may be performed in response to a single control signal from a control system. In other embodiments, various steps of the priming procedure may be performed in response to unique control signals. In some embodiments, priming of each unique interventional device can be performed in response to a unique control signal.

In the priming sequence described herein with respect to FIGS. 21A and 21B, the catheters are primed in order starting with the catheter 2906, followed by the catheter 2904, and then followed by the catheter 2902. However, it is contemplated that the catheters may be primed in any order. The catheters may be primed in series as described above with respect to FIGS. 21A and 21B. Alternatively, two or more of the catheters or each of the catheters may be primed in parallel.

In certain embodiments, priming the catheters can include decreasing a depth of axial insertion (i.e., axial overlap) of a second interventional device into the lumen of a first interventional device through which fluid is to be injected (e.g., a length of the second interventional device into its concentrically adjacent lumen), as described with respect. to FIGS. 20A-20C, and also generating relative reciprocal movement, axially, rotationally or both, between first interventional device and the second interventional device during priming, as discussed with respect to FIGS. 21A and 21B.

In some implementations, priming of a catheter can include vibrating at least a portion of the catheter and/or its associated hub when included. Vibration can be induced, for example, by an electric motor incorporated into a hub of the catheter, or by a separate electric motor or source of vibration put against the catheter when priming. In some implementations, at least a portion of the support table on which the catheters and/or their associated hubs are placed upon can vibrate during priming of any one or more catheters to aid in removal of air and/or microbubbles of air. Such vibration can be performed by an electric motor.

### EXAMPLES

Additional embodiments are disclosed in further detail in the following examples, which are not in any way intended to limit the scope of the claims.

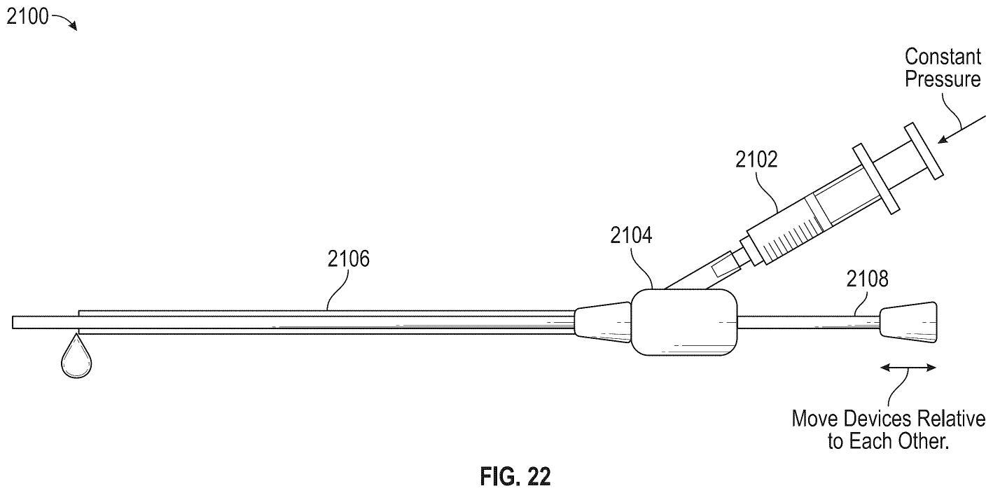

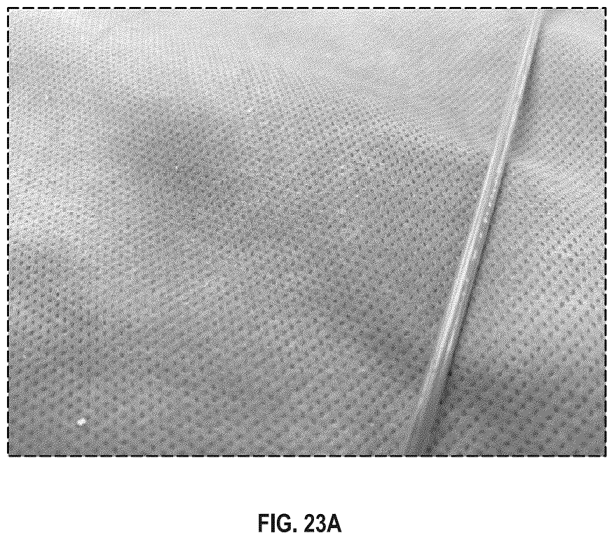

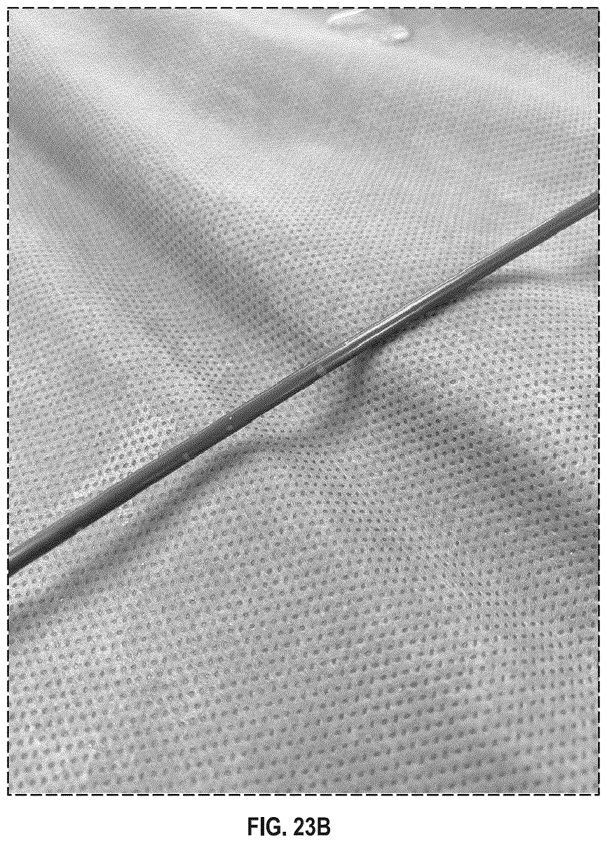

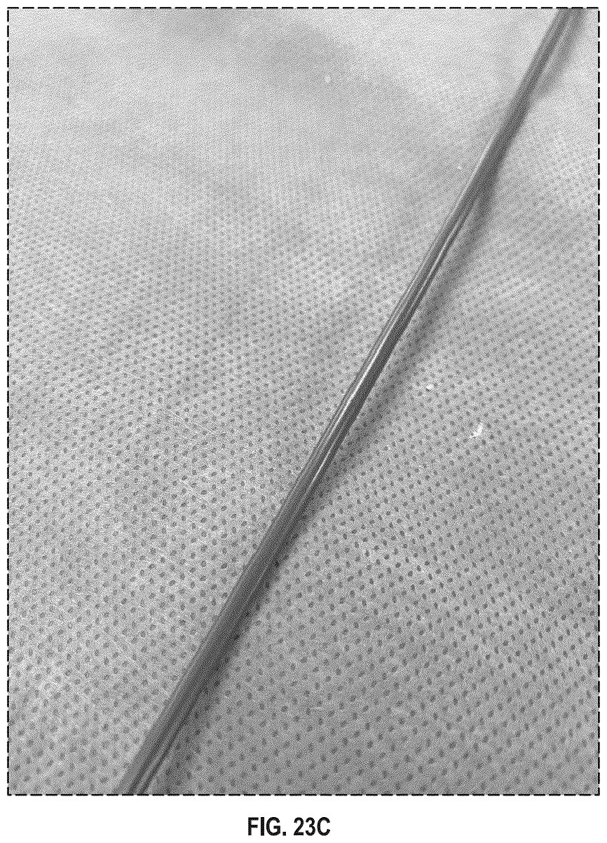

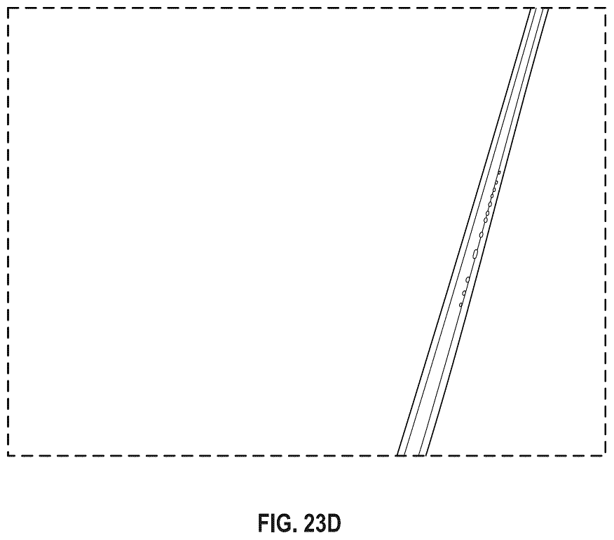

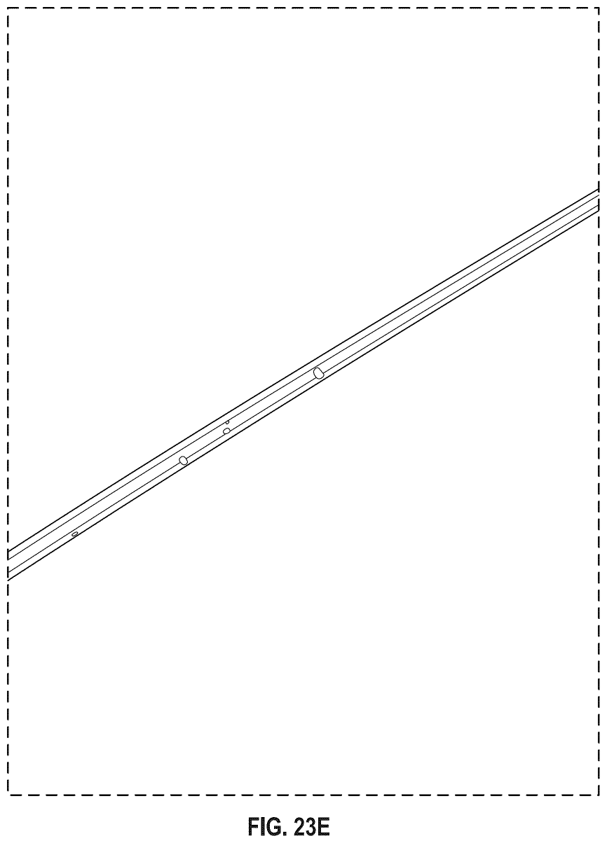

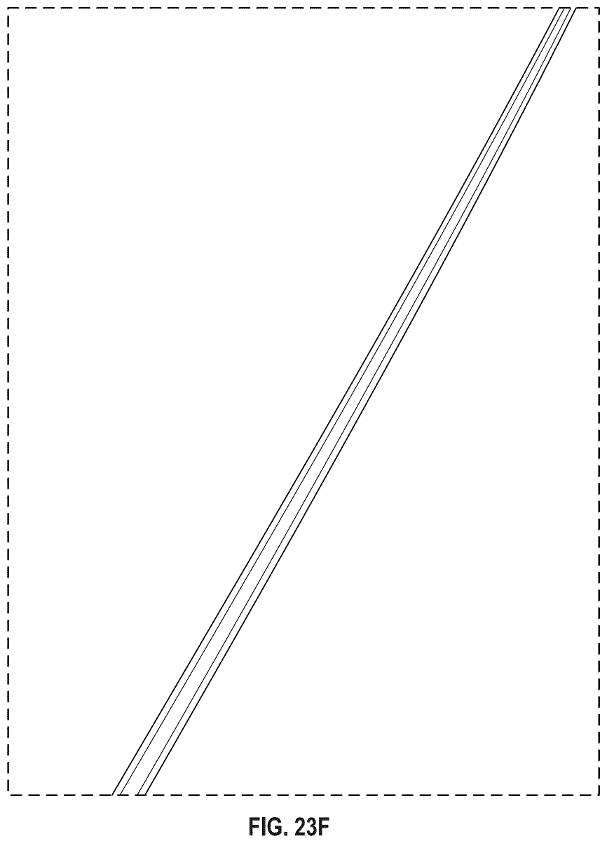

FIG. 22 is a diagram of a test system that was used for detecting the removal of air bubbles between concentrically stacked catheters. The test system included an inner catheter 2108 positioned within an interior lumen of an outer catheter 2106 in a concentric stack. The outer catheter 2106 was coupled to a rotating hemostasis valve 2104. The hemostasis valve 2104 was coupled to a syringe 2102 so that fluid injected using the syringe would flow through the lumen between the inner catheter 2108 and the outer catheter 2016. In the test system, the inner catheter 2108 had a diameter of about 0.071 inches. The outer catheter 2106 had a diameter of about 0.088 inches. The outer catheter 2106 was transparent to permit visualization of bubbles within the lumen. A distal end of the outer catheter 2108 allowed for small volumes of fluid to exit the outer catheter. FIG. 23A is a photograph showing the catheter 2106 and catheter 2108 in a concentric stack, prior to injection of fluid. FIG. 23D is an illustration thereof.

### Example 1

In a first example, the syringe 2102 was used to inject water at a constant pressure of about 150 psi through the hemostasis valve 2104 without moving the catheter 2106 or the catheter 2108. FIG. 23B is a photograph showing the catheter 2106 and catheter 2108 following the injection of water. FIG. 23E is an illustration thereof. As shown in FIG. 23B, bubbles are present within the lumen between the catheter 2106 and the catheter 2108.

### Example 2

In a second example, the syringe 2102 was used to inject water at a constant pressure of about 150 psi through the hemostasis valve 2104. Shortly after beginning to inject water, axial reciprocal movement of the inner catheter 2108 was performed for about 10 seconds. The reciprocal movement was performed at a frequency of about 1 Hz (or less) and a stroke length of about 20 mm (or more). FIG. 23C is a photograph showing the catheter 2106 and the catheter 2108 following the axial reciprocal movement. FIG. 23F is an illustration thereof. As shown in FIG. 23C, the lumen between the catheter 2106 and the catheter 2108 was substantially free of bubbles.

### Example 3

In a third example, an outer catheter having a diameter of about 0.071 inches and an inner catheter having a diameter of about 0.035 inches were used in the test system 2100 instead of the outer catheter 2106 and the inner catheter 2108 described with respect to Examples 1 and 2. A syringe 2102 was used to inject water at a constant pressure of about 150 psi through a hemostasis valve 2104 coupled to the outer catheter. Shortly after beginning to inject water, axial reciprocal movement of the inner catheter was performed for about 10 seconds. The reciprocal movement was performed at a frequency of about 1 Hz (or less) and a stroke length of about 20 mm (or more). Following the axial reciprocal movement, the lumen between the outer and inner catheters was found to be substantially free of bubbles by visual inspection.

### Control System

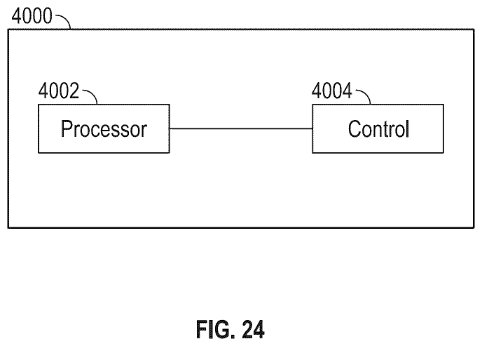

FIG. 24 illustrates a schematic view of an example of a control system 4000 that may be used to electronically control the systems and components described herein and/or perform the methods described herein. The control system 4000 may be configured to automatically adjust various motors, hub adapters, hubs, interventional devices, fluidics components (e.g., valves, pumps, etc.), and/or any other components described herein in response to commands input by an operator such as a physician. In response to command inputs by an operator, the control system 4000 may cause a series of responsive events to automatically occur.

In certain embodiments, the control system 4000 can include one or more processors 4002. For example, the control system 400 may have one processor or more than one processor that is configured to execute instructions to perform control system functionality and operations. For ease of reference in the description, sometimes herein the “one or more processors 4002” may be referred to as simply “a processor” or “the processor” such terminology or the illustration of a processor teaches there can be one or more processors, unless explicitly indicated otherwise. The one or more processors 4002 can be configured to automatically adjust the various system components described herein in response to commands input by an operator, for example, using one or more controls 4004 of the control system 4000. A single control 4004 is shown in FIG. 24. However, any suitable number of controls may be provided to correspond to various functions of the systems described herein. For example, in certain embodiments, each interventional device may have its own unique control 4004 or set of controls 4004 that can control various functions of the interventional device (e.g., axial movement, rotational movement, supply of fluids (e.g., saline, contrast, etc.), aspiration, etc.).

In certain embodiments, one or more controls 4004 may control priming functions for one or more interventional devices. For example, one or more controls 4004 can be operated to cause the interventional devices to perform a priming procedure, as described for example, with reference to FIGS. 20A-C. For example, one or more controls 4004 can be operated to cause axial movement of one or more interventional devices relative to one or more other interventional devices (e.g., by causing axial movement of corresponding hubs and/or hub adapters). One or more controls 4004 can be operated to cause introduction of fluid into the lumen of an interventional device to prime the interventional device.

In certain embodiments, one or more controls 4004 may be operated to cause the interventional devices to perform a priming procedure, as described for example, with reference to FIGS. 21A-21B. For example, one or more controls 4004 can be operated to cause reciprocal movement (e.g., axial and/or rotational reciprocal movement) of one or more interventional devices relative to one or more other interventional devices (e.g., by causing reciprocal movement of corresponding hubs and/or hub adapters). One or more controls 4004 can be operated to cause introduction of fluid into the lumen of an interventional device to prime the interventional device (e.g., during relative reciprocal movement).

The one or more processors 4002 may receive signals from the one or more controls 4004 and in response, initiate corresponding actions in the components of the systems described herein. For example, the one or more processors 4002 may be configured to generate output signals that cause responsive actions to be performed by the components of the described herein.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

The foregoing represents one specific implementation of a robotic control system. A wide variety of different robotic control system constructions can be made, for supporting and axially advancing and retracting two or three or four or more assemblies to robotically drive interventional devices, as will be appreciated by those of skill in the art in view of the disclosure herein.

While the foregoing describes interventional devices that are driven by a drive table, other suitable robotic drive systems or mechanisms may be used to drive the interventional devices, as will be appreciated by those of skill in the art in view of the disclosure herein.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

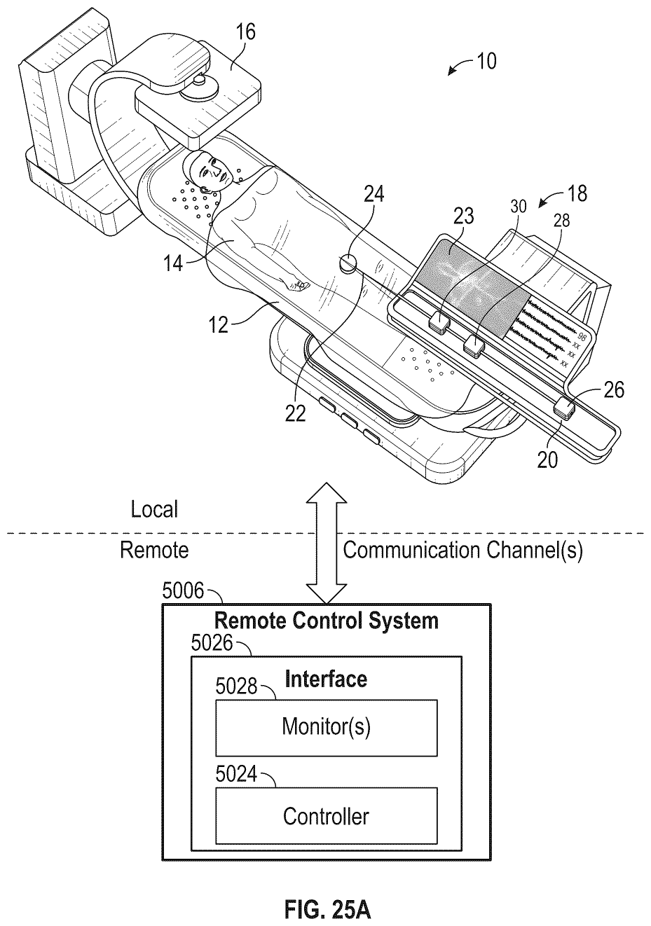

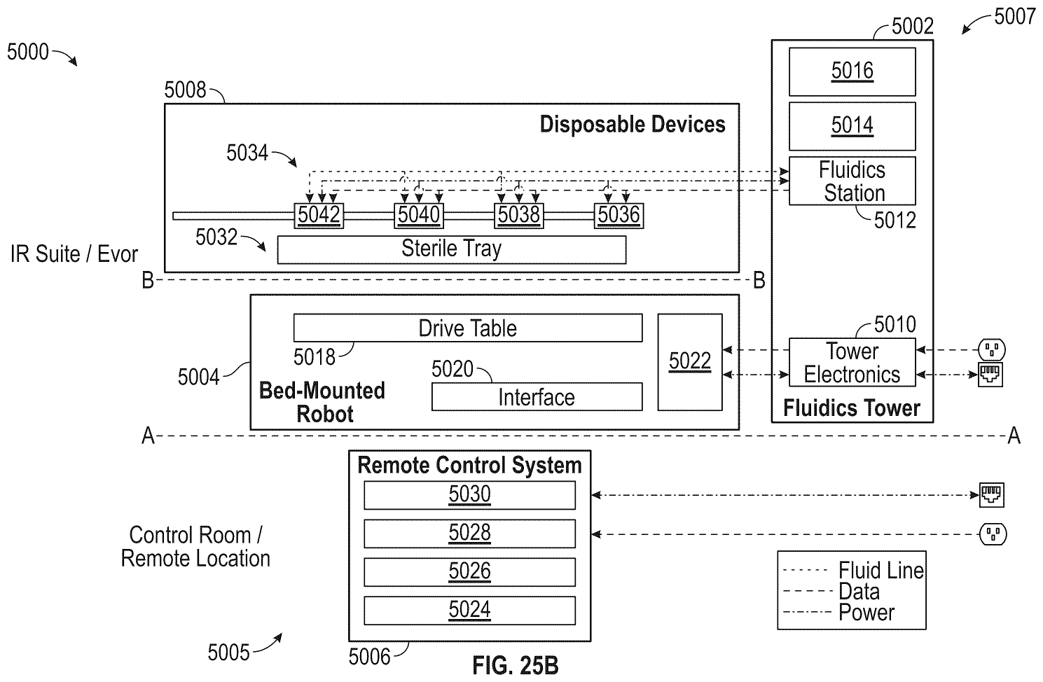

FIG. 25A illustrates a diagram of an example communication channel of a robotic medical system 5000. The systems of the robotic medical system 5000 may be divided between a local system 5007 and a remote system 5005 located remotely from the local system 5007. As shown in FIG. 25B, the remote system 5005 is separated from the local system 5007 by the line A-A. The local system 5007 can be positioned within an operating room. The remote system 5005 can be separated from the local system 5007. For example, the remote system 5005 can be in the operating room but separated from the local system 5007 by a fluoroscopic barrier, in another room in the same building, in another building, in another part of a local geographical area (for example, another part of a town or city), or in another part of a global geographical area (for example, another state or country). The local system 5007 may include the robotic interventional device drive system 18 in accordance with the present disclosure.

The robotic interventional device drive system 18 may include a fluidics tower, a robotic drive system, and one or more disposable devices described in greater detail below.

The remote system 5005 can include a remote control system 5006. The remote control system 5006 may include a control device 5024, one or more monitors 5028, an interface 5026, and one or more remote communication devices 5030 as described in greater detail below. The remote control system 5006 can further include a control console or may be in the form of a control console. The control console can be a workstation incorporating one or more of the control device 5024, the one or more monitors 5028, and/or the interface 5026. In some embodiments, the remote control system 5006 can include a handheld device. For example, the handheld device can be a handheld control device or a tablet.

The remote control system 5006 may be in electronic communication with the robotic interventional device drive system 18 over one or more communication channels as described in greater detail below. The remote control system 5006 may be configured for controlling the outputs of the robotic interventional device drive system 18. Accordingly, a remotely located physician may advantageously control a locally located medical device for providing a medical intervention to a patient.

FIG. 25B illustrates a system diagram of an example of a robotic medical system 5000. The system diagram shows fluid and electrical connectivity between the subsystems of the robotic medical system 5000. The robotic medical system 5000 may include the control system 4000 described above with respect to FIG. 24. As described herein, the robotic medical system 5000 can include one or more local devices and a local control system. The one or more local devices can include a fluidics tower 5002, a robotic drive system 5004, and one or more disposable devices 5008. The local control system can include components configured to be electrically connected to the remote control system 5006. In some embodiments, the local control system can include any of the same or similar components and/or functionality of the remote control system 5006. For example, the local control system may be configured to fully operate the local system 5007 without the remote control system 5006 (e.g., if communication with the remote control system 5006 is interrupted or inaccessible). In some embodiments, the local control system can include one or more electrical components in the local system. For example, the local control system can include tower electronics interface 5010, and/or an interface 5020. The components of the local control system may be located locally in the operating room and in close proximity to or be integrated within the fluidics tower 5002 and the robotic drive system 5004.

The fluidics tower 5002 may be a housing or console including a fluidics management system for controlling the administration or removal of contrast, saline and/or bodily fluids to and/or from an interventional device. The fluidics tower 5002 may further include tower electronics interface 5010, a fluidics station (or “system”) 5012, a monitor 5014, and one or more local communication devices 5016. Although illustrated in FIG. 25B as part of the fluidics tower 5002, in other embodiments the tower electronics interface 5010 may be housed separately from the fluidics tower 5002 while still being in electrical communication with the fluidics tower 5002, the one or more disposable devices 5008, the robotic drive system 5004, and the remote control system 5006.

The tower electronics interface 5010 may be a housing configured to contain system electronics such as one or more processors and memory. The one or more processors and memory may be organized into one or more computer devices. The tower electronics interface 5010 may include a power cord configured to be operatively coupled to a power source, such as a battery, a generator, or an outlet. In some embodiments, the tower electronics interface 5010 may draw power from a power source providing 110/220 volts of alternating current (VAC) power. In some embodiments, the tower electronics interface 5010 may contain the control system 4000 described above. In some embodiments, the one or more processors may include a streaming processor. The streaming processor can be configured to stream imaging data in real-time with ultra-low latency. In some embodiments, the streaming processor can provide a secure connection to allow only authorized users to securely access and stream the data. In some embodiments, the tower electronics interface 5010 can be included in a local control system.

The tower electronics interface 5010 may be a central hub for the robotic medical system 5000 interconnecting the electronic devices from various components as described in greater detail below. The system electronics of the tower electronics interface 5010 may be configured to transmit and receive electronic signals and/or data to operate components of the robotic medical system 5000. The tower electronics interface 5010 may include means for connecting to other devices. The tower electronics interface 5010 may transmit and/or receive electronic signals and/or data wirelessly or over a wired connection. In some embodiments, the tower electronics interface 5010 may include an ethernet port for connecting the system electronics to a network. In some embodiments, the tower electronics interface 5010 may include one or more ports for tethering to nearby electronic devices via a wired connection. For example, the tower electronics interface 5010 may have ports to run cables between the fluidics tower 5002 and the robotic drive system 5004 and/or the remote control system 5006. Alternatively, the tower electronics interface 5010 may be configured to connect wirelessly to nearby electronic devices. For example, the tower electronics interface 5010 may include a personal area network (PAN) module, such as Bluetooth®, or other network capabilities to transmit data wirelessly.

In some embodiments, electronic signals and/or data may include instructions for a system, subsystem, component, or device to perform a particular task. Additionally and/or alternatively, electronic signals and/or data may include indicators or data measured from sensors for processing by a computing device.

In some embodiments, the tower electronics interface 5010 may connect to other devices over a communication network (“network”). The network may cover a small geographic area such as a particular room or building, a medium geographic area such as a city, or a large geographic area so long as there is access to a network. For example, the network may be a local area network (LAN) or wireless local area network (WLAN) comprising a series of devices linked together to form a network within a hospital or clinic. Alternatively, the network may be a wide-area network (WAN) comprising a series of devices linked together to form a network within a medical campus comprising two or more buildings. Alternatively, the network may be an intranet or internet for providing global connectivity. Connecting over the network advantageously connects the operating room to physicians located around the world, including experts located across the nation or in other countries, without requiring the physician to travel to the operating room. This advantageously connects patients to physicians without the time or cost required for the physician to physically travel to the operating room. In some procedures, every minute of delay before a procedure is performed can increase the chance of a bad outcome, and thus such surgical systems can help mitigate damage to the patient due to a delay in starting the surgical procedure.

The fluidics system 5012 may include one or more fluidic subsystems comprising one or more containers, one or more tubes, and one or more pumps. The subsystems may be divided and organized into a contrast subsystem, a saline subsystem, and/or an aspiration (or “vacuum”) subsystem. The fluidics system 5012 may be the fluidics system described herein.

A contrast subsystem may be configured for supplying contrast to a patient. The contrast subsystem may include one or more containers, one or more fluid communication channels (“tubes”), one or more valves, and a high-pressure pump.

The one or more containers may be configured to store contrast and act as a contrast source for supplying contrast to an intervening device such as a catheter as described above. In some embodiments, the one or more containers may have an outer shell defining an interior cavity. In some embodiments, the interior cavity may be enclosed such that the interior cavity is not exposed to the environment. In some embodiments, the one or more containers may be selectively sealed such that the one or more containers may be selectively opened. In such embodiments, the one or more containers may be selectively unsealed to supply contrast and may be selectively sealed to apply a pressure within the interior cavity.

The one or more tubes may have a proximal end and a distal end with a lumen extending therebetween. The one or more tubes may extend from and be in fluid communication with the one or more containers. In some embodiments, the one or more tubes may extend one or more containers from the distal end. In some embodiments, the one or more tubes may extend from a bottom side of the one or more containers. In some embodiments, the one or more tubes may be coupled together forming a plurality of branches. Each of the plurality of branches may be configured to couple with a catheter. In some embodiments, each of the plurality of branches may couple to a distinct catheter.

The one or more valves may operatively be coupled to the one or more tubes and/or the one or more containers. The one or more valves may be configured to selectively control the flow of contrast through the tubes. In some embodiments, each branch of the plurality of branches may include a valve for selectively controlling the flow of contrast through that branch.

The high-pressure pump may be in fluid communication with the one or more containers and the one or more tubes and configured to direct contrast into a patient. The high-pressure pump may be a syringe pump, a high-pressure positive displacement pump, or a contrast injection pump.

A saline subsystem may be configured for supplying saline to a patient. The saline subsystem may similarly include one or more containers, one or more tubes, one or more valves, and one or more pumps, which may be high pressure pumps.

The one or more containers may be configured to store saline and act as a saline source for supplying saline to an intervening device such as a catheter as described above. In some embodiments, the one or more containers may have an outer shell defining an interior cavity. In some embodiments, the interior cavity may be enclosed such that the interior cavity is not exposed to the environment. For example, the one or more containers may be a saline bag. In some embodiments, the one or more containers may be selectively sealed such that the one or more containers may be selectively opened. In such embodiments, the one or more containers may be selectively unsealed to supply saline and may be selectively sealed to apply a pressure within the interior cavity.

The one or more tubes may have a proximal end and a distal end with a lumen extending therebetween. The one or more tubes may extend from and be in fluid communication with the one or more containers. In some embodiments, the one or more tubes may extend one or more containers from the distal end. In some embodiments, the one or more tubes may extend from a bottom side of the one or more containers. The one or more tubes may be coupled together forming a plurality of branches. Each of the plurality of branches may be configured to couple with a catheter. In some embodiments, each of the plurality of branches may couple to a distinct catheter.

The one or more valves may operatively be coupled to the one or more tubes and/or the one or more containers. The one or more valves may be configured to selectively control the flow of saline through the tubes. In some embodiments, each branch of the plurality of branches may include a valve for selectively controlling the flow of saline through that branch.

The one or more pumps may be in fluid communication with the one or more containers and one or more tubes and configured to direct saline out of the one or more containers and through the one or more tubes. In some embodiments, one pump is in fluid communication with a corresponding branch of the one or more tubes. The one or more pumps may be a peristaltic pump.

An aspiration subsystem may be configured for removing biological samples from a patient. The aspiration subsystem may include one or more containers, one or more tubes, one or more valves, and a vacuum pump. In some embodiments, the aspiration system may have two vacuum pumps that produce different amounts of vacuum. In some embodiments, the aspiration subsystem is configured to produce different levels of vacuum, for example, a low vacuum level and a high vacuum level (e.g., higher than the low vacuum level).

The one or more containers may be configured to receive biological samples and act as a destination for an intervening device such as a catheter as described above. In some embodiments, the one or more containers may have an outer shell defining an interior cavity. In some embodiments, the interior cavity may be enclosed such that the interior cavity is not exposed to the environment. In such embodiments, the pressure within the interior cavity may be controlled.

The one or more tubes may have a proximal end and a distal end with a lumen extending therebetween. In some embodiments, the one or more tubes may be a catheter, such as the aspiration catheter described above. The one or more tubes may extend from and be in fluid communication with the one or more containers. In some embodiments, the one or more tubes may extend one or more containers from the distal end. In some embodiments, the one or more tubes may extend from an upper side of the one or more containers. In some embodiments, the one or more tubes may be coupled together forming a plurality of branches. Each of the plurality of branches may be configured to couple with a catheter. In some embodiments, each of the plurality of branches may couple to a distinct catheter.

The one or more valves may operatively be coupled to the one or more tubes and/or the one or more containers. The one or more valves may be configured to selectively control the flow of saline through the tubes. In some embodiments, each branch of the plurality of branches may include a valve for selectively controlling the flow of saline through that branch.

The vacuum pump may be in fluid communication with the one or more containers and one or more tubes and configured to direct biological samples, such as bodily fluids, clots, and/or blood, out of the patient and into the one or more containers using vacuum produced by the vacuum pump.

In some embodiments, one or more saline lines may be in fluid communication with the contrast and aspiration subsystems via one or more valves to allow for flushing of the contrast and aspiration subsystems. In some embodiments, the saline subsystem may also be in fluid communication with a femoral sheath used for introducing an interventional device assembly into the body.

The one or more pumps and containers of the contrast, saline, and aspiration subsystems may be contained with the fluidics tower 5002. The one or more tubes of the contrast, saline, and aspiration subsystems may extend out of the fluidics tower 5002 for interacting with and coupling to other devices.

The monitor 5014 may be any electronic visual computer display (or displays) that includes a screen and circuitry configured to interpret electronic signals to display one or more images. For example, the monitor 5014 may include an imaging window, a speed indicator, a rotational indicator, an axial position bar, a telescopic position window, one or more axial position indicators, and/or other graphical user interfaces or windows. In some embodiments, the monitor 5014 may be configured to display fluoroscopic images, catheter data, fluidics information (e.g., information relating to a contrast injection subsystem including its current operation status, information relating to a saline subsystem including its current operation status, and/or information relating to a aspiration subsystem including its current operation status) including current state information) providing saline, providing vacuum for aspiration), and patient data including vital signs. Additionally and/or alternatively, the monitor 5014 may be configured to display a remotely working physician during a teleoperated procedure, or information related to the remotely working physician. Displaying the working physician during a teleoperated procedure may advantageously enhance communication between the remotely located physician and the technicians and/or assistants located in the operating room thereby increasing the efficiency and safety of teleoperated procedures. In some embodiments, the monitor 5014 may be the display 23 described above.

The one or more local communication devices 5016 may be one or more microphones, one or more cameras, and/or one or more audio output devices such as a speaker and/or a headset. The one or more microphones may be configured to measure and transmit audio from the operating room to a physician working remotely during a teleoperated procedure. Alternatively, the one or more microphones may be configured to measure and save audio from the operating room to a secure database. Similarly, the one or more cameras may be configured to measure and transmit video from the operating room to a physician working remotely during a teleoperated procedure. Alternatively, the one or more cameras may be configured to measure and save video from the operating room to a secure database. The one or more audio output devices may be configured to output an audio signal from a physician working remotely during a teleoperated procedure. In some embodiments, the one or more local communication devices 5016 may include at least one microphone, at least one camera, and at least one audio output device. In some embodiments, the one or more local communication devices 5016 may include a plurality of cameras configured to capture multiple angles and fields of view. In certain embodiments, at least some of the one or more local communication devices 5016 may be housed separately from the fluidics tower 5002. For example, one or more cameras may be positioned within the operating room to provide one or more views of a medical procedure. The one or more cameras may provide a view of a patient, a patient access site, the one or more disposable devices 5008, and/or the robotic drive system 5004. In certain embodiments, one or more microphones may be positioned within the operating room to capture audio from one or more technicians. In some embodiments, the one or more local communication devices 5016 can include a video capture device configured to capture fluoroscopic images. For example, the one or more local communication devices 5016 can include a clot camera.

The fluidics system 5012, the monitor 5014, and the one or more local communication devices 5016 may be electrical communication with the tower electronics interface 5010 and configured to receive and/or transmit electronic signals and/or data therebetween. In some embodiments, the electronic signals and/or data may include instructions to activate one or more pumps and/or one or more valves of the fluidics system 5012. For example, the instructions may direct the fluidics system 5012 to provide saline and/or contrast to the one or more disposable devices 5008. In some embodiments, the data may include video and/or audio inputs and audio outputs for the monitor 5014 and one or more local communication devices 5016. For example, the data may be one or more images to be displayed on the monitor 5014 and/or audio-visual data captured by the one or more local communication devices 5016.

The fluidics tower 5002 may be further configured to be operatively coupled with the one or more disposable devices 5008. In some embodiments, the fluidics system 5012 may be mechanically coupled to and/or in fluid communication with the one or more hubs or hub assemblies 5034. Accordingly, activating the fluidics system 5012 may provide contrast, saline, and/or suction to the one or more hub assemblies 5034 and corresponding interventional devices.

The robotic drive system 5004 may include a plurality of components to drive one or more access systems such as catheters and guidewires during a procedure. The robotic drive system 5004 may be the drive system 18 described above. The robotic drive system 5004 may include a drive table 5018, an interface 5020, and a joint setup 5022.

The drive table 5018 may support the one or more disposable devices 5008 (e.g., a catheter) configured to be advanced to access a patient for performing a surgical procedure and/or for introducing saline, contrast media or therapeutic agents, or providing aspiration. The drive table 5018 may further support a sterile barrier.

The drive table 5018 may be the support table 20 described above. The drive table 5018 may be positioned over or alongside a patient, and configured to axially advance, retract, and in some cases rotate two or three or more different concentrically oriented intravascular devices of an interventional device assembly. The drive table 5018 may include electronics and motors for controlling the location of the interventional devices and actuation of fluidics components. The drive table 5018 may include an outer shell and inner components housed within the outer shell. In some embodiments, the drive table 5018 may fix the outer shell in place after one or more interventional devices engage with a patient. The inner components may continue to move even after one or more interventional devices engage with a patient. In some embodiments, the drive table 5018 may not absorb shock or account for movement of a patient. In such embodiments, a flexible section of the one or more interventional devices, such as a flexible sheath, may absorb forces resulting from minor movements of the patient. In some embodiments, the drive table 5018 may function as a shock absorber. For example, a portion of the drive table 5018 (e.g., the outer shell of the drive table 5018) may not be fixed and may track minor movements of the patient.

In some embodiments, the drive table 5018 may include one or more hub adapters. The one or more hub adapters may include the drive magnets described above. Movement of the drive magnets may be driven by a drive system carried by the drive table 5018. Movement of the drive magnets may be configured to drive one or more hub assemblies 5034 of the one or more disposable devices 5008. The drive table 5018 and the one or more disposable devices may be separated such that the one or more hub assemblies 5034 may not mechanically couple to the drive table 5018 as shown by axis B-B.

Certain embodiments of hub assemblies described herein, such as hub assembly (“hub”) 36 shown in FIG. 4 or one or more hub assemblies 5034, include a housing (e.g., housing 38) for coupling an interventional device thereto, components (e.g., rollers 53 and 55) for directly coupling to and moving along a drive table, and magnet(s) (e.g., magnet 69) for magnetically coupling to a hub adapter across a sterile barrier. A hub (or hub assembly) can refer to a single assembly with a housing, or a hub (or hub assembly) can generally refer to an apparatus having two (or more) subassemblies (e.g., a first subassembly and a second subassembly). In some embodiments of a hub assembly having two subassemblies, a hub can refer to a first subassembly that can be configured to couple to and house an interventional device, and that may be removably attachable to a second subassembly (or mount) configured to magnetically couple to a hub adapter across a sterile barrier and move along a drive table. Such a hub and mount may together form a hub assembly. Such hub assemblies may allow for a hub (first subassembly) to be removed from a mount (second subassembly) which can be advantageous, for example, so that a different hub can be coupled to the same mount or so that the hub may be used separately from the mount (e.g., for a manual procedure).

An arrangement of a hub assembly having a hub that is releasably couplable to mount can allow for replacement of a hub with a different hub having a different interventional device coupled thereto without breaking a magnetic connection with a hub adapter. For example, such an arrangement may allow for a hub coupled to an access catheter to be removed from a mount and replaced with a hub coupled to a procedure catheter without breaking a magnetic connection between active and passive magnetic sides of the coupling of the hub adapter and hub assembly (e.g., between the hub adapter and the mount). In some embodiments, the mount may be a magnetically driven member, an axially driven member, a puck, a slider, a shuttle, or a stage. The robotic drive systems described herein can relate to various embodiments of systems that include a hub, or a hub and a mount, regardless of whether they are described in reference to a hub, or a hub and mount, unless explicitly indicated or indicated by context. In some embodiments, a mount may be magnetically coupled to hub adapter across a sterile barrier prior to coupling a hub to the mount, for example, when preparing the drive table for a medical procedure.

As described herein, the hub assemblies can include intravascular devices that can access the vascular system of a patient via at least one artery and/or vein (e.g., the femoral artery) and be driven within the vascular system to perform a vascular procedure.

The interface 5020 may be any device configured to interact with and/or display information to personnel locally situated within an operating room during a procedure, such as a bedside user. For example, a bedside user may be nurse or surgical technician staffed within the operating room. The interface 5020 may include an imaging window, a speed indicator, a rotational indicator, an axial position bar, a telescopic position window, and/or one or more axial position indicators. The interface 5020 may be the display 23 described above configured to display fluoroscopic images, catheter data, pressure values of the fluidics system, and/or other patient data. In some embodiments, the interface 5020 may be a touchscreen device such as a tablet computer. The interface 5020 may display information to a bedside user. The information displayed to the bedside user may include directions and/or prompts for the bedside user to follow. For example, the information may describe what steps to perform next, how to position the robotic drive system 5004, when to deploy the drapes, whether the system is malfunctioning or whether an error is detected, and/or prompt the bedside user to otherwise interact with the system. In some embodiments, the interface 5020 is configured to accept user input to control one or more components, for example, position of the drive table.

The interface 5020 may be in communication with one or more portions of the robotic medical system 5000 (for example, the robotic drive system 5004, the fluidics tower 5002, the one or more disposable devices 5008, etc.). In some embodiments, the interface 5020 may be mechanically coupled to the robotic drive system 5004, be housed separately, or be mechanically coupled to another part of the robotic medical system 5000. The interface 5020 may control the joint setup 5022 of the robotic drive system 5004. For example, the interface 5020 may control the transition processes between a storage position and a deployed position, engaging a priming sequence, or controlling fine motor adjustments for providing minor adjustments to the positions of the hubs. Controlling the joint setup 5022 and motors with the interface 5020 advantageously provides greater precision and setup before an operation by individuals present in the operating room.

The interface 5020 may advantageously provide a backup control mechanism to interact with and provide input to control the robotic medical system 5000, for example, in the event the remote control system 5006 is rendered incapable of performing an operation. In some embodiments the interface 5020 is part of the local control system described herein. In some embodiments, the interface 5020 can be included in the tower electronics interface 5010.

The joint setup 5022 may include a plurality of joints and motors for controlling the positioning and movements of the robotic drive system 5004. In some embodiments, the joint setup 5022 may initialize the robotic drive system 5004 into a starting position. The initialization process may include transitioning the robotic drive system 5004 from a storage position to a deployed position and vice versa. For example, the joint setup 5022 may be configured to transition the robotic drive system 5004 from a storage position the robotic drive system 5004 to a deployed position such that at least a portion of the robotic drive system 5004 transitions from a compact state to a position where at least a portion of the robotic drive system 5004 is positioned either over or alongside a patient.

Within the robotic drive system 5004, the drive table 5018 may be mechanically coupled with the interface 5020 and the joint setup 5022. The joint setup 5022 may also be electrically connected to the drive table 5018 and the interface 5020. The robotic drive system 5004 may be configured for the joint setup 5022 to transmit electronic signals and data to the drive table 5018 and the interface 5020. Additionally and/or alternatively, the robotic drive system 5004 may be configured for the joint setup 5022 to receive electronic signals and data from the drive table 5018 and the interface 5020.

The remote control system 5006 may be a collection of components configured to control and operate the supra-aortic access robotic control system described above. In some embodiments, the remote control system 5006 can include a control console such as a workstation, a handheld controller, and/or a tablet computer. The remote control system 5006 may include a control device 5024, an interface 5026, one or more monitors 5028, and one or more remote communication devices 5030. The remote control system 5006 can be remotely positioned. For example, the remote control system 5006 may be located in the operating room separated from the local system 5007 components by a fluoroscopic barrier, in a control room within the hospital/clinic with the operating room, in a control room within a hospital/clinic remote from the operating room, or located at another remote location as shown separated from the local system comprising the fluidics tower 5002, the robotic drive system 5004, and the one or more disposable devices 5008 as illustrated by line A-A. The remote control system 5006 may include system electronics comprising one or more processors and one or more memory components (“memory”). The system electronics may be configured to electrically connect the control device 5024, the interface 5026, the one or more monitors 5028, and the one or more remote communication devices 5030.

The remote control system 5006 may include means for connecting to other devices. The remote control system 5006 may transmit and/or receive electronic signals and/or data wirelessly or over a wired connection. In some embodiments, the remote control system 5006 may include an ethernet port for connecting the system electronics to a network. In some embodiments, the remote control system 5006 may include one or more ports for tethering to nearby electronic devices via a wired connection. For example, the remote control system 5006 may have ports to run cables between the remote control system 5006 and the fluidics tower 5002 and/or robotic drive system 5004. Alternatively, the remote control system 5006 may be configured to connect wirelessly to nearby electronic devices. For example, the remote control system 5006 may include a Bluetooth® module or other network capabilities to transmit data wirelessly.

In some embodiments, the remote control system 5006 may connect to other devices over a network as described herein.

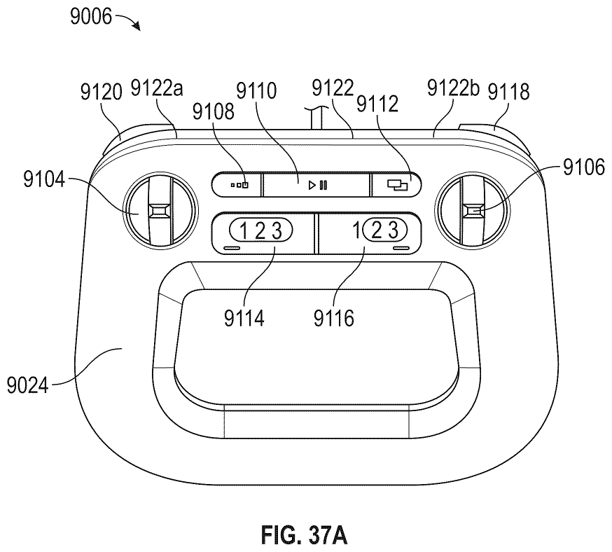

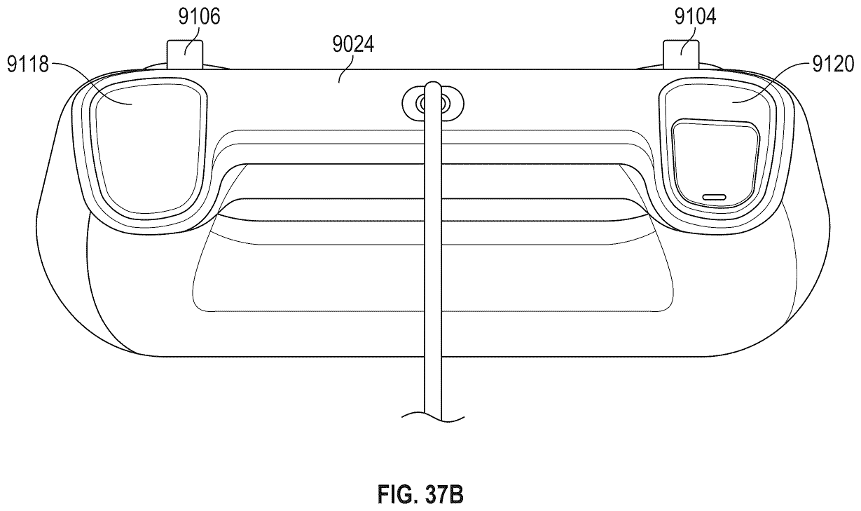

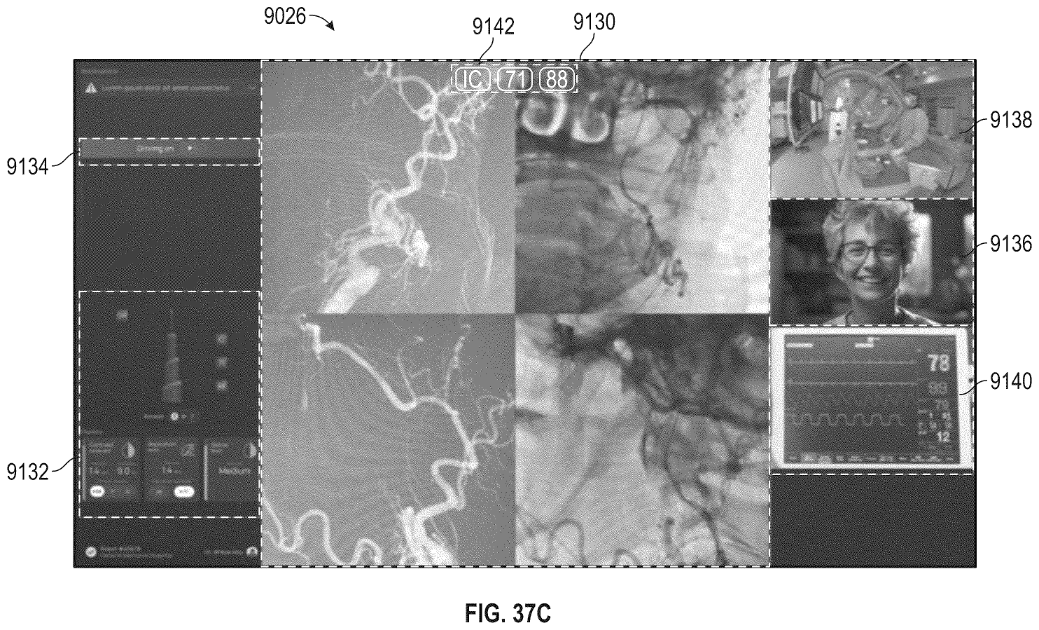

The control device 5024 may be any device configured to enable a surgeon to control portions of the robotic medical system 5000 in the same location as the patient. In some embodiments, the control device 5024 may be part of or coupled to a control console. Although a single control device 5024 is described herein, in certain embodiments, multiple control devices 5024 may be used to control different portions of the robotic medical system 5000. That is, the fluidics tower 5002, the one or more disposable devices 5008, and the robotic drive system 5004. For example, the control device 5024 may be configured to move the hub assemblies to desired positions to perform a procedure on a patient. In some embodiments, the control device 5024 may include input devices where each of the input devices correspond to a particular one of the one or more hub assemblies 5034. For example, in some embodiments, the control device 5024 may include a plurality of input devices such as sliders wherein each of the plurality of sliders correspond to a particular one of the one or more hub assemblies 5034. In another example, the control device 5024 may include a plurality of input devices such as joysticks. In some embodiments, each joystick may be configured to control a corresponding one of the one or more hub assemblies 5034. In some embodiments, the joysticks may bundle controls for one or more hub assemblies 5034 as described below. In other implementations, each input device of the control device 5024 may be a knob that is axially translatable and rotational relative to a support structure. In some embodiments, the control device 5024 may include an input device such as a touchscreen including a graphical user interface (GUI) to control one of the one or more hub assemblies 5034. In some embodiments, the control device 5024 may include an input device such as one or more touchpads to control a corresponding one of the one or more hub assemblies 5034. In some embodiments, the control device 5024 may include a combination of input devices to control a corresponding one of the one or more hub assemblies 5034. In some embodiments, the control device 5024 may include the control mechanism 2200 illustrated in FIGS. 16A-16C. In some embodiments, the control device 5024 may include the control mechanism 9006 illustrated in FIGS. 37A-37B.

The interface 5026 may be configured to display information to the surgeon. The interface 5026 may be the display 23 described above configured to display fluoroscopic images, catheter data, or other patient data. The interface 5026 may be a touchscreen device.

The one or more monitors 5028 may include one or more electronic displays. The one or more monitors 5028 may be any electronic visual computer display that includes a screen and circuitry configured to interpret electronic signals to display one or more images. In some embodiments, the one or more monitors 5028 may be configured to display fluoroscopic images, catheter data, or other patient data. Alternatively, the one or more monitors 5028 may be configured to display one or more views of the operating room. For example, the one or more monitors 5028 may be configured to display the working area during a procedure by displaying only the surgical site. In another example, the one or more monitors 5028 may display the entire operating room including the surgical technicians. In another example, the one or more monitors 5028 may display more than one view. Displaying a plurality of views to capture the entire operating room may advantageously enhance communication and understanding between the remotely located physician and the technicians and/or assistants located in the operating room thereby increasing the efficiency and safety of teleoperated procedures.

The one or more remote communication devices 5030 may be any one or more microphones, one or more cameras, and/or one or more audio output devices. The one or more microphones may be configured to measure and transmit audio from a physician working remotely during a teleoperated procedure to the operating room. Alternatively, the one or more microphones may be configured to measure and save audio from the physician to a secure database. The one or more cameras may be configured to measure and transmit video of a physician working remotely during a teleoperated procedure to the operating room. A visual display of the physician may be advantageous in aiding communication between the physician and the technicians within the operating room. Alternatively, the one or more cameras may be configured to measure and save video of a physician to a secure database. The one or more audio output devices may be configured to output an audio signal from the operating room including vocal signals from technicians or other audio signals that may be used to aid the physician. In some embodiments, the one or more remote communication devices 5030 may include at least one microphone, at least one camera, and at least one audio output device. In some embodiments, the one or more remote communication devices 5030 may include a plurality of cameras configured to capture multiple angles and fields of view.

As shown in FIG. 25B, the fluidics tower 5002, the robotic drive system 5004, the remote control system 5006, and the one or more disposable devices 5008 are connected to a power source. In some embodiments, the fluidics tower 5002 and the remote control system 5006 may be directly connected to a power source such as an outlet. In some embodiments, the robotic drive system and the one or more disposable devices 5008 may indirectly connect to a power source. For example, the robotic drive system 5004 and the one or more disposable devices 5008 may receive power from the fluidics tower 5002. In such embodiments, the joint setup 5022 of the robotic drive system 5004 may be electrically connected to the tower electronics interface 5010 of the fluidics tower and the one or more hub assemblies 5034 of the one or more disposable devices 5008 may be electrically connected to the fluidics system 5012 of the fluidics tower 5002 such that power may be transmitted therebetween.

Furthermore, as shown in FIG. 25B, the fluidics tower 5002, the robotic drive system 5004, the remote control system 5006, and the one or more disposable devices 5008 may be electrically connected and configured to share electrical signals and/or data. In some embodiments, the fluidics tower 5002 may be electrically connected and configured to share electrical signals and/or data with the robotic drive system 5004, the remote control system 5006, and the one or more disposable devices 5008. For example, the tower electronics interface 5010 of the fluidics tower 5002 may be electrically connected with the joint setup 5022 of the robotic drive system 5004 and the remote control system 5006 while the fluidics system 5012 of the fluidics tower 5002 may be electrically connected with the one or more hub assemblies 5034 of the one or more disposable devices 5008. In some embodiments, the tower electronics interface 5010 may be electrically connected with the remote control system 5006 via a network, as shown in FIG. 25B.

The electrical connections of the robotic medical system 5000 are described in greater detail below.

The one or more disposable devices 5008 may be single use objects for use during an operation. The one or more disposable devices 5008 may be configured to interact with a patient and/or form a barrier to prevent biological contamination. The one or more disposable devices 5008 may further include a sterile tray 5032 and one or more hub assemblies 5034.

The sterile tray 5032 may separate the one or more hub assemblies 5034 and corresponding interventional devices from a support table. In some embodiments, the sterile tray 5032 forms a sterile barrier, such as the sterile barrier 32 described above.

The one or more hub assemblies 5034 may be driven for guiding one or more interventional devices, such as the interventional devices 22, to one or more access points for accessing a variety of arteries or veins, such as the femoral artery, radial artery, the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures). In such embodiments, the one or more interventional devices may have an elongate, flexible body, having a proximal end and a distal end. For example, the one or more interventional devices may be a catheter, a guidewire, and/or other suitable devices. In such embodiments, the one or more hub assemblies 5034 may be carried by a support table and moveable along the support table to advance the one or more interventional devices into and out of the patient. For example, the one or more hub assemblies 5034 may be a catheter hub, a guidewire hub, a guide catheter hub, and/or other suitable devices. The one or more hub assemblies 5034 may be the interventional hubs 26, 28, 30 described herein or hubs 2909, 2910, 2912, and 2914 described herein.

The one or more hub assemblies 5034 may include a first hub assembly 5036, a second hub assembly 5038, a third hub assembly 5040, and a fourth hub assembly 5042. In some embodiments, the one or more hub assemblies 5034 may be aligned sequentially such that the first hub assembly 5036 may be positioned at a first end and the fourth hub assembly 5042 may be positioned at a second end opposite the first end. In some embodiments the first end may be a proximal end furthest from a patient and the second end may be a distal end closest to the patient.

In some embodiments, the first hub assembly 5036 may be a guidewire hub, such as the guidewire hub 26 or guidewire hub 2909 described herein; the second hub assembly 5038 may be a first catheter hub, such as the insert catheter or access catheter hub 2910 described herein; the third hub assembly 5040 may be a second catheter hub configured to engage with and guide a procedure catheter, such as the procedure catheter hub 2912 described herein; and the fourth hub assembly 5042 may be a third catheter hub configured to engage with and guide a guide catheter, such as the guide catheter hub 2914 described herein. In some embodiments, the guide catheter may extend distally from the fourth hub assembly 5042.

In certain alternative embodiments, one or more of the one or more disposable devices 5008 may instead be non-disposable. For example, in certain embodiments, the sterile tray 5032 may be part of the drive table. In certain embodiments, one or more of the one or more hub assemblies 5034 and/or sterile tray 5032 may be reusable. [0388] Any audio, video, or other recording that may be captured by the one or more local communication devices 5016 and/or the one or more remote communication devices 5030 may used to identify a patient may be processed and/or saved in compliance with applicable laws and/or regulations such as the Health Insurance Portability and Accountability Act of 1996 (HIPAA), Health Information Technology for Economics and Clinical Health Act (HITECH), and/or the Twenty First Century Cures Act. For example, any audio, video, or other recording captured by the one or more local communication devices 5016, or one or more remote communication devices 5030 may be encrypted, transmitted via a secure virtual private network (VPN), stored on a secure server or may be saved locally on a secured computer and/or hard drive. The fluidics tower 5002 and/or the tower electronics interface 5010 may be configured to mark the data to enable a patient to request its destruction. In some embodiments, the robotic medical system 5000 may insert metadata to identify and/or mark the recordings to enable a patient to request its destruction.

The fluidics tower 5002 may be electrically connected to the robotic drive system 5004, the remote control system 5006, and the one or more hub assemblies 5034 wherein electrical signals and/or data may be transmitted therebetween as discussed in greater detail below. The local system may transmit information about the fluidics system 5012, the robotic drive system 5004, and the one or more hub assemblies 5034 to the remote control system 5006 via the local control system, for example, the tower electronics interface 5010.

The one or more remote communication devices 5030 may be in electrical communication with a power source. The one or more remote communication devices 5030 may further be in electrical communication with the tower electronics interface 5010 and configured to receive and/or transmit electronic signals and/or data therebetween. In some embodiments, the one or more remote communication devices 5030 is in electrical communication with the tower electronics interface 5010 via a network. For example, the one or more remote communication devices 5030 may be electrically coupled to an ethernet cable configured to connect the one or more remote communication devices 5030 to a network, wherein the tower electronics interface 5010 may be electrically coupled to the network.

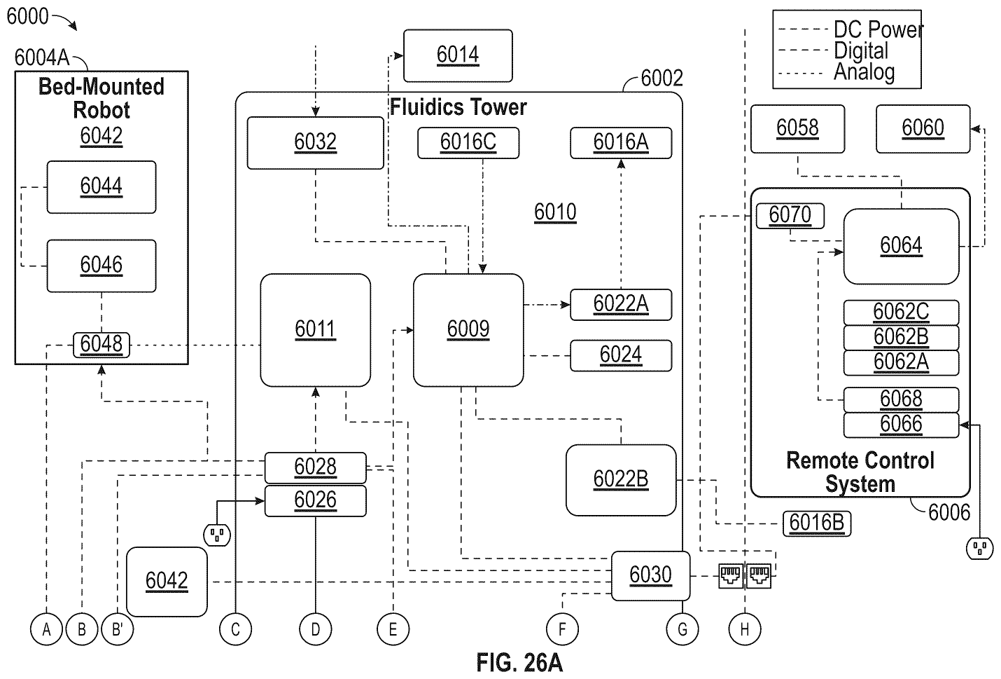

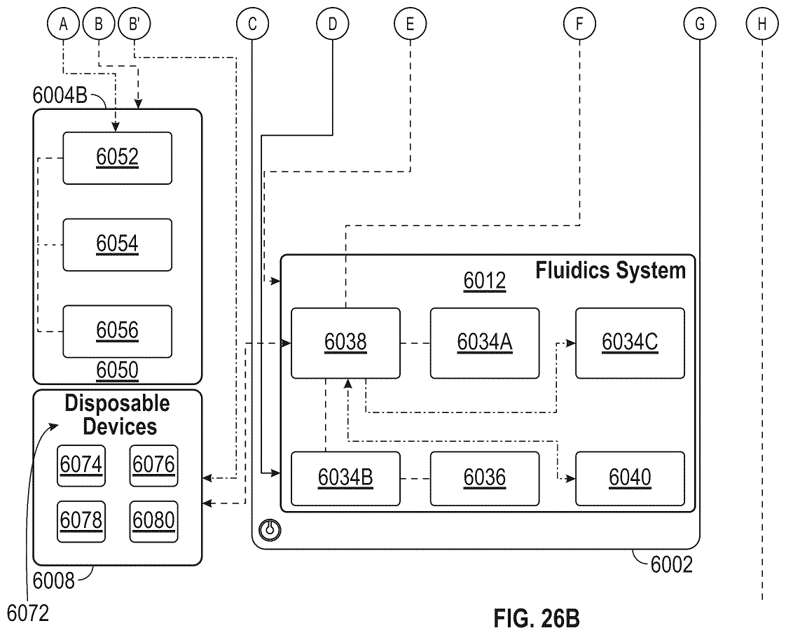

FIGS. 26A and 26B illustrate an electrical system diagram of an example of a robotic medical system 6000. The robotic medical system 6000 may be the robotic medical system 5000 described above with respect to FIGS. 25A and 25B. The robotic medical system 6000 may include a fluidics tower 6002, a robotic drive system 6004 (separated into 6004A and 6004B across FIGS. 26A and 26B), a remote control system 6006, and one or more disposable devices 6008. The robotic medical system 6000 can include a local system and a local control system. The local system can include the devices and components located within an operating room. For example, the local system can include the fluidics tower 6002, the robotic drive system 6004A, the one or more disposable devices 6008, and the fluidics system 6012. The local control system can include the electronic components configured to control the robotic medical system 6000. For example, the local control system can include the tower electronics interface described herein and/or an interface of the robotic drive system.

The fluidics tower 6002 may include an tower electronics interface 6010, a fluidics system 6012, a monitor 6014, one or more local communication devices, one or more computer devices such as a computer 6009, a controller 6011, one or more intermediate devices, a cellular card 6024, a battery 6026, a power distribution unit (PDU) 6028, a switch 6030, a video capture device 6032, one or more fluidic subsystems, one or more valves 6036, a controller 6038, and one or more sensors 6040. In some embodiments, the fluidics tower 6002 may be the fluidics tower 5002 described above. Accordingly, the tower electronics interface 6010, the fluidics system 6012, the monitor 6014, and the one or more local communication devices may be the tower electronics interface 5010, the fluidics system 5012, the monitor 5014, and the one or more local communication devices 5016, respectively.

The tower electronics interface 6010 may be configured to include the one or more local communication devices, the computer 6009, the controller 6011, the one or more intermediate devices, the cellular card 6024, the battery 6026, the PDU 6028, the switch 6030, and the video capture device 6032. In some embodiments, the tower electronics interface 6010 may be physically stored in a housing that forms a base for the fluidics tower 6002.

The one or more local communication devices may include a first local communication device 6016A, a second local communication device 6016B, and a third local communication device 6016C. The one or more local communication devices may be any device and/or sensor capable of providing communication between a two or more people such as a camera, an audio output device, a microphone, and/or a display. In some embodiments, the first local communication device 6016A may be an audio output device; the second local communication device 6016B may be a camera; and the third local communication device 6016C may be a microphone.

The computer 6009 may be a computing device configured to process, transmit, and/or receive electrical signals and/or data. In some embodiments, the computer 6009 may serve as a computing hub for one or more components of the robotic medical system 6000. For example, the computer 6009 may be a computing hub for the fluidics tower 6002 comprising the monitor 6014, the one or more local communication devices, the cellular card 6024, and/or video capture device 6032. The computer 6009 may include a plurality of ports configured to be electrically connected with other electrical components. In some embodiments the computer 6009 may receive, process, and/or transmit electrical signals and/or data such as audio, video, image, cellular, and/or other data. The computer 6009 may receive electrical power from a power source. The power source may be an outlet, a battery, or other power sources known in the art.

In some embodiments, the computer 6009 may include an artificial intelligence (AI) logic for processing input data, calculating responses, and transmitting outputs. In some embodiments, the AI may be implemented in software, hardware, or a combination of hardware and software. In some embodiments, the AI may be implemented to scan incoming x-ray or fluoroscope images to detect one or more interventional devices, for example, the location of the distal tip of one or more of the interventional devices. In some embodiments, the AI may be implemented to provide status information to the remote console or the robotic system, based on the detection of the interventional devices, for example, the location of the distal tips of the interventional devices. In some embodiments, the AI may be implemented to provide control information to the surgical system based on the detection of the interventional devices, for example, the detection of the location of the distal tips of the interventional devices. In some embodiments, the AI is configured to scan generated x-ray or fluoro images to detect one or more interventional devices. The computer 6009 may be configured to process large amounts of data. For example, the computer 6009 may be a NVIDIA® AGX system configured to process computationally intensive applications and/or data sets. Furthermore, the computer 6009 may run any operating system capable of simultaneously managing several components. For example, the computer 6009 may be a Linux® based operating system such as Ubuntu®. The computer 6009 may further include chips configured for coding and/or encoding to compress video and conserve bandwidth. The conserved bandwidth may improve latency.

The controller 6011 may be a hardware device such as a processor or other computer device configured to manage or direct data flow between two components. For example, the controller 6011 may be configured to manage and direct data flow between the fluidics tower 6002 and the robotic drive system 6004A, 6004B. The controller 6011 may include one or more cores, microchips, and/or separate hardware devices for controlling a peripheral device. In some embodiments, the controller 6011 may be an Advantech® MIO-5375 system controller.

The one or more intermediate devices may be any device configured to monitor, manipulate, and/or capture data in connection with a communication device such as a camera, microphone, and/or audio output device. The one or more intermediate devices may be an analog-to-digital converter (ADC) for converting analog signals to digital signals. Alternatively, the one or more intermediate devices may be a digital-to-analog converter (DAC) for converting digital signals to analog signals. In some embodiments the one or more intermediate devices may be a video capture device and/or an audio amplifier.

The one or more intermediate devices may include a first intermediate device 6022A and a second intermediate device 6022B. In some embodiments, the first intermediate device 6022A may be an audio amplifier configured to reproduce input audio signals at sound-producing output elements with minimal distortion. The audio amplifier may be a DAC configured to convert digital audio signals into analog audio signals to be output by a audio output device. In some embodiments, the second intermediate device 6022B may be a video capture device configured to capture individual still frames from a video signal or video stream, as discussed in greater detail below. In some embodiments, the video capture device may be a video capture card for capturing frames in a video format. In some embodiments, the frames may be digital. The second intermediate device 6022B may include onboard buffers to temporarily store image data for manipulating an image prior to transferring the images to a computing device.

The cellular card 6024, may be a smart card and/or device configured to connect the fluidics tower 6002 to a cellular network. The cellular card 6024 may provide a backup communication channel in the event the primary network is unavailable. The cellular card 6024 may store identification information about the fluidics tower 6002. Additionally, the cellular card 6024 may connect the fluidics tower 6002 to an internet of things (IoT) describing physical objects comprising sensors, processing ability, software, and/or other technologies that connect and exchange data with other devices and systems over the internet or other communication networks. For example, the cellular card 6024 may be a subscriber identity module (SIM) card. The cellular card 6024 may thus advantageously provide a backup communication channel to mitigate safety concerns of unplanned termination of a teleoperated procedure resulting from failure of a network.

The battery 6026 may be configured to store electrical power for use by the fluidics tower 6002. The battery 6026 may be any electrical power storage medium and/or device.

In some embodiments, the battery 6026 may be a portable power supply (PPS) configured to provide primary power to the fluidics tower 6002. A PPS may be a mobile energy source that does not require a connection to the power grid. In such embodiments, the battery 6026 may store sufficient power required for operating the fluidics tower 6002 for the duration of a procedure. For example, the PPS may store sufficient power to run for several hours and/or days at a time. A PPS advantageously allows the fluidics tower 6002 to be used remotely and can be implemented in a rural or other environment with either no power grid, or a limited/unreliable power grid.

In some embodiments, the battery 6026 may be an uninterruptible power supply (UPS) configured to provide backup and/or secondary power in the event of an electrical disturbance of the primary power source such as a blackout, a brownout, or other power grid failure. In such embodiments, the battery 6026 may store sufficient power to allow the fluidics tower 6002 to keep running for at least a period of time beyond the occurrence of the electrical disturbance. The UPS may be configured to store less electrical power than the PPS. For example, the UPS may store sufficient power to run from several minutes to a couple of hours. Furthermore, in such embodiments, the battery 6026 may be activated and provide power to the fluidics tower 6002 nearly instantaneously in response to an electrical disturbance. Accordingly, a UPS may advantageously protect the electrical components of the fluidics tower 6002 in the event of an electrical disturbance and provide sufficient power to safely conclude and/or terminate a procedure.

The PDU 6028 may be a device configured to manage, control and/or distribute electrical power. The PDU 6028 may be a power strip comprising a plurality of electrical outlets for supplying power to multiple components simultaneously. The PDU 6028 may advantageously ensure that the fluidics tower 6002 and corresponding components are equally and/or adequately powered. In some embodiments, the PDU 6028 may provide surge protection. In some embodiments, the PDU 6028 may include real-time monitoring and provide remote access capabilities. The electrical power may be alternating current (AC) or direct current (DC). The electrical power distributed by the PDU 6028 may be received from the battery 6026, a utility supplier (e.g., the power grid), or a generator or other secondary power source.

The switch 6030 may be a network connecting device configured to organize the flow of electronic signals and data on the same network and prevent traffic between two devices from getting mixed up. The switch 6030 may be a switch for connecting several devices on a singular network or may be a router for connecting several devices across a plurality of networks.

The video capture device 6032 may be an electronic device configured to acquire high-resolution digital images or still frames from a continuous video feed with specific synchronization features in real-time. For example, the video capture device 6032 may be a video capture card. The video capture device 6032 may capture digital still images of either an analog video signal or a digital video stream. Alternatively, the video capture device 6032 may begin transmitting a signal before a full frame is captured such that the captured image is not a still image. Accordingly, the video capture device 6032 may manage the image data from a video source and store the image data to a memory of a computing device. In some embodiments, the video source may be a camera configured to capture radiographic images. For example, the video capture device 6032 may capture images of a radiographic x-ray video feed captured by an x-ray machine, a fluoroscopic machine, a computed tomography (CT) scanner, and/or magnetic resonance imaging (MRI) device and send the images to the computer 6009. For example, the video capture device 6032 can capture fluoroscopic images. In some embodiments, the video capture device 6032 may continually capture digital “still” images of the video stream. For example, the video capture device 6032 may capture digital images of the video stream between 30 and 60 frames per second. Continually capturing images advantageously allows for capturing images as the effectiveness of contrast fluctuates. In some embodiments, the video capture device 6032 can be a clot camera.

Additionally and/or alternatively, image data from a video source may be transmitted through a hospital network. In some embodiments, radiographic image data captured by an x-ray machine, fluoroscopic machine, CT scanner, and/or MRI device may be transmitted through a picture archiving and communication system (PACS). The remote control system 6006 may be connected to the hospital network. In some embodiments, the remote control system 6006 may be configured to pull data from the hospital network. For example, the remote control system 6006 may pull pre-operative patient data from a PACS server. In such embodiments, image data may not be transmitted through the video capture device 6032.

In some embodiments, the fluidics tower 6002 may include a plurality of video capture devices 6032 each electrically connected to a corresponding video stream captured by a distinct video source. Each video source may be uniquely oriented to capture a distinct view of an operating area. For example, a first video source may be oriented to capture a top view of a patient's anatomy, a second video source may be oriented to capture a front view of the patient's anatomy, and a third video source may be oriented to capture a side view of the patient's anatomy. To minimize network bandwidth usage, fewer than all views may be transmitted to the remotely located physician. In some embodiments, video capture devices 6032 may only capture images when the unique view of the video capture device 6032 is transmitted to the remotely located physician.

The fluidics system 6012 may be configured to include the one or more fluidic subsystems, the one or more valves 6036, the controller 6038, and the one or more sensors 6040.

The one or more fluidic subsystems may include a first fluidic subsystem 6034A, a second fluidic subsystem 6034B, and a third fluidic subsystem 6034C. In some embodiments, the first fluidic subsystem 6034A may be a contrast subsystem as described above; the second fluidic subsystem 6034B may be a saline subsystem as described above; and the third fluidic subsystem 6034C may be an aspiration subsystem as described above.

The one or more valves 6036 may be the valves described above. In some embodiments, the one or more valves 6036 may be configured to control the flow on saline through the second fluidic subsystem 6034B.

The controller 6038 may be similar to the controller 6011 but configured to control the one or more fluidic subsystems, and one or more sensors 6040.

The one or more sensors 6040 may be configured to measure pressure and/or flow rates of the contrast, saline, and/or vacuum, fluid levels, and/or position of one or more valves. Additionally the one or more sensors 6040 may include a pressure sensor positioned near an interventional device for monitoring pressure of the interventional device. In some embodiments, the pressure sensor is a hemodynamic pressure sensor. The measured pressure may be used to detect pressure waves indicative of the interventional device's position relative to a vessel wall, to determine whether the interventional device entered a clot, or to determine whether a clot blocks the lumen of the interventional device.

The robotic drive system 6004A, 6004B may include a plurality of components to drive one or more access systems such as catheters and guidewires during a procedure. The robotic drive system 6004 may include a drive table 6042 and joint setup robot 6050. The robotic drive system 6004 may be the robotic drive system 5004 described above. Accordingly, the drive table 6042 may be the drive table 5018 described above, and the joint setup robot 6050 may be the joint setup 5022 described above.

The drive table 6042 may be configured for carrying a drive components of the robotic drive system 6004A, 6004B. In some embodiments, the drive table 6042 may be the drive table 5018 or as otherwise described above. The drive table 6042 may further include one or more motor axis hubs (“pucks”) 6044, one or more sub-controllers 6046, and a switch 6048. The drive table 6042 may be configured to expand. In some embodiments, the drive table 6042 may be translated axially. An axial translation of the drive table 6042 may translate the one or more motor axis hubs 6044 coupled to the drive table 6042 axially during a procedure in embodiments where the one or more motor axis hubs 6044 are fixed relative to the drive table 6042.

At least a portion of the drive table 6042 may be expandable. For example, the drive table 6042 may expand in an axial direction. In some embodiments, the drive table 6042 may include a plurality of drive table segments arranged in a telescopic arrangement. The plurality of drive table segments may vary in size, each defining a cavity. In such embodiments, the plurality of drive table segments may be sized to slidably receive another drive table segment such that the drive table segments may collapse into the largest sized drive table segment for having a minimum size and/or expand to the combined length of the collective plurality of drive table segments for having a maximum size. The drive table 6042 may be extended and/or retraced to various lengths between the maximum size and the minimum size. Accordingly, the drive table segments may be configured in a nesting and/or telescopic arrangement. A drive table 6042 configured to telescope may reduce the overall length of the drive table in comparison to a drive table that does not axially translate or have an axially extendable portion.

The one or more motor axis hubs 6044 may be configured to travel along a linear path of the robotic drive system 6004A. The one or more motor axis hubs 6044 may cause linear actuation of catheters and/or guide wires to a patient. In some embodiments, the one or more motor axis hubs 6044 may be the one or more interventional hubs 26, 28, 30, 5036, 5038, 5040, 5042 described above. In some embodiments, the one or more motor axis hubs 6044 may include four motor axis pucks. For example, the one or more motor axis hubs 6044 may include a first hub; a second hub; a third hub; and a fourth hub. In some embodiments, the first hub may be a guidewire hub; the second hub may be a first catheter hub, such as an access catheter hub; the third hub may be a second catheter hub configured to engage with and guide a procedure catheter, such as a procedure catheter hub; and the fourth hub may be a third catheter hub configured to engage with and guide a guide catheter, such as a guide catheter hub.

The one or more sub-controllers 6046 may be configured to control the one or more motor axis hubs 6044. The one or more sub-controllers 6046 may include the same or similar components as the controllers 6011, 6038 described above.

The switch 6048 may be a network connecting device configured to organize the flow of electronic signals and data on the same network and prevent traffic between two devices from getting mixed up. In some embodiments, the switch 6048 may be similar to the switch 6030. The switch 6048 may be implemented on the robotic drive system 6004A for connecting components of and organizing data from for the robotic drive system 6004A.

The joint setup robot 6050 may be the joint setup 5022, described above. In some embodiments, the joint setup robot 6050 may further include one or more sub-controllers 6052, a one or more motor axis telescopic drives 6054, and a motor axis setup joint 6056.

The one or more sub-controllers 6052 may be configured to control the one or more motor axis telescopic drives 6054 and the motor axis setup joint 6056. The one or more sub-controllers 6052 may receive control instructions from the controller 6011, computer 6009, and/or remote control system 6006.

The one or more motor axis telescopic drives 6054 may include a plurality of drives configured for extending the drive table 6042. For example, the one or more motor axis telescopic drives 6054 may linearly actuate the drive table 6042 between a maximum size and a minimum size. In some embodiments, the one or more motor axis telescopic drive include at least two drives. In some embodiments, each of the one or more motor axis telescopic drives 6054 may be coupled to a distinct drive table segment. Additionally and/or alternatively, the one or more motor axis telescopic drives 6054 may be coupled to a proximal most and/or distal most drive table segment.

The motor axis setup joint 6056 may include electronic components for controlling the setup joint comprising the physical hardware such as motors and links.

The remote control system 6006 may include a control device 6058, an interface, a monitor 6060, and one or more remote communication devices. In some embodiments, the remote control system 6006 may be the remote control system 5006 described above. Accordingly, the control device 6058, the interface, the monitor 6060, and the one or more remote communication devices may be the control device 5024, the interface 5026, the one or more monitors 5028, and the one or more remote communication devices 5030, respectively. The remote control system 6006 may further include one or more computer devices, such as a computer 6064, a battery 6066, a PDU 6068, and a switch 6070.

The computer 6064 may be a computing device configured to process, transmit, and/or receive electrical signals and/or data. In some embodiments, the computer 6064 may serve as a computing hub for one or more components of the robotic medical system 6000. For example, the computer 6064 may be a computing hub for the remote control system 6006 comprising the control device 6058, the interface, the monitor 6060, and/or the one or more remote communication devices. The computer 6064, may be similar to the computer 6009.

The battery 6066 may be configured to store electrical power for use by the remote control system 6006. The battery 6066 may be any electrical power storage medium and/or device. In some embodiments, the battery 6066 may be the same type as the battery 6026. In other embodiments, the battery 6066 may be a different type as the battery 6026. For example, the battery 6026 may be a UPS and the battery 6066 may be a PPS. In such embodiments, the battery 6026 may insulate the fluidics tower 6002 from a power disruption and the battery 6066 may supply all of the power to the remote control system 6006 for remote use. Accordingly, the remote control system 6006 may be used off the power grid wherever the surgeon may be located.

The PDU 6068 may be a device configured to manage, control and/or distribute electrical power. The PDU 6068 may be a power strip comprising a plurality of electrical outlets for supplying power to multiple components simultaneously. In some embodiments, the PDU 6068 may be the same as or similar to the PDU 6028 but configured to distribute electrical power to the remote control system 6006.

The switch 6070 may be a network connecting device configured to organize the flow of electronic signals and data on the same network and prevent traffic between two devices from getting mixed up. The switch 6070 may be a switch for connecting several devices on a singular network or may be a router for connecting several devices across a plurality of networks. In some embodiments, the switch 6070 may be the same as or similar to the switch 6030 but configured to organize the flow of electronic signals and data to, from, and/or within the remote control system 6006.

The one or more disposable devices 6008 may be single use objects for use during an operation. The one or more disposable devices 6008 may be configured to interact with a patient and/or form a barrier to prevent biological contamination. The one or more disposable devices 6008 may further include a sterile tray and one or more hub assemblies 6072. The one or more hub assemblies 6072 may include a first hub assembly 6074, a second hub assembly 6076, a third hub assembly 6078, and a fourth hub assembly 6080. In some embodiments, the one or more hub assemblies 6072 may be the one or more hub assemblies 5034 described above. Accordingly, the first hub assembly 6074, the second hub assembly 6076, the third hub assembly 6078, and the fourth hub assembly 6080 may be the first hub assembly 5036, the second hub assembly 5038, the third hub assembly 5040, and the fourth hub assembly 5042 described above, respectively.

The following is a description of the electrical connections of the robotic medical system 6000 and how instructions may be transmitted from the remote control system 6006 to and implemented by the fluidics tower 6002 and/or robotic drive system 6004A, 6004B.

The computer 6009 may be a hub for the fluidics tower 6002 and may be electrically connected to the monitor 6014, the one or more local communication devices, the cellular card 6024, the switch 6030, and the video capture device 6032, and a power source such as an outlet, the battery 6026 and/or the PDU 6028. In some embodiments, the computer 6009 may be further electrically connected to the one or more intermediate devices. The one or more intermediate devices may be positioned electrically between the computer 6009 and the one or more local communication devices.

The computer 6009 may be configured to receive, transmit, and process data signals between the monitor 6014, the one or more local communication devices, the cellular card 6024, the switch 6030, and the video capture device 6032. In some embodiments, the computer 6009 may be configured to only receive, transmit, and process digital signals and data, as shown in FIG. 26A. Accordingly, the electronic signals between the computer 6009 and the monitor 6014, the one or more local communication devices, the cellular card 6024, the switch 6030, and the video capture device 6032 may be digital. In some embodiments, one or more of the connected devices (e.g., the monitor 6014, the one or more local communication devices, the cellular card 6024, the switch 6030 and/or the video capture device 6032) may receive, transmit, and/or process analog signals. Accordingly, one or more intermediate devices may be implemented between the computer 6009 and the one or more connected devices for converting the analog signal into a digital signal to be received and processed by the computer 6009.

The computer 6009 may be electrically connected to the monitor 6014, the one or more local communication devices, the cellular card 6024, the switch 6030, and the video capture device 6032 via a wired connection and/or a wireless connection. A wired connection may include an ethernet cable, a high-definition multimedia interface (HDMI) cable, a peripheral component interconnect express (PCIe) cable, a control area network (CAN) cable, a general purpose input/output (GPIO) cable, an advanced digital cable (ADC), and/or a current source inverter (CSI). Additionally and/or alternatively, the computer 6009 may implement protocols for transmitting data. In some embodiments, a protocol may be implemented to control motor axes. For example, the computer 6009 may implement a controller area network (CAN) protocol and/or ethernet for control automation technology (EtherCAT®) to control motor axes from the subcontrollers. In some embodiments, the computer 6009 may be connected to one or more wired connection type simultaneously. For example, the computer 6009 may be in electrical communication with: the switch 6030 via an ethernet cable; the cellular card 6024 and video capture device 6032 via a PCIe cable; the monitor 6014 via an HDMI cable; and the second intermediate device 6022B via a CSI. A wireless connection may include radio frequency transmission such as Bluetooth® and/or WLAN such as WiFi®, infrared transmission, microwave transmission, and/or lightwave transmission such as fiber optics and/or TAT-8. In some embodiments, the computer 6009 simultaneously implement a wired connection and a wireless connection.

The computer 6009 may be configured to transmit electrical signals and/or data as an output from the computer 6009 to the monitor 6014 and the first local communication device 6016A. In some embodiments, the computer 6009 may transmit electrical signals to the first local communication device 6016A via the first intermediate device 6022A, as shown in FIG. 26A. The computer 6009 may be configured to receive electrical signals and/or data as an input from the second local communication device 6016B via the second intermediate device 6022B, the third local communication device 6016C, and the video capture device 6032. In some embodiments, the computer 6009 may be configured to transmit and/or receive electrical signals and/or data between the computer 6009 and the cellular card 6024 and switch 6030.

The computer 6009 may receive captured video from the video capture device 6032, encode the captured video into a compressed state, and transmit the encoded captured video to the remote control system 6006. Additionally and/or alternatively, the computer 6009 may receive encoded video from the remote control system 6006, decode the encoded video, and display the decoded video on a monitor 6014.

The computer 6009 may be electrically connected to a power source such as an outlet, a battery, and/or a PDU. In some embodiments, the computer 6009 may be electrically connected to the PDU 6028. The PDU 6028 may provide electrical power to the computer 6009, as shown in FIG. 26A. In some embodiments, the PDU 6028 may provide 5 to 12 volts to the computer 6009. The electrical connection between the computer 6009 and the PDU 6028 may be DC current.

The controller 6011 may be electrically connected to the robotic drive system 6004, the switch 6030, and a power source such as an outlet, the battery 6026 and/or the PDU 6028.

The controller 6011 may be configured to receive, transmit, and/or process electrical signals. The electrical signals may be digital and/or analog signals. In some embodiments, the controller 6011 may be configured to simultaneously receive, transmit, and/or process digital and analog signals. For example, the controller 6011 may receive, transmit, and/or process analog signals between the controller 6011 and the robotic drive system 6004 and may receive, transmit, and/or process digital signals between the controller 6011 and the switch 6030. Alternatively, the controller 6011 may receive, transmit, and/or process digital signals between the robotic drive system 6004 and the switch 6030.

The controller 6011 may be electrically connected to the robotic drive system 6004 and the switch 6030 via a wired connection and/or a wireless connection. A wired connection may include an ethernet cable, an HDMI cable, a PCIe cable, and/or a CSI. In some embodiments, the controller 6011 may exclusively implement wired connection, exclusively implement wireless connections, or simultaneously implement wired connections and wireless connections. For example, the controller 6011 may be electrically connected to the switch 6030 via a wired connection and to the robotic drive system 6004 via a wireless connection. Alternatively, the controller 6011 may be electrically connected to the robotic drive system 6004 and the switch 6030 via a wired connection. For example, the controller 6011 may be electrically connected to the robotic drive system 6004 and the switch 6030 via an ethernet cable. Additionally, and/or alternatively, the controller 6011 may be wirelessly connected to the remote control system 6006. In some embodiments, the controller 6011 may be wirelessly connected to the computer 6064 of the remote control system 6006. For example, the controller 6011 may wireless connect with the computer 6064 over WLAN such as WiFi® or radio frequency transmission such as Bluetooth®.

The controller 6011 may be configured to transmit and/or receive electrical signals and/or data between the controller 6011 and the switch 6030 and the robotic drive system 6004. In some embodiments, the electrical signals and/or data may flow from the switch 6030 to the controller 6011 and from the controller 6011 to the robotic drive system 6004.

The controller 6011 may be electrically connected to a power source such as an outlet, the battery 6026, and/or the PDU 6028. In some embodiments, the controller 6011 may be electrically connected to the PDU 6028. The PDU 6028 may provide electrical power to the controller 6011, as shown in FIG. 26A. In some embodiments, the PDU 6028 may provide 12 volts to the controller 6011. The electrical connection between the controller 6011 and the PDU 6028 may be DC current.

The monitor 6014 may be electrically connected to the computer 6009. In some embodiments, the monitor 6014 may be configured display a digital image. In such embodiments, the monitor 6014 may be configured to receive and/or process electronic signals sent from the computer 6009. The monitor 6014 may be electrically connected to the computer 6009 via an HDMI cable. In some embodiments, the electrical signals and/or data received by the monitor 6014 may be one or more digital images. The one or more digital images may be in any format capable of being processed and displayed on the monitor 6014. The monitor may be configured to display real time video. In some embodiments, the real time video may be streamed. For example, the real time video may be streamed via Web Real-Time Communication (webRTC), Real Time Streaming Protocol (RTSP), Secure Reliable Transport (SRT), HTTP Live Streaming (HLS), and/or Real-Time Messaging Protocol (RTMP).

The monitor 6014 may electrically connected to a power source such as an outlet, a battery, and/or a PDU. In some embodiments, the monitor 6014 may be electrically connected to the PDU 6028.

The one or more local communication devices may be electrically connected to the computer 6009. In some embodiments, the one or more local communication devices may be electrically connected to the computer 6009 via one or more intermediate devices.

The one or more local communication devices may be configured to receive, transmit, and/or process electrical signals and/or data. The electronic signals and/or data may be digital and/or analog signals. In some embodiments, the one or more local communication devices may streamed electrical signals and/or data. For example, the electrical signals and/or data may be streamed in real time via wert, RTSP, SRT, HLS, and/or RTMP. In some embodiments, the first local communication device 6016A may be configured to receive and/or process electrical signals and/or data from the computer 6009; the second local communication device 6016B may be configured to process and/or transmit electrical signals and/or data to the computer 6009; and the third local communication device 6016C may be configured to process and/or transmit electrical signals and/or data to the computer 6009. In such embodiments, the first local communication device 6016A may receive electrical signals and/or data from the computer 6009 via the first intermediate device 6022A and the second local communication device 6016B may transmit electrical signals and/or data to the computer via the second intermediate device 6022B. In some embodiments, the first local communication device 6016A may be configured to receive analog signals such as an analog audio signal. In some embodiments, the second local communication device 6016B may be configured to transmit digital signals such as a digital image or video signal. In some embodiments, the third local communication device 6016C may be configured to transmit digital signals such as a digital audio signal.

The one or more local communication devices may be electrically connected to a power source such as an outlet, a battery, and/or a PDU. In some embodiments, the one or more local communication devices may be electrically connected to the PDU 6028.

The one or more intermediate devices may be electrically connected to the computer 6009 and the one or more local communication devices. In some embodiments, the one or more intermediate devices may be electrically connected to the computer 6009 via a CSI cable.

The one or more intermediate devices may be configured to receive, transmit, and/or process electrical signals and/or data. The electronic signals and/or data may be digital and/or analog signals. In some embodiments, the first intermediate device 6022A may be configured to: receive electrical signals and/or data from the computer 6009; process the electrical signals and/or data received from the computer 6009; and transmit the processed electrical signals and/or data to the first local communication device 6016A. In some embodiments, the second intermediate device 6022B may be configured to: receive electrical signals and/or data from the second local communication device 6016B; process the received electrical signals and/or data received from the second local communication device 6016B; and transmit the processed electrical signals and/or data to the computer 6009. In some embodiments, the first intermediate device 6022A may be configured to receive digital signals such as a digital audio signal and output an analog signal such as an analog audio signal. In some embodiments, the second intermediate device 6022B may be configured to receive a first digital signal such as a digital video signal and output a second digital signal different from the first digital signal such as a digital image signal. A digital video signal may include a plurality of continuous images forming a video. A digital image signal may include a subset of images taken from the digital video signal.

The one or more intermediate devices may be electrically connected to a power source such as an outlet, a battery, and/or a PDU. In some embodiments, the one or more intermediate devices may be electrically connected to the PDU 6028.

The cellular card 6024 may be electrically connected to the computer 6009 via a high-speed serial computer expansion bus. For example, the cellular card 6024 may be connected to the computer 6009 via a PCIe cable.

The battery 6026 may be electrically connected to the electrical components of the fluidics tower 6002, the robotic drive system 6004, the one or more disposable devices 6008, and/or the PDU 6028. The electrical components of the fluidics tower 6002 may include the electronic components of the tower electronics interface 6010 and the fluidics system 6012. The electrical components of the fluidics system may include: the computer 6009, the controller 6011, the monitor 6014, the one or more local communication devices, the one or more intermediate devices, the cellular card 6024, the switch 6030, the video capture device 6032. The electrical components of the fluidics system 6012 may include the one or more fluidic subsystems, the one or more valves 6036, the controller 6038, and/or the one or more sensors 6040. In some embodiments, the battery 6026 may be electrically connected to either the electrical components of the fluidics tower 6002 or to the PDU 6028. The battery 6026 may be further electrically connected to a power source such as an outlet, generator, solar panel, and/or other sources of electrical power. The battery 6026 may be configured to receive electrical power from the power source; store the electrical power; and transmit the electrical power to the electrical components of the fluidics tower 6002. In some embodiments, the electrical power may be indirectly transmitted to the electrical components of the fluidics tower 6002 via the PDU 6028.

The battery 6026 may be configured to receive 110/220 volts of AC. The battery 6026 may be configured to transmit: 5 to 12 volts to the computer 6009; 12 volts to the controller 6011; 5 to 48 volts to the fluidics system 6012; 5 to 48 volts to the robotic drive system 6004, and 5 volts to the one or more disposable devices 6008.

The PDU 6028 may be electrically connected to the electrical components of the fluidics tower 6002, to the robotic drive system 6004, the one or more disposable devices 6008, and the battery 6026. The PDU 6028 may be configured to receive electrical power from the battery 6026 and transmit the electrical power to the electrical components of the fluidics tower 6002.

The PDU 6028 may be configured to receive sufficient power from the battery for distributing power to the connected components. For example, the PDU 6028 may distribute: 5 to 12 volts to the computer 6009; 12 volts to the controller 6011; 5 to 48 volts to the fluidics system 6012; 5 to 48 volts to the robotic drive system 6004, and 5 volts to the one or more disposable devices 5008.

The switch 6030 may be electrically connected to the computer 6009, the controller 6011, the controller 6038, the robotic drive system 6004, and the remote control system 6006. In some embodiments, the switch 6030 may be electrically connected to the computer 6009, controller 6011, the controller 6038, and the robotic drive system 6004 via corresponding ethernet cables. In some embodiments, the switch 6030 may be electrically connected to the remote control system 6006 via a network.

The switch 6030 may be configured to receive and/or transmit electrical signals and/or data between the various connected components. The electronic signals and/or data may be digital signals representing data and/or control signals for operating the interventional setup 10, described above. In some embodiments, the switch 6030 may receive electrical signals and/or data from the remote control system 6006 and transmit the electrical signals received from the remote control system 6006 to the computer 6009 of the fluidics tower 6002. Accordingly, the remote control system 6006 may be electrically connected with the computer 6009 of the fluidics tower 6002 thereby enabling the fluidics tower 6002 and the remote control system 6006 to communicate with one another over a network to enable a physician to perform an operation remotely from the operating room. For example, the switch 6030 may forward instructions from the remote control system 6006 to the one or more fluidic subsystems, and/or to the robotic drive system 6004.

The switch 6030 may be electrically connected to a power source such as an outlet, a battery, and/or a PDU. In some embodiments, the switch 6030 may be electrically connected to the PDU 6028.

The video capture device 6032 may be electrically connected to the computer 6009 and a video source such as an x-ray video device. The video capture device 6032 may be electrically connected to the computer 6009 via a PCIe cable and to the video source via a DVI cable.

The video capture device 6032 may be configured to convert a continuous video stream into one or more high-definition still images and/or capture the continuous video stream and transmit the captured still images and/or video to the computer 6009.

The video capture device 6032 may be electrically connected to a power source such as an outlet, a battery, and/or a PDU. In some embodiments, the video capture device 6032 may be electrically connected to the PDU 6028.

The one or more fluidic subsystems may be electrically connected to the one or more valves 6036 and/or the controller 6038. In some embodiments, the one or more fluidic subsystems may be electrically connected to the controller 6038 via a CAN cable configured to treat all devices equally thereby allowing for fast data transmission. In some embodiments the second fluidic subsystem 6034B may be electrically connected to one or more valves 6036.

The one or more fluidic subsystems may be configured to receive and/or transmit electrical signals and/or data between the one or more fluidic subsystems and the controller 6038. The electronic signals and/or data may be control signals and/or data for using the interventional devices to provide contrast, saline, and/or suction as described above. In some embodiments, the second fluidic subsystem 6034B may receive electrical signals and/or data from the remote control system 6006 and transmit the electrical signals received from the controller 6038 to the one or more valves 6036. Accordingly, the controller 6038 may be control the actuation of the one or more valves 6036 for providing saline.

The one or more fluidic subsystems may be electrically connected to a power source such as an outlet, a battery, and/or a PDU. In some embodiments, the one or more fluidic subsystems may be electrically connected to the PDU 6028.

The one or more valves 6036 may be electrically connected to the second fluidic subsystem 6034B. In some embodiments, the one or more valves 6036 may be electrically connected to the controller 6038 via the second fluidic subsystem 6034B.

The electronic signals and/or data transmitted by the second fluidic subsystem 6034B may be control signals for activating the valve actuators to provide saline in a controlled manner as described above.

The one or more valves 6036 may be electrically connected to a power source such as an outlet, a battery, and/or a PDU. In some embodiments, the one or more valves 6036 may be electrically connected to the PDU 6028.

The controller 6038 may be electrically connected to the switch 6030, the one or more fluidic subsystems, and the one or more sensors 6040.

The controller 6038 may be configured to receive, transmit, and/or process electrical signals. The electrical signals may be digital signals.

The controller 6038 may be electrically connected to the switch 6030, one or more fluidic subsystems via a wired connection and/or a wireless connection. A wired connection may include an ethernet cable, a CAN cable, a GPIO cable, and/or an ADC cable. In some embodiments, the controller 6038 may exclusively implement wired connection, exclusively implement wireless connections, or simultaneously implement wired connections and wireless connections.

The controller 6038 may be configured to transmit and/or receive electrical signals and/or data between the controller 6038 and the switch 6030, the one or more fluidic subsystems, and/or the robotic drive system 6004. In some embodiments, the electrical signals and/or data may flow from the switch 6030 to the controller 6038 and from the controller 6038 to the one or more fluidic subsystems.

The controller 6038 may be electrically connected to a power source such as an outlet, a battery, and/or a PDU. In some embodiments, the controller 6038 may be electrically connected to the PDU 6028. In some embodiments, the PDU 6028 may provide 5 to 48 volts to the fluidics system 6012 which includes the controller 6038. The electrical connection between the controller 6038 and the PDU 6028 may be DC current.

The one or more sensors 6040 may be electrically connected to the controller 6038. In some embodiments, the one or more sensors 6040 may be electrically connected to the controller 6038 via a GPIO and/or an ADC cable.

The electronic signals transmitted by the controller 6038 may be control signals for activating the one or more sensors 6040. The electronic signals transmitted by the one or more sensors 6040 to the controller 6038 may be data measured by the one or more sensors 6040.

The one or more sensors 6040 may be electrically connected to a power source such as an outlet, a battery, and/or a PDU. In some embodiments, the one or more sensors 6040 may be electrically connected to the PDU 6028.

The robotic drive system 6004A, 6004B may be electrically connected to the fluidics tower 6002 and interconnect the electronic components of the robotic drive system 6004A, 6004B including the drive table 6042, the one or more motor axis hubs 6044, the one or more sub-controllers 6046, the switch 6048, the joint setup robot 6050, the one or more sub-controllers 6052, and/or the one or more motor axis telescopic drives 6054.

The robotic drive system 6004A, 6004B may be electrically connected to a power source such as an outlet, a battery, and/or a PDU. In some embodiments, the robotic drive system 6004A, 6004B may be electrically connected to the PDU 6028. The PDU 6028 may provide electrical power to the robotic drive system 6004A, 6004B, as shown in FIGS. 26A and 26B. In some embodiments, the PDU 6028 may provide 5 to 48 volts to the robotic drive system 6004A, 6004B. The electrical connection between the robotic drive system 6004A, 6004B and the PDU 6028 may be DC current.

The robotic drive system 6004A, 6004B may be electrically connected to the fluidics tower 6002 and interconnect the one or more motor axis hubs 6044, the one or more sub-controllers 6046, and/or the switch 6048.

The robotic drive system 6004A, 6004B may be electrically connected to a power source such as an outlet, a battery, and/or a PDU. In some embodiments, the robotic drive system 6004A, 6004B may be electrically connected to the PDU 6028. The PDU 6028 may provide electrical power to the robotic drive system 6004A, 6004B, as shown in FIGS. 26A-B. In some embodiments, the PDU 6028 may provide 5 to 48 volts to the robotic drive system 6004A, 6004B. The electrical connection between the robotic drive system 6004A, 6004B and the PDU 6028 may be DC current.

The one or more motor axis hubs 6044 may be electrically connected to the one or more sub-controllers 6046. In some embodiments, the robotic drive system 6004A, 6004B may include the same number of motor axis hubs 6044 as sub-controllers 6046 such that each of the one or more motor axis hubs 6044 have a corresponding sub-controller 6046.

The one or more motor axis hubs 6044 may be configured to receive electrical signals and/or data from the one or more sub-controllers 6046, as shown in FIG. 26A. In some embodiments, the electronic signals between the one or more motor axis hubs 6044 and the one or more sub-controllers 6046 may be digital. In some embodiments, the one or more motor axis hubs 6044 may be electrically connected to the one or more sub-controllers via one or more CAN cables.

The one or more motor axis hubs 6044 may be electrically connected to a power source such as an outlet, a battery, and/or a PDU. In some embodiments, the one or more motor axis hubs 6044 may be electrically connected to the PDU 6028 via the robotic drive system 6004A, 6004B.

The one or more sub-controllers 6046 may be electrically connected to the one or more motor axis hubs 6044 and the switch 6048. In some embodiments, the robotic drive system 6004A, 6004B may include the same number of sub-controllers 6046 as motor axis hubs 6044 such that each of the one or more sub-controllers 6046 corresponds to a distinct motor axis hub 6044.

The one or more sub-controllers 6046 may be configured to receive electrical signals and/or data from the switch 6048 and transmit one or more electrical signals and/or data to the one or more motor axis hubs 6044, as shown in FIG. 26A. In some embodiments, the electronic signals between the one or more motor axis hubs 6044 and the one or more sub-controllers 6046 may be digital. In some embodiments, the one or more sub-controllers 6046 may be electrically connected to the one or more motor axis hubs 6044 via one or more CAN cables.

The one or more sub-controllers 6046 may be electrically connected to a power source such as an outlet, a battery, and/or a PDU. In some embodiments, the one or more sub-controllers 6046 may be electrically connected to the PDU 6028 via robotic drive system 6004A, 6004B.

The switch 6048 may be electrically connected to the controller 6011, the one or more sub-controllers 6046, and the one or more sub-controllers 6052.

The switch 6048 may be configured to receive electrical signals and/or data from the controller 6011 and transmit one or more electrical signals and/or data to the one or more sub-controllers 6046, 6052, as shown in FIGS. 26A and 26B. In some embodiments, the electronic signals and/or data between the switch 6048 and the controller 6011 and/or the one or more sub-controllers 6046, 6052 may be digital. Furthermore, the electronic signals and/or data may include instructions for controlling the robotic drive system 6004A, 6004B. In some embodiments, the switch may be electrically connected to the controller 6011 and the sub-controllers 6046, 6052 via one or more ethernet cables.

The switch 6048 may be electrically connected to a power source such as an outlet, a battery, and/or a PDU. In some embodiments, the switch may be electrically connected to the PDU 6028 via the robotic drive system 6004A, 6004B.

The joint setup robot 6050 may be electrically connected to the fluidics tower 6002 and be electrically interconnected with the robotic drive system 6004A, 6004B. The joint setup robot 6050 may further electrically interconnect the electronic components of the robotic drive system 6004A, 6004B including the joint setup robot 6050, the one or more sub-controllers 6052, and/or the one or more motor axis telescopic drives 6054.

The joint setup robot 6050 may be electrically connected to a power source such as an outlet, a battery, and/or a PDU. In some embodiments, the joint setup robot 6050 may be electrically connected to the PDU 6028. The PDU 6028 may provide electrical power to the joint setup robot 6050, as shown in FIGS. 26A and 26B. In some embodiments, the PDU 6028 may provide 5 to 48 volts to the joint setup robot. The electrical connection between the joint setup robot 6050 and the PDU 6028 may be DC current.

The one or more sub-controllers 6052 may be electrically connected to the switch 6048, the one or more motor axis telescopic drives 6054 and the motor axis setup joint 6056. In some embodiments, the joint setup robot 6050 may include the same number of sub-controllers 6052 as motor axis telescopic drives 6054 such that each of the one or more sub-controllers 6052 corresponds to a distinct motor axis telescopic drive 6054.

The one or more sub-controllers 6052 may be configured to receive electrical signals and/or data from the switch 6048 and transmit one or more electrical signals and/or data to the one or more motor axis telescopic drives 6054 and/or motor axis setup joint 6056, as shown in FIGS. 26A and 26B. In some embodiments, the electronic signals between the one or more motor axis telescopic drives 6054 and/or motor axis setup joint 6056 and the one or more sub-controllers 6052 may be digital. In some embodiments, the one or more sub-controllers 6052 may be electrically connected to the one or more motor axis telescopic drives 6054 and/or motor axis setup joint 6056 via one or more CAN cables. The one or more sub-controllers 6052 may be electrically connected to the switch 6048 via an ethernet cable.

The one or more sub-controllers 6052 may be electrically connected to a power source such as an outlet, a battery, and/or a PDU. In some embodiments, the one or more sub-controllers 6052 may be electrically connected to the PDU 6028 via the joint setup robot 6050.

The one or more motor axis telescopic drives 6054 may be electrically connected to the one or more sub-controllers 6052. In some embodiments, the joint setup robot 6050 may include the same number of sub-controllers 6052 as motor axis telescopic drives 6054 such that each of the one or more sub-controllers 6052 corresponds to a distinct motor axis telescopic drive 6054.

The one or more motor axis telescopic drives 6054 may be configured to receive electrical signals and/or data from the one or more sub-controllers 6052, as shown in FIG. 26B. In some embodiments, the electronic signals between the one or more motor axis telescopic drives 6054 and the one or more sub-controllers 6052 may be digital. In some embodiments, the one or more motor axis telescopic drives 6054 may be electrically connected to the one or more sub-controllers 6052 via one or more CAN cables.

The one or more motor axis telescopic drives 6054 may be electrically connected to a power source such as an outlet, a battery, and/or a PDU. In some embodiments, the one or more motor axis telescopic drives 6054 may be electrically connected to the PDU 6028 via the joint setup robot 6050.

The motor axis setup joint 6056 may be electrically connected to the one or more sub-controllers 6052.

The motor axis setup joint 6056 may be configured to receive electrical signals and/or data from the one or more sub-controllers 6052, as shown in FIG. 26B. In some embodiments, the electronic signals between the motor axis setup joint 6056 and the one or more sub-controllers 6052 may be digital. In some embodiments, the motor axis setup joint 6056 may be electrically connected to the one or more sub-controllers 6052 via one or more CAN cables.

The motor axis setup joint 6056 may be electrically connected to a power source such as an outlet, a battery, and/or a PDU. In some embodiments, the motor axis setup joint 6056 may be electrically connected to the PDU 6028 via the joint setup robot 6050.

The computer 6064 may be a hub for the remote control system 6006 and may be electrically connected to the control device 6058, an interface, the monitor 6060, the one or more remote communication devices, and the switch 6070. In some embodiments, the computer 6064 may be further electrically connected to the one or more intermediate devices. In such embodiments, one or more intermediate devices may be positioned electrically between the computer 6064 and the one or more remote communication devices.

The computer 6064 may be configured to receive, transmit, and process data signals between the control device 6058, an interface, the monitor 6060, the one or more remote communication devices, and the switch 6070. In some embodiments, the computer 6064 may be configured to only receive, transmit, and process digital signals and data, as shown in FIG. 26A. Accordingly, the electronic signals between the computer 6064 and the control device 6058, an interface, the monitor 6060, the one or more remote communication devices and the switch 6030 may be digital. In some embodiments, one or more of the connected devices (e.g., the control device 6058, the monitor 6060, the one or more remote communication devices and/or the switch 6030) may receive, transmit, and/or process analog signals. Accordingly, one or more intermediate devices may be implemented between the computer 6064 and the one or more connected devices for converting analog signals into a digital signals to be received and processed by the computer 6009 or converting digital signals into an analog signal to be received and processed by the one or more connected devices.

The computer 6064 may be electrically connected to the control device 6058, an interface, the monitor 6060, the one or more remote communication devices, and/or the switch 6030 via a wired connection and/or a wireless connection. For example, the computer 6064 may be connected to the monitor 6060 via an HDMI cable.

The computer 6064 may be configured to transmit electrical signals and/or data as an output from the computer 6064 to the monitor 6060 and the first communication device 6062A. The computer 6064 may be configured to receive electrical signals and/or data as an input from the control device 6058, the second communication device 6062B, and/or the third communication device 6062C. In some embodiments, the computer 6064 may be configured to transmit and/or receive electrical signals and/or data between the computer 6064 and the switch 6070.

The computer 6064 may receive captured video from the video capture device, encode the captured video into a compressed state, and transmit the encoded captured video to the fluidics tower 6002. Additionally and/or alternatively, the computer 6064 may receive encoded video from the fluidics tower 6002, decode the encoded video, and display the decoded video on a monitor 6060.

The computer 6064 may be electrically connected to a power source such as an outlet, a battery, and/or a PDU. In some embodiments, the computer 6064 may be electrically connected to the PDU 6068. The PDU 6068 may provide electrical power to the computer 6064, as shown in FIG. 26A. In some embodiments, the PDU 6068 may provide 5 to 12 volts to the computer 6064. The electrical connection between the computer 6064 and the PDU 6068 may be DC current.

The battery 6066 may be electrically connected to the electrical components of the remote control system 6006, and/or the PDU 6068. The electrical components of the remote control system 6006 may include the computer 6064, the control device 6058, an interface, the monitor 6060, the one or more remote communication devices, the computer 6064, and/or the switch 6070. In some embodiments, the battery 6066 may be electrically connected to either the electrical components of the remote control system 6006 or to the PDU 6028.

The battery 6066 may be further electrically connected to a power source such as an outlet, generator, solar panel, and/or other sources of electrical power. The battery 6066 may be configured to receive electrical power from the power source; store the electrical power; and transmit the electrical power to the electrical components of the remote control system 6006. In some embodiments, the electrical power may be indirectly transmitted to the electrical components of the remote control system 6006 via the PDU 6068.

The battery 6066 may be configured to receive 110/220 volts of AC. The battery 6066 may be configured to transmit: 5 to 12 volts to the computer 6064.

The PDU 6068 may be electrically connected to the electrical components of the remote control system 6006 and the battery 6066. The PDU 6068 may be configured to receive electrical power from the battery 6066 and transmit the electrical power to the electrical components of the remote control system 6006.

The PDU 6068 may be configured to receive sufficient power from the battery 6066 for distributing power to the connected components. For example, the PDU 6068 may distribute: 5 to 12 volts to the computer 6064.

The switch 6070 may be configured to receive and/or transmit electrical signals and/or data between the various connected components. The electronic signals and/or data may be digital signals representing data and/or control signals for operating the interventional setup 10, described above. In some embodiments, the switch 6070 may transmit electrical signals and/or data from the computer 6064 of the remote control system 6006 and transmit the electrical signals to the computer 6009 of the fluidics tower 6002. Accordingly, the remote control system 6006 may be electrically connected with the computer 6009 of the fluidics tower 6002 thereby enabling the fluidics tower 6002 and the remote control system 6006 to communicate with one another over a network to enable a physician to perform an operation remotely from the operating room.

The switch 6070 may be electrically connected to a power source such as an outlet, a battery, and/or a PDU. In some embodiments, the switch 6070 may be electrically connected to the PDU 6068.

The one or more disposable devices 6008 may be electrically connected to a power source such as an outlet, a battery, and/or a PDU. In some embodiments, the one or more disposable devices 6008 may be electrically connected to the PDU 6028.

The subsystems of the robotic medical system 6000 may thus be electrically interconnected for transmitting electrical signals and/or data therebetween. For example, the remote control system 6006 may be in electrical communication with the fluidics tower 6002 such that an input into the remote control system 6006 can be transmitted as instructions to the fluidics tower 6002. The fluidics tower 6002 may be in further electrical communication with the robotic drive system 6004. Accordingly, the instructions from the remote control system 6006 may be implemented by the fluidics tower 6002 and/or the robotic drive system 6004A, 6004B.

The transmission of electrical signals and/or data between two or more components of the robotic medical system 6000 may include a latency. Although generally it is best to minimize latency, for some physicians to properly move the interventional devices as desired and needed to navigate through vasculature to a desired location (e.g., the location of a clit) it is easier to operate the system when the latency is consistent or nearly consistent, rather than having a varying latency. In some embodiments, the latency may be present between an input at the remote control system 6006 and a corresponding output from a local system comprising the fluidics tower 6002, the robotic drive system 6004A, 6004B, and the one or more disposable devices 6008. For example, the latency may be a delay between a physician's input at the remote control system 6006 and the corresponding movement of an interventional hub of the robotic drive system 6004.

The latency may be determined in a variety of ways. In some embodiments, the latency may be determined automatically by the robotic medical system 6000, for example, by performing a “ping” test measuring the round trip communication time between the bedside robotic system and the remotely located control console, by a movement test performed by the remote operator, by dynamically monitoring a bandwidth test of the communication channel, or it may be determined based on one or more factors. The one or more factors used for estimating the latency may include but is not limited to: the distance between the remote control system 6006 and the operating room; and the known bandwidth of the network or communication channel used for transmitting electronic signals and/or data between the components of the robotic medical system 6000.

Fluctuations in system latency may present additional challenges to a physician operating on a patient remotely. Physicians have less control over a procedure when the latency fluctuates significantly and presents safety risks to the patient. The latency may be advantageously manipulated by the robotic medical system 6000 to minimize fluctuations and create a more uniform latency. In some embodiments, an acceptable latency threshold may be predetermined. For example, 450 milliseconds between moving a control device of the remote control system 6006 and having an hub move or displaying a corresponding movement of an hub on the monitor of the remote control system 6006, may be an acceptable threshold for system latency. In some embodiments, the robotic medical system 6000 is configured to have a latency such that when a control signal is generated by moving a control device of the remote control system 6006 and when a hub correspondingly moves the latency is 50 milliseconds, 100 millisecond, 150 milliseconds, 200 millisecond, 250 milliseconds, 300 milliseconds, 350 milliseconds, 400 milliseconds, 450 milliseconds, 500 milliseconds, 550 milliseconds, 600 milliseconds, 650 milliseconds, 700 milliseconds, 750 milliseconds, 800 milliseconds, 850 milliseconds, or 900 milliseconds, plus or minus 50 milliseconds. In an example, the robotic medical system 6000 can have a latency of between 300 and 600 milliseconds. In another example, the system is configured to have a maximum latency of about 450 milliseconds, plus or minus 100 milliseconds. In another example, the control system is configured to have a latency of 450 milliseconds. The robotic medical system 6000 may increase a detected latency to a predetermined value when the detected latency is less than the predetermined latency threshold. In some embodiments, the robotic medical system 6000 may manipulate the detected and/or inherent latency of the robotic medical system 6000 by incorporating internal signal time delays. For example, the robotic medical system 6000 may delay transmitting signals for 100 milliseconds when the detected inherent latency is 350 milliseconds for a robotic medical system 6000 having an acceptable latency threshold of 450 milliseconds. this advantageously allows a physician to train on and use the surgical system where the latency is consistent. In some embodiments, latency can be monitored by robotic medical system 6000 and if too high, the control system may prioritize control signals being communicated from the remote control console and/or de-prioritize other information being communicated (e.g., images) to decrease latency. For example, the robotic medical system 6000 may dynamically prioritize communicating control information and de-prioritize communicating non-control information until the latency is below a predetermined latency level or is at a minimum.

Further, the inherent latency of the robotic medical system 6000 may be determined by the computer 6009 of the fluidics tower 6002, wherein the computer 6009 may calculate and incorporate a signal time delay into the transmission path of electronic signals and/or data. For example, the computer 6009 determine an inherent latency is less than or equal to a predetermined latency threshold, calculate a signal time delay to match the predetermined latency threshold, and send the calculated signal time delay to the switch 6030 to delay transmitting signals by the calculated signal time delay. As discussed above, the latency of the robotic medical system 6000 may fluctuate. Accordingly, the signal time delay may not be constant and may vary as the detected latency fluctuates.

Alternatively, the robotic medical system 6000 may mitigate challenges resulting from the detected latency of the robotic medical system 6000 when the detected latency is above the predetermined latency threshold by minimizing transmission bandwidth. In some embodiments, the robotic medical system 6000 may minimize the effects of the inherent latency by limiting the transmitting electronic signals and/or data based on a determined prioritization. The robotic medical system 6000 may determine an associated category for each electronic signal and/or data. The robotic medical system 6000 may organize the electronic signals and/or data into a prioritized list based on the associated categories of each electronic signal and/or data. In some embodiments, electronic signals and/or data comprising the essential functions of a medical procedure may be prioritized while non-essential communications may be de-prioritized. For example, control communications to control the fluidics system 6012 and/or image communications of image data in the working area of one or more catheter tips may be prioritized while image data of the entire operating room may be deprioritized. Furthermore, control communications to control the fluidics system 6012 may be prioritized over image communications of image data in the working area of the one or more catheter tips. Similarly, lesser relevant views of either the operating room or workspace may be further deprioritized under more relevant views. In some embodiments, electronic signals and/or data may be prioritized based on the stage of a medical procedure. For example, image data of the working area of one or more catheter tips may be prioritized when the catheter is positioned within a sensitive anatomy of a patient. Alternatively, image data of the working area of one or more catheter tips may be deprioritized when the catheter is positioned exterior to a patient. Additionally and/or alternatively, the resolution of the electronic signals and/or data may be modified based on the associated prioritization such that all streams continue to be transmitted but at varying degrees of resolution. For example, higher priority electronic signals and/or data may be transmitted at a higher resolution than lower priority electronic signals and/or data.

Additionally and/or alternatively, in some embodiments, the controller may implement an algorithm that dynamically adjusts the resolution of certain media streams and/or entirely cut off certain media streams based on a function of latency and bandwidth compared to acceptable values. For example, the algorithm may adjust media streams when the latency is greater than a predetermined threshold, as discussed above, and/or when bandwidth is reduced below a predetermined threshold. Accordingly, the algorithm monitors detected latency and bandwidth compared to corresponding predetermined thresholds. When either the latency exceeds an acceptable threshold and/or when bandwidth falls under an acceptable threshold, the algorithm manipulates the media streams to preserve the resolution of higher prioritized media streams over lower prioritized media streams.

In some embodiments, the prioritization of electronic signals and/or data may be performed automatically by the robotic medical system 6000. In some embodiments, the prioritization of electronic signals and/or data may be performed by an operator such as a surgical technician and/or the physician.

In some embodiments, only prioritized electronic signals and/or data may be transmitted thereby reducing used bandwidth and associated latency to the predetermined latency level.

The robotic medical system 6000 may provide a latency indicator configured to inform an operator of a latency. In some embodiments, the latency may displayed when the latency is above a predetermined threshold. The displayed latency may be a general indicator or a specific indicator. A general indicator may be a light, sound, image, general ping value, or other indicator configured to convey that the latency is above a predetermined threshold. In some embodiments, the robotic medical system 6000 may have two or more predetermined thresholds. In such embodiments, the robotic medical system 6000 may display a distinct general indicator for each predetermined threshold. A specific indicator may the specific latency in units of time such as milliseconds. The latency may be the inherent latency of the robotic medical system 6000 and/or an adjusted latency including both the determined inherent latency and a calculated signal time delay. In some embodiments, the latency may be displayed on the remote control system 6006 for informing a physician of the effective delay between an input and the corresponding output.

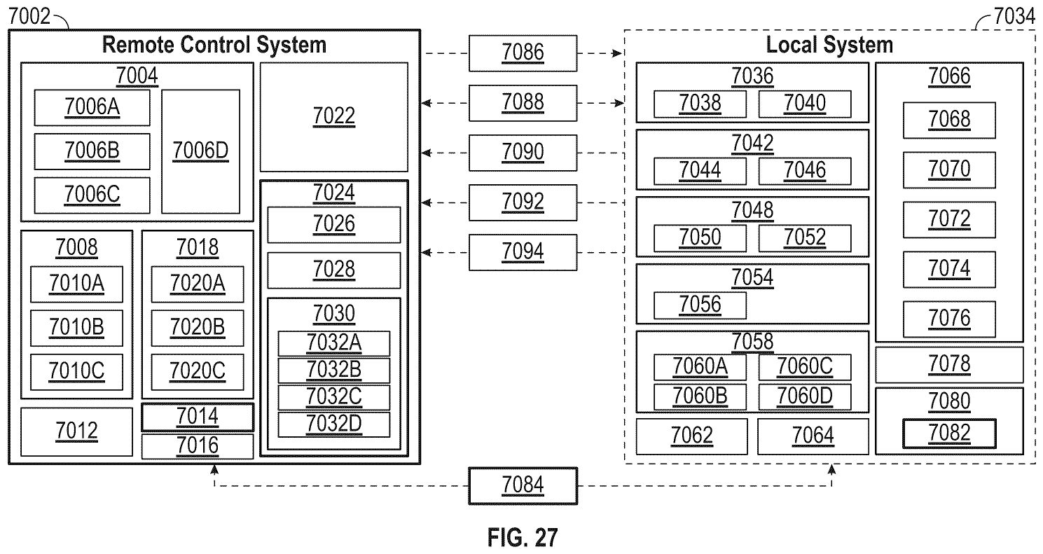

FIG. 27 illustrates an electrical system diagram of an example of a robotic medical system 7000. The robotic medical system 7000 may be the robotic medical system 5000 or 6000 described above with respect to FIGS. 25A-26B, respectively. The robotic medical system 7000 may include a remote control system 7002, one or more operational devices 7034, and an operating room integration module 7084 configured to transmit and/or receive electronic messages between the remote control system 7002 and the one or more operational devices 7034. In some embodiments, the remote control system 7002 may be remote from an operating room while the one or more operational devices 7034 may be local to the operating room.

The robotic medical system 7000 may be configured to transmit instructions (commands/controls/inputs), audio-visual feeds 7088, operating room (OR) status updates 7090, procedure feedback 7092, and system status updates 8094 between the remote control system 7002 and the one or more operational devices 7034. In some embodiments, the one or more operational devices 7034 may transmit status information about the fluidics, the robotic drive, and the plurality of interventional devices.

The remote control system 7002 may be the remote control system 5006, 6006 described above. The remote control system 7002 may further include an input 7004, a communication module 7008, an image processor 7012, a processor 7014, a memory 7016, a feedback logic 7018, a handoff module 7022, and/or one or more displays 7024. In some embodiments, the remote control system 7002 may be positioned remotely from an operating room. For example, the remote control system 7002 may be in a first hospital and/or clinic located remotely from an operating room in a second hospital and/or clinic.

The input 7004 may be the control device 5024, 6058 described above. The input 7004 may further include one or more controls. The one or more controls may include a first control 7006A, a second control 7006B, a third control 7006C, and a fourth control 7006D. In some embodiments, the first control 7006A may be a robotics control for controlling the robotic drive system 5004, 6004 described above; the second control 7006B may be a table control for controlling an extendable table such as the drive table 5018 of the robotic drive system 5004, 6004A, 6004B as described above; the third control 7006C may be a fluidics control for controlling the flow of contrast and/or saline as described above, and/or the application of vacuum as described above; and the fourth control 7006D may be an imaging control for controlling an imaging device to enable a remotely located surgeon to control a particular view. For example, the fourth control 7006D may control the one or more local communication devices of the fluidics tower 5002, 6002 described above.

The communication module 7008 may be the one or more remote communication devices described above. The communication module 7008 may further include one or more signal processors (e.g., computer hardware processors). The one or more signal processors may include a first signal processor 7010A, a second signal processor 7010B, and a third signal processor 7010C. In some embodiments, the first signal processor 7010A may be a secure audiovisual (AV) app; the second signal processor 7010B may be an AV input/output (I/O); and the third signal processor 7010C may be a streaming logic.

The secure AV app may be a computer program configured to provide or supplement transmissions of an AV signal. The secure AV application may be secured and/or protected from unauthorized access. In some embodiments, the secure AV application may require a passcode, implement a virtual private network, encrypt data, implement multi-factor authentication and/or require a security key. Accordingly, the secure AV application 7072 may prevent unauthorized access to the AV data and may protect patients' privacy from unauthorized disclosure of the patients' health information or other information that can be used to identify an individual patient. Thus, the secure AV application 7072 may comply with HIPAA and other privacy regulations.

The AV I/O may be a device configured to receive audio and visual inputs, process and/or use said audio and visual inputs, and output the processed audio and visual inputs. In some embodiments, the AV I/O may receive AV inputs from the one or more remote communication devices described above and output the AV inputs to the one or more operational devices 7034.

The third signal processor 7010C can be a streaming logic configured to format the transmission of one or more electronic signals and/or data. The one or more electronic signals and/or data may be transmitted and/or streamed from the remote control system 7002 to one or more operational devices 7034. The third signal processor 7010C may include an algorithm for prioritizing electronic signals and/or data based on available bandwidth and measured latency. The third signal processor 7010C may default to streaming all electronic signals and/or data. In some embodiments, the streaming logic may minimize the resolution of lower priority electronic signals and/or data. In some embodiments, the third signal processor 7010C may drop video signals and transmit audio signals.

The image processor 7012 may be an image processing unit specialized in processing digital images for enhancing visual images. The image processor 7012 may be configured to evaluate the color and brightness of a given pixel and compare the data of the given pixel to the data of neighboring pixels. The image processor 7012 may use an algorithm to produce an appropriate color and brightness value for the given pixel. The image processor 7012 may employ parallel computing. In some embodiments, the image processor 7012 may employ parallel computing with single instruction multiple data (SMID) and/or multiple instructions multiple data (MIMD) technologies to increase speed and efficiency. In some embodiments, the first signal processor 7010A may be included within the computer 6064 described above.

The processor 7014 may be the logic circuitry of remote control system 7002. The processor 7014 may respond to and process instructions by the surgeon. In some embodiments, the image processor 7012 may be included within the computer 6064 described above.

The memory 7016 may be the electronic holding place for the instructions and data processed by the processor 7014. In some embodiments, the memory 7016 may be included within the computer 6064 described above.

The feedback logic 7018 may be configured to use an output or result of one or more systems of the robotic medical system 7000 as an input response. In some embodiments, the feedback logic 7018 may provide the input response to the surgeon. After receiving the input response the surgeon may respond accordingly. In some embodiments, the robotic medical system 7000 may solely execute instructions based on the surgeon's movements and controls. In such embodiments, the feedback may be directed solely toward the surgeon. In some embodiments, the robotic medical system 7000 may execute instructions based on the surgeon's movements and controls and the feedback. In some embodiments, the feedback logic 7018 may direct the feedback to both the surgeon and the robotic medical system 7000. In some embodiments, the feedback logic 7018 may be included within the computer 6064 described above.

The feedback logic 7018 may further include one or more responses. The one or more responses may include a first response 7020A, a second response 7020B, and a third response 7020C. In some embodiments, the first response 7020A may include haptics; the second response 7020B may be a fluidics response; and the third response 7020C may be a robotics response. A haptics response may direct a physical response to the surgeon such as minimizing control device motion and/or providing a vibration to notify a user of excessive force at the proximal end of an interventional device. A fluidics response may direct fluid levels to the robotic medical system 7000. In such embodiments, the first response 7020A may provide sensory feedback to the surgeon.

The handoff module 7022 may be a software and/or hardware device configured to facilitate transferring control of the robotic medical system 7000 between the remote control system 7002 and a control device of one or more operational devices 7034 such as the interface 5020 described above, a second control console located locally, or manual control. For example, the handoff module 7022 may transfer control of the robotic medical system 7000 from the remote control system 7002 to a control device of one or more operational devices 7034. In some embodiments, the handoff module 7022 may transfer control of the robotic medical system 7000 from a control device of one or more operational devices 7034 to the remote control system 7002. In some embodiments, the handoff module 7022 may terminate control of the initial control device before activating control of the subsequent control device.

In some embodiments, the handoff module 7022 may be activated if an error is detected and control needs to be transferred locally. For example, the handoff module 7022 may be activated if latency exceeds a predetermined threshold, a malfunction of the remote control system 7002 is detected, or another challenge presenting safety concerns to the patient is detected requiring local control of the one or more operational devices 7034.

The one or more displays 7024 may be the monitor 6014, 6060 and/or part of the interface 5026 described above. The one or more displays 7024 may include and/or execute an algorithm 7026 for controlling the contents of the one or more displays 7024. The one or more displays 7024 may further include an operating room view module 7028 and a feedback system 7030. The feedback system 7030 may provide visual feedback and/or warnings to the surgeon and further include one or more display modules. The one or more display modules may include a first display module 7032A, a second display module 7032B, a third display module 7032C, and a fourth display module 7032D. In some embodiments, the first display module 7032A may display navigation feedback for robotic drive system including the hubs and/or devices such as catheters as they transverse through a patient; the second display module 7032B may display errors for informing the surgeon of problems detected by the robotic medical system 7000, for example if air (e.g., air bubbles) is detected within an interventional device; the third display module 7032C may display a complication experienced by the patient, for example, a prolapse of an organ; and the fourth display module 7032D may display a failure of one or more physical components of the system, for example, buckling or collapse of an interventional device.

The one or more operational devices 7034 may be a combination of the fluidics tower 5002, 6002, the robotic drive system 5004, 6004, and/or the one or more disposable devices 5008, 6008 described above. The one or more operational devices 7034 may be in an operating room in a hospital and/or clinic. The one or more operational devices 7034 may further include a surgical robotics system 7036, a fluidics system 7042, one or more patient imaging systems 7048, an operating table 7054, one or more room monitoring systems 7058, an inventory management system 7062, a records management system 7064, a communication module 7066, an alert system 7078, and a room monitoring system 7080. In some embodiments, the surgical robotics system 7036, the fluidics system 7042, the one or more room monitoring systems 7058, the inventory management system 7062, the records management system, the communication module 7066, the alert system, and/or the room monitoring system 7080 may be included in the fluidics tower 5002.

The surgical robotics system 7036 may be the electronics tower described above. The surgical robotics system 7036 may further include a control module 7038 and one or more devices 7040 for controlling to robotics of the robotic medical system 7000. The control module 7038 may be a controller for controlling the physical actuation of the robotic system. In some embodiments, the control module 7038 may be the controller 6011 described above. The one or more devices 7040 may include a joystick, other mechanical or electrical control devices of a user interface, a control touch interface, and/or hardware to facilitate using a voice command.

The fluidics system 7042 may be the fluidics system 5012, 6012 described above. The fluidics system 7042 may further include a control module 7044 and one or more fluids 7046. The control module 7044 may be the controller 6038. The one or more fluids 7046 may include contrast and/or saline. In some embodiments, the one or more fluids 7046 may be contained within the one or more fluidic subsystems.

The one or more patient imaging systems 7048 may be any imaging system configured to capture subdermal images of a patient. The one or more patient imaging systems 7048 may further include a control module 7050 and one or more imaging devices 7052. The control module 7050 may be configured to control the one or more imaging devices 7052. For example, the control module 7050 may control the orientation of the patient and/or an imaging beam. The one or more imaging devices 7052 may be any device configured to capture subdermal images of a patient. For example, the one or more imaging devices 7052 may be an x-ray machine, a fluoroscopy machine, an MRI device, and/or a CT scanner.

The operating table 7054 may be any surface configured to receive a patient during an operation. The operating table 7054 may be configured to engage with the one or more patient imaging systems 7048. The operating table may further include a control module 7056. The control module 7056 may control the orientation and/or motion of the operating table. In some embodiments, the control module 7056 may control the elevation of the operating table may instructing the operating table to raise or lower. The control module 7056 may further control a rotation of the operating table. For example, the control module may control a rotation about an axis along the width of the operating table (pitch), a rotation about an axis along the length of the operating table (roll), and/or a rotation of about an axis along the height of the operating table (yaw). Accordingly, the control module 7056 can control the orientation of the operating table. In some embodiments, the operating table may be motorized and configured to move within the operating room. In such embodiments, the control module 7056 may be configured to control the motion of the operating table 7054.

The one or more room monitoring systems 7058 may monitor the operating room. In some embodiments, the one or more room monitoring systems 7058 may be configured to monitor for complications and/or anomalies. In some embodiments, the one or more room monitoring systems 7058 may monitor the operating room under standard procedure regardless of the status of the operating room and/or procedure. The one or more room monitoring systems 7058 may further include one or more sensory devices. The one or more sensory devices may include a first sensory device 7060A, a second sensory device 7060B, a third sensory device 7060C, and a fourth sensory device 7060D. The one or more sensory devices may be the one or more local communication devices described above. In some embodiments, the first sensory device 7060A may be one or more cameras; the second sensory device 7060B may be a microphone and/or audio output device; the third sensory device 7060C may be one or more displays; and the fourth sensory device 7060D may be other sensors configured to monitor the operating room and/or patient.

The inventory management system 7062 may monitor and track the use and availability of inventory including the use of interventional devices, fluids of the fluidics system, or any other physical component used during a procedure. The inventory may include an identification number, bar code, QR code, or any other scannable feature such that each piece of inventory used during the procedure may be logged and tracked by the inventory management system 7062. The inventory management system 7062 may advantageously monitor the use of inventory and may be configured to auto populate billing information thereby optimizing coding and billing for healthcare administrative professionals.

The records management system 7064 may track and store system information regarding the procedure. For example, the records management system 7064 may constantly log the status of the robotic medical system 7000, the position of the interventional devices/hubs, and/or sensor information and measurements. The records management system 7064 may be used for playback simulation of a procedure or for troubleshooting. Accordingly, the records management system 7064 may advantageously provide valuable training for healthcare workers including physicians, nurses, and surgical technicians and data to aid in maintenance of the robotic medical system 7000. Additionally, the records management system 7064 may record location and time signatures to be used for billing and coding. Accordingly, the records management system 7064 may advantageously auto populate billing information thereby optimizing coding and billing for healthcare administrative professionals.

The communication module 7066 may be configured to foster communication between electronic devices and/or with human operators. The communication module 7066 may be included in the computer 6009 described above. The communication module 7066 may further include an AV I/O 7068, a remote transfer handler 7070, a secure AV application 7072, an I/O device 7074, and a streaming logic 7076.

The AV I/O 7068 may be software running on the remote control system 7002 and the one or more operational devices 7034 for receiving audio and visual inputs, process and/or use said audio and visual inputs, and output the processed audio and visual inputs. In some embodiments, the AV I/O 7068 may receive AV inputs from the one or more room monitoring systems 7058 and output the AV inputs to the remote control system 7002. In some embodiments, the AV I/O 7068 may be similar to the AV I/O described above. The visual inputs can include fluoroscopy images. The fluoroscopy images can be transmitted to the remote control system 7002 from the one or more operational devices 7034.

The remote transfer handler 7070 can be a remote communication service that is configured to transmit data. In some embodiments, the remote transfer handler 7070 can be a streaming processor. For example, the remote transfer handler 7070 can be configured to transmit one or more commands 7086 (or controls/inputs) between remote and local systems (i.e., the remote control system 7002 and the one or more operational devices 7034) in real time with ultra-low latency. The remote transfer handler 7070 may provide a secure transmission by encrypting the commands/controls/input data over a network.

The secure AV application 7072 may be a computer program configured to provide or supplement transmissions of an AV signal. The secure AV application 7072 may be secured and/or protected from unauthorized access by the same means as the secure AV application. In some embodiments, the secure AV application 7072 may be the same as the secure AV application. In some embodiments, the secure AV application 7072 may connect to the secure AV application. For example, the secure AV application 7072 and the secure AV application may create an encrypted VPN tunnel. Accordingly, the secure AV application 7072 may prevent unauthorized access to the AV data and may protect patients' privacy from unauthorized disclosure of the patients' health information or other information that can be used to identify an individual patient. Thus, the secure AV application 7072 may comply with HIPAA and other privacy regulations.

The I/O device 7074 may be a device configured to receive an input and output. For example, the I/O device 7074 may be the one or more local communication devices described above.

The streaming logic 7076 may be the same as the streaming logic of the third signal processor 7010C.

The room monitoring system 7080 may further include a status module 7082. The status module 7082 may be configured to determine the status of the operating room. In some embodiments, the status of the operating room may be include an indication of whether the operating room is ready for an operation. An operating room may be ready for an operation when the operating room is sanitized, the requisite instruments are sanitized and present, and lighting is sanitized and activated, and/or the interventional setup 10 described above is sanitized and primed for an operation. In some embodiments, an operating room may be ready after the patient is prepped for an operation. The patient may be prepped for an operation after being dressed for the operation, positioned on an operating table, and/or provided anesthesia.

The following discussion relates to the flow of electronic signals and/or data between the remote control system 7002 and the one or more operational devices 7034.

The operating room integration module 7084 may be configured to electronically connect the remote control system 7002 and the one or more operational devices 7034. In some embodiments, the operating room integration module 7084 may be third-party device or service. For example, a third party may offer a support/proctor product for electronically connecting the remote control system 7002 and the one or more operational devices 7034. The operating room integration module 7084 may facilitate the transmission of one or more commands 7086, AV streams 7088, real time operating room status 7090, procedure feedback 7092, real time system status 7094, and/or fluoroscopy images. The fluoroscopy images may be transmitted from the one or more operational devices 7034 to the remote control system 7002.

The one or more commands 7086 may be transmitted from the remote control system 7002 to the one or more operational devices 7034. The one or more commands 7086 may be instructions, controls, and or inputs for controlling the robotic drive system 5004, 6004A, 6004B and/or fluidics system 5012, 6012 described above. Accordingly, a remotely located surgeon can use the remote control system 7002 to control the robotic operational devices located in the operating room.

The AV streams 7088 may be transmitted both ways between the remote control system 7002 and the one or more operational devices 7034. In some embodiments, the transmission direction may depend on which device captured the AV data. For example, if the one or more sensing devices captured AV data then AV stream 7088 comprising the captured AV data may be transmitted from the one or more operational devices 7034 to the remote control system 7002. If one or more remote communication devices of the remote control system 7002 capture AV data, then the captured AV data may be transmitted from the remote control system 7002 to the one or more operational devices 7034. Accordingly, AV data may be transmitted between the remote control system 7002 and the one or more operational devices 7034 thereby advantageously sharing additional data between the remotely located surgeon and the surgical technicians in the operating room.

The real time operating room status 7090 may be transmitted from the one or more operational devices 7034 to the remote control system 7002. The real time operating room status 7090 may include data regarding the current status of the operating room such as whether the operating room is ready for operation. In some embodiments, the real time operating room status 7090 may include two indicators, the status of the operating room and the status of the patient. For example, the real time operating room status 7090 may indicate: unready operating room and unready patient; ready operating room and unready patient; and/or ready operating room and ready patient. The operating room may be unready during pre-operation preparation (e.g., sterilization of the operating room, assembling surgical technicians, assembling surgical tools, initializing robotic drive systems, etc.). The patient may similarly be unready during pre-operation preparation (e.g., dressing the patient, anesthesia is administered but not effective). Accordingly, the real time operating room status 7090 may indicate that: the operating room is unready and the patient is unready; the operating room is ready and the patient is unready; and the operating room is ready and the patient is ready. If any indicator is unready, the real time operating room status 7090 may show an unready status. The real time operating room status 7090 may be transmitted in real time.

Real time may mean that the data is transmitted virtually immediately from the actual time during which the process and/or event occurs. Ideally, real time may be the actual time during which a process or event occurs such that the surgeon is appraised of the actual status as it occurs. However, due to the latency, real time may be subject to a delay when capturing data, processing the data, transmitting the data to the remote control system 7002, and displaying the results to the surgeon. Accordingly, real time may be a time period within a few milliseconds of the actual dime during which the process and/or event occurs.

The procedure feedback 7092 may be transmitted from the one or more operational devices 7034 to the remote control system 7002. The procedure feedback 7092 may be directed to the feedback logic 7018 of the remote control system 7002 and provide feedback to the surgeon and/or remote control system 7002. In some embodiments, the procedure feedback 7092 may be captured by the one or more room monitoring systems 7058. The procedure feedback 7092 may be any data that would aid a surgeon in an operation. For example, the procedure feedback 7092 may include force data and/or position data. In some embodiments, the procedure feedback 7092 may check whether the robotic drive device is aligned and connected properly and/or monitor whether steps have been performed correctly.

The real time system status 7094 may be transmitted from the one or more operational devices 7034 to the remote control system 7002. The real time system status 7094 may include data regarding the current status of the physical and/or electrical components of the interventional setup 10 described above including the robotic medical system 5000, 6000, 7000. The current status of the physical and/or electrical components may include whether the components are activated, inactivated, ready, unready, in standby, or if there is an error. Real time transmission of the system status may advantageously provide a remotely located physician with necessary information for a safe procedure.

The robotic medical system 7000 may include latency between a user input at the remote control system 7002 and a corresponding output of the one or more operational devices 7034 as described above. The robotic medical system 7000 may organize the one or more commands 7086, AV streams 7088, real time operating room status 7090, procedure feedback 7092, real time system status 7094 into essential and non-essential categories. The robotic medical system 7000 may further compare a plurality of essential electrical signals and/or data to one another for prioritizing the transmission of electronic signals and/or data.

Additionally and/or alternatively, the robotic medical system 5000, 6000, 7000 may include a second control console. The second control console may be the same as the remote control system 5006, 6006, 7002 and include a control device, one or more monitors, an interface, and one or more remote communication devices. The second control console may be an auxiliary or backup control console. In some embodiments, the second control console may be used if connection between the remote control system 5006, 6006, 7002 and the local system is disrupted. In some embodiments, the second control console may be part of the local system and located within the operating room. In some embodiments, the robotic medical system 5000, 6000, 7000 may only include the second control console.

A connection establishment system can provide a system and method that establishes and maintains a connection between the remote control system 7002 and the one or more operational devices 7034 throughout the entire procedure. The connection establishment system may implement robust security mechanisms including encryption protocols, client and server authentication measures such as Transport Layer Security (TLS) and digital certificates. For example, the connection establishment system may safeguard the connection and data by encrypting the data transmitted between the remote control system 7002 and the one or more operational devices 7034 against interception and tampering.

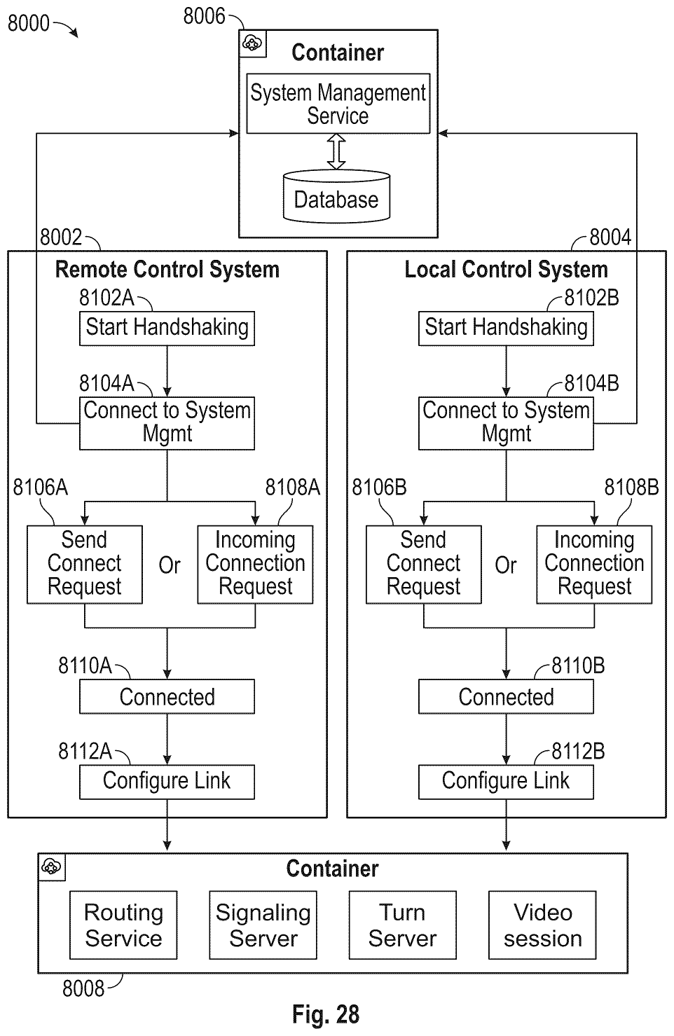

FIG. 28 illustrates a schematic diagram illustrating an example of a handshake workflow 8000 between a remote control system 8002 and a local control system 8004. In some embodiments, the remote control system 8002 can be a control console or physician console. In some embodiments, the remote control system 8002 can be positioned remotely from a robotic surgical system (for example, positioned remotely from an operating room where the robotic surgical system is located). For example, the remote control system 8002 may be the remote control system 5006, 6006, 7002 described herein. In some embodiments, the local control system 8004 can be a bedside computing device or system positioned within the operating room. For example, the local control system 8004 can be the tower electronics system 5010 and/or interface 5020 described herein.

The handshake workflow 8000 also illustrates a system management service 8006. The system management service 8060 may be a software service. In some embodiments, the software service can be configured to electronically connect the remote control system 8002 to the local control system 8004. In some embodiments, the electrical connection between the remote control system 8002 and the local control system 8004 can be a digital connection to transfer data. In some embodiments, the system management service 8006 can be accessible via a network. For example, the system management service can be accessible by a wide area network (WAN) for secure communication and application deployment. In some embodiments, the system management service 8006 may be operated by a third party. The handshake workflow 8000 can prevent unauthorized access and maintain security between the remote control system 8002 and the local control system 8004.

The handshake workflow 8000 can further include a secondary cloud device 8008. The secondary cloud device 8008 may be operatively connected to the system management service 8006. In some embodiments, the secondary cloud device 8008 can include a routing service, a signaling server, a turn server, and/or a video session.

As shown in FIG. 28, the handshake workflow may include various steps described herein for establishing a connection between the remote control system 8002 and the local control system 8004. The steps, when performed, may provide a method for establishing a connection between the remote control system 8002 and the local control system 8004. In some embodiments, the steps may be performed in the order depicted in the handshake workflow 8000. However, the sequence of steps is not limited to the order shown in FIG. 28. Additionally, in some embodiments, a handshake workflow may include one or more additional or alternative steps, and/or may omit one or more of the steps described with respect to FIG. 28.

In some embodiments, the handshake workflow 8000 can be establish a connection between the (patient-side) local control system 8004 and the (physician-side) remote control system 8002. The handshake workflow 8000 can ensure seamless operation of tele-remote procedures and maintain patient safety. In some embodiments, the handshake workflow 8000 can maintain a connection between the remote control system 8002 and the local control system 8004. The system management service 8006 can efficiently manage connections and provide timely status notifications to all systems involved. The handshake workflow 8000 may prevent potential conflicts and unauthorized access thereby enhancing the overall security of the system.

In some embodiments, the handshake workflow 8000 may employ encryption protocols and authentication measures such as Transport Layer Security (TLS) and/or Secure Sockets Layer (SSL) digital certificates. These measures can safeguard the connection and data by encrypting the data transmitted between the local control system 8004 and the remote control system 8002. For example, these measures can protect against data interception and/or tampering. The implementation can ensure server and client authentication and guarantee that the remote control system 8002 is connected to the intended local control system 8004, and vice versa. Additionally, a firewall and network settings may be configured to allow connections from only configured and/or trusted systems. In some embodiments, the handshake workflow 8000 can include a comprehensive failure and recovery mechanism. The failure and recovery mechanism can ensure quick procedure recovery by persisting system states and connection details. One or more of these features can enhance the durability and robustness of the system and contributes to ensuring patient safety, even in the event of system failures.

The handshake workflow 8000 can connect the remote control system 8002 to the local control system 8004 via a system management service 8006.

In some embodiments, the remote control system 8002 can perform a step 8102A of initiating a handshake. Initiating a handshake from the remote control system 8002 under step 8102A can indicate that the remote control system 8002 is ready to connect to its counterpart device, e.g., the local control system 8004. In some embodiments, the local control system 8004 can perform a step 8102B of initiating a handshake. Initiating a handshake from the local control system 8004 under step 8102B can indicate that the local control system 8004 is ready to connect to its counterpart device, e.g., the remote control system 8002.

The remote control system 8002 can perform a step 8104A of connecting to the system management service 8006. The local control system 8004 can perform a step 8104B of connecting to the system management service 8006. In some embodiments, the step 8104A can include checking whether the remote control system 8002 is connected to the system management service 8006. In some embodiments, the step 8104B can include checking whether the local control system 8004 is connected to the system management service 8006. If the remote control system 8002 and/or the local control system 8004 are not connected to the system management service 8006, then the handshake workflow 8000 may proceed by notifying the remote control system 8002 and/or the local control system 8004 of a connection failure.

The handshake workflow 8000 can include handling connection requests. In some embodiments, handling connection requests can include a step of sending a connection request and a step of receiving an incoming connection request. In some embodiments, a system can alternatively either send or receive a connection request. Accordingly, the system that sends a connection request may not receive a connection request. In some embodiments, the remote control system 8002 can perform a step 8106A of sending a connection request to the local control system 8004 via the system management service 8006. In such embodiments, the local control system 8004 can perform a step 8108B of receiving the connection request from the remote control system 8002 via the system management service 8006. Alternatively, in some embodiments, the local control system 8004 can perform a step 8106B of sending a connection request to the remote control system 8002 via the system management service 8006. In such embodiments, the remote control system 8002 can perform a step 8108A of receiving the connection request from the local control system 8004 via the system management service 8006.

The handshake workflow 8000 can include the step of checking whether a system is connected to the counterpart system. In some embodiments, the remote control system 8002 can perform a step 8110A to check whether the remote control system 8002 is connected to the local control system 8004. In some embodiments, the local control system 8004 can perform a step 8110B to check whether the local control system 8004 is connected to the remote control system 8002.

The handshake workflow 8000 can include the step of configuring a link between the remote control system 8002 and the local control system 8004. The remote control system 8002 can perform a step 8112A of configuring a link from the remote control system 8002. The local control system 8004 can perform a step 8112B of configuring a link from the local control system 8004. In some embodiments, a signal may be transmitted to the system management service 8006 to proceed with linking the remote control system 8002 and the local control system 8004. In some embodiments, the step can include setting up a link between the system management service 8006 and the secondary cloud device 8008. The step can include configuring security policies. In some embodiments, the step can include TLS/SSL digital certificates.

In some embodiments, the steps can be performed in numerical order. For example, step 8102A can be performed before step 8104A, step 8104A can be performed before step 8106A, step 8106A can be performed before step 8108A, step 8108A can be performed before step 8110A, step 8110A can be performed before step 8112A. However, the order of steps is not limited by the order depicted in FIG. 28.

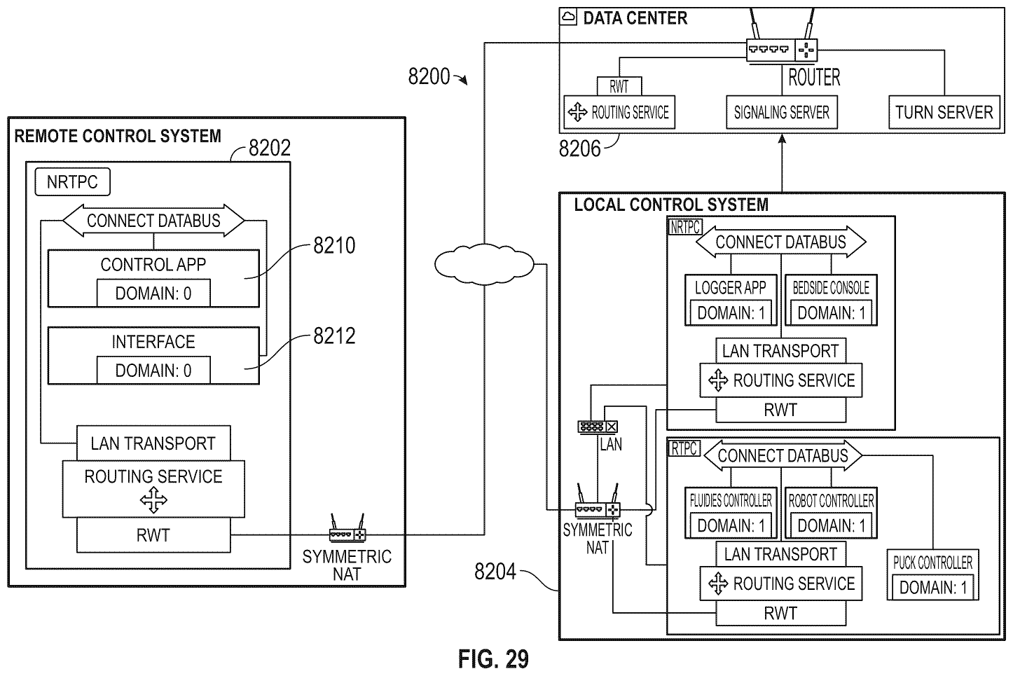

FIG. 29 illustrates a network connection 8200 between a remotely located remote control system 8202 (for example, a remote control console), a local control system 8204, and a system management service 8206. As shown in FIG. 29, the remote control system 8202, the local control system 8204, and the system management service 8206 can be connected over a network. In some embodiments, the network can be a wide area network (WAN).

In some embodiments, the remote control system 8202 can be the same or similar to the remote control system 8002 described herein. Accordingly, the remote control system 8202 can include features described for the remote control system 5006, 6006, 7002 described herein. In some embodiments, the remote control system 8202 can include a process (e.g., software) 8210 that interfaces with a control device. Accordingly, inputs to a control device can be received by the remote control system 8202 and transmitted over the network connection 8200. In some embodiments, the remote control system 8202 can include a display and/or an interface 8212, which can include one or more input devices (for example, a keyboard, joystick, touchpad, touchscreen, and/or other controls that can provide input to the remote control system 8202. The remote control system 8202 can receive video and/or audio signals and present them to a user. For example, the remote control system 8202 can include one or more displays that provides for depicting one or more images of video (e.g., optical, fluoroscopic) and/or audio to convey information relating to a procedure being performed by the local control system 8204 to a physician.

The local control system 8204 can be the same or similar to local control system 8004 described herein. Accordingly, the local control system 8204 can include portions of all of the fluidics tower 5002, 6002 and/or one or more operating room devices 7034, for example as described herein. In some embodiments, the local control system 8204 can includes a robot controller, a hub assembly and/or mount controller, and a fluidics controller. In some embodiments, the local control system 8204 can include one or more displays.

The system management service 8206 can be the system management service 8006 described above. In some embodiments, the system management service 8206 can be a third-party data center.

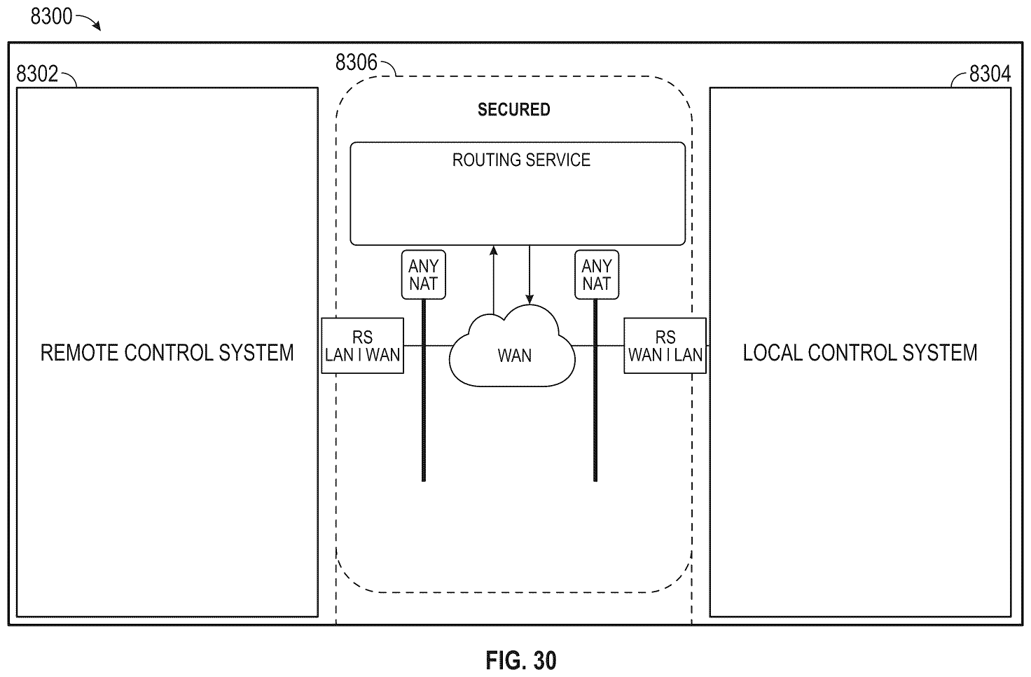

FIG. 30 illustrates a remote communication service that can be configured to transmit one or more commands or controls/inputs between a remote control system 8302 and a local control system 8304 in real time using the RTI routing service 8306 with ultra-low latency. The remote control system 8302 can be any of the remote control systems described herein. The local control system 8304 can be any of the local control systems described herein. The RTI routing service 8306 can be the same or similar to the system management service 8006 described herein. In some embodiments, the commands or controls/inputs can be the commands 7086 described herein. The remote communication service 8306 can provide secure transmission by encrypting the commands/controls/inputs/data over a network. The remote communication service 8306 can interconnect applications across domains, LANs and WANs, including firewall and NAT traversal. This service can beneficially enable remote physicians to securely control and monitor patient-side robots from a remote workstation over the internet with ultra-low latency. To ensure real-time communication and maximum security, this communication service uses DDS protocol and RTI security plugins, encrypting data transmitted over the network. This service conducts health checks of the remote connection service and promptly triggers fault notifications if the connection is disrupted, thus enhancing patient safety and system reliability.

FIG. 31 illustrates a process 8400 for transmitting media data from a transmitting device 8402 to a receiving device 8410. The transmitting device 8402 and the receiving device 8410 can be operatively connected. In some embodiments, the transmitting device 8402 can be operatively connected to the receiving device 8410 over a network 8408. For example, the transmitting device 8402 can be operatively connected to the receiving device 8410 over a wide area network (WAN) 8408.

The transmitting device 8410 may leverage a WebRTC protocol for media streaming. Adaptive streaming in the context of WebRTC can involve dynamically adjusting the media stream quality based on real-time WebRTC statistics. In some embodiments, implementing a WebRTC statistics monitoring library can use a built-in statistics API to periodically gather metrics from both the transmitting device 8402 and the receiving device 8410. In some embodiments, the transmitting device 8402 can be part of the remote control system, for example, a control console or a handheld control device, and the receiving device 8410 can be part of a local control system. In some embodiments, the transmitting device 8402 can be part of the local control system and the receiving device 8410 can be part of the remote control system. In some embodiments, both devices 8402 and 8410 can operate as a transmitting device and a receiving device.

An adaptive streaming algorithm 9406 can analyze the information/statistics relating to the video streams statistics comparing them against predefined thresholds. The statistics can include, for example, one or more of packet loss ratio, jitter, and/or round-trip time. Based on this comparison, the algorithm can determine whether to switch to a higher or lower quality output stream. The transmitting device 8402 can be configured to adjust one or more streaming parameters (for example, video resolution, frame rate, and/or encoding bitrate) to adapt to the changing network bandwidth in real-time.

The process 8400 can include a feedback loop. The feedback loop can include computing quality metrics 8404 of the transmission signal between the transmitting device 8402 and the receiving device 8410. In some embodiments, the quality metrics of the transmission signal can include the bandwidth and/or latency. Accordingly, the quality metrics computation can monitor detected latency and bandwidth compared to corresponding predetermined thresholds. The feedback loop can further include an adaptive streaming algorithm 8406. The adaptive streaming algorithm 8406 can receive the quality metrics of the transmission signal. Based on the received quality metrics, the adaptive streaming algorithm 8406 can dynamically adjust the resolution of certain data signals. For example, the adaptive streaming algorithm 8406 can dynamically adjust the resolution of media streams and/or completely stop providing certain media streams for transmission based on a function of latency and bandwidth compared to acceptable values. For example, the adaptive streaming algorithm 8406 may adjust media streams when the latency is greater than a predetermined threshold, as discussed herein, and/or when bandwidth is reduced below a predetermined threshold. When either the latency exceeds an acceptable threshold and/or when bandwidth falls under an acceptable threshold, the adaptive streaming algorithm 8406 may manipulate the media streams to preserve the resolution of higher prioritized media streams over lower prioritized media streams.

The modified signals output from the adaptive streaming algorithm 8406 can be sent to the transmitting device 8402 to adjust what is transmitted to the receiving device 8410.

### Drive Table

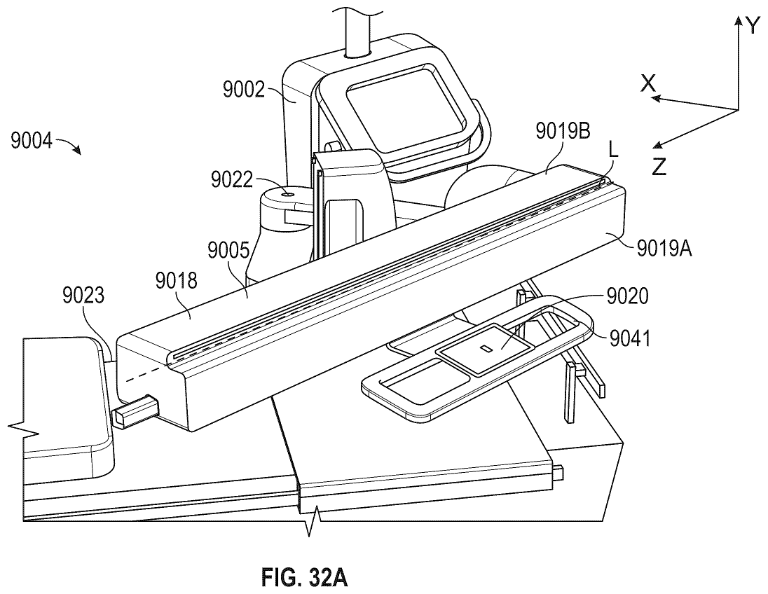

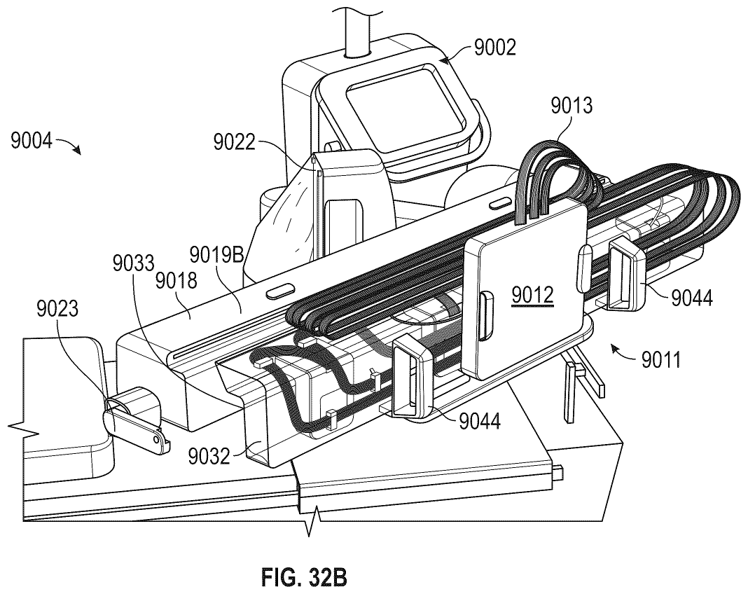

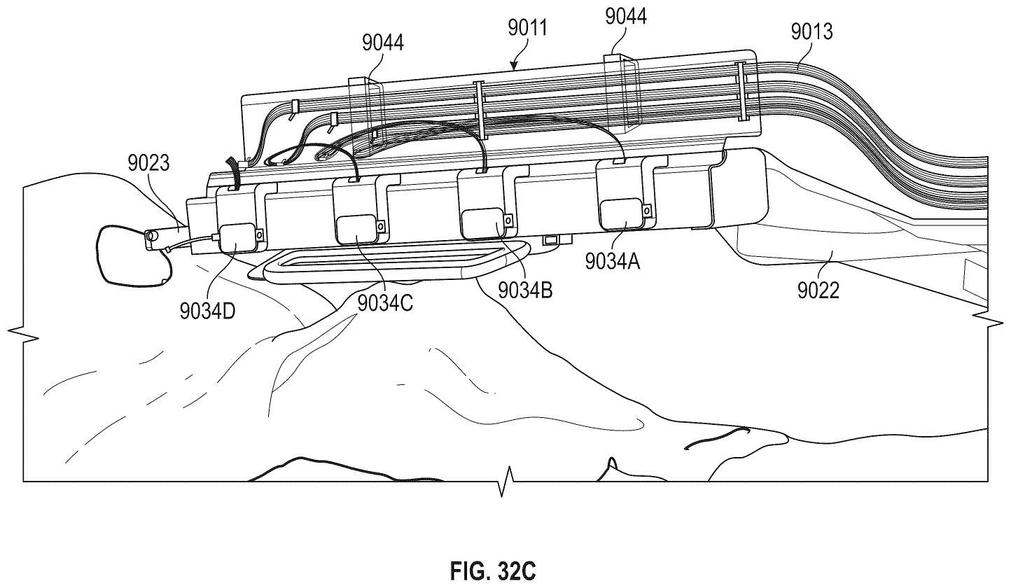

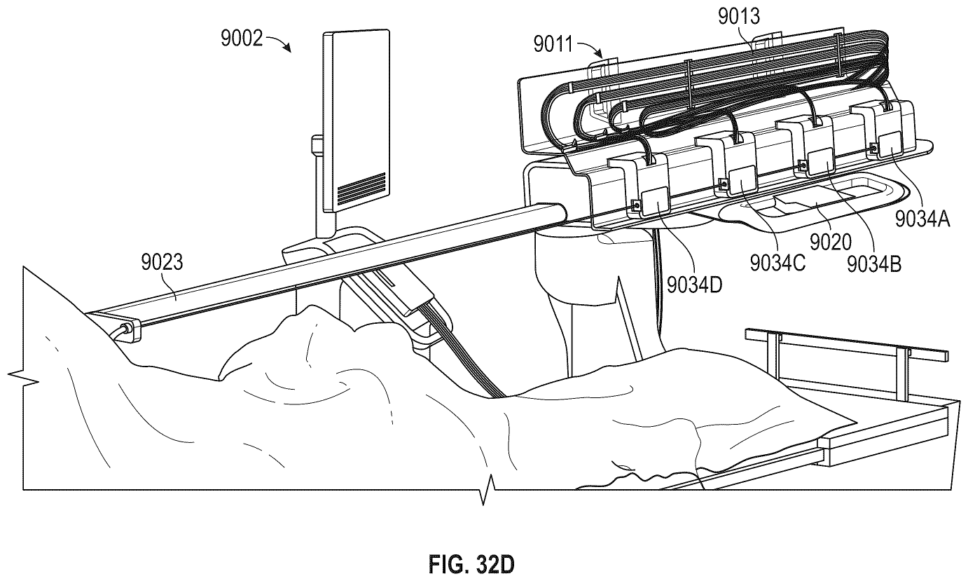

FIGS. 32A-32D illustrate embodiments of a bedside robotic system 9004 for performing a medical procedure. In some embodiments, the bedside robotic system 9004 can be a bed mounted robot. The bedside robotic system 9004 can include a drive table 9018 and a drive assembly 9011. In some embodiments, the drive table 9018 and drive assembly can together form the bedside robotic system 9004 for performing a medical procedure. FIGS. 32A-32D illustrate various states of the drive table 9018 and drive assembly 9011 for preparation of and performance of a robotic surgical procedure. Additionally, an exemplary method of preparing for and performing a robotic surgical procedure is described with respect to FIGS. 32A-32D.

FIG. 32A illustrates the drive table 9018 in a cartesian coordinate frame. The cartesian coordinate frame includes an ordered triplet of lines X, Y, Z illustrating directional orientation of three-dimensional space. The directional orientation of the cartesian coordinate frame includes an X direction, a Y direction, and a Z direction corresponding to the ordered triplet of lines X, Y, Z respectively. The X direction, Y direction, and Z direction are mutually orthogonal. As shown in FIG. 32A the X direction corresponds to a width in a horizontal orientation, the Y direction corresponds to a height in a vertical orientation, and the Z direction corresponds to a length in a horizontal orientation.

FIG. 32A illustrates an embodiment of a fluidics tower 9002 and the bedside robotic system 9004 described above.

The fluidics tower 9002 can include an electronics tower, one or more fluidic subsystems, a monitor, and one or more local communication devices. The fluidics tower 9002 can correspond to the fluidics tower 5002 described above. Accordingly, the electronics tower, the one or more fluidic subsystems, a monitor, and one or more local communication devices can correspond to the tower electronics interface 5010, the fluidics system 5012, a monitor 5014, and one or more local communication devices 5016. The tower electronics interface 5010 can be in electrical communication with a power source and configured to receive data or information from a control console. The one or more fluidic subsystems can include a contrast subsystem, a saline subsystem, and an aspiration subsystem.

The bedside robotic system 9004 can include a drive table 9018, In some embodiments, the bedside robotic system 9004 can include an interface 9020. In some embodiments, the bedside robotic system 9004 can include a base or joint setup 9022. The bedside robotic system 9004 can correspond to the robotic drive system 5004 described above. In some embodiments, the drive table 9018, the interface 9020, and the joint setup 9022 can correspond to the drive table 5018, the interface 5020, and the joint setup 5022 described above.

The drive table 9018 can include a body with a drive surface 9019 for supporting one or more hub assemblies as described in greater detail below. The drive table 9018 can include interior drive elements configured to magnetically couple with the one or more hub assemblies as described in greater detail below.

In some embodiments, the drive table 9018 can include one or more planar support surfaces 9019A-B. In some embodiments, the drive table 9018 can include an extendable or telescoping member 9023. The drive table 9018 can be further coupled to the joint setup 9022. In some embodiments, the drive table 9018 can further include a handle 9041. The drive table 9018 can be configured to position a drive assembly proximal to an access point on a patient.

The main body 9005 can be a longitudinal body having one or more walls defining an interior cavity. One of the one or more walls of the main body 9005 can be a planar support surface. The interior cavity can be configured to house and/or support the telescoping member 9023. The interior cavity can further include a drive system as described in greater detail herein.

The one or more planar support surfaces 9019A-B may be non-angled relative to a cartesian coordinate system. In some examples, the one or more planar support surfaces 9019A-B may extend along a vertical and/or horizontal axis. For example, the support surface 9019A can be a generally planar support surface oriented along a vertical plane (e.g., a Y-Z). The support surface 9019B can be a generally planar support surface oriented along a horizontal plane (e.g., an X-Z). The one or more planar support surfaces 9019A-B can be planar surfaces extending along the longitudinal axis of the drive table 9018. The one or more planar support surfaces 9019A-B can be configured to support one or more elements of the drive assembly 9011. In some examples, the one or more planar support surfaces 9019A-B can be configured to support a plurality of hub assemblies of the drive assembly 9011. In some examples, one or more of the planar support surfaces 9019A-B may be horizontal or oriented at an angle between a vertical and horizontal plane.

The telescoping member 9023 can be a longitudinal body configured to extend from and/or retract into the main body 9005. In some embodiments, the telescoping member 9023 can be a support bracket. In some embodiments, the telescoping member 9023 may be configured to support one or more interventional devices at a position proximal to a patient. In some embodiments, an interventional device can extend from the one or more mounts or hub assemblies through a distal end of the telescoping member 9023. In some embodiments, the telescoping member 9023 (e.g., distal end thereof) can be coupled to an access sheath (e.g., a femoral access sheath) at a patient access point. In some embodiments, the robotic drive system can also include an anti-buckling feature (which may be the same or similar to any of the anti-buckling features described herein) that can couple to the distal end of the telescoping member 9023. For example, the anti-buckling feature may extend from a hub assembly to the distal end of the telescoping member 9023. The anti-buckling feature can be configured to stiffen a portion of an interventional device supported by the drive table 9018.

The handle 9041 can be configured to provide a grasping location to provide for manipulating the drive table 9018 (e.g., between stowed configuration and/or one or more operational configurations). In some embodiments, the handle 9041 can extend from the main body 9005. In some embodiments, a force cell or load cell can be positioned within the handle or at a location where the handle couples to the main body 9005 or other portion of the drive table 9018. In some embodiments, forces applied by a user to the handle 9041 to move the drive table 9018 can be detected by the force cell or load cell, and can be processed by the control system. The control system can cause the robotic arm of the joint setup 9022 to move in response to the detected forces, for example, to cause the drive table 9018 to move in accordance to the detected forces (e.g., in the same direction).

In some embodiments, the handle 9041 may include a display screen (such as display 23). In some embodiments, the handle 9041 may include the interface 9020. The interface 9020 can include a display and input device for receiving an input from a user. In some embodiments, the interface 9020 may be separate from the handle 9041.

The drive assembly 9011 can include a plurality of hub assemblies. In some embodiments, the drive assembly 9011 can additionally include sterile field barrier. In some embodiments, the drive assembly 9011 may be disposable and/or include disposable devices, such as one or more disposable devices 5008.

In certain embodiments, the drive assembly 9011 can include a fluidics cassette 9012. In other embodiments, the fluidics cassette 9012 may be separate from and couplable to the drive assembly 9011. In certain embodiments, the drive assembly 9011 can include a plurality of fluid conduits or fluid communication channels 9013. In other embodiments, the fluid communication channels 9013 can be separate from and couplable to the drive assembly 9011. The fluidics cassette 9012 and/or fluid communication channels 9013 can be configured to access the vascular system of a patient to supply fluids (e.g., saline, contrast, drugs or components thereof) to a patient and/or remove fluids from the patient. For example, the done or more interventional devices can be guided to a target site and provide fluids and/or aspiration to the patient.

The plurality of hub assemblies can be the same or similar any of the hubs or hub assemblies described herein. The plurality of hub assemblies can include a first hub assembly 9034A, a second hub assembly 9034B, a third hub assembly 9034C, and/or a fourth hub assembly 9034D. Each of the plurality of hub assemblies can be configured to translate along the one or more planar support surfaces 9019A-B of the drive table 9018 or a drive surface placed over one or more of the planar support surfaces 9019A-B of the drive table (e.g., a drive surface of a sterile barrier placed over one or more of the planar support surfaces 9019A-B). In some embodiments, the plurality of hub assemblies can be magnetically coupled to a corresponding carriage or hub adapter of the drive system located within the main body 9005. Each of the one or more hub assemblies can be coupled to an interventional device. In some examples, each of the plurality of hub assemblies can be coupled to a particular interventional device. For example, the fourth hub assembly 9034D can be coupled to a catheter (e.g., a guide catheter) having a first diameter, the third hub assembly 9034C can be coupled to a catheter (e.g., a procedure catheter) having a second diameter, the second hub assembly 9034B can be coupled to a catheter (e.g., an access catheter) having a third diameter, and the first hub assembly 9034A can be coupled to a guide wire. In some embodiments, the one or more interventional devices can be coaxially nested within one another. For example, the first diameter can be larger than the second diameter, the second diameter can be larger than the third diameter, and the third diameter can be larger than the diameter of the guidewire. Accordingly, the plurality of hub assemblies can be longitudinally driven relative to one another (e.g., along the Z axis).

The one or more hub assemblies can include one or more magnets or magnet elements. Each magnet element may be a single magnet or a set or array of magnets. A magnet element may be a set or array of magnets configured to move (e.g., rotate) together.

The one or more magnet elements may correspond to a number of degrees of freedom within the one or more hub assemblies. In some examples, the one or more magnet elements may include a first magnet element, a second magnet element, a third magnet element, and a fourth magnet element. In an operable configuration, the rotation of each of the one or more magnet elements can result in an actuation of a corresponding degree of freedom of the hub assembly. For example, rotation of the first magnet element can actuate a first degree of freedom, rotation of the second magnet element can actuate a second degree of freedom, rotation of the third magnet element can actuate a third degree of freedom, and rotation of the fourth magnet element can actuate a fourth degree of freedom. The degrees of freedom of the one or more hub assemblies can include actuating a stopcock, actuating a valve (e.g., a hemostasis valve), and/or rotating an interventional device. The one or more magnet elements may be operatively coupled to a corresponding gear train for transferring a rotational motion of the one or more driven magnet elements. In some embodiments, as described herein, a magnetic coupling between the one or more magnet elements and one or more magnet elements of a hub adapter can be used to provide a shear force to drive the one or more hub assemblies along the length of the main body 9005.

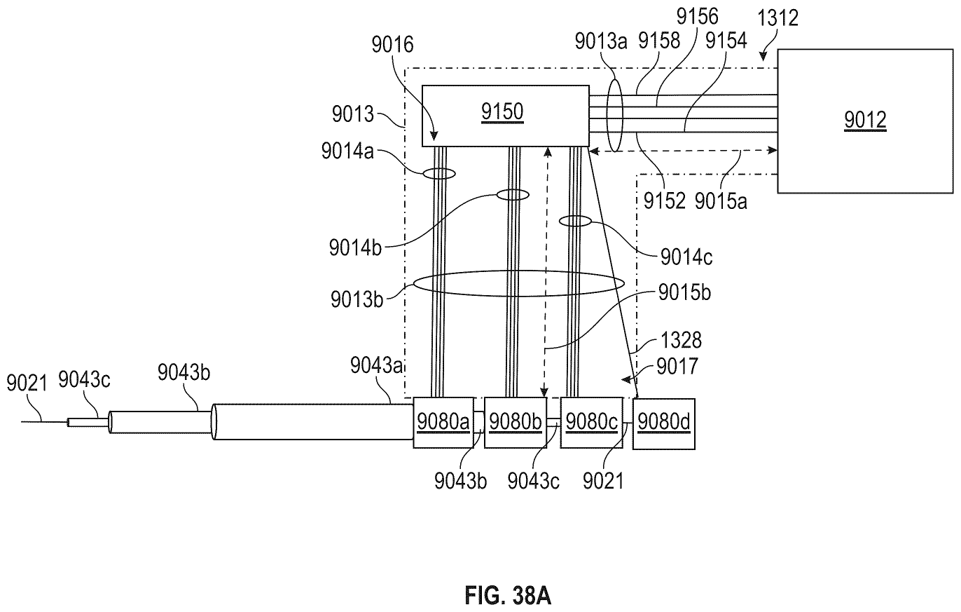

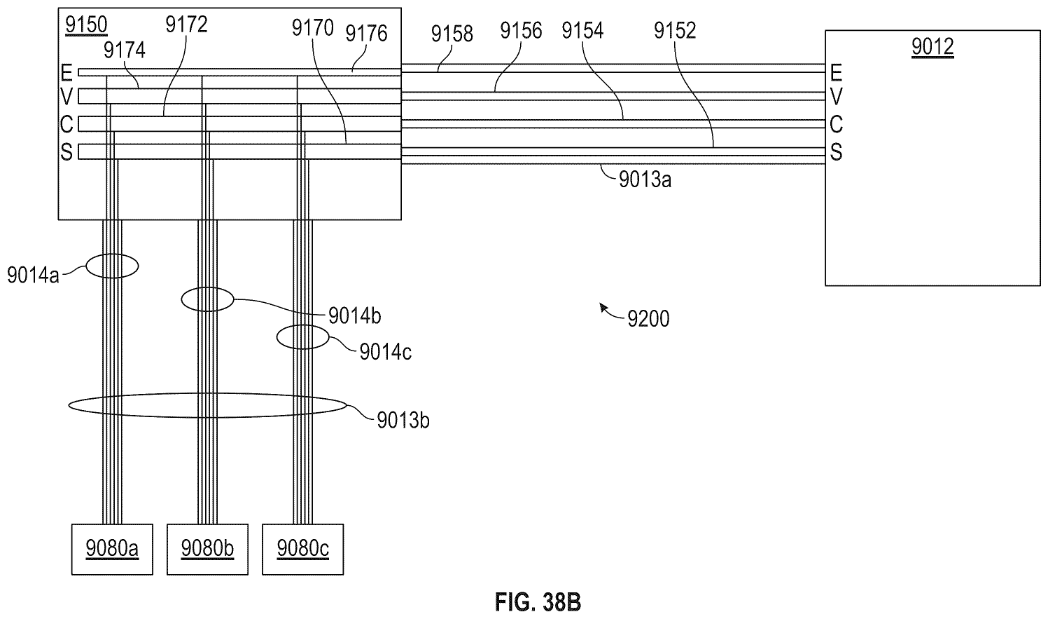

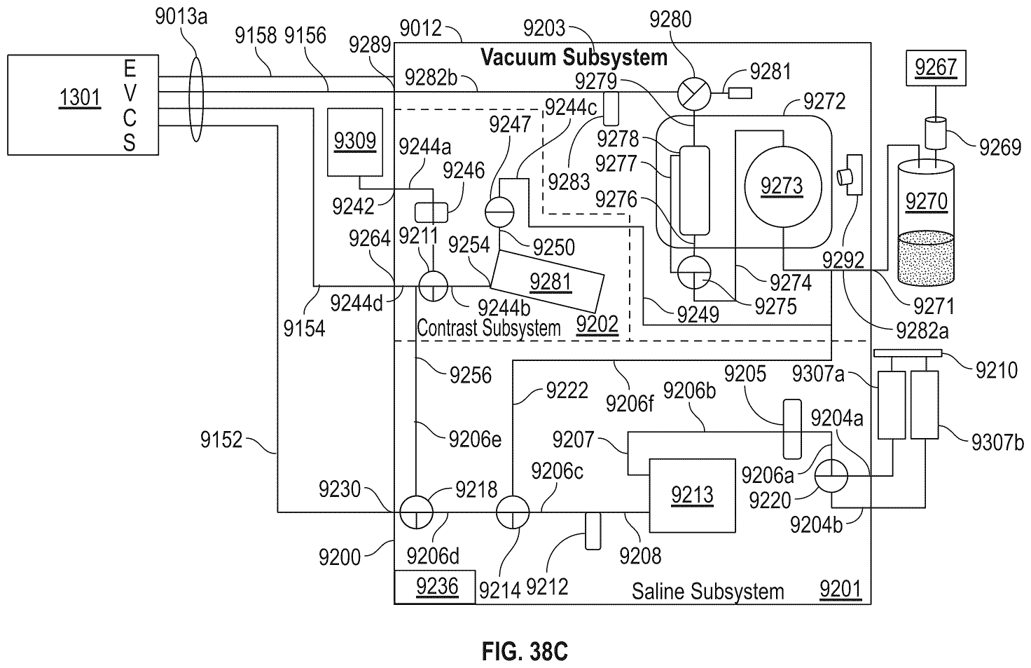

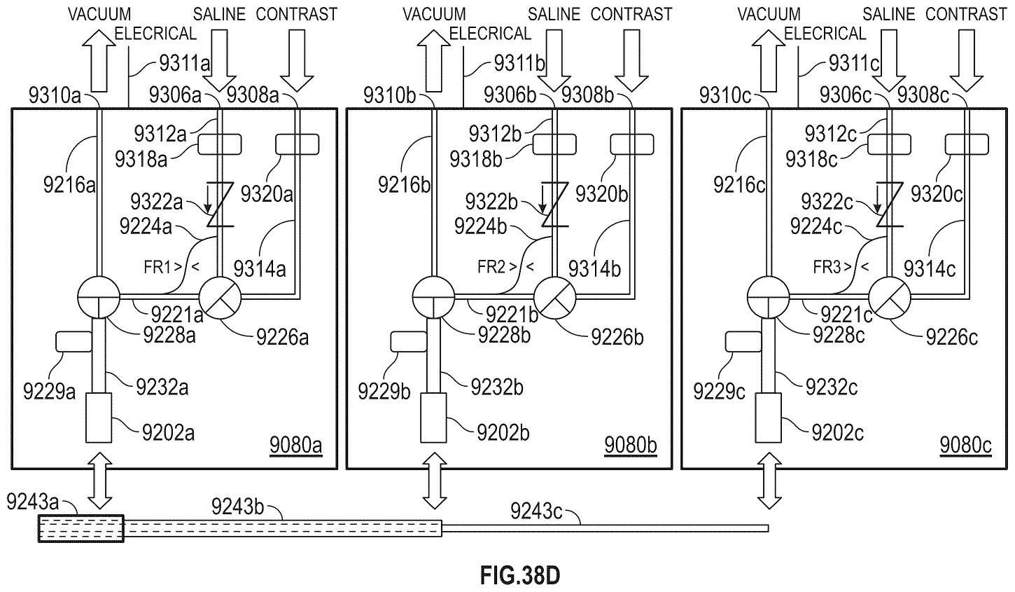

The fluidics cassette 9012 can be configured to be releasably coupled to a pump station, and configured to receive saline from a saline source, receive contrast from a contrast source, and receive vacuum from a vacuum source. The fluidics cassette 9012 can include a saline subsystem having a first saline flow-path, a contrast subsystem having a first contrast flow-path, and a vacuum subsystem having a first vacuum flow-path. The fluidics cassette 9012 can further include a splitter that has as in input a single saline line, a single contrast line, and a single vacuum line, and splits the single saline line into multiple lines, splits the single contrast line into multiple lines, and splits the single vacuum line into multiple lines. Accordingly, the splitter is configured to have a second saline flow-path, a second contrast flow-path, and a second vacuum flow-path, each of the second saline, contrast, and vacuum flow-paths having a single proximal end (i.e., as input fluidic channels to the splitter) and a plurality of distal ends (i.e., as output fluidic channels extending from the splitter). In some embodiments, the splitter may be separate from the fluidics cassette 9012, for example, in the form of a relay cassette (e.g., splitter 9150 of FIG. 38A). In some embodiments, the splitter may be part of or coupled to the drive assembly 9011.

In some embodiments, the fluidics cassette 9012 may be in fluid communication with a splitter having a number of saline flow-paths corresponding to the number hub assemblies or a subset of the hub assemblies, contrast flow-paths corresponding to the number of hub assemblies or a subset of hub assemblies, and vacuum flow-paths corresponding to the number of hub assemblies or a subset of hub assemblies. The fluidics cassette 9012 can further include a first tubing set having a first length and coupled to the fluidics cassette 9012 and the splitter 9150, the first tubing set including a single saline channel coupled to the first saline flow-path and the proximal end of the second saline flow-path, a single contrast channel coupled to the first contrast flow-path and the proximal end of the second contrast flow-path, and a single vacuum channel coupled to the first vacuum flow-path and the proximal end of the second vacuum flow-path.

The plurality of fluid communication channels 9013 can be configured to fluidly couple the one or more hub assemblies 9034A-D to the fluidics cassette 9012. In some examples, the plurality of fluid communication channels 9013 can be organized into one or more sets of fluid conduits or fluid communication channels. For example, the plurality of fluid communication channels 9013 can be organized into a number of sets corresponding to the number of hub assemblies or a subset of hub assemblies. In some embodiment, the plurality of fluid communication channels 9013 may include three sets of fluid conduits or fluid communication channels corresponding to the first hub assembly 9034A, the second hub assembly 9034B, the third hub assembly 9034C. In some embodiments of a system with four interventional devices, the plurality of fluid communication channels 9013 may include four sets of fluid conduits or fluid communication channels corresponding to the first hub assembly 9034A, the second hub assembly 9034B, the third hub assembly 9034C, and the fourth hub assembly 9034D, respectively. In some embodiments, each set of fluid communication channels may include a saline conduit, a contrast conduit, and a vacuum conduit. In some embodiments, each set of fluid communication channels can further include an electrical conduit or channel, which can be used to provide electrical signals from components (e.g., sensors, valves, etc.) in a hub assembly to a controller, and/or to provide electric control signals to valves in a hub assembly.

In some embodiments (e.g., where there is a catheter coupled to the fourth hub assembly), at least some of the plurality of fluid communication channels 9013 can extend between the fourth hub assembly 9034D and the fluidics cassette 9012. In some embodiments, at least some of the plurality of fluid communication channels 9013 can extend between the third hub assembly 9034C and the fluidics cassette 9012. In some embodiments, at least some of the plurality of fluid communication channels 9013 can extend between the second hub assembly 9034B and the fluidics cassette 9012. In some embodiments, at least some of the plurality of fluid communication channels 9013 can extend between the fourth hub assembly 9034D and the fluidics cassette 9012. Accordingly, in some embodiments, each of the one or more hub assemblies can be in fluid communication with the fluidics cassette 9012. Such embodiments, may allow for replacement of a hub of any of the hub assemblies with one having a different interventional device. In other embodiments, only a subset of the hub assemblies may be in fluid communication with the fluidics cassette. For example, one of the hub assemblies may be configured for use with a guidewire. In some embodiments, such a hub assembly may not be in fluid communication with the fluidics cassette and may not couple to any fluid conduits or fluid communication channels 9013.

In some embodiments, the drive assembly 9011 can include a casing 9032. In some embodiments, the casing 9032 can act as a sterile packaging assembly. For example, the casing 9032 can act as a sterile packaging assembly for transporting components of the robotic surgery system (e.g., interventional devices, hub assemblies, fluid communication channels or portions thereof, a relay cassette, etc.) to a robotic surgery site. The casing 9032 may enclose such components within a sterile volume.

The casing 9032 can include a plurality of walls. The plurality of walls may enclose a sterile volume. In some examples, a stowed configuration can correspond to a packaged, closed, and/or preset configuration. For example, the casing 9032 can be assembled at the end of manufacturing and/or prior to shipping.

In some examples, the components of the drive assembly 9011 can be set within the casing 9032. In some embodiments, an operational configuration can correspond to an open configuration. For example, the casing can be opened to allow for set up of the drive assembly 9011. In some embodiments, a wall 9033 of the casing 9032 can form a base for the drive assembly 9011. For example, the wall 9033 can be configured to be positioned along one or more of the planar support surfaces 9019A-B. In some embodiments, the wall 9033 can provide a sterile field barrier. For example, the wall 9033 may abut one or more of the planar support surfaces 9019A-B of the drive table 9018. In some embodiments, the casing 9032 can extend along the length of the one or more planar support surfaces 9019A-B of the drive table 9018.

In some embodiments, the drive assembly 9011 can further include one or more handles 9044. The one or more handles 9044 can be configured to be grasped for manipulating and/or moving the drive assembly 9011. In some embodiments, the drive assembly 9011 may be wholly confined within the casing in the closed configuration. For example, the drive assembly 9011 can be enclosed within the casing in the closed configuration prior to shipping. A user may grasp the one or more handles 9044 and position the drive assembly 9011 on top of the drive table 9018. In some examples, a user may grasp the one or more handles 9044 to position the drive assembly 9011 along the one or more planar support surfaces 9019A-B.

In some embodiments, the fluidics cassette 9012 may be attached (e.g., mounted) to an exterior of the casing 9032. In some embodiments, at least a portion of the plurality of fluid communication channels 9013 can be positioned exterior to the casing 9032. In some embodiments, the plurality of fluid communication channels 9013 can extend form the fluidics cassette 9012 exterior to the casing 9032 and into the casing 9032 to couple with the hub assemblies contained therein.

In some embodiments, the drive assembly 9011 can be disposable. The drive assembly 9011 can be shipped as a unit and then coupled to the drive table 9018 for performing a robotic surgical procedure. In some embodiments, the fluidics cassette 9012 can be coupled to one or more fluid and/or vacuum sources prior to beginning a robotic procedure. In some embodiments, the fluidics cassette 9012 can be detached from the casing 9032 and attached to a fluidics tower, wherein it can be coupled to one or more fluid and/or vacuum sources.

The joint setup 9022 can be a device for supporting and/or orienting the drive table 9018 to a desired position. In some embodiments, the base of the joint setup 9022 can be a dynamic support system configured to selectively orient the drive table 9018 between two or more positions. The joint setup 9022 can include one or more robotic arms configured to support and orient the drive table 9018 in a desired configuration. For example, the joint setup 9022 can include a robot arm, such as a Selective Compliance Assembly Robot Arm (“SCARA”). The joint setup 9022 can include a first end and a second end. The first end of the joint setup 9022 can couple to the bed and the second end of the joint setup 9022 can couple to the drive table 9018.

In certain embodiments, the drive table 9018 can be stowed in a stowed configuration when not in use. The stowed configuration can conserve space. In some examples, the drive table 9018 may be positioned vertically in the stowed configuration. The drive table 9018 can be configured to transition from a stowed configuration to an operational configuration.

FIG. 32A illustrates the drive table 9018 positioned in an operational configuration. In the operational configuration of FIG. 32A, the drive table 9018 may be horizontal or generally horizontal. In some embodiments, in the operational configuration of FIG. 32A, a longitudinal axis of the drive table 9018 may be perpendicular to or generally transverse to the Y-axis. In some embodiments, the drive table 9018 may be driven to the operational configuration of FIG. 32A via a robotic base or joint setup 9022. In some embodiments, the drive table may be driven to the operation configuration via manipulation of the handle 9041. As shown in FIG. 32A, the telescoping member 9023 may be positioned entirely or mostly within the drive table 9018.

In certain embodiments, the drive table 9018 may be positioned at a setup position, as shown in FIG. 32A. In the setup position, the drive table 9018 can be prepared for a procedure. For example, in the setup position, one or more sterile coverings can be applied to the drive table 9018. In the setup position, the drive assembly 9011 can be coupled to the drive table 9018. In some embodiments, the drive table 9018 can include one or more anchors configured to secure the drive assembly 9011 to the drive table 9018.

FIG. 32B illustrates the drive assembly 9011 being applied to the drive table 9018. As shown in FIG. 32B, the drive assembly can be placed onto the drive table 9018 in a stowed configuration so that the wall 9033 mates with or contacts the one or more planar support surfaces 9019A-B. The drive assembly can include one or more connectors configured to couple with the anchors of the drive table 9018 to secure the drive assembly 9011 to the drive table 9018. For example, in some embodiments, the casing 9032 can include one or more connectors configured to couple with the anchors. The drive assembly 9011 can transition from the stowed configuration to the operational configuration after being placed on one or more of the planar support surfaces 9019A-B of the drive table 9018. Once secure, the drive assembly can transition from the stowed configuration to an operational configuration. In some examples, the drive assembly can transition from the stowed configuration to the operational configuration by rotating a second wall and a third wall about a hinge portion. In some embodiments, the casing 9032 can be a sterile tray.

After setup of the drive table 9018 and drive assembly 9011, the drive table 9018 can be moved to as position over the patient support table so that the interventional devices of the hub assemblies are generally positioned over the patient support table (e.g., by rotating and lifting the drive table 9018 from the setup position.

After the drive table 9018 is positioned over the support table the drive table 9018 can be advanced to a patient access point, as shown in FIG. 32C. The telescoping member 9023 can couple to an access sheath (e.g., a femoral access sheath). For example, the distal end of the telescoping member 9023 can couple to the access sheath. In certain embodiments, an anti-buckling system or device may be coupled to the telescoping member 9023 (e.g., at the distal end) and to the fourth hub assembly 9034D while the drive table 9018 is positioned adjacent the patient access point as shown in FIG. 32C.

In certain embodiments, after the telescoping member 9023 is coupled to the access sheath, the telescoping member 9023 may be extended from the drive table 9018. In some embodiments, the drive table 9018 may be driven proximally away from the distal end of the telescoping member 9023 For example, the distal end of the telescoping member 9023 can remain at a position adjacent to the patient access point and coupled to the access sheath while the main body translates proximally away from the distal end of the telescoping member, for example, to the position shown in FIG. 32D.

As shown in FIG. 32D, one or more interventional devices can extend distally from the fourth hub assembly 9034D through the distal end of the telescoping member 9023. In certain embodiments, the one or more interventional devices are inserted into the access sheath (e.g., through the distal end of the telescoping member) after the main body is retracted relative to the patient access point as shown in FIG. 32D. For example, in certain embodiments, prior to retracting the main body 9005 the interventional devices may be coupled to their respective hub assembly but maintained in a stowed configuration. After retraction of the main body 9005 the interventional devices may be transitioned to an operational configuration and inserted through the access sheath. In some embodiments, after the interventional devices are inserted through the access sheath, an anti-buckling system or device may be coupled to the telescoping member (e.g., at the distal end) and to the fourth hub assembly 9034D to provide anti-buckling support. In certain embodiments, anti-buckling systems or devices may also be coupled between adjacent hub assemblies for interventional device support. Anti-buckling systems or devices may be provided coupled between adjacent hub assemblies in the drive assembly, may be coupled between adjacent hub assemblies prior to retracting the main body, or may be coupled between adjacent hub assemblies after retracting the main body.

The configuration of FIG. 32D may be referred to as a procedure configuration. In the procedure configuration, the one or more interventional devices may be driven by their respective hub assemblies through the vasculature of the patient.

In certain embodiments, the main body 9005 may be translated distally and/or proximally during a procedure to adjust the position of one or more hub assemblies and/or interventional devices. The one or more hub assemblies may be independently and/or simultaneously driven with the main body to provide relative movements. For example, if a hub assembly is at its distal most position along the drive table 9018, but further advancement of the interventional device coupled to the hub assembly is desired, the main body may be translated distally to translate the hub assembly distally and the interventional device distally within the vasculature. In certain embodiments, if the main body moves to adjust the position a particular hub assembly, but similar movement one or more other hub assemblies is not instructed, the one or more other hub assemblies 9034A-D may automatically move to retain their desired relative positions to the particular hub assembly. For example, the one or more other hub assemblies may move axially along the Z-axis in an opposite direction and by a same amount as the movement of the main body.

As shown in FIGS. 32A-32D, the support surface 9019A can be a generally planar support surface oriented along a vertical plane (e.g., a Y-Z plane). The support surface 9019B can be a generally planar support surface oriented along a horizontal plane (e.g., an X-Z plane). A portion of each of the hub assemblies 9034A-D can translate along the support surface 9019A or a sterile barrier positioned thereon and a portion of each of the hub assemblies 9034A-D can translate along the support surface 9019B or a sterile barrier positioned thereon.

The arrangement of hub assemblies 9034A-D and support surfaces 9019A-B can facilitate positioning of the interventional devices coupled to the hub assemblies 9034A-D at a relatively low position relative to the patient access point. As shown in FIG. 32D, the one or more interventional devices can be positioned near a bottom end of the support surface 9019A. For example, the hub assemblies 9034A-D can include a first portion that extends generally vertically and translates along the support surface 9019A or a sterile barrier positioned thereon. The interventional devices may extend from a lower section of the first portion of the hub assemblies. A low position of the one or more interventional devices relative to the patient access point can reduce the dead length of the interventional devices and provide a shallow approach angle.

In some embodiments, a magnetic coupling between the magnets of the hub adapters and corresponding hub assemblies can hold the hub assemblies 9034A-D onto the support surface 9019A of the drive table 9018. In certain embodiments, a second portion of the hub assemblies 9034A-D can extend generally horizontally and translate against the support surface 9019B or a sterile barrier positioned thereon. The support surface 9019B may provide support to the hub assemblies to prevent misalignment of the hub assemblies 9034A-D due to vertical forces (e.g., gravity) acting on the hub assemblies. Accordingly, the hub assemblies 9034A-D can maintain a lower position for the interventional devices while being securely mounted to the drive table 9018.

Additional details regarding drive tables are disclosed in U.S. Patent Application Ser. No. 63/656,547, entitled DRIVE TABLE, filed Jun. 5, 2024, and U.S. patent application Ser. No. 18/524,879, entitled ROTATABLE DRIVE TABLE, filed Nov. 30, 2023, each of which is hereby expressly incorporated by reference in its entirety herein.

### Hub Assembly Coupling and Alignment

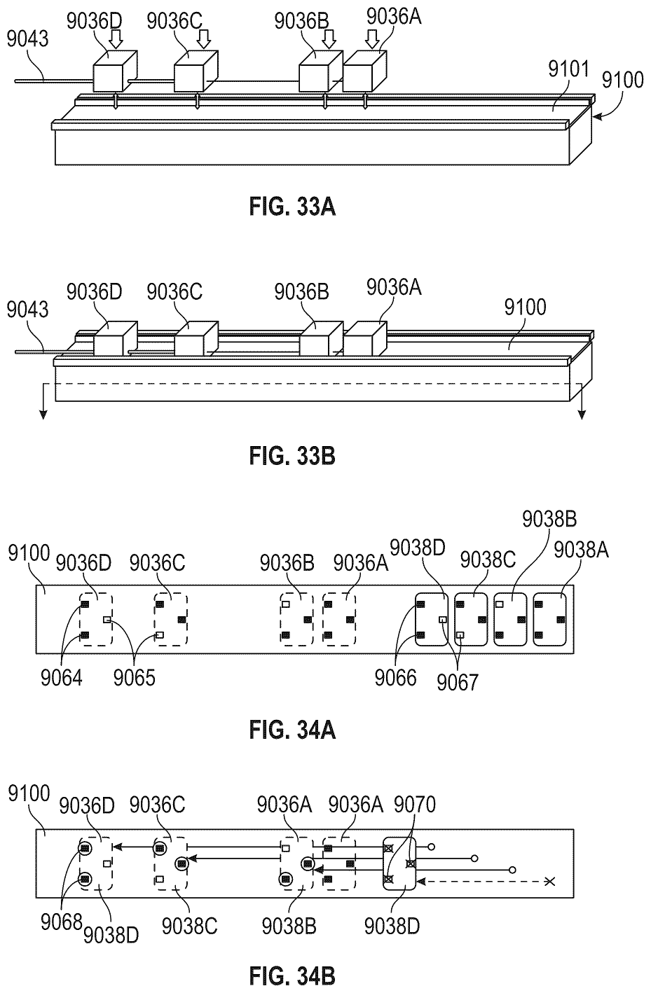

FIGS. 33A-33B and 34A-B depict methods and systems for aligning and coupling one or more hub assemblies with one or more hub adapters across a sterile barrier.

In some embodiments, one or more hub assemblies may be configured to couple to a respective one of a plurality of hub adapters positioned within the body of the drive table 9100. The one or more hub assemblies can include a first hub assembly 9036A, a second hub assembly 9036B, a third hub assembly 9036C, and a fourth hub assembly 9036D. The one or more hub assemblies can be the same or similar to the hub assemblies 9034A-D described above. The drive table 9100 can be the same or similar to the drive table 9018 described above. The plurality of hub adapters can include the same number of hub adapters as the number of hub assemblies. For example, the plurality of hub adapters can include a first hub adapter 9038A, a second hub adapter 9038B, a third hub adapter 9038C, and a fourth hub adapter 9038D.

In some embodiments, each of the one or more hub assemblies (e.g., mounts thereof) can include one or more identifiers that may be detected, for example, by a sensor. The identifiers may be used to determine the identity of a hub assembly for coupling the hub assembly with a desired hub adapter. In some embodiments, the identifiers may be detected by one or more sensors 9066 of the hub adapters or otherwise associated with the hub adapters.

In some embodiments, the one or more identifiers can be detectable objects 9064 (e.g., identifier magnets). The detectable objects 9064 may be arranged in an identifying configuration. For example, as shown in FIGS. 34A-34BD, each of one or more hub assemblies can include two or more detectable objects 9064 arranged in a unique pattern. Each of the plurality of hub adapters can include a corresponding arrangement of sensors 9066 configured to measure the detectable objects 9064.

FIGS. 33A-33B show one or more hub assemblies being placed on a drive surface 9101 (e.g., a planar drive surface or substantially planar drive surface) of the drive table 9100 to be magnetically coupled to corresponding hub adapters. In some embodiments, the drive surface may be part of a barrier formed by a surface of the drive table. The barrier may be a sterile barrier. In other embodiments, the drive surface may be part of a separate barrier that is placed over the drive table and acts as a sterile barrier. For example, the drive surface 9101 may be a surface of the casing 9032.

In the setup or operable configuration, the one or more hub assemblies can be driven along the drive surface 9101 of the drive table 9100 by the shear force of a magnetic coupling between the one or more hub assemblies and the corresponding hub adapter.

Each of the one or more hub assemblies can include an intervascular device. For example, FIGS. 33A and 33B depict an intravascular device such as a catheter 9043. The intervascular devices coupled to the one or more hub assemblies can be devices configured to access the vascular system of a patient. For example, the intervascular devices can be catheters and/or guidewires. In some embodiments, the intervascular devices of the one or more hub assemblies can be coaxially nested, as shown in FIGS. 33A-33B.

In some embodiments, an intravascular device having the smallest diameter may be coupled to the first hub assembly 9036A, an intravascular device having a lumen with an inner diameter greater than the intravascular device of the first hub assembly 9036A may be coupled to the second hub assembly 9036B, an intravascular device having a lumen with an inner diameter greater than the outer diameter of the intravascular device of the second hub assembly 9036B may be coupled to the third hub assembly 9036C, and an intravascular device having a lumen with an inner diameter greater than the outer diameter of the intravascular device of the third hub assembly 9036C may be coupled to the fourth hub assembly 9036D. For example, a guidewire may be coupled to the first hub assembly 9036A, a catheter (e.g., an access catheter) may be coupled to the second hub assembly 9036B, another catheter (e.g., a procedure catheter) may be coupled to the third hub assembly 9036C, and another catheter (e.g., a guide catheter) may be coupled to the fourth hub assembly 9036D.

In some embodiments, for example, during a setup of a robotic surgical system, the one or more hub assemblies can be placed (e.g., manually) on the drive surface 9101 of the drive table 9100. In some embodiments, the one or more hub assemblies may be placed at a designated location along the drive surface 9101 of the drive table 9100. For example, one or more indicators (e.g., colors, lines, images, etc.) may indicate a location along the drive surface at which to place the one or more hub assemblies. In some embodiments, the designated locations for the one or more hub assemblies may correspond to designated locations for the hub adapters in a setup configuration (e.g., at which the hub adapters are positioned or to which the hub adapters may be automatically driven). In such embodiments, placing the one or more hub assemblies in their designated locations may align the one or more hub assemblies with their corresponding hub adapters.

In some embodiments, one or more hub assemblies may not be accurately placed over the corresponding hub adapters during the initial setup or if one or more hub assemblies is replaced during a procedure. In such embodiments, the hub adapters can be configured to perform an automated alignment process to align with the one or more hub assemblies. In other embodiments, the one or more hub assemblies can be placed anywhere along the drive surface of the drive table 9100 regardless of the position of the plurality of hub adapters and/or a designated position for the one or more hub assemblies, and the hub adapters can be configured to perform an automated alignment process to align with the one or more hub assemblies. In such embodiments, the plurality of hub adapters can detect whether the corresponding one or more hub assemblies are aligned with the hub adapters. If one or more hub assemblies are not aligned with the hub adapters, the hub adapters can perform an automated alignment process.

FIGS. 34A-34B illustrate an embodiment of driving the plurality of hub adapters beneath the barrier to align with and magnetically couple with a corresponding one of the one or more hub assemblies. In some embodiments, the first hub adapter 9038A may be paired with and/or programmed to identify the first hub assembly 9036A, the second hub adapter 9038B may be paired with and/or programmed to identify the second hub assembly 9036B, the third hub adapter 9038C may be paired with and/or programmed to identify the third hub assembly 9036C, and the fourth hub adapter 9038D may be paired with and/or programmed to identify the fourth hub assembly 9036D.

The one or more hub assemblies are shown in dashed lines and are located on a first side (e.g., above) of the barrier (e.g., on a drive surface). The one or more hub adapters are shown in solid lines and are located on a second side (e.g., below) of the barrier (e.g., on an interior drive surface).

In certain embodiments, each of the one or more hub assemblies can include one or more detectable objects 9064 (e.g., identifier magnets, specific colors, light diodes, or other detectable objects) that may be detected by one or more sensors 9066 of one of the hub adapters to confirm alignment between the one or more hub assemblies 9102A-D and hub adapters. The detectable objects 9064 of each of the one or more hub assemblies 9102A-D may be arranged in unique positions (e.g., patterns) relative to the other hub assemblies to allow for identification of the individual hub assembly 9102A-D. Additionally or alternatively, at least some of the one or more hub assemblies may include a different number of detectable objects 9064 relative to one another (e.g., two objects vs. three objects) to allow for identification of the individual hub assemblies.

The one or more hub adapters may include sensors 9066 configured to align with the positions of the detectable objects 9064 to detect the detectable objects 9064 of the one or more hub assemblies and identify the one or more hub assemblies.

The detectable objects 9064 may be placed at multiple points along an hub assembly to allow for detection of proper alignment of the one or more hub assemblies. For example, each of the one or more hub assemblies may include detectable objects 9064 at multiple locations. If a hub adapter, only detects a detectable object 9064 at a single location, it may indicate that the corresponding hub assembly is misaligned. For example, it may indicate that the hub assembly is misaligned rotationally relative to the hub adapter (e.g., due to an undesired yaw rotation).

In FIGS. 34A and 34B, the one or more hub assemblies are shown with a plurality of squares 9065 identifying examples of potential positions of detectable objects 9064 (e.g., identifiers) on the one or more hub assemblies. As shown, in this example, each of the one or more hub assemblies has the same arrangement of potential positions, although in other embodiments, different arrangements are possible. As shown, empty squares can represent locations in which a detectable object is not present, and filled in squares can represent detectable objects 9064 (e.g., identifiers). For example, the filled squares of the one or more hub assemblies can represent identifier magnets, specific colors, light diodes, or other detectable objects. The detectable objects 9064 can be arranged in a pattern.

The hub adapters are also shown with a plurality of squares 9067 identifying examples of potential positions of sensors 9066 on the hub adapters for detecting detectable objects 9064 of the one or more hub assemblies. As shown, in this example, each of the hub adapters has the same arrangement of potential positions, although in other embodiments, different arrangements are possible. As shown, empty squares can represent locations in which a detectable object 9064 is not present, and filled in squares can represent a sensor position on the hub adapters. The sensors 9066 may be hall effect sensors, imaging sensors, or any other suitable sensors. The sensors 9066 of the plurality of hub adapters can be arranged in a pattern. In some embodiments, each of the hub adapters can share a common general pattern of possible positions for the sensors 9066. The common general pattern of the hub adapters may correspond to the common general pattern of the one or more hub assemblies. For example, each of the hub adapters can include sensors 9066 arranged along a chevron pattern.

As shown in FIG. 34B, each of the hub adapters may be driven beneath the barrier until the hub adapter arrives at a position of a corresponding hub assembly. For example, each of the hub adapters may drive along the length of the drive table 9100 until the hub adapter identifies its corresponding hub assembly and/or detects that it is aligned with its corresponding hub assembly, respectively.

Each of the plurality of hub adapters may be independently driven. Accordingly, it is possible to drive a single hub adapter to align with its corresponding hub assembly. As shown in FIG. 34B, the fourth hub adapter 9038D can be driven past the first hub assembly 9036A, the second hub assembly 9036B, and the third hub assembly 9036C until the fourth hub adapter 9038D is aligned with the fourth hub assembly 9036D (e.g., alignment between the fourth hub assembly 9036D and the fourth hub adapter 9038D is detected via the sensors 9066 of the fourth hub adapter 9038D). In some embodiments, the sensors 9066 of the fourth hub adapter 9038D may scan for detectable objects 9064 as it is advanced within the drive table 9100 until alignment is detected. Similarly, the third hub adapter 9038C can be driven past the first hub assembly 9036A and the second hub assembly 9036B until the third hub adapter 9038C arrives at the third hub assembly 9036C. The second hub adapter 9038B can also be driven past the first hub assembly 9036A until the second hub adapter 9038B arrives at the second hub assembly 9036B. The plurality of hub adapters may be driven simultaneously or one at a time. Under either approach, the plurality of hub adapters may be driven independently.

As further shown in FIG. 34B, circles 9068 are provided to illustrate detectable objects 9064 of the one or more hub assemblies that are aligned with corresponding sensors 9066 of the hub adapters indicating an alignment between those hub assemblies and hub adapters. For example, circles 9068 are shown over the detectable objects 9064 of the fourth hub assembly 9036D, the third hub assembly 9036C, and the second hub assembly 9036B because they are successfully aligned with their corresponding hub adapters. In FIG. 34B, “x” symbols 9070 are provided over the sensors 9066 of the first hub adapter 9038A to indicate that none of the sensors 9066 of the first hub adapter 9038A have successfully detected a detectable object 9064 of a hub assembly.

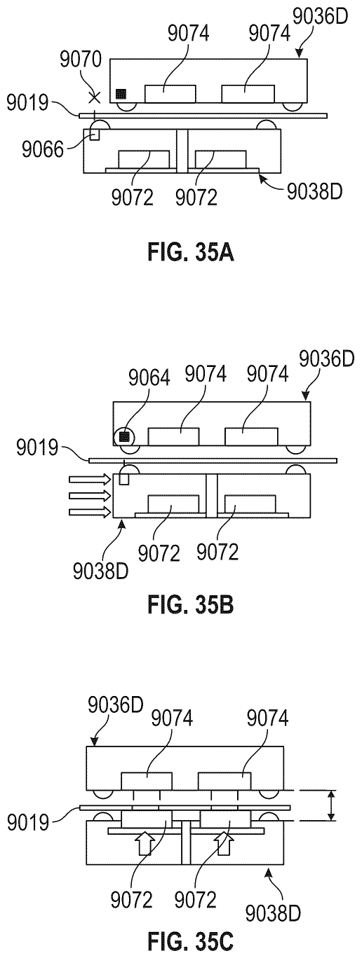

FIGS. 35A-35C illustrate a method of aligning a hub adapter with a hub assembly. The fourth hub assembly 9036D and the fourth hub adapter 9038D are provided as examples. As shown in FIGS. 35A-35C, the fourth hub adapter 9038D may align with the fourth hub assembly 9036D by sensing an identifier, such as a detectable object 9064 as described with respect to FIGS. 34A and 34B. As shown in FIG. 34A, the sensor(s) 9066 of the fourth hub adapter 9038D are not aligned with the detectable object(s) 9064 of the fourth hub assembly 9036D. Accordingly, the fourth hub adapter 9038D may be translated within the drive table 9100 (e.g., sweep) until the sensor(s) 9066 of the fourth hub adapter 9038D detect the detectable object(s) 9064 of the fourth hub assembly 9036D, as shown in FIG. 34B.

The hub adapters can include one or more magnetic elements 9072. The hub assemblies can include one or more magnetic elements 9074. The one or more magnetic elements 9074 can drive an interventional device in one or more degrees of freedom. The hub assemblies can have the same number of magnetic elements 9074 as the hub adapters have magnetic elements 9072. The magnetic elements 9074 can be configured to magnetically couple with a respective one of the magnetic elements 9072. Accordingly, the hub adapters can drive the respective hub assemblies along the drive table 9100 and drive the interventional devices across a sterile barrier. The hub adapters can selectively magnetically couple and uncouple from a corresponding hub assembly by moving the magnets 9072 closer or further from the magnets 9074 of the hub assembly. This may allow for alignment of the hub adapters and hub assemblies (and their corresponding magnets) before establishing a magnetic coupling. For example, as shown in FIG. 35C, the one or more magnetic elements 9072 can be raised to minimize the distance between the magnetic elements 9072 and the magnetic elements 9074 to establish a magnetic coupling.

Additional details regarding sensors, hub assemblies, and alignment of hub assemblies are disclosed in U.S. Provisional Application Ser. No. 63/612,272, entitled Robotic Catheter Hub Assembly, filed Dec. 19, 2023, U.S. patent application Ser. No. 18/389,628, entitled Hub Sensing Through A Sterile Barrier In A Robotic Catheter Assembly, filed Dec. 19, 2023, U.S. patent application Ser. No. 18/545,687, entitled System With Removable Hubs For Manual And Robotic Procedure, filed Dec. 19, 2023, and U.S. patent application Ser. No. 18/678,766, entitled Magnetic Coupling Through a Sterile Field Barrier, filed May 30, 2024, each of which is hereby expressly incorporated by reference in its entirety herein.

### Hub Assemblies

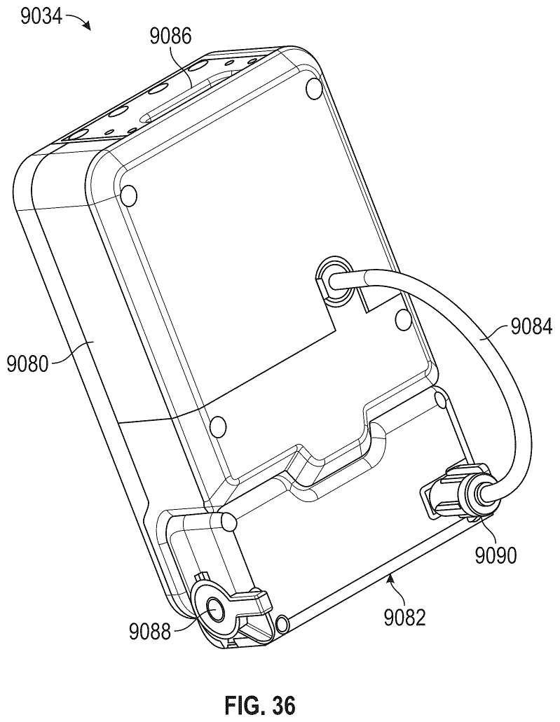

FIG. 36 illustrates an example of a hub assembly 9034. The hub assembly 9034 may be representative of the other hub assemblies. The hub assembly 9034 may include any of the same or similar features and/or functions as the other embodiments of hub assemblies and hubs described herein and vice versa.

In some embodiments, the hub assembly 9034 can be a multi-part hub assembly (e.g., a two-part hub), and include a first subassembly, puck, or mount 9080, and a second subassembly or hub 9082. The mount 9080 can also be referred to as a catheter puck, a hub mount, and/or a first hub member. The mount 9080 can be configured to couple to and move along a drive table, for example, be driven by the drive table as described herein. The hub assembly 9034 can be configured to be positioned on a sterile side (e.g., a disposable equipment side) of a sterile barrier.

In some embodiments, the hub 9082 is a second subassembly and has an interventional device (e.g., catheter or guidewire) coupled to the hub 9082. The hub 9082 can be coupled to the mount 9080 (e.g., for moving the hub 9082 with the drive table).

As described herein, in certain embodiments the hub assembly 9034 may be coupled to a fluidics management system to receive fluids such as contrast or saline, and/or to receive vacuum for aspiration. In some embodiments, the mount 9080 can be coupled to the fluidics management system. In some embodiments, a fluidics connector 9084 can extend between and fluidly couple the mount 9080 and the hub 9082.

The mount 9080 can further include a first housing. The first housing can define one or more openings 9086 and a plurality of internal components described in greater detail below. The first housing can form an outer shell to protect the internal components of the mount 9080. The first housing can include at least one side shaped and/or dimensioned (e.g., having a contour) for receiving the hub 9082.

The one or more openings 9086 can provide access for fluidics and/or electrical connections into the mount 9080. In some embodiments, a contrast tube, a saline tube, and/or an aspiration tube may extend through the one or more openings 9086 into the mount 9080. Additionally, in some embodiments, a power line may extend through the one or more openings 9086 to provide electrical power into the mount 9080. The mount 9080 can be configured to receive an input from one or more active torque elements of an active torque subsystem. In some embodiments, the inputs from the one of more active torque elements may be a magnetic rotary force as described herein. The mount 9080 can be configured to transmit one or more outputs to the hub 9082. In some embodiments, the mount 9080 may transform one or more rotary inputs of the one or more active torque elements into corresponding linear and/or rotary outputs. In some embodiments, the mount 9080 may be configured to translate linearly along a drive table (e.g., in response to linear movement of hub adapter within the drive table due to a magnetic coupling between mount 9080 and the hub adapter).

The hub 9082 can further include a second housing. The hub 9082 can include a lumen 9088 for receiving an interventional device therein. The hub 9082 can include a luer 9090. The hub 9082 can further include a plurality of internal components described in greater detail below. The second housing can form an outer shell to protect the internal components of the hub 9082. In some embodiments, the second housing may include at least one side shaped and/or dimensioned (e.g., having a contour) to correspond to shape of the first housing. For example, the contour of the second housing can correspond to the contour of the first housing of the mount 9080. The hub 9082 can be configured to receive one or more inputs from the mount 9080. The hub 9082 can be configured to transmit one or more outputs. In some embodiments, the hub 9082 may transform the outputs of the mount 9080 into corresponding linear and/or rotary motion of components within or coupled to the hub 9082 (e.g., the interventional device coupled to the hub 9082 and/or one or more fluidics components).

The fluidics connector 9084 can be a tubular body defining an interior lumen extending from one end of the fluidics connector 9084 to a second end of the fluidics connector 9084. In some embodiments, the fluidics connector 9084 may be configured to transport fluids between the mount 9080 and the hub 9082. For example, the fluidics connector 9084 may facilitate the flow of contrast, saline, bodily fluids, and/or air between the mount 9080 and the hub 9082. The fluidics connector 9084 can transport fluids from the mount 9080 to the hub 9082, or vice versa. The fluidics connector 9084 may form an airtight seal.

The hub 9082 may be removably coupled to the mount 9080. In some embodiments, the hub 9082 can be mounted to a mounting element defined by the mount 9080. The fluidics connector 9084 may be coupled to both the mount 9080 and the hub 9082. In some embodiments, the hub 9082 may be in fluid communication with the mount 9080 via the fluidics connector 9084. Accordingly, fluids may be transferred between the mount 9080 and the hub 9082 via the fluidics connector 9084.

In certain embodiments, a mount 9080 can include at least a portion of a passive torque subsystem. In certain embodiments, a portion of a passive torque subsystem may be included in a mount 9080, and another portion of a passive torque subsystem may be included in a hub 9082 coupled to the mount 9080.

### Control Mechanism

FIGS. 37A-37C illustrate one or more aspects of a control mechanism as described herein. A control mechanism may provide one or more user interfaces to allow for user operation of one or more features of a robotic medical system. A control mechanism may also be referred to as an interface mechanism. In some embodiments, a control mechanism may include one or more control devices having one or more controls (e.g., buttons, switches, dials, joysticks, and/or any other suitable actuators) that are configured to send control signals in response to actuation by a user. A control device may also be referred to as an interface device.

Additional details regarding control devices and/or control mechanisms are disclosed in U.S. Patent Application Ser. No. 63/656,545, entitled Method for Robotically Controlling Interventional Device Assembly, filed Jun. 5, 2024, and U.S. patent application Ser. No. 18/525,267, entitled Method for Robotically Controlling Subsets of Interventional Device Assembly, filed Nov. 30, 2023, each of which is hereby expressly incorporated by reference in its entirety herein.

FIGS. 37A-38B illustrate an example control mechanism 9006 for manipulating interventional devices coupled to, and driven by (or otherwise associated with), hub assemblies of a robotic catheter system. For ease of reference, certain aspects of the control mechanism 9006 functionality may be described as controlling movement of interventional devices that are coupled to hub assemblies of the robotic catheter system, where the hub assemblies are configured to cause movement of the interventional devices in an axial direction and/or rotational direction. Certain aspects of the control mechanism 9006 functionality may also be described as controlling the fluidic system of a robotic catheter system to provide saline, contrast, or aspiration to a patient via one or more of the interventional devices. In the example of FIGS. 37A-37B, the control mechanism 9006 includes a control device 9024, which may include any of the same or similar features and/or functions as any of the other control devices described herein. The control device 9024 may be a handheld control device. The control device 9024 can be connected to a remotely located control console (e.g., remote control system 5006) via a wired connection or wirelessly, and communicate to a robotic catheter system located locally (near the patient) via the remotely located control console.

The control device 9024 can be configured to control movement of one or more of a plurality of interventional devices individually, or as a set of two or more of the interventional devices. For controlling the movement of a set of interventional devices, the control device 9024 can select which of the devices to belong to the set of devices. For example, in a robotic catheter system having a guide catheter, a procedure catheter, an access (insert) catheter, and a guidewire (each coupled to a hub assembly), the control device 9024 can select to include any one, two, three, or four of the interventional devices in a set and control the axial and/or rotational movement of the set of devices (e.g., to advance the set of devices, to retract the set of devices, and/or to rotate the set of devices).

In some embodiments, the fluidics system is configured to “normally” provide a saline flow through the catheters being used in the system (e.g., when they are not being used to inject contrast or provide aspiration). The control device 9024 can also be configured to control a fluidic system coupled to the catheters to select any one of the catheters for providing a contrast injection. When a control on the control device 9024 is actuated to provide contrast injection via a selected catheter, the control device 9024 generates a corresponding signal to control the fluidics system to align valves in the fluidic system to provide the desired contrast injection. For example, align valves in a hub assembly (for example, as illustrated in FIG. 38D) coupled to the selected catheter to provide a contrast channel from a contrast pump (e.g., contrast pump 9281, FIG. 38C) to the selected catheter and actuate the contrast pump. The control device 9024 can also be configured to control a fluidic system coupled to the catheters to select any one of the catheters for providing aspiration. When a control on the control device 9024 is actuated to provide aspiration via a selected catheter, the control device 9024 generates a corresponding signal to control the fluidics system to align valves in the fluidic system to provide vacuum via the selected catheter. For example, align valves in a hub assembly (for example, as illustrated in FIG. 38D) coupled to the selected catheter to provide a vacuum channel from a vacuum pump (e.g., vacuum source 9267, FIG. 38C) to the selected catheter and actuate the vacuum pump (if not already actuated).

The controls can include, for example, a combination of buttons, switches, dials, joysticks, and/or any other suitable actuators configured to provide control signals. In the embodiment illustrated in FIGS. 37A-37B, the control device 9024 can include a first control (e.g., first joystick 9104) and a second control (e.g., second joystick 9106). As shown in this embodiment, control device 9024 can also include a plurality of buttons positioned on a surface of the control device 9024, and a plurality of “shoulder” buttons positioned on an edge surface of the control device 9024. The plurality of buttons can include a first button 9108, a second button 9110, a third button 9112, a fourth button 9114, and a fifth button 9116. The plurality of shoulder buttons can include a first shoulder button 9120, a second shoulder button 9118. The shoulder buttons are located in a position near the first and second control (e.g., the first joystick 9104 and the second joystick 9106, respectively), such that the first shoulder button 9120 can be easily actuated by a user's hand operating the first control (e.g., the first joystick 9104), and the second shoulder button 9118 can be easily actuated by a user's hand operating the second control (e.g., the second joystick 9106). Certain examples of functionality that can be controlled by certain controls are described below. In other embodiments, the control device 9024 can provide similar or the same functionality with other controls. The control device 9024 can also include a light bar 9122 having a first portion 9122a and a second portion 9122b.

In some cases, the first shoulder button 9120 can be pressed and/or held to inject fluids, such as contrast or saline, via one or more of the interventional devices. The first shoulder button 9120 can be pressed to provide a small increment of a fluid (such as contrast and/or saline) puff (e.g., about 1 mL per puff). In some embodiments, the first shoulder button 9120 can be pressed and held to provide a longer fluid injection (e.g., greater than 1 mL). The amount of fluid injection may be proportional to the amount of time the first shoulder button 9120 is pressed and held. For example, a short press may deliver a puff or burst while a long press may deliver a longer injection. In some embodiments, the control device 9024 may include a separate button or other control that can be actuated (e.g., pressed or held) to cause a fluid injection. In some embodiments, as shown in FIG. 37B, the first shoulder button 9120 can have a surface split with distinct surfaces that can be pressed for separate injection and puff functions as described herein. In some embodiments, each of the first joystick 9104 and the second joystick 9106 may correspond to and be actuated to drive movement of one or more hub assemblies and/or interventional devices. Each joystick 9104 and 9106 can be configured to be actuated to cause corresponding rotational and/or axial movements of one or more hub assemblies and/or interventional devices linked to that joystick

In some embodiments, the first joystick 9104 can be linked to a first particular hub assembly and/or interventional device, such as a hub assembly associated with a guidewire, such that actuation of the first joystick 9104 causes (e.g., by generation of a control signal to control the robotic drive system) a corresponding action by first particular hub assembly and/or interventional device. For example, with respect to the drive system shown in FIG. 32D, which includes four hub assemblies, the first joystick 9104 may be linked to a hub assembly (e.g., first hub assembly 9034A) and/or its corresponding interventional device (e.g., a guidewire). The second joystick 9106 can be selectively linked to one or more of the remaining hub assemblies (e.g., the second hub assembly 9034B, the third hub assembly 9034C, and/or the fourth hub assembly 9034D) and/or interventional devices (e.g., an insert or access catheter coupled to the second hub assembly 9034B, a procedure catheter coupled to the third hub assembly 9034C, and a guide catheter coupled to the fourth hub assembly 9034D), for example, by a user making a selection on the control device 9024 (e.g., by pressing one or more buttons) such that actuation of the second joystick 9106 causes (e.g., by generation of a control signal to control the robotic drive system) a corresponding action by one or more remaining hub assemblies.

For example, in some embodiments, the fourth button 9114 is configured to, when actuated cause the second joystick 9106 to be linked to all of the remaining hub assemblies and/or their corresponding interventional devices (e.g., the hub assemblies and/or interventional devices not linked to the first joystick 9104). Actuation of the second joystick 9106 when the second joystick 9106 is linked to three hub assemblies and/or interventional devices will cause all of the linked hub assemblies and interventional devices to translate as commanded by the second joystick 9106. Movement of an access catheter, procedure catheter, and guide catheter together may be desirable during a first stage or access stage of a procedure, as described herein.

In some embodiments, pressing the fifth button 9116 may cause the second joystick 9106 to be linked with at least two hub assemblies and/or at least two interventional devices such as the third hub assembly 9034C and the fourth hub assembly 9034D and/or their corresponding interventional devices (e.g., a procedure catheter and a guide catheter) such that actuation of the second joystick 9106 causes (e.g., by generation of a control signal to control the robotic drive system) a corresponding action by one or more remaining hub assemblies. Movement of a procedure catheter and guide catheter together may be desirable during a second stage or procedure stage of a procedure as described herein.

In some embodiments, it may be desirable to move each of the hub assemblies coupled to a catheter (e.g., the second hub assembly 9034B, the third hub assembly 9034C, and the fourth hub assembly 9034D) and/or corresponding interventional devices individually during certain portions of a procedure as described herein. Pressing the second shoulder button 9118 can beneficially allow users to toggle between interventional devices. For example, if the second joystick is linked to a second hub assembly and/or interventional device, pressing the second shoulder button 9118 once may link the second joystick 9106 to a third hub assembly and/or interventional device so that actuation of the second joystick 9106 causes (e.g., by generation of a control signal to control the robotic drive system) a corresponding action by the third hub assembly and/or interventional device. Pressing the second shoulder button 9118 a second time may link the second joystick 9106 to a fourth hub assembly and/or interventional device so that actuation of the second joystick 9106 causes (e.g., by generation of a control signal to control the robotic drive system) a corresponding action by the fourth hub assembly and/or interventional device. Pressing the second shoulder button 9118 a third time may link the second joystick 9106 to the second hub assembly and/or interventional device again so that actuation of the second joystick 9106 causes (e.g., by generation of a control signal to control the robotic drive system) a corresponding action by the second hub assembly and/or interventional device. Thus, users can easily toggle between hub assemblies and/or interventional devices by pressing the second shoulder button 9118 as needed.

In this embodiment, the second button 9110 is configured to provide a signal to activate and deactivate control of the hub assemblies and/or interventional devices (“hub assemblies”). For example, actuating the second button 9110 provides a signal to pause or stop movement of the hub assemblies, and/or pause or stop control of the hub assemblies, such that the hub assemblies will not move in response to actuation of controls of the handheld control device 9024. Actuating the second button 9110 can generate a signal to cause control of the hub assemblies by the handheld control device 9024 to resume.

During a procedure, images (still or video) can be communicated from the local operating room to the remotely located control system. Such images can include, for example, overall images of the operating room, images of certain equipment (e.g., drip chamber, fluidic channels, clot pod), and/or one or more sets of fluoroscopic images, for example, from one or more fluoroscopy (X-Ray) machines. To obviate the need for a physician to select an image to view via another means (e.g., by a touch screen user interface on a display of the remote control system, the control device 9024 can be configured to select a desired set of images the remote control system is receiving. For example, the third button 9112 can configured to toggle between two or more fluoroscopic views displayed on a user interface.

In some cases, aspiration may be controlled via a user interface instead of the handheld control device 9024. As shown in FIGS. 37A-37B, the handheld control device 9024 may not include a button to control aspiration. Instead, aspiration may be controlled via the user interface. In some cases, a button may be included on the handheld control device 9024 and used to start and/or stop aspiration via one or more of the interventional devices. For example, pressing a button once may start aspiration. Pressing the button again may stop aspiration. In some cases, aspiration can be applied only when the button is actively being pressed.

The light bar 9122 can provide a visual indication about the status of the hub assemblies and/or the interventional devices. In some embodiments, the light bar 9122 can provide an indication that one or more hub assemblies and/or interventional devices can be driven by the control device 9024 or if control and/or movement of the one or more hub assemblies and/or interventional devices has been paused or stopped, for example, by pressing the second button 9110. In some embodiments, lights extending at least partially around each joystick 9104 and 9106 may indicate that one or more hub assemblies and/or interventional devices are able to be driven by those joysticks.

In other embodiments, for example, a first portion 9122a of the light bar 9122 can provide a visual indication about the status of the fluidics system associated with the hubs and/or interventional devices. The first portion 9122a may display a first color to indicate a first status of the fluidics system (e.g., the fluidics system is ready and/or available) and a second color different than the first color to indicate a second status of the fluidics system (e.g., actively being used, not available, etc.). A second portion 9122b of the light bar 9122 can provide a visual indication about the drive modes associated with the hubs and/or interventional devices, as described further herein. For example, the second portion 9122b may display a first color to indicate that the control device 9024 is in a specific drive mode (e.g., two hubs/devices are linked to the second joystick 9106, three hubs/devices are linked to the second joystick 9106, etc.) and a second color different than the first color to indicate a status of the hubs/devices (e.g., actively being driven, not available, etc.).

In some embodiments, the first button 9108 is configured to activate one or more of drive modes. Different drive modes can define different behaviors of one or more hubs and/or interventional devices in response to actuation of one or more controls of the control device 9024.

For example, pressing the first button 9108 once can activate an auto stacking drive mode. When the auto stacking drive mode is activated, actuation of a control (e.g., the second joystick 9106), can cause the robotic drive system (e.g., by generating one or more corresponding control signals to control the robotic drive system) to drive one or more of the interventional devices at different relative velocities and/or over different relative distances to achieve a desired overall stiffness profile and/or one or more desired local stiffnesses of the interventional device assembly.

An interventional device assembly or stack having multiple concentrically nested interventional devices can have an overall stiffness profile that depends on the relative positions of the concentrically nested interventional devices. For example, the stack may have a different stiffness at a first point along the length of the stack at which only an access catheter is present (e.g., because a guide catheter, procedure catheter, and/or guidewire are all positioned proximally to the point) than at a second point where both an access and procedure catheter are present. Similarly, each of the individual interventional devices may have stiffness profiles that vary along their length. Accordingly, a first point or segment along an interventional device stack at which a proximal segment of a procedure catheter and a proximal segment of an access catheter are located may have a different local stiffness than a second point or segment along the interventional device stack at which a distal segment of the procedure catheter and a distal segment of the access catheter are located. In some embodiments, a stiffness profile can characterize local stiffnesses at a plurality of points or segments along a length of an interventional device or interventional device stack and/or rates of stiffness change along a length of an interventional device or interventional device stack.

In some embodiments, interventional device stiffness or flexibility or interventional device stack stiffness or flexibility can be determined using the cantilever beam test. The cantilever beam test can be done along the length of an interventional device or interventional device stack to characterize the local segment stiffness and the rate of stiffness change. For example, in certain embodiments, the peak load may be measured at one or more increments along an interventional device or interventional device stack. In some embodiments, an interventional device of interventional device stack can be secured at a 5 mm distance from a target location to be measured. A force can then be applied to cause a 4 mm displacement at the target location of the interventional device or interventional device stack. The amount of force applied is measured at each sampling location (e.g., every 6 mm) to create a flexibility profile for the interventional device or interventional device stack. For example, for each sampling location, a peak load can be measured during the application of force to cause a 4 mm displacement. The stiffness profile can illustrate the peak load (gF) experienced along each sampling location (e.g., every 6 mm).

In some embodiments, it may be possible to provide a desired overall stack stiffness profile and/or one or more desired local stiffnesses or stiffness profiles at particular points or segments along the length of the interventional device stack by adjusting the relative positions of the interventional devices within the stack, for example, via one or more control algorithms. In some embodiments, the desired overall stack stiffness profile and/or desired local stiffnesses or stiffness profiles of the stack may change as the stack is advanced into different portions of the anatomy. In some embodiments, the positions of the interventional devices can be changed to adjust the overall stiffness profile of the stack and/or local stiffnesses or stiffness profiles of the stack by adjusting the velocities of the individual interventional devices to achieve particular relative positions between the interventional devices of the stack. In some embodiments, it may be desirable to maintain a leading (e.g., distally most positioned) interventional device at a velocity commanded by operation of a control mechanism as described herein. In such embodiments, the velocities of only the trailing (e.g., more proximally positioned) interventional devices can be changed (e.g., to achieve particular relative positions between the interventional devices of the stack) to adjust the overall stiffness profile of the stack and/or local stiffnesses or stiffness profiles of the stack.

During a procedure, it may be desirable to maintain the distal ends of the interventional devices within a predefined distance from each other (for example, when a user actuates an input, such as the second joystick 9106, to move multiple of the interventional devices) or to move the interventional devices so that their distal ends will arrive at predefined distances from one another at particular sections of the anatomy or during different portions of a procedure. In some embodiments, maintaining at least some of the distal ends of the interventional devices within a predefined distance from each other can beneficially maintain a desired overall stack stiffness profile and/or one or more desired local stiffnesses or stiffness profiles at one or more positions or segments along the length of the concentric stack of interventional devices. This can provide better support for the stack of interventional devices and/or allow for easier translation and/or navigation of the stack along the vasculature of a patient. In some embodiments, the predefined distances may be desirable for performing a particular step of a procedure.

Predefined distances can be adjusted and/or include different thresholds for different stages of the procedure. For example, in some embodiments, it may be desirable for the distal ends of a plurality of interventional devices to be arranged at particular positions relative to one another for a particular step of a surgical procedure. In some embodiments, it may be desirable that the distal ends of the plurality of interventional devices may be arranged at different particular positions relative to one another at a different step of the surgical procedure. In some embodiments, a plurality of interventional devices linked to a single control (e.g., the second joystick 9106) can be driven from a first set of positions to a second set of positions in which at least some of the interventional devices are at different relative positions to one another in comparison to the first set of positions (e.g., by moving the linked interventional devices different distances relative to one another and/or at different axial velocities relative to one another).

In some embodiments, when two or more of the interventional devices are driven together (e.g., in response to actuation of a single user input or control, such as the second joystick 9106) the velocities of the two or more interventional devices can be automatically adjusted to maintain desired separation distances. In some embodiments, the velocities of different interventional devices can be adjusted as percentage of a velocity of one of the interventional devices (e.g., of a leading interventional device). For example, in certain embodiments, one of the interventional devices may be driven at a first velocity that corresponds to an amount of actuation of a control, such as the second joystick 9106, and the other interventional devices may be driven at velocities that are a percentage of the first velocity. In some embodiments, the velocities of one or more of the interventional devices can be adjusted as a percentage of commanded velocity by the actuation of a control, such as the second joystick 9106. In some embodiments, the velocities of one or more interventional devices can be adjusted based on the distance over which the adjustment is performed. In some embodiments, the velocities of one or more interventional devices can be adjusted based as a percentage of a commanded velocity of one of the interventional devices and adjusted based on the distance over which the adjustment is performed.

In some embodiments, relative positions and relative velocities of different interventional devices can be changed for different procedure stages, for example, in response to a selection of a particular procedure stage using a user input of a control device. In some embodiments, relative positions and relative velocities of different interventional devices can be changed at different segments of a single procedure stage.

The auto stacking drive mode can be applied to and/or otherwise be incorporated into any of the control mechanisms described herein and in connection with any of the respective hubs and/or interventional devices described herein. Pressing the first button 9108 again may deactivate the auto stacking drive mode. Although reference is made to pressing the first button 9108 to activate the auto stacking dive mode, the first button 9108 can be configured to activate/deactivate any of the drive modes disclosed herein and/or to toggle between drive modes.

While particular combinations of controls (e.g., buttons, joysticks, etc.) and indicators are described above with respect to the handheld control device 9024, different arrangements and combinations of controls and/or indicators may used to perform the functions described above.

### Display

FIG. 37C is an embodiment of a user interface 9026 for facilitating control of the control mechanisms for interventional devices described herein. The user interface 9026 can include any of the same or similar features and/or functions as any of the user interfaces described herein.

The user interface 9026 can include a plurality of windows. For instance, the user interface 9026 can include an imaging window 9130, an instrument window 9132, a notification window 9134, a first video feed window 9136, a second video feed window 9138, a patient vitals window 9140, and/or a toggle indicator window 9142. In some cases, only some of the windows shown in FIG. 37C are displayed. In some cases, the user interface 9026 can be displayed on a capacitive screen, a resistive screen, a touch pad, or other touch-based sensing device. This can beneficially allow users to interact with the visual elements displayed on the user interface 9026. For example, in some embodiments, in addition to the user interface 9026 being displayed, users can adjust each of a plurality of parameters (e.g., velocity, rotational direction, axial position) displayed on the user interface 9026 by interacting with the user interface 9026. The plurality of parameters can be displayed on the instrument window 9132. Users can interact with the user interface 9026 by, for example, using basic gestures such as tapping, swiping, and/or dragging the components displayed on the capacitive screen, resistive screen, touch pad, or touch-based sensing device. Additionally or alternatively, the parameters displayed on the user interface 9026 can be adjusted by manipulating one or more physical buttons/components positioned adjacent the display in which the user interface 9026 is displayed.

The patient vitals window 9140 can display a patient's vital signs (e.g., body temperature, pulse rate, respiration rate, blood pressure, blood oxygen, etc.). In some cases, at least one window of the plurality of windows can include user profiles and/or settings. In some cases, instructions on how to move and/or operate the interventional devices associated with the user interface 9026 can be shown on the user interface 9026. The notification window 9134 can beneficially indicate whether a control mechanism, such as the control mechanism 9006 is active.

In some cases, the toggle indicator window 9142 can indicate which hub assemblies and/or interventional devices are available to be linked to a control, such as a second joystick 9106, and/or which hub assemblies and/or interventional devices are currently linked to the control. For example, as shown in FIG. 37C, the toggle indicator window 9142 can indicate that three interventional devices are available to be linked with a control, such as the second joystick 9106, and that all three interventional devices are currently linked to the control. In some embodiments, the configuration of toggle indicator window 9142 may highlight which of the hub assemblies and/or interventional devices are linked with the control. In some embodiments, the toggle indicator window 9142 can have a different configuration for providing a visual indication regarding that the control is linked to more than one hub assembly and/or interventional device.

### Fluidics System

FIGS. 38A-38D illustrate one or more aspects of a fluidics system as described herein. Additional details regarding fluidics systems are disclosed in U.S. patent application Ser. No. 18/666,217, entitled Fluidics Control System for Multi Catheter Stack, filed May 16, 2024, which is hereby expressly incorporated by reference in its entirety herein.

FIG. 38A is a schematic of the example of a fluidics assembly. In FIG. 38A, the fluidics system includes a fluidics cassette 9012 that couples to the pump station and to a saline source, a contrast source, and a vacuum source. The fluidics system also includes a plurality of fluid communication channels 9013 to provide saline, contrast, and vacuum to one or more mounts 9080a-c and to the hubs (not shown) and one or more catheters 9043a-c coupled to the one or more mounts 9080a-c. The fluid communication channels 9013 also can include a plurality of electrical leads that connect components in the mounts (e.g., bubble sensors, pressure sensors, etc.) to the electrical interface of the fluidics cassette 9012 to provide signals to the remote control system 5006 when the cassette 9012 is coupled to a pump station. In systems that include a plurality of hub assemblies, different hub assemblies may be configured differently or may have different fluids communicated to them, which are provided to a catheter. For example, in a system with a plurality of hub assemblies, one or more hub assemblies may be configured to only provide saline to a catheter, and/or one hub assemblies may be configured to only provide saline and contrast to a catheter, and/or one or more hub assemblies may provide saline and vacuum to a catheter, and/or one hub assemblies may provide saline, contrast, and vacuum to a catheter.

One challenge of a fluidics system that provides fluids (e.g., saline and contrast) that enter a patient via a catheter air-free (or bubble-free) fluid flows. As described herein, typically when the cassette, fluid communication channels, hubs/mounts, and catheters are provided for use in an operating room, initially they are not filled with fluids, and thus require priming to remove air from the saline and contrast flow-paths before operational use to prevent air bubbles entering a patient during a medical procedure. Once primed, air bubble detectors in the cassette and the mounts (illustrated in FIGS. 38C and 38D) can help mitigate risks associated with air bubbles residing within saline and contrast flow-paths. However, because of the dangerous consequences of air entering a patient's bloodstream, where it is possible the fluid communication channels are visually inspected for air bubbles by a medical practitioner after the saline and contrast flow-paths are primed. Because of flexibility and mobility requirements for providing fluids from static saline and contrast fluid sources across a certain distance to moving mounts/catheters, at least a portion of the fluid communication channels between the cassette and the mounts can include flexible clear tubing which allows for visual inspection of the fluid communication channels. During a visual inspection, each centimeter of the clear tubing supplying saline or contrast from the cassette 9012 to the mounts 9080 is carefully inspected to determine if air bubbles are present, and if so the lines can be re-primed. The length of time it takes to inspect the clear tubing for air bubbles directly relates to the total length of the contrast and saline tubing between the cassette and the mounts. In some examples, the pump station is located several feet away from the set of mounts 9080 to provide room around the patient, and accordingly the saline and contrast fluid communication channels 9013 between the cassette 9012 and mounts 9080 may be, for example, up to about 14 feet long. If (as illustrated in FIG. 38A) there are three mounts (a mount 9080a, a mount 9080b, and a mount 9080c) coupled to the communication channels 9013 and the communication channels include individual tubes for saline and contrast for each of three mounts 9080a-c, this can result in about 84 linear feet of saline and contrast tubing that requires visual inspection by a medical practitioner. To make inspection of the tubing easier, the communication channels can be configured in a “flat” ribbon-like design with the saline and contrast communication channels next to each other such that the contrast and saline tubing can be inspected together or nearly together, which effectively reduces the length of tubing requiring inspection to about 42 feet (14′ per mount). During the inspection, as the lines are each handled from end-to-end, the medical practitioner may change gloves for each line (e.g., three glove changes). Such an inspection can take between 7 and 10 minutes, which is a long delay before proceeding with the operation when every minute of delay of performing a thrombectomy can result in further harm to the patient.

To reduce the inspection time, the total amount of tubing that needs to be inspected can be reduced. In the embodiment illustrated in FIG. 38A, the first tubing set 9013a includes a one saline tube (channel) 9152 in fluid communication with a first saline flow-path in the cassette 9012 and a second saline flow-path in the splitter 9150, one contrast tube (channel) 9154 in fluid communication with a first contrast flow-path in the cassette 9012 and a contrast flow-path in the splitter 9150, and one vacuum tube (channel) 9156 in fluid communication with a first vacuum flow-path in the cassette 9012 and a second vacuum flow-path in the splitter 9150. An example of a first saline flow-path, first contrast flow-path, and first vacuum flow-path, in the cassette 9012, are illustrated by the saline subsystem 9201, the contrast subsystem 9202, and the vacuum subsystem 9203 illustrated in FIG. 38C (separated by dashed lines). The splitter 9150 provides saline, contrast, and vacuum from the first tubing set 9013a to the second tubing set 9013b. The splitter 9150 can be configured to include channels forming a second saline flow-path, a second contrast flow-path, and a second vacuum flow-path, the second saline flow-path, the second contrast flow-path, and the second vacuum flow-path each having a plurality of branches structured to provide outputs of saline, contrast, and vacuum to each mount 9080a-c from the single inputs of saline, contrast, and vacuum from the first tubing set 9013a. In some embodiments, the first tubing set 9013a can be structured in a ribbon-like design. In some embodiments, the second tubing set 9013b can be structured in a plurality of ribbon-like designs 9014a, 9014b, 9014c. In some embodiments, the length 9015a of the first tubing set 9013a can be between about 5 feet and about 17 feet in length, based on the specific embodiment. In some examples, the ratio of the first length to the second length is greater than about 1:2 (e.g., about 1:3, 1:4, 1:5, 1:6, of 1:7) to minimize the amount of tubing that needs to be visually inspected. In a specific embodiment, the length of the first tubing set is about 12 feet in length. The tubes in the first tubing set have inner diameters to suitably provide saline, contrast and vacuum to the catheters. As a nonlimiting examples, the inner diameter of saline and contrast tubes (channels) in the first tubing set and the second tubing set can be about 0.071 inches. As a nonlimiting example, the inner diameter of vacuum tubes in the first tubing set 9013a and the second tubing set 9013b can be about 0.110 inches. The first tubing set 9013a can also include an electrical channel 9158. The electrical channel 9158 includes multiple electrical leads to communicate electrical signals between the mounts 9080a-c and the cassette 9012, and can be coupled to the electrical interface. Sensors and electrical components in the cassette 9012 can also be coupled to the electrical interface.

The second tubing set 9013b includes one or more tube groups 9014a-c, each tube subgroup configured to provide a mount 9080a-c with saline, contrast, and vacuum. Each tube group 9014a-c can also provide each mount 9080a-c with one or more electrical leads. In the example in FIG. 38A, each tube group 9014a-c includes a proximal end 9016 coupled to the splitter 9150 and a distal end 9017 coupled to one of the one or more mounts 9080a-c, respectively. Each tube group 9014a-c includes a saline subchannel in communication with the second saline flow-path in the splitter 9150, a contrast subchannel in communication with the second contrast flow-path in the splitter 9150, and a vacuum subchannel in communication with the second vacuum flow-path in the splitter 9150. Each tube group 9014a-c can be configured in a ribbon-like design. In some embodiments, the length 9015b of the second tubing set can be between about 1 foot and about 4 feet. In a specific embodiment, the length 9015b of the second tubing set is about 2 feet. In this embodiment, the length of tubing in the first tubing set and the second tubing set needed to be inspected is significantly reduced from 42 feet to 18 feet, which correspondingly drastically reduces the inspection time to about 3-4.5 minutes, advantageously reduces set-up time such that the medical procedure (e.g., a thrombectomy) can begin much sooner, which increases the patient's likelihood of a more successful recovery. In addition, this significant reduction in the amount of tubing to inspect also minimizes glove changes by the medical practitioner inspecting the tubing set from three glove changes to one glove change. The second tubing set 9013b can also provide one or more electrical leads to another mount that does not require fluids, for example, a guide wire mount 9080d configured to axially move and rotate a guide wire 9021 coupled to the guide wire mount 9080d, the guide wire 9021 configured to be positioned partially or fully in the lumen of access catheter 9043c. In some embodiments, connectors may be used to couple the second tubing set 9013b to the cassette 9012 and/or the splitter 9150. However, use of connectors in fluidic systems can cause air bubbles to be formed and/or trapped in a portion of the connector. In this embodiment, advantageously connectors are not used, rather the second tubing set 9013b is coupled to the splitter 9150 and the mounts 9080a-c such that the second tubing set 9013b is not meant to be decoupled prior to or during a medical procedure (for example, permanently, semi-permanently).

FIG. 38B is a schematic of an example of the fluidics assembly illustrated in FIG. 38A illustrating some additional features. The fluidics assembly includes cassette 9012, the splitter 9150, and the one or more mounts 9080a-c, first tubing set 9013a coupled to the cassette 9012 and splitter 9150, and second tubing set 9013b coupled between the splitter 9150 and the one or more mounts 9080a-c, the first tubing set 9013a having a saline channel 9152, a contrast channel 9154, and a vacuum channel 9156 for providing saline, contrast, and vacuum to the splitter 9150. In this example, the fluidics assembly is shown having three mounts 9080a-c that receive saline, contrast, and vacuum from the cassette 9012. The first tubing set 9013a also includes an electrical channel 9158 having a plurality of electrical leads for connecting electrical components in the mounts 9080a-c to an electrical interface of the cassette 9012. The second tubing set 9013b includes tube groups 9014a-c providing saline, contrast, and vacuum to mounts. Each of the tube groups 9014a-c includes a saline subchannel, a contrast subchannel, a vacuum subchannel, and an electrical subchannel.

The splitter 9150 is structured to provide fluid communication of saline from the saline channel 9152 to the multiple saline subchannels in the tube groups 9014a-c, to provide fluid communication of contrast from the contrast channel 9154 to the multiple contrast subchannels in the tube groups 9014a-c, to provide fluid communication of saline from the single saline channel 9152 to the multiple saline subchannels in the tube groups 9014a-c, and to provide fluid communication of vacuum from the vacuum channel 9156 to the multiple vacuum subchannels in the tube groups 9014a-c. Also, the splitter 9150 is structured to provide electrical connection between the electrical channel 9158 and the electrical subchannels in the tube groups 9014a-c. As illustrated in FIG. 38B, the splitter 9150 includes a saline manifold 9170 coupled to the saline channel 9152 and the saline subchannels of each tube group 9014a-c in the second tubing set 9013b, a contrast manifold 9172 coupled to the contrast channel 9154 and the contrast subchannels of each tube group 9014a-c in the second tubing set 9013b, a vacuum manifold 9174 coupled to the vacuum channel 9156 and the vacuum subchannel of each tube group 9014a-c in the second tubing set 9013b, and an electrical bus 9176 coupled to the electrical subchannels of each tube group 9014a-c in the second tubing set 9013b. The saline manifold 9170, contrast manifold 9172, and vacuum manifold 9174 can be structured in various configurations that include tubes, splitters, manifolds, or any other structures to provide fluid communication from a single channel in the first tubing set 9013a to multiple subchannels in the second tubing set 9013b. The electrical bus 9176 can be any electrical structure that provides electrical communication between the electrical subchannels of the tub groups 9014a-c in the second tubing set 9013b and the electrical channel 9158 in the first tubing set, including separate electrical wires (leads) or other electrical connections. For example, the electrical subchannels in the tube groups 9014a-c can include a plurality of electrical leads which are bundled together in the splitter 9150 and form the electrical channel 9158 in the first tubing set 9013a. As shown in FIG. 38B, the electrical bus 9176 can also electrically connect the electrical channel 9158 of the first tubing set 9013a to the electrical connection 1328 to guide wire mount 1400d. Other embodiments having more or fewer mounts and corresponding connections are also possible. In some embodiments, the splitter 9150 may be referred to as a relay cassette. In some embodiments, the splitter 9150 may be located outside of the sterile field or within the sterile field. In some embodiments, the splitter 9150, second tubing set 9013b, and associated hub assemblies (e.g., mounts 9080a, 9080b, and 9080c and associated hubs) can be positioned within the sterile field.

The embodiment of communication channels illustrated in FIGS. 38A and 38B is designed to minimize the time needed for inspecting the fluid lines. In this example, the fluid communication channels include a first tubing set 9013a having a proximal end coupled to the cassette 9012 and a distal end coupled to a splitter 9150. The fluid communication channels also include a second tubing set 9013b having one or more tube groups 9014a-c, each tube group 9014a-c having a proximal end coupled to the splitter 9150 and a distal end coupled to one of the mounts 9080a-c. However, use of connectors in fluidic systems can cause air bubbles to be formed and/or trapped in a portion of the connector. In some preferred embodiments, advantageously connectors (i.e., releasable connectors) are not used to couple the first tubing set 9013a to the cassette 9012 and the splitter 9150, and are not used to connect the second tubing set 9013b to the splitter 9150. Instead, the first tubing set 9013a is coupled to the cassette 9012 and the splitter 9150 in a persistent coupling (e.g., permanent connection, semi-permanent coupling using glue, clamped fittings, integrally molded portions, and/or other suitable couplings) that it is not meant to be decoupled prior to or during a medical procedure. In preferred embodiments, the first tubing set 9013a and the second tubing set 9013b includes transparent tubing. The cassette 9012 can include fluid communication channels (including portions of components) configured to communicate saline, contrast, and vacuum in first saline, contrast, and vacuum flow-paths, respectively. For example, as described in reference to FIG. 38C. The splitter 9150 can include fluid communication channels configured to communicate saline, contrast, and vacuum in the second saline, contrast, and vacuum flow-paths, respectively. For example, as described in reference to FIG. 38B. The fluid communication channels in the cassette 9012 and the splitter 9150 can include one or more tubes, channels (e.g., molded channels), portions of components (e.g., a drip chamber, clot pod, at least a portion of peristaltic saline pump, at least a portion of a contrast pump, robotically controlled valves, etc.) and other suitable fluid communication structures. The proximal end of the first tubing set can be coupled to the cassette 9012 using a persistent coupling. In some embodiments, the proximal end of the first tubing set 9013a extends into the cassette 9012 and forms a portion of a channel in the cassette 9012. The distal end of the first tubing set can be coupled to the splitter 9150 using a persistent coupling. In some embodiments, the distal end of the first tubing set 9013a extend into the splitter 9150 and can form a portion of a channel in the splitter 9150. In some embodiments, a portion of, or all of, the fluid communication channels of the first tubing set 9013a are integrally formed with a portion of, or all of, and the fluid communication channels of the splitter 9150. In an example, tubes forming each of a saline channel 9152, a contrast channel 9154, and a vacuum channel 9156 of the first tubing set 9013a are integrally formed with tubes forming saline, contrast, and vacuum fluid communication channels of the splitter 9150. In some embodiments, a portion of, or all of, the fluid communication channels of the second tubing set 9013b are integrally formed with a portion of, or all of, and the fluid communication channels of the splitter 9150. In an example, a portion, or all of, tubes forming a saline subchannel, a contrast subchannel, and a vacuum subchannel in each of one or more tube groups 9014a-c are formed to be integral with structures (e.g., tubes) forming the fluid communication channels of the splitter (e.g., saline channel 9152, contrast channel 9154, vacuum channel 9156, saline manifold 9170, contrast manifold 9172, and vacuum manifold 9174). In some embodiments, all or part of the fluid communication channels of the first tubing set 9013a, the splitter 9150, and the second tubing set 9013b are formed as in integral structure and are persistently connected. In some embodiments, all or part of the fluid communication channels of the first tubing set 9013a, the splitter 9150, the second tubing set 9013b and at least some of the fluid communication structures in the mounts 9080a-c are formed as an integral structure such that they are persistently connected. In some embodiments, all of the fluid communication channels in the first tubing set 9013a, the splitter 9150, the second tubing set 9013b include tubing. For example, tubing that is coupled to the cassette 9012, split into multiple tube groups in the splitter, and tubing that is coupled to the splitter and the mounts. In some embodiments, all of the fluid communication channels in the first tubing set 9013a, the splitter 9150, the second tubing set 9013b and the mounts 9080a-c include tubing. Some embodiments can include non-persistent coupling of fluid communication channels, for example, it is determined that such connections do not heighten the likelihood trapping air bubbles. In certain embodiments, tubes that are integrally formed with one another may be referred to as “tube sections” that together form a tube.

FIG. 38C is a schematic illustrating another example of a cassette 9012 of the fluidics assembly having a housing 9200 supports portions of a saline subsystem 1201, a contrast subsystem 1202, and a vacuum subsystem 1203 enclosed in, or partially enclosed in, the housing 9200. FIG. 38C also illustrates components of an example of a saline subsystem 9201, a contrast subsystem 9202, and a vacuum subsystem 9203 coupled to the cassette 9012, according to some embodiments. Other examples of cassettes may have different components, for example, fewer or more components. The control valves in the saline subsystem 9201, contrast subsystem 9202, and vacuum subsystem 9203 are robotically controlled to be actuated based on a user input, sensor signals received by a controller, and/or executable instructions that one or more hardware processors of a controller are configured to execute to prime the saline subsystem 9201, the contrast subsystem 9202, and the vacuum subsystem 9203, to prime fluid communication channels coupled to the saline subsystem 9201, the contrast subsystem 9202, and the vacuum subsystem 9203, prime fluidic channels in hubs/mounts, and provide saline, contrast, and vacuum from the saline subsystem 9201, the contrast subsystem 9202, and the vacuum subsystem 9203 to the hubs/mounts and ultimately to catheters connected to the hubs/mounts.

One or more saline sources can be coupled to the cassette 9012 to be selectively placed in fluid communication with the saline subsystem 9201. Flow-paths communicate saline through the saline subsystem 9201. In this example, two saline sources, a saline source 9307a and a saline source 9307b (e.g., saline bags) are coupled to saline input ports 9294a, 9294b for providing a flow of saline to the saline subsystem 9201. The cassette 9012 includes a saline flow-path 9206a that receives saline through input ports 9294a-b and provides saline to the first tubing set 9013a through saline output port 9230, and also provides saline to the contrast subsystem 9202. Control valve 9214 can be actuated to connect channel 9206c to channel 9206f which is coupled to the vacuum subsystem 9203 vacuum channel 9282a to evacuate saline and air from the saline subsystem 9201 (i.e., channel 9206f can be used as a waste line). Input ports 9294a, 9294b are in fluid communication with a saline first control valve 9220, which can be controlled to select the saline source 9307a, 9307b to receive saline from. In some embodiments the saline sources are supported by a weight sensor 9210 that is connected to a controller (e.g., in pump station) and provides a signal associated with the weight of each saline source to the controller. The saline subsystem 9201 also includes a peristaltic pump 9213 having an inlet 9207 and an outlet 9208, and an air sensor 9205 positioned between the peristaltic pump 9213 and the first control valve 9220. In some embodiments, the air sensor 9205 is incorporated in the cassette 9012. In other embodiments, the air sensor 9205 is capital equipment (not disposable) and is not included in the cassette 9012, instead it can be included in the pump station and is positioned near channel 9206a-b (e.g., near a piece of tubing 9206a-b that engages with/into the air sensor 9205. In this embodiment, the portion of the peristaltic pump 9213 in the cassette includes tubing that, when the cassette 9012 is coupled to the pump station, the tubing interfaces with a movable portion of a peristaltic pump on the pump station which is configured to move saline through the saline flow-path 9206 of the saline subsystem 9201. An air sensor 9205 is positioned to sense air in the saline flow-path (channel) 9206a-b between the peristaltic pump 9213 and the first control valve 9220, and is configured to generate a signal indicative of air detected. In some embodiments, the air sensor 9205 is capital equipment and located (for example, on the pump station). In such embodiments, channel 9206a-b can include a portion of tubing that engages into the air sensor. In some embodiments, the air sensor is located in the cassette 9012 and is connected to the electrical interface 9236 such that a signal generated by the air sensor 9205 can be provided to the pump station (or another controller).

The saline subsystem 9201 also includes a pressure sensor 9212, positioned downstream of the peristaltic pump 9213, and configured to generate a signal indicative of pressure in the saline flow-path. The saline subsystem further includes a saline second control valve 9214 that is configured to selectively, as controlled by a controller, provide saline to a channel 9222 which is connected to the vacuum subsystem 9203. In operation, a controller may align the second control valve 9214 to diverge the saline flow-path to the vacuum subsystem 9203 to prime the upstream portion of the saline flow-path 9206a-c, for example, when the saline sources are first connected to the saline flow-path or when the saline source is switched from the first saline source 9307a a second saline source 9307b. The saline subsystem 9201 also includes a saline third control valve 9218 that is configured to selectively, as controlled by a controller, provide saline to a channel 9256 which is connected to the contrast subsystem 9202 for priming the contrast subsystem 9202 with saline. In a normal operational configuration for providing saline to catheters, a controller aligns the first control valve 9220 to receive saline from one of the two connected saline sources, aligns the second control valve 9214 to provide saline from peristaltic pump 9213 to the third control valve 9218, and the lines the third control valve 9218 to provide saline to the saline output port 9230 to flow into the first tubing set 9302.

Still referring to the example illustrated in FIG. 38C, the contrast subsystem 9202 includes a contrast flow-path 9244a-d that receives contrast from a contrast source 9309 through contrast inlet port 9242 along flow-path 9244ab, communicates contrast to the first tubing set 9013a via contrast outlet port 9264 along flow-path 9244b-d, and communicates air and/or contrast from a contrast pump 9281 to the vacuum subsystem 9203 along flow-path 9244c. In some embodiments, the contrast subsystem 9202 includes a control valve 9211 coupled to channels 9244a-b-d that can be actuated to connect the contrast pump 9281 to the contrast source 9309 (connecting channels 9244b and 9244a), or connecting the contrast pump 9281 to channel 9244d and the contrast channel of the first tubing set 9013a. An air sensor 9246 is positioned along the contrast flow-path between the contrast source 9309 and the contrast pump 9281, and is configured to sense air in the contrast flow-path (channel) 9244a and generate a signal indicative of air being in the contrast flow-path. In some embodiments, the air sensor 9246 is capital equipment and located (for example, on the pump station). In such embodiments, channel 9244a can include a portion of tubing that engages into the air sensor 9246. In some embodiments, the air sensor 9246 is located in the cassette 9012 and is connected to the electrical interface 9236 such that a signal generated by the air sensor 9246 can be provided to the pump station (or another controller). In some embodiments, based on receiving a signal from the air sensor 9246 indicating presence of air in flow-path 9244a, the controller may pause contrast pump operation, indicate another contrast source is needed on a user display or with an audible alarm. When a new contrast source is attached to contrast inlet port 9242, in some embodiments the controller can initiate a priming sequence that includes actuating the contrast control valve 9247 to provide vacuum to the contrast pump 9281 (connecting channel 9244c to port 9250) and actuating valve 9211 to connect channels 9244a and 9244b to expel air in the contrast pump 9281 and channels 9244ab through channel 9244c. When changing contrast sources (bottles), channel 9244b and the contrast pump (syringe) 9281 are already full of contrast, but 9244a has air in it. Instead of pulling air through the primed syringe, control valve 9247 can be actuated to block channel 9244c from being in fluid communication with port 9250 (if not already so aligned) and control valve 9211 can be actuated to connect channel 9244a to be in fluid communication with contrast pump outlet port 9254, and the contrast pump 9281 can be actuated to push air in channel 9244a into the contrast source 9309.

The contrast pump 9281 receives contrast into a chamber in the contrast pump 9281 from the contrast source 9309 through contrast (outlet) port 9254 to fill the contrast pump 9281. A contrast pump port 9250 connects the contrast pump 9281 to channel 9244c, 9249 which is connected to the vacuum subsystem 9203, and may be used to remove air from the contrast pump 9281. Contrast control valve 9247 is positioned between contrast pump port 9250 and the vacuum subsystem, and is selectively controlled by a controller to align the contrast control valve 9247 to open or to shut the air/contrast flow-path between the contrast pump 9281 and the vacuum subsystem 9203. When filling the contrast pump 9281 with contrast from contrast source 9309, in some embodiments, the contrast control valve 9247 can be opened to provide vacuum to the contrast pump 9281 through port 9250 to evacuate air in the contrast pump 9281 along flow-path 9244c, and help cause contrast from the contrast source to flow along flow-paths 9244a and 9244b into port 9254 to fill the pump 9281. In an example, the contrast control valve 9247 is opened for a predetermined amount of time to prime the contrast pump 9281. In other examples, vacuum is not used during the priming process. For example, once the contrast pump 9281 is at least partially filled with contrast, the contrast pump can be actuated to expel any air in the pump through channel 9244c and/or through channel 9244b (e.g., to the contrast source). The contrast control valve 9247 is closed for operation of the contrast pump 9281. FIG. 38C illustrates one example of a contrast subsystem 9202, but other configurations are also contemplated.

The vacuum subsystem 9203 of the cassette 9012 includes a vacuum port 9271 coupled to a vacuum canister 9270, which can be coupled to a vacuum filter or vacuum regulator 9269, and a vacuum source assembly (collectively “vacuum source”) 9267 which can include a vacuum pump, a vacuum regulator, and other components for providing a consistent vacuum controlled robotically. The vacuum source 9267 can be controlled by a controller to provide a desired amount of vacuum to a vacuum flow-path. As illustrated in FIG. 38C, in this example the vacuum canister 9270, and vacuum source 9267 are positioned outside of the cassette 9012. Vacuum flow-path 9282 extends from the vacuum inlet port 9271, through clot pod assembly 9272 to vacuum port 9289, which is coupled to the first tubing set 9013a. A portion of the vacuum flow-path 9282 extends from the vacuum port 9271 to the clot pod assembly 9272. The contrast subsystem 9202 and the saline subsystem 9201 are connected to the vacuum flow-path 9282 through channels 9249 and 9222, respectively, connecting between the clot pod assembly 9272 and vacuum port 9271 such that fluid and air from the saline subsystem 9201 and the contrast subsystem 9202 do not go through the clot pod assembly 9272, instead flowing through vacuum port 9271 and into vacuum canister 9270. The clot pod assembly 9272 includes clot pod 9273, drip chamber 9278, and a first three-way control valve 9275 positioned in the vacuum flow-path 9282 between the drip chamber 9278 and the clot pod 9273. The first control valve 9275 is controlled by a controller to align the vacuum flow-path 9282 to connect clot pod inlet line 9274 to either the drip pod outlet line 9276 or the drip pod bypass line 9277. The first control valve 9275 can also be controlled by a controller to align the vacuum flow-path 9282 to neither the drip pod outlet line 9276 or the drip pod bypass line 9277. Drip pod inlet line 9279 connects the vacuum flow-path to second three-way control valve 9280, which is controlled by a controller to align the vacuum flow-path 9282 to vacuum port 9289 and the first tubing set 9013a, or to the local atmosphere. A pressure sensor 9283 is positioned along the flow-path 9282 between control valve 9280 and vacuum port 9289. Pressure sensor 9283 also connected to the electrical interface 9236, and is configured to detect pressure in the vacuum flow-path 9282 and provide a signal indicative of the detected pressure to the electrical interface 9236.

At least a portion of the clot pod assembly 9272 can be transparent to allow a medical practitioner to view the drip chamber 9278 and the clot pod 9273. For example, to see a fluid flow into the drip chamber and to determine a level of fluid accumulated in the drip chamber, and to view and assess material captured in the clot pod. In an example, the clot pod assembly 9272 can be structured to be positioned along a surface of the cassette 9012. In some embodiments, one side of the clot pod assembly 9272 can be transparent to such that the drip chamber and the clot pod are visible to a medical practitioner when the cassette is mounted on the pump station, and the opposite side is transparent or translucent to allow light illuminate the drip chamber and/or clot pod for easier viewing. An imaging sensor (e.g., a camera) 9292 can be positioned to generate an image of the clot pod 9273 and/or the drip chamber 9278, and the image can be displayed locally or remotely to provide information on the contents of the clot pod 9273 and/or the drip chamber 9278. In this embodiment, the imaging sensor 9292 is not included in the cassette 9012, but instead the imaging sensor 9292 can be incorporated in the pump station or another suitable location (locally) such that it can generate an image of the clot pod 9273 and/or drip chamber 9278. In some embodiments, the imaging sensor 9292 can be incorporated in the cassette, however, having the imaging sensor positioned in the disposable cassette may increase cost. In embodiments where the imaging sensor is disposed external to the cassette 9012, a portion of the cassette 9012 can be transparent to allow the imaging sensor 9292 to image the clot pod assembly 9272, or a portion thereof. In some embodiments, the imaging sensor 9292 can be the video capture device 6032 described above.

Saline subsystem 9201 can include air sensor 9205 and pressure sensor 9212, the contrast subsystem 9202 includes air sensor 9246, and the vacuum subsystem 9203 includes pressure sensor 9283. Signals from these sensors are received by a controller, which can use these signals to align control valves in the cassette 9012 to vary the saline, contrast, and vacuum flow-paths in the cassette and perform processes described herein, and other processes. A purpose of these sensors is to detect potentially hazardous situations, for example, running out of a fluid source, a kink or occlusion in a fluid line, etc. Although the saline and contrast air sensors 9205, 9246 can be located in a pump station and engage a portion of saline or contrast tubing in the cassette, or they can be located in the cassette, such air sensors can be considered and referred to as being a part of the saline subsystem 9201 or the contrast subsystem 9202.

The configuration of the cassette 9012 in FIG. 38C has a certain different features than cassettes described above, which can provide operational and cost advantages. For example, in some embodiments, the vacuum canister may be positioned in the cassette, where in FIG. 38C the vacuum canister 9270 (and vacuum regulator 9269 and the vacuum source 9267) are positioned outside of the cassette in the line from the vacuum canister 9270 is coupled to the cassette at port 9271. This may allow the disposable cassette to be less expensive, and allow various configurations of vacuum source assemblies (e.g., the vacuum canister, the vacuum regulator, and the vacuum pump) to be used with the system, and any changes or improvements to the vacuum canister will not affect the design of the cassette. The configuration of the cassette 9012 in FIG. 38C includes two saline sources 9307a, 9307b in a control valve 9220 that a controller can align to select either one of the saline sources, either based on a manual input or automatically based on the sensed condition (e.g., the signal from weight sensor 9210 that a saline source is depleted below a predetermined threshold, a signal from air sensor 9205 that air is present in the saline flow-path 9206). The saline subsystem 9201 in FIG. 38C includes channel 9222 from the saline subsystem to the vacuum subsystem 9203 which allows for a simplified priming procedure compared to other configurations. In some embodiments, if air is detected, the controller can be configured to automatically, or based on user input, to prime the saline subsystem by directing the saline flow-path 9206 upstream of the second control valve 9214 to vacuum such that saline and the air in the saline subsystem 9201 upstream of the second control valve 9214 is evacuated into the vacuum canister 9270. Also, the contrast subsystem 9202 has advantageously been simplified and no longer includes a contrast manifold and the three control valves can be aligned to select the catheter to receive contrast. Instead, control valves that the controller can align to provide contrast from the contrast subsystem 9202 to a catheter are located in the mounts as shown in an example, in FIG. 38C (e.g., control valve 9426). Similarly, control valves that the controller can align to provide saline from the saline subsystem 9201 a catheter located in the mounts (e.g., FIG. 38C). Having these control valves in the mounts also advantageously obviates the need for having multiple individually controllable saline lines and contrast lines extending from the cassette 9012 to the mounts 9080a-c, such that the fluid communication channels 9013a-b (FIG. 38B) are also simplified and include less tubing. Also, unlike some of the previous embodiments described above, the example configuration illustrated in FIG. 38C advantageously does not include saline lines or control valves for providing saline to a femoral sheath, thus further simplifying the design of the cassette 9012 and may lower manufacturing costs. Instead, in this example, saline can be provided to a femoral sheath from a separate system (not shown here).

In reference to FIGS. 38A-38B, examples of a fluidics management system can include a cassette 9012 configured to receive saline from a saline source 9307 (e.g., two or more saline sources), receive contrast from a contrast source 9309, and receive vacuum from a vacuum source (e.g., a vacuum pump) 9267. The cassette 9012 can include a saline subsystem 9201 having a first saline flow-path; a contrast subsystem 9202 having a first contrast flow-path; and a vacuum subsystem 9203 including a first vacuum flow-path. The first saline, contrast, and vacuum flow-paths can include channels, tubes, line, and portions of other components (e.g., one or more of a valve, drip chamber, clot pod, pump, etc. in the cassette 9012). The fluidics management system can also include a splitter having a second saline flow-path, a second contrast flow-path, and a second vacuum flow-path. Each of the second saline, contrast, and vacuum flow-paths has single proximal end and a plurality of distal ends. The single proximal end of the second saline flow-path is in fluid communication with a single saline channel in a first tubing set 9013a, the proximal end of the second contrast flow-path is in fluid communication with a single channel of the first tubing set 9013a. The second vacuum flow-path proximal end is in fluid communication with the single vacuum channel of the first tubing set 9013a. The first tubing set 9013a has a first length, as measured between the cassette 9012 and the splitter 9150, and is coupled to the cassette 9012 and splitter 9150. The first tubing set 9013a includes a single saline channel coupled to the first saline flow-path of the cassette 9012 and the proximal end of the second saline flow-path of the splitter 9150, the single contrast channel coupled to the first contrast flow-path of the cassette 9012 and the proximal end of the second contrast flow-path of the splitter 9150, and a single vacuum channel coupled to the first vacuum flow-path of the cassette 9012 and the proximal end of the second vacuum flow-path of the splitter 9150. The fluidics management system can also include two or more mounts, for example, three mounts, or four mounts. The mounts can be configured in various ways to provide saline, contrast, and vacuum to catheters coupled to one or both of each respective mount. Each mount can be configured to have a third saline flow-path, a third contrast flow-path, and a third vacuum flow-path to provide saline, contrast and vacuum to a catheter via a connector configured to couple to a catheter. In some embodiments, the catheter is coupled to another assembly (e.g., a hub) which is coupled to the mount, and the lumen of the catheter is coupled to the connector to be in fluid communication with the connector. A mount can include a plurality of robotically actuated control valves controlled by a control system to selectively align the third saline flow-path, the third contrast flow-path, and the third vacuum flow-path to be in fluid communication with the connector. The fluidics management system can also include a second tubing set having a second length, the second tubing set comprising a plurality of tube groups, each tube group coupled to the splitter on a proximal end of the tube group and to one of the two or more mounts on a distal end of the tube group. Each tube group including a saline subchannel coupled to the distal end of the second saline flow-path of the splitter, a contrast subchannel coupled to the distal end of the second contrast flow-path of the splitter, and a vacuum subchannel coupled to the distal end of the second vacuum flow-path of the splitter. The first tubing set has a first length and the second tubing set has a second length, the first length being greater than the second length to minimize the amount of tubing that needs to be visually inspected for air bubbles. In some examples, first tubing set has a first length and the second tubing set has a second length, and the first length of the first tubing set is at least two times as long as the second length of the second tubing set.

Referring to FIG. 38C, priming the saline subsystem 9201 can include robotically actuating control valves of the saline subsystem 9201, robotically actuating a peristaltic saline pump, and robotically controlling vacuum in the vacuum subsystem to perform a priming process. In one example of priming of the saline subsystem 9201, the cassette 9012 is coupled to a pump station and the saline sources 9307a-b are coupled to the saline subsystem. To be able to switch from one saline source to another connected saline source seamlessly, channels connecting both saline sources to the saline subsystem 9201 must be primed. Control valve 9220 can be robotically controlled to couple a first saline source to peristaltic pump inlet 9207. The control valve 9214 can be aligned to connect the peristaltic pump outlet 9208 to channel 9222 which is connected to the vacuum subsystem 9203 to purge air from the saline subsystem. The vacuum subsystem can be robotically controlled to provide vacuum to channel 9222 to purge air during priming, or to not provide vacuum and instead channel 9222 and canister 9270 is used to purge air without applying vacuum using channel 9222. In this configuration, the saline peristaltic pump can be run (e.g., for a predetermined time) to evacuate air in the saline flow-path from the first saline source to control valve 9214. During this priming, signals from air sensor 9205 should eventually indicate no air is detected. The control valve can be actuated to connect a second saline source to peristaltic pump inlet 9207. The control valve 9214 can continue to be aligned to connect the peristaltic pump outlet 9208 to channel 9222 which is connected to the vacuum subsystem 9203. The vacuum subsystem can again be robotically controlled to provide vacuum to channel 9222. In this configuration, the saline peristaltic pump can be run (e.g., for a predetermined time) to evacuate air in the saline flow-path from the second saline source to control valve 9214. During this priming, signals from air sensor 9205 should eventually indicate no air is detected. Control valve 9214 can be actuated to connect the outlet port 9208 to the second control valve 9214, which can be actuated to connect the outlet port 9208 to the contrast subsystem 9202 for priming the contrast subsystem, or connect outlet port 9208 to a saline communication channel to provide saline to a plurality of mounts 9080a-c.

Priming of the contrast subsystem 9202 can include robotically actuating control valve 9247, robotically actuating contrast pump 9281, and robotically controlling the vacuum subsystem 9203 to perform a priming process. In an example of a priming process of the contrast subsystem 9202, the contrast subsystem 9202 is connected to a contrast source 9309. Control valve 9247 is robotically actuated to connect outlet port 9250 to the vacuum subsystem 9203. In some embodiments of a contrast priming process, the vacuum pump 9267 is robotically actuated to provide vacuum to the contrast pump 9281 and the contrast flow-path 9244, which evacuates air from the contrast flow-path (channel) 9244 and the contrast pump 9281 as it fills with contrast. In other embodiments of a contrast priming process, vacuum is not provided to the contrast pump 9281 via channel 9244, instead channel 9244 is used to evacuate air which evacuate air from channels 9244 and the contrast pump 9281 as the pump is filled with contrast. Subsequently, control valve 9247 is closed and the contrast pump 9281 can be robotically actuated to provide contrast to a prime a contrast communication channel, to prime the plurality of mounts, and to provide contrast to the plurality of mounts 9080a-c. As part of priming the contrast communication channels connect the cassette to the mounts, control valve 9218 can robotically actuated to connect outlet port 9208 to the contrast flow-path 9244, and the saline peristaltic pump can be actuated to provide saline to the contrast subsystem such that it fills the contrast communication channels to the mounts and in the mounts. The vacuum subsystem 9203 can be robotically controlled to provide vacuum to the mounts 9080a-c via a vacuum communication channel. In each mount, one or more control valves can be robotically actuated to place a contrast channel in the mount, that is being provided saline from the cassette, in fluid communication with a vacuum channel to facilitate priming the contrast communication channel between the cassette and the mount, and in the mount, with saline to evacuate all air in these channels. Subsequently, control valve 9218 can be actuated to connect the outlet port 9208 with saline outlet port 9230 and the saline communication channel that is coupled to the mounts.

As indicated above for other embodiments of a fluidics system, saline and contrast channels in the fluidics assembly and in hub assemblies (e.g., mounts and/or hubs) are also primed prior to use in a medical procedure. In an example, once the saline subsystem 9201 is primed, the control valve 9218 be actuated to connect the outlet of the saline pump to the saline channels in the fluidics assembly and the saline pump 9213 can be actuated to provide saline to fill the saline channel 9152, the saline manifold 9170, the saline subchannels of each tube group in the second tubing set 9013b, and the saline channel of each mount 9080a-c with saline. To facilitate the flow of saline through these saline channels, the control valves 9426 and 9428 can be actuated to connect the saline channel 9152 with the vacuum channel 9156 while controlling the vacuum subsystem 9203 to provide vacuum to the mounts 9080a-c. Once the saline subsystem 9201 is primed, the control valve 9218 be actuated to connect the outlet of the saline pump 9213 to the contrast subsystem 9202, and the saline pump 9213 can be actuated to provide saline into the contrast subsystem 9202, to fill the contrast channel 9154, the contrast manifold 9172, the contrast subchannels of the second tubing set, and the contrast channel of each mount 9080a-c with saline. To facilitate the flow of saline through the contrast channels, the control valves can be actuated to connect the contrast channel 9154 with the vacuum channel 9156 while controlling the vacuum subsystem to provide vacuum to the mount. A controller can use signals received from the saline and contrast air sensors to determine when no air is detected in the saline channel and the contrast channel, indicating the saline and contrast channels between the mounts and the cassette are primed with saline. Similar to the priming processes described above, once the contrast channels from the cassette to the mounts are primed with saline, and the contrast channel is primed with saline, channels of the fluidic assembly can also be prime.

FIG. 38D is a schematic illustrating an example of a fluidic system components in mounts 9080a-c, which are coupled to the second tubing set 9013b. This example includes three mounts 9080a-c that have the same components relating to the fluidic system. Each mount 9080a-c has fluidic connections to the second tubing set 9013b to receive saline, contrast, and vacuum, and an electrical connection that includes one or more electrical leads, which are coupled to one or more sensors in the mounts 9080a-c to communicate signals from one or more sensors in the mount to the cassette, and ultimately to a controller when the cassette is coupled to the pump station. A controller in the local system uses the received signals to align valves in the mounts 9080a-c individually to provide saline, contrast, and aspiration (vacuum) automatically or semi-automatically (e.g., based on a user input) as needed during different portions of a medical procedure. In this example, mount 9080a has a common connector 9209a for providing saline, contrast, and vacuum to the lumen of catheter 9243a; mount 9080b has a connector 9209b for providing saline, contrast, and vacuum to the lumen of catheter 9243b; and mount 9080c has a connector 9209c for providing saline, contrast, and vacuum to the lumen of catheter 9243c. The connectors 9202a-c can be in fluid communication with the lumens of catheters 9243a-c respectively by coupling the connectors to a corresponding connector on a hub (or other assembly) that the catheters 9243a-c are respectively coupled to, where the hubs include a fluid communication channel from the connector to the lumen of the catheter coupled to the hub. Such hubs can be removably coupled to the mounts 9080. The hub can include structure that operates as a hemostasis valve, such that the hub can provide fluid to the lumen of a first catheter coupled to the hub that for discharging at the distal end of the first catheter, or can provide vacuum to the lumen of a first catheter coupled to the hub for providing aspiration at the distal end of the first catheter, while another elongated device is positioned partially or fully in the lumen of the first catheter in a concentric nested configuration. For example, when catheter 9243b is positioned in the lumen of catheter 9243a, when catheter 9243c is positioned in the lumen of catheter 9243b, or when an elongated device (e.g., a catheter, a guidewire, etc.) is positioned in the lumen of catheter 9243c. In some embodiments, catheter 9204a is a guide catheter, catheter 9243b is a procedure catheter, and catheter 9243c is an access (or insert) catheter.

Still referring to the example embodiment of FIG. 38D, each mount 9080a-c includes a saline port 9306a-c, a contrast port 9308a-c, a vacuum port 9310a-c, and an electrical connection 9311a-c that are coupled to saline manifold 9170, contrast manifold 9172, vacuum manifold 9174, and electrical bus 9176, respectively, of a tube group (of second tubing set 9013b) coupled to the mount 9080a-c. Preferably, each mount 9080a-c is coupled to the tube group 9014a-c without using connectors, for example, in a persistent connection that is not meant to be decoupled before or during its use in a medical procedure. An air sensor 9318a-c is positioned along the saline channel (flow-path) 9312a-c and is in electrical communication with electrical connection 9311a-c. The air sensor 9318a-c is configured to detect air in the saline channel 9312a-c and generate a signal indicative of detecting air which is provided to the cassette and ultimately a controller, via the second tubing set 9013b, splitter 9150, first tubing set 9013a, and the cassette 9012. An air sensor 9320a-c is positioned along contrast channel (flow-path) 9314a-c and is in electrical communication with electrical connection 9311a-c. The air sensor 9320a-c is configured to detect air in the contrast channel 9314a-c and generate a signal indicative of detecting air which is provided to the cassette 9012 and ultimately a controller via the second tubing set 9013b, splitter 9150, first tubing set 9013a, and the cassette 9012.

Mount 9080a-c includes a first control valve 9226a-c that is controllable by a controller to align saline channel 9312a-c, or contrast channel 9314a-c, to a second control valve 9228a-c. Check valve 9322a-c in the saline channel 9312a-c prevents any upstream fluid flow on the saline channel 9312a-c. Along the saline channel 9312a-c between the first control valve 9226a-c and the check valve 9322a-c is a saline drip channel (saline restricted-flow channel) 9224a-c which connects the saline channel 9312a-c upstream of the first control valve 9226a-c a fluid flow-path to the second control valve 9228a-c, bypassing the first control valve 9226a-c. In this configuration, at least some saline that enters the mount 9080a-c can flow to via the saline restricted-flow channel 9224a-c to the second control valve 9228a-c regardless of the position of the first control valve 9226a-c. The saline drip flow-path includes a saline restricted-flow channel 9224a-c designed to allow a desired flow-rate of saline (e.g, mL/minute) to flow to the connector 9209a-c and to the lumen of a catheter in fluid communication with the connector 9209a-c when the second control valve 9228 is aligned to provide saline or contrast to the connector 9209a-c regardless of the alignment of the first control valve 9226a-c. The saline restricted-flow channel 9224a-c is configured to provide a lower flow-rate of saline than the saline channel 9312a-c provides to the saline-contrast channel 9221a-c through the first control valve 9226a-c. The saline restricted-flow channel 9224a-c can have, for example, a smaller cross-sectional dimension then the cross-sectional dimension of the flow-path from the saline channel 9312a-c to the saline-contrast channel 9221a-c through the first control valve 9226a-c. As an example, the saline restricted-flow channel 9224a-c can have narrow portion of the saline drip flow-path designed such that the saline restricted-flow channel 9224a-c provides about 1 mL/minute of saline to the lumen of an associated catheter. For example, in the range of about 0.85 mL/minute to about 1.35 mL/minute. In systems with multiple catheters, the catheters can be positioned in a concentric configuration, that is, such that catheter 9243c can be positioned at least partially in the lumen of catheter 9243b, and catheter 9243b can be positioned at least partially in the lumen of catheter 9243a. In the example illustrated in FIG. 38D, flow restrictor FR1 is associated with catheter 9243a, flow restrictor FR2 is associated with catheter 9243b, and flow restrictor FR3 is associated with catheter 9243c. As an example, the saline restricted-flow channel 9224a can have a flow restrictor (value, size) FR1 designed such that the saline restricted-flow channel 9224 provides about 1 mL/minute of saline to the lumen of a catheter having a lumen inner diameter (ID) of about 0.088 inches (for example, a Zoom 88 catheter). As an example, the saline restricted-flow channel 9224b can have a flow restrictor FR2 designed such that the saline restricted-flow channel 9224b provides about 1 mL/minute of saline to the lumen of a catheter having an ID of about 0.071 inches (for example, a Zoom 71 catheter). As an example, the saline restricted-flow channel 9224c can have a flow restrictor FR3 designed such that the saline restricted-flow channel 9224c provides about 1 ml of saline to the lumen of a catheter having a lumen inner diameter (ID) of about 0.035 inches (for example, a Zoom 35 catheter). The flow restrictor FR1, FR2, FR3 can be all about the same size (e.g., having the same or about the same ID), or can be sized differently based on the catheter size a flow restrictor is associated with such that a saline drip flow of about 1 mL/minute is achieved to the catheter in fluid communication with the connector 9209a-c.

The second control valve 9228 is a three-way valve that connects either vacuum to or saline/contrast to connector 9209a-c via a fluid primary channel 9232. The primary channel provides saline, contrast and vacuum to a catheter coupled to the mount 9080, for example, provides saline, contrast and vacuum to the lumen of the catheter through connector 9209a-c. The primary channel also receives material (e.g., fluids, clots, etc.) from a catheter coupled to the mount 9080a-c when vacuum is provided to the catheter. Pressure sensor 9229a-c (e.g., a hemodynamic pressure sensor) is positioned to detect pressure in the primary channel 9232a-c and is in electrical communication with electrical connection 9311a-c. Pressure sensor 9229a-c is configured to generate a signal, indicative of the detected pressure, which is provided to cassette 9012 and ultimately a controller via the second tubing set 9013b, splitter 9150, first tubing set 9013a, and the cassette 9012. The configuration of mounts 9080 includes several advantages over previous configurations described above. For example, in this configuration a mount 9080 includes an air sensor 9318a-c in the saline channel 9312a-c and includes air sensor 9320a-c and the contrast channel 9314a-c, instead of a bubble filter in a combined saline/contrast fluid channel providing extra safety detecting air in any fluid provided to catheter 9243a-c. If the controller receives a signal from the saline air sensor 9318a-c or the contrast air sensor 9320a-c, it can provide an alarm and/or align the second control valve 9228a-c to evacuate fluid in saline-contrast channel 9221a-c and upstream of saline-contrast channel 9221a-c via vacuum channel (flow-path) 9216a-c. Subsequently, the saline and/or the contrast channels can be re-primed. In some embodiments, the controller may also align the first control valve 9226a-c to prevent contrast and saline to flow through the first control valve 9226a-c.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

The foregoing represents one specific implementation of a robotic control system. A wide variety of different robotic control system constructions can be made, for supporting and axially advancing and retracting two or three or four or more assemblies to robotically drive interventional devices, as will be appreciated by those of skill in the art in view of the disclosure herein.

While the foregoing describes interventional devices that are driven by a drive table, other suitable robotic drive systems or mechanisms may be used to drive the interventional devices, as will be appreciated by those of skill in the art in view of the disclosure herein.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

Various fluidics systems and methods are described herein primarily in the context of providing saline, contrast, and vacuum to an interventional device. However, in certain embodiments, other fluids, such as one or more drugs or components thereof (e.g., therapeutic drugs, anti-complication drugs, lytic drugs, non-lytic drugs, post thrombectomy drugs, interventional oncology drugs), can be provided using the fluidics systems and/or methods described herein. For example, a drug may be delivered through an interventional device alternatively to saline, contrast, and/or vacuum or additionally to saline, contrast, and/or vacuum using a subsystem that is the same as or generally similar to the saline subsystem or the contrast subsystem.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A robotic medical system for performing a vascular procedure, comprising:
a remote system; and
a local system comprising:
one or more computing devices;
a robotic drive system in communication with the one or more computing devices, the remote system located remotely from the robotic drive system;
a fluidics system in communication with the one or more computing devices; and
a plurality of interventional devices at least partially arranged in a concentric nested configuration, each of the plurality of interventional devices being independently moveable along a common axial direction by the robotic drive system, wherein one or more of the plurality of interventional devices are in fluid communication with the fluidics system;
wherein the local system is configured to:
receive first information from the remote system, determine movement control signals for moving the plurality of interventional devices based on the first information, and provide the movement control signals to the robotic drive system for independently moving one or more of the plurality of interventional devices, and
receive second information from the remote system, determine fluidics control signals from the second information, and provide the fluidics control signals to the fluidics system for independently providing one or more fluids and/or vacuum to one or more of the plurality of interventional devices.

2. The robotic medical system of claim 1, wherein the fluidics system comprises a saline subsystem, a contrast subsystem, and an aspiration subsystem, wherein the plurality of interventional devices comprises three catheters and a guidewire, and wherein the fluidics system is configured to independently provide saline, contrast, and vacuum to the three catheters based on the fluidics control signals.

3. The robotic medical system of claim 2, wherein the local system further comprises three mounts, wherein each mount is coupled to one of the catheters, wherein each mount is movable in the axial direction by the robotic drive system, and wherein each mount includes saline, contrast, and vacuum channels for providing saline, contrast, and vacuum to the catheter coupled thereto.

4. The robotic medical system of claim 1, wherein the plurality of interventional devices includes two or more catheters, and wherein the second information can indicate one or more of the two or more catheters to provide fluids and/or vacuum to.

5. The robotic medical system of claim 1, wherein the remote system comprises:
a control console having at least one monitor; and
one or more control devices in communication with the control console, wherein the remote system is configured to generate control signals for moving one or more of the plurality of interventional devices in response to actuation of one or more controls of the one or more control devices.

6. The robotic medical system of claim 1, wherein the local system further comprises a user interface, and wherein each of the plurality of interventional devices are axially and/or rotationally movable based on inputs from the user interface.

7. The robotic medical system of claim 1, wherein the plurality of interventional devices comprises at least two catheters, and the fluidics control signals configure the fluidics system to provide contrast or vacuum to one of the at least two catheters.

8. The robotic medical system of claim 1, wherein the plurality of interventional devices comprises at least two catheters, and the fluidics control signals configure the fluidics system to provide contrast or vacuum to both of the at least two catheters.

9. The robotic medical system of claim 1, wherein the plurality of interventional devices comprises at least three catheters, and the fluidics control signals configure the fluidics system to provide contrast or vacuum to one of the at least three catheters.

10. The robotic medical system of claim 1, wherein the plurality of interventional devices comprises three catheters and a guidewire, and wherein each catheter is in fluid communication with the fluidics system to independently receive saline, contrast, and vacuum from a saline subsystem, a contrast subsystem, and aspiration subsystem, respectively, based on the fluidics control signals.

11. The robotic medical system of claim 1, wherein the remote system receives third information from the local system, the third information comprising one or more fluoroscopic images of a working area.

12. The robotic medical system of claim 1, wherein the first information is generated by the remote system, represents a movement of a control of control device, and is indicative of a desired movement for one or more of the plurality of interventional devices.

13. The robotic medical system of claim 1, wherein the second information is generated by the remote system and is indicative of a desired fluidics action to occur at the fluidics system and one or more of the plurality of interventional devices.

14. The robotic medical system of claim 1, wherein the remote system receives third information from the local system wherein the third information comprises media streams of an operating room to be displayed on at least one monitor.

15. The robotic medical system of claim 1, wherein the remote system receives third information from the local system, wherein the third information comprises instructions to manipulate one or more control devices.

16. The robotic medical system of claim 1, further comprising one or more hardware processors configured to execute instructions to detect and manipulate latency in robotic medical system.

17. The robotic medical system of claim 1, wherein the remote system separated from the robotic drive system by a fluoroscopic barrier.

18. The robotic medical system of claim 1, wherein the remote system and the robotic drive system are located in separate rooms.

19. The robotic medical system of claim 1, wherein the remote system and the robotic drive system are located in separate geographic areas.

20. The robotic medical system of claim 1, wherein the local system comprises a streaming system in communication with the remote system.
